(19) United States
(12) Patent Application Publication
(10) Pub. No.: US 2024/0018127 A1
(43) Pub. Date: Jan. 18, 2024

(54) ORGANIC MOLECULES FOR OPTOELECTRONIC DEVICES

(71) Applicant: Samsung Display Co., Ltd., Yongin-si, Gyeonggi-do (KR)

(72) Inventors: Damien THIRION, Karlsdorf-Neuthard (DE); Damien JOLY, Beinheim (FR); Michael DANZ, Eggenstein- Leopoldshafen (DE)

(21) Appl. No.: 18/006,574

(22) PCT Filed: Jul. 22, 2021
(86) PCT No.: PCT/EP2021/070472

§ 371 Date: Jan. 23, 2023

(30) Foreign Application Priority Data
Jul. 24, 2020 (EP) 20187601.8

(51) Int. Cl. C07D 403/14; C07D 405/14; C07D 487/04; H10K 85/60; H10K 50/00

(52) U.S. Cl. CPC C07D 403/14; C07D 405/14; H10K 50/401; H10K 85/654; C07D 487/04

(57) ABSTRACT

The invention relates to a light emitting organic molecule, in particular for the application in optoelectronic devices. According to the invention, the organic molecule consists of
a first chemical moiety with a structure of Formula I.

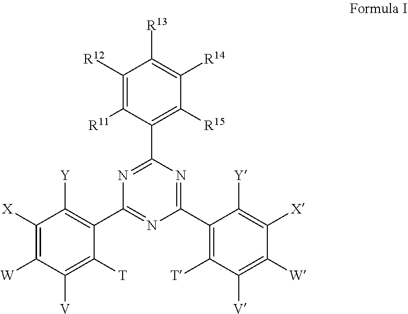

and
two second chemical moieties with a structure of Formula II,

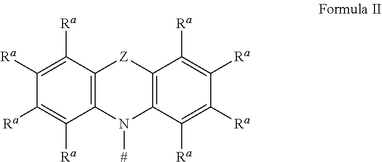

wherein
#represents the binding site of a single bond linking the first chemical moiety to the second chemical moiety;
exactly one substituent selected from the group consisting of W, X, and Y is cyanophenyl;
exactly one substituent selected from the group consisting of T, V and W represents the binding site of a single bond linking the first chemical moiety and one of the two second chemical moieties;
exactly one substituent selected from the group consisting of W′, X′, and Y′ is CN or CF₃; and
exactly one substituent selected from the group consisting of T′, V′ and W′ represents the binding site of a single bond linking the first chemical moiety and one of the two second chemical moieties.

### CROSS-REFERENCE TO RELATED APPLICATIONS

This application is a U.S. National Phase Patent Application of International Patent Application Number PCT/EP2021/070472, filed on Jul. 22, 2021, which claims priority to European Patent Application Number 20187601.8, filed on Jul. 24, 2020, the entire content of all of which is incorporated herein by reference.

The invention relates to light-emitting organic molecules and their use in organic light-emitting diodes (OLEDs) and in other optoelectronic devices.

### DESCRIPTION

The object of the present invention is to provide molecules which are suitable for use in optoelectronic devices.

This object is achieved by the invention which provides a new class of organic molecules.

The organic molecules of the invention are purely organic molecules, i.e. they do not contain any metal ions in contrast to metal complexes known for use in optoelectronic devices.

The organic molecules exhibit emission maxima in the sky blue, green or yellow spectral range. The organic molecules exhibit in particular emission maxima between 490 and 600 nm, more preferably between 510 and 560 nm, and even more preferably between 520 and 540 nm. The photoluminescence quantum yields of the organic molecules according to the invention are, in particular, 10% or more. The molecules of the invention exhibit in particular thermally activated delayed fluorescence (TADF). The use of the molecules according to the invention in an optoelectronic device, for example, an organic light-emitting diode (OLED), leads to higher efficiencies of the device. Corresponding OLEDs have a higher stability than OLEDs with known emitter materials and comparable color and/or by employing the molecules according to the invention in an OLED display, a more accurate reproduction of visible colors in nature, i.e. a higher resolution in the displayed image, is achieved. In particular, the molecules can be used in combination with a fluorescence emitter to enable so-called hyper-fluorescence.

The organic molecules according to the invention include or consist of one first chemical moiety including or consisting of a structure of Formula I,

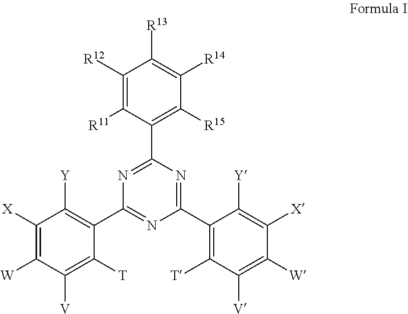

and two second chemical moieties, each independently including or consisting of a structure of Formula II.

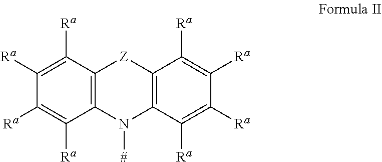

wherein the first chemical moiety is linked to each of the second chemical moieties via a single bond.
T is the binding site of a single bond linking the first chemical moiety to one of the two second chemical moieties, or is R^{I}.
V is the binding site of a single bond linking the first chemical moiety to one of the two second chemical moieties, or is R^{I}.
W is the binding site of a single bond linking the first chemical moiety to one of the two second chemical moieties, or is selected from the group consisting of R^{I} and R^{A}.
X is selected from the group consisting of R^{I} and R^{A}.
Y is selected from the group consisting of R^{I} and R^{A}.
T′ is the binding site of a single bond linking the first chemical moiety to one of the two second chemical moieties, or is R^{II}.
V′ is the binding site of a single bond linking the first chemical moiety to one of the two second chemical moieties, or is R^{II}.
W′ is the binding site of a single bond linking the first chemical moiety to one of the two second chemical moieties, or is selected from the group consisting of R^{II}, CN, and CF₃.
X′ is selected from the group consisting of R^{II}, CN, and CF₃.
Y′ is selected from the group consisting of R^{II}, CN, and CF₃.
Z is at each occurrence independently from one another selected from the group consisting of a direct bond, CR³R⁴, C═CR³R⁴, C═O, C═NR³, NR³, O, SiR³R⁴, S, S(O) and S(O)₂.
#represents the binding site of the first chemical moiety to the second chemical moiety.

R^{A} includes or consists of a structure of Formula BN-I,

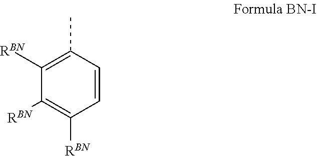

which is bonded to the structure of Formula I via the position marked by the dashed line and wherein exactly one R^{BN} group is CN while the other two R^{BN} groups are both hydrogen, i.e. R^{A} includes or consists of a structure according to any of the Formulas BN-Ia, BN-Ib or BN-Ic:

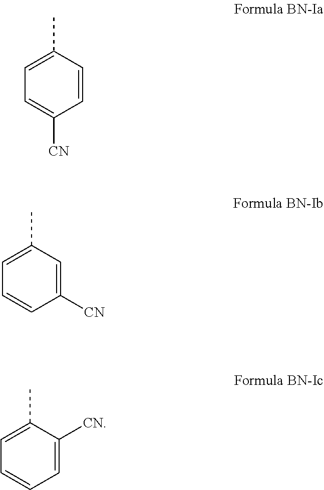

R^{I} is at each occurrence independently from one another selected from the group consisting of:
hydrogen, deuterium;
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₆-C₁₈-aryl.

R^{II} is at each occurrence independently from one another selected from the group consisting of:
hydrogen, deuterium;
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₆-C₁₈-aryl.

R¹¹, R¹², R¹³, R¹⁴ and R¹⁵ are at each occurrence independently selected from the group consisting of: hydrogen, deuterium, CN, CF₃, phenyl (Ph);
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₅-C₁₈-aryl.

R^{e}, R³, and R⁴ are at each occurrence independently selected from the group consisting of: hydrogen, deuterium, N(R⁵)₂, OR⁵, Si(R⁵)₃, B(OR⁵)₂, OSO₂R⁵, CF₃, CN, F, Br, I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄O-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵.

R⁵ is at each occurrence independently from one another selected from the group consisting of: hydrogen, deuterium, N(R⁶)₂, OR⁶, Si(R⁶)₃, B(OR⁶)₂, OSO₂R⁶, CF₃, CN, F, Br, I;
C₁-C₄₀-alkyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-alkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-thioalkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkenyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkynyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₆-C₆₀-aryl,
which is optionally substituted with one or more substituents R⁶; and
C₃-C₅₇-heteroaryl,
which is optionally substituted with one or more substituents R⁶.

R⁶ is at each occurrence independently from one another selected from the group consisting of: hydrogen, deuterium, OPh, CF₃, CN, F;
C₁-C₅-alkyl,
wherein one or more hydrogen atoms are optionally, independently from each other substituted by deuterium, CN, CF₃, or F;
C₁-C₅-alkoxy,
wherein one or more hydrogen atoms are optionally, independently from each other substituted by deuterium, CN, CF₃, or F;
C₁-C₅-thioalkoxy,
wherein one or more hydrogen atoms are optionally, independently from each other substituted by deuterium, CN, CF₃, or F;
C₂-C₅-alkenyl,
wherein one or more hydrogen atoms are optionally, independently from each other substituted by deuterium, CN, CF₃, or F;
C₂-C₅-alkynyl,
wherein one or more hydrogen atoms are optionally, independently from each other substituted by deuterium, CN, CF₃, or F;
C₆-C₁₈-aryl,
which is optionally substituted with one or more C₁-C₅-alkyl substituents;
C₃-C₁₇-heteroaryl,
which is optionally substituted with one or more C₁-C₅-alkyl substituents;
N(C₆-C₁₈-aryl)₂;
N(C₃-C₁₇-heteroaryl)₂, and
N(C₃-C₁₇-heteroaryl)(C₆-C₁₈-aryl).

Optionally, any of the substituents R^{a}, R³, R⁴ or R⁵ independently form a mono- or polycyclic, aliphatic, aromatic and/or benzo-fused ring system with one or more other substituents R^{a}, R³, R⁴ or R⁵.

According to the invention, exactly one substituent selected from the group consisting of W, X, and Y is R^{A}, and exactly one substituent selected from the group consisting of T, V, and W represents the binding site of a single bond linking the first chemical moiety and one of the two second chemical moieties.

Furthermore, according to the invention, exactly one substituent selected from the group consisting of W′, X′, and Y′ is CN or CF₃, and exactly one substituent selected from the group consisting of T′, V′ and W′ represents the binding site of a single bond linking the first chemical moiety and one of the two second chemical moieties.

In certain embodiments of the invention, the two second chemical moieties are identical.

In one embodiment of the invention, W and W′ are each a binding site of a single bond linking the first chemical moiety and one of the two second chemical moieties.

In one embodiment of the invention, W and W′ are each a binding site of a single bond linking the first chemical moiety and one of the two second chemical moieties, X is R^{A} and X′ is CN.

In one embodiment of the invention, W and W′ are each a binding site of a single bond linking the first chemical moiety and one of the two second chemical moieties, X is R^{A} and X′ is CF₃.

In one embodiment of the invention, W and W′ are each a binding site of a single bond linking the first chemical moiety and one of the two second chemical moieties, Y is R^{A} and Y′ is CN.

In one embodiment of the invention, W and W′ are each a binding site of a single bond linking the first chemical moiety and one of the two second chemical moieties, Y is R^{A} and Y′ is CF₃.

In one embodiment of the invention, V and V′ are each a binding site of a single bond linking the first chemical moiety and one of the two second chemical moieties.

In one embodiment of the invention, V and V′ are each a binding site of a single bond linking the first chemical moiety and one of the two second chemical moieties, W is R^{A} and W′ is CN.

In one embodiment of the invention, V and V are each a binding site of a single bond linking the first chemical moiety and one of the two second chemical moieties, W is R^{A} and W′ is CF₃.

In one embodiment of the invention, V and V are each a binding site of a single bond linking the first chemical moiety and one of the two second chemical moieties, X is R^{A} and X′ is CN.

In one embodiment of the invention, V and V are each a binding site of a single bond linking the first chemical moiety and one of the two second chemical moieties, X is R^{A} and X′ is CF₃.

In one embodiment of the invention, V and V are each a binding site of a single bond linking the first chemical moiety and one of the two second chemical moieties, Y is R^{A} and Y′ is CN.

In one embodiment of the invention, V and V are each a binding site of a single bond linking the first chemical moiety and one of the two second chemical moieties, Y is R^{A} and Y′ is CF₃.

In a preferred embodiment of the invention, T and T′ are each a binding site of a single bond linking the first chemical moiety and one of the two second chemical moieties.

In one embodiment of the invention, T and T′ are each a binding site of a single bond linking the first chemical moiety and one of the two second chemical moieties, W is R^{A} and W′ is CN.

In one embodiment of the invention, T and T′ are each a binding site of a single bond linking the first chemical moiety and one of the two second chemical moieties, W is R^{A} and W′ is CF₃.

In a particularly preferred embodiment of the invention, T and T′ are each a binding site of a single bond linking the first chemical moiety and one of the two second chemical moieties, X is R^{A} and X′ is CN.

In one embodiment of the invention, T and T′ are each a binding site of a single bond linking the first chemical moiety and one of the two second chemical moieties, X is R^{A} and X′ is CF₃.

In another particularly preferred embodiment of the invention, T and T′ are each a binding site of a single bond linking the first chemical moiety and one of the two second chemical moieties, W is R^{A} and X′ is CN.

In a further embodiment of the invention, T and T′ are each a binding site of a single bond linking the first chemical moiety and one of the two second chemical moieties, W is R^{A} and X′ is CF₃.

In one embodiment of the invention, T and T are each a binding site of a single bond linking the first chemical moiety and one of the two second chemical moieties, X is R^{A} and W′ is CN.

In a further embodiment of the invention, T and T′ are each a binding site of a single bond linking the first chemical moiety and one of the two second chemical moieties, X is R^{A} and W′ is CF₃.

In one embodiment of the invention, T and T′ are each a binding site of a single bond linking the first chemical moiety and one of the two second chemical moieties, Y is R^{A} and Y′ is CN.

In one embodiment of the invention, T and T′ are each a binding site of a single bond linking the first chemical moiety and one of the two second chemical moieties, Y is R^{A} and Y′ is CF₃.

In one embodiment of the invention, R^{I} is at each occurrence independently selected from the group consisting of hydrogen, methyl, iso-propyl, tert-butyl, mesityl, xylyl, tolyl, and phenyl.

In one embodiment of the invention, R^{I} is at each occurrence independently selected from the group consisting of hydrogen, methyl, mesityl, tolyl, and phenyl.

In another embodiment of the invention, R^{I} is at each occurrence hydrogen.

In one embodiment of the invention, R^{II} is at each occurrence independently selected from the group consisting of hydrogen, methyl, iso-propyl, tert-butyl, mesityl, xylyl, tolyl, and phenyl.

In one embodiment of the invention, R^{II} is at each occurrence independently selected from the group consisting of hydrogen, methyl, mesityl, tolyl, and phenyl.

In another embodiment of the invention, R^{II} is at each occurrence hydrogen.

In one embodiment of the invention, R^{A} is represented by Formula BN-Ia.

In one embodiment of the invention, R^{A} is represented by Formula BN-Ib.

In one embodiment of the invention, R^{A} is represented by Formula BN-Ic.

In another embodiment of the invention, R³, R⁴, R⁵, and R⁶ are at each occurrence independently selected from the group consisting of hydrogen, deuterium, halogen, Me, ⁱPr, ^{t}Bu, CN, CF₃, SiMe₃, SiPh₃, and
C₆-C₁₈-aryl,
wherein optionally one or more hydrogen atoms are independently substituted by C₁-C₅-alkyl, CN, CF₃ and Ph.

In another embodiment of the invention, R³, R⁴, R⁵, and R⁶ are at each occurrence independently selected from the group consisting of hydrogen, deuterium, halogen, Me, ⁱPr, ^{t}Bu, CN, CF₃, SiMe₃, SiPh₃, and
phenyl (Ph),
wherein optionally one or more hydrogen atoms are independently substituted by C₁-C₅-alkyl, CN, CF₃ or Ph.

In another embodiment of the invention, R³, R⁴, R⁵, and R⁶ are at each occurrence independently selected from the group consisting of hydrogen, deuterium, halogen, Me, ⁱPr, ^{t}Bu, CN, CF₃, SiMe₃, SiPh₃, and
phenyl,
wherein optionally one or more hydrogen atoms are independently substituted by Me, ⁱPr, ^{t}Bu, CN, CF₃ or Ph.

In one embodiment of the invention, the second chemical moiety includes or consists of a structure of Formula IIa:

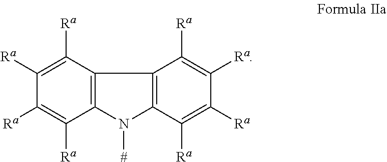

In one embodiment of the invention, R^{a} is at each occurrence independently from one another selected from the group consisting of: hydrogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

In a further embodiment of the invention, R^{a} is at each occurrence independently from one another selected from the group consisting of: hydrogen, Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph.

In a further embodiment of the invention, the second chemical moiety includes or consists of a structure of Formula IIb, a structure of Formula IIb-2, a structure of Formula IIb-3 or a structure of Formula IIb-4:

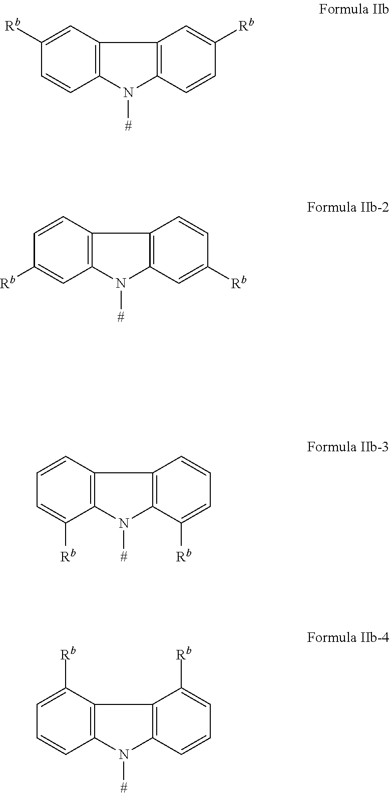

wherein

R^{b} is at each occurrence independently from one another selected from the group consisting of deuterium, N(R⁵)₂, OR⁵, Si(R⁵)₃, B(OR⁵)₂, OSO₂R⁵, CFs, CN, F, Br, I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵.

In other embodiments of the invention, the second chemical moiety includes or consists of a structure of Formula IIc, a structure of Formula IIc-2, a structure of Formula Ic-3 or a structure of Formula Ic-4

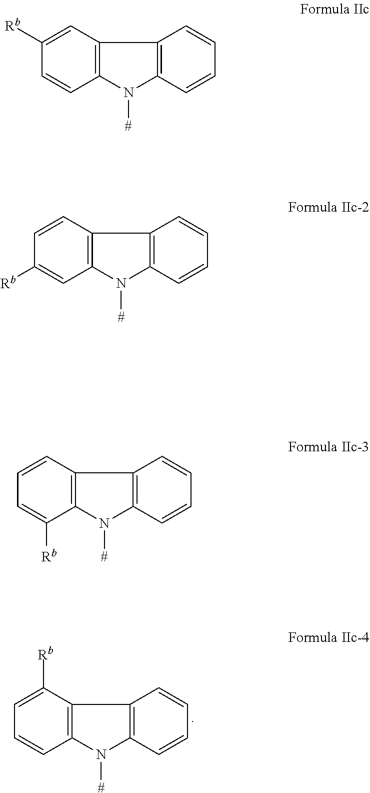

In a further embodiment of the invention, R^{b} is at each occurrence independently from one another selected from the group consisting of: Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

In a further embodiment of the invention, R^{b} is at each occurrence independently from one another selected from the group consisting of: Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph.

Below, examples of the second chemical moiety are shown:

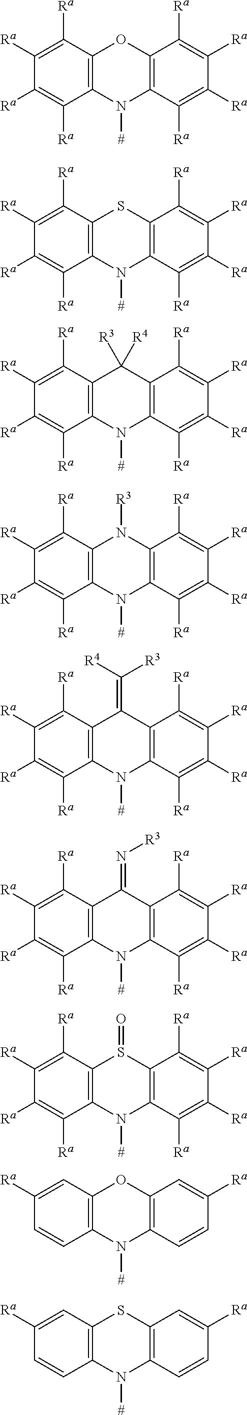

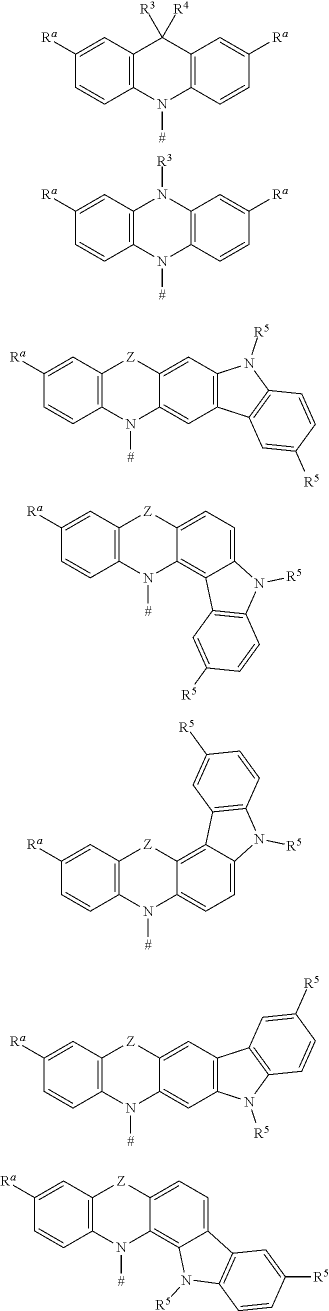

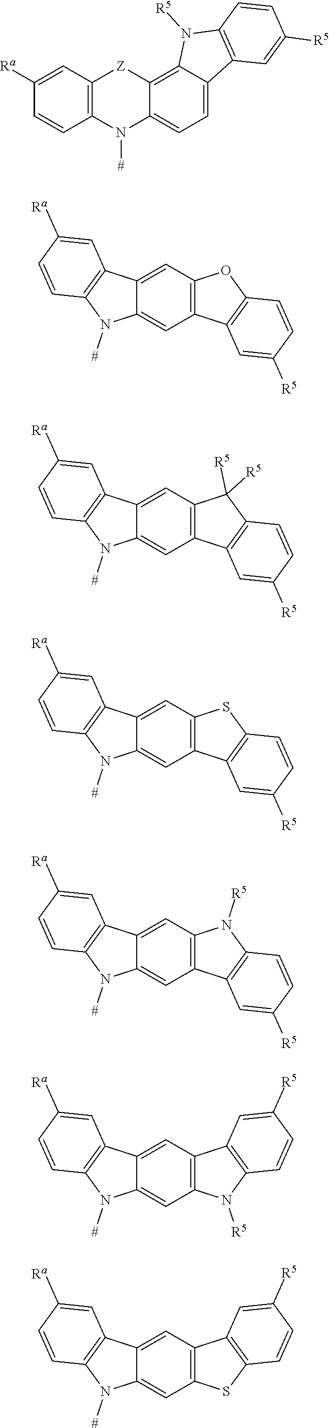

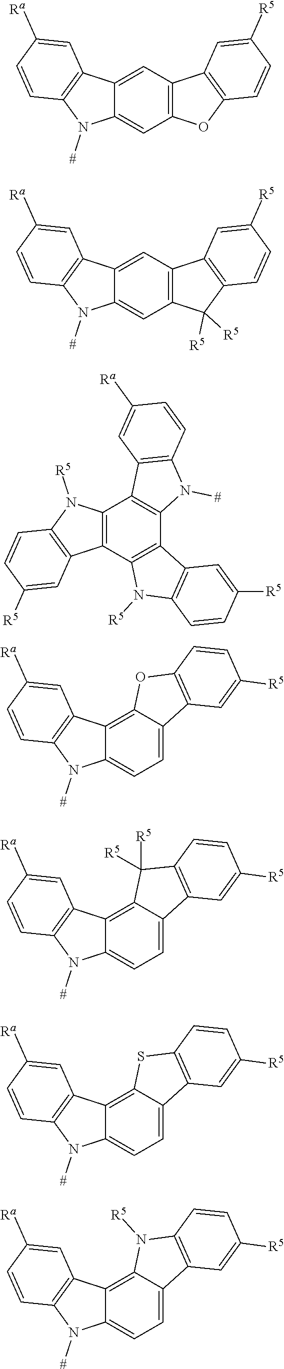

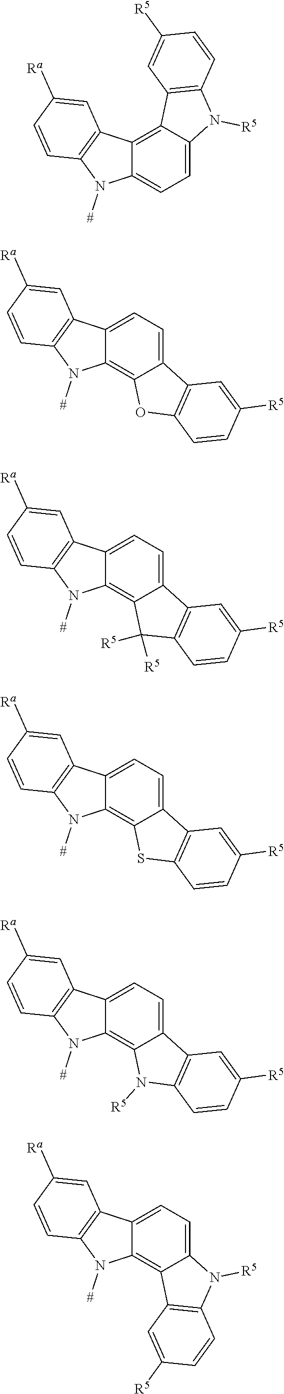

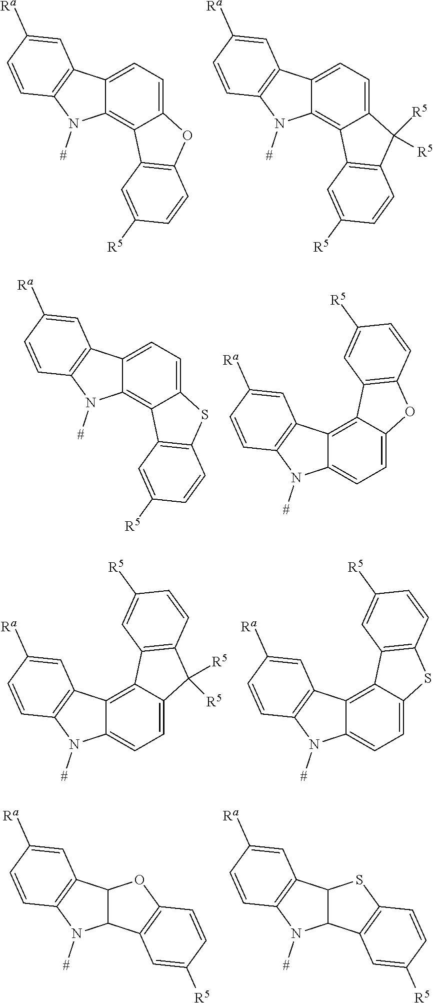

In one embodiment, R^{a} and R⁵ are at each occurrence independently from one another selected from the group consisting of hydrogen (H), methyl (Me), i-propyl (CH(CH₃)₂) (ⁱPr), t-butyl (^{t}Bu), phenyl (Ph), CN, CF₃, and diphenylamine (NPh₂).

In one embodiment of the invention, the organic molecule includes or consists of a structure according to any of the Formula III, IV, V, VI, VII, VIII, IX, X, or XI:

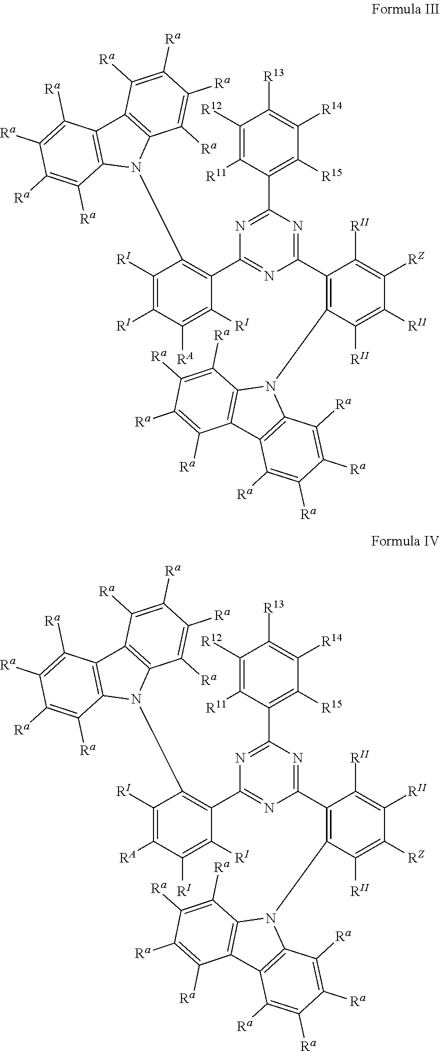

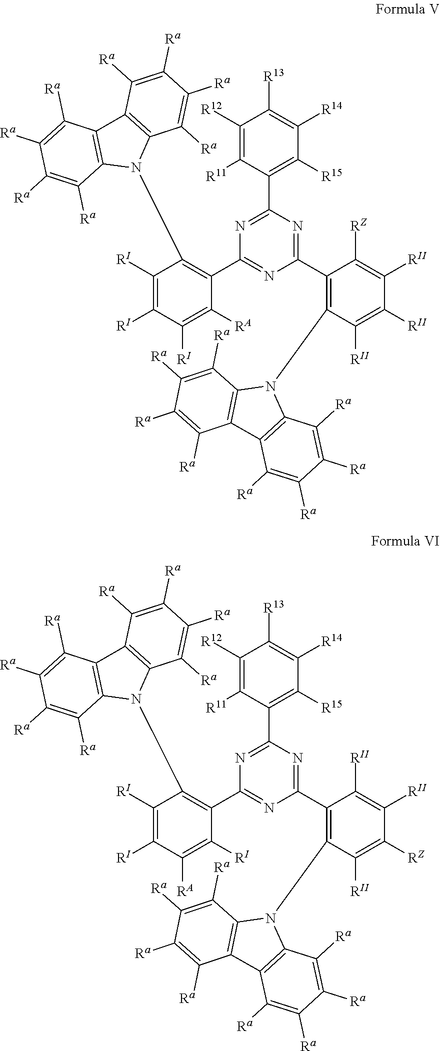

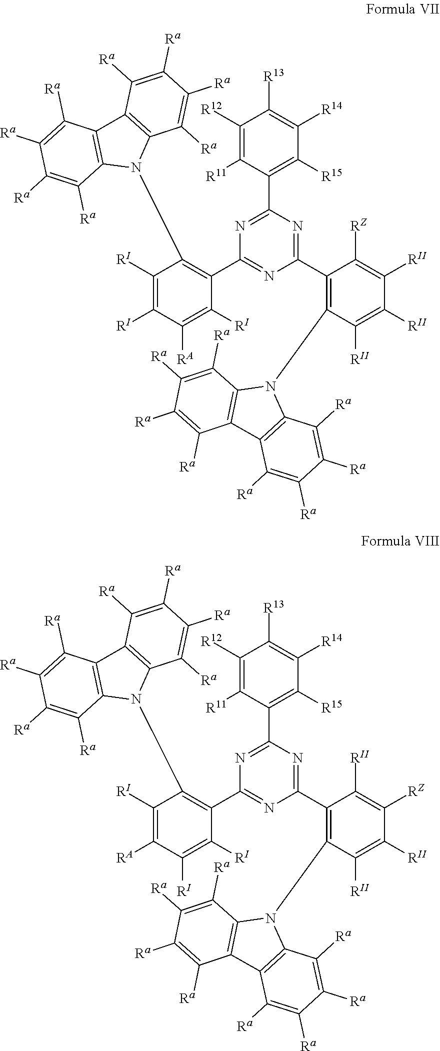

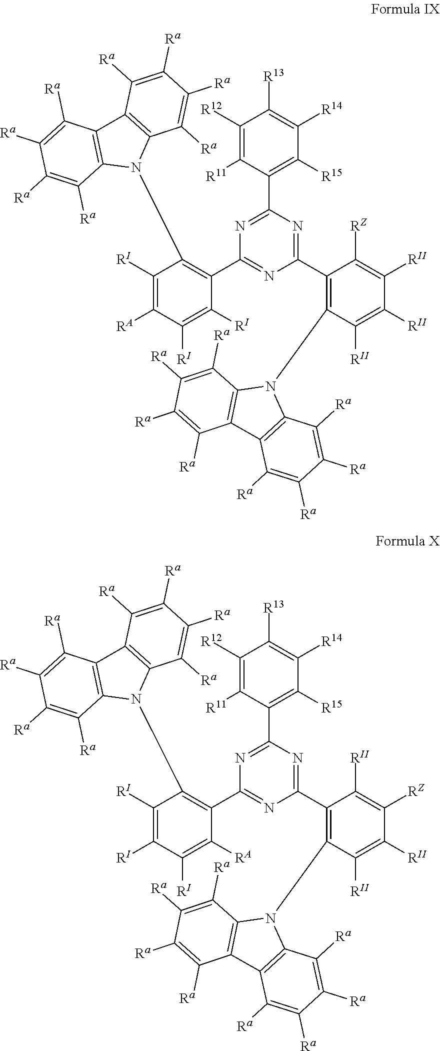

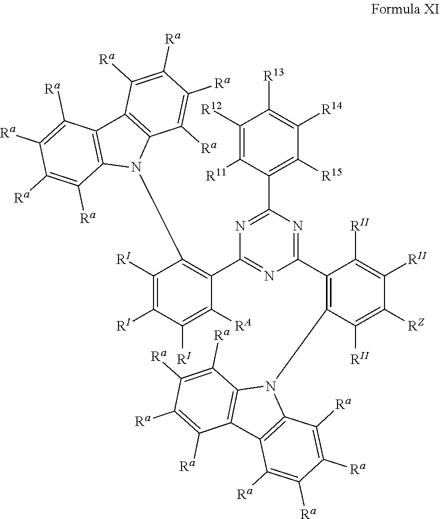

wherein R^{Z} is CN or CF₃.

In one embodiment of the invention, the organic molecule includes or consists of a structure according to any of Formula III, IV, V, VI, VII, VIII, IX, X, or XI, wherein R^{Z} is CN.

In one embodiment of the invention, the organic molecule includes or consists of a structure according to any of Formula III, IV, V, VI, VII, VIII, IX, X, or XI, wherein R^{Z} is CF₃.

In one embodiment of the invention, the organic molecule includes or consists of a structure according to any of Formula III, IV, V, VI, VII, VIII, IX, X, or XI, wherein R^{I} and R^{II} are at each occurrence hydrogen.

In one embodiment of the invention, the organic molecule includes or consists of a structure according to any of Formula III, IV, V, VI, VII, VIII, IX, X, or XI, wherein R¹¹, R¹², R¹³, R¹⁴, and R¹⁵ are hydrogen.

In one embodiment of the invention, the organic molecule includes or consists of a structure according to any of Formula III, IV, V, VI, VII, VIII, IX, X, or XI, wherein R^{I}, R^{II}, R¹¹, R¹², R¹³, R¹⁴, and R¹⁵ are at each occurrence hydrogen.

In one embodiment of the invention, the organic molecule includes or consists of a structure according to any of Formula III, IV, V, VI, VII, VIII, IX, X, or XI, wherein R^{A} is represented by Formula BN-Ia.

In one preferred embodiment of the invention, the organic molecule includes or consists of a structure according to any of Formula III, IV, V, VI, VII, VIII, IX, X, or XI, wherein R^{A} is represented by Formula BN-Ib.

In one embodiment of the invention, the organic molecule includes or consists of a structure according to any of Formula III, IV, V, VI, VII, VIII, IX, X, or XI, wherein R^{A} is represented by Formula BN-Ic.

In a preferred embodiment of the invention, the organic molecule includes or consists of a structure according to Formula III.

In an even more preferred embodiment of the invention, the organic molecule includes or consists of a structure according to Formula III, wherein R^{Z} is CN.

In a particularly preferred embodiment of the invention, the organic molecule includes or consists of a structure according to Formula III, wherein R^{Z} is CN and wherein R^{I}, R^{II}, R¹¹, R¹², R¹³, R¹⁴, and R¹⁵ are at each occurrence hydrogen.

In another preferred embodiment of the invention, the organic molecule includes or consists of a structure according to Formula VIII.

In an even more preferred embodiment of the invention, the organic molecule includes or consists of a structure according to Formula VIII, wherein R^{Z} is CN.

In a particularly preferred embodiment of the invention, the organic molecule includes or consists of a structure according to Formula VIII, wherein R^{Z} is CN and wherein R^{I}, R^{II}, R¹¹, R¹², R¹³, R¹⁴, and R¹⁵ are at each occurrence hydrogen.

In one embodiment of the invention, the organic molecule includes or consists of a structure according to any of the Formula IIIa, IIIb, VIIIa, or VIIIb:

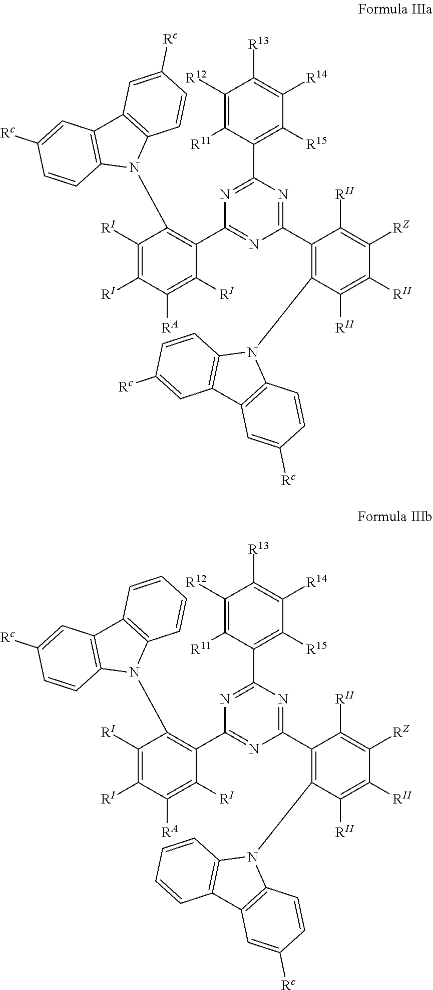

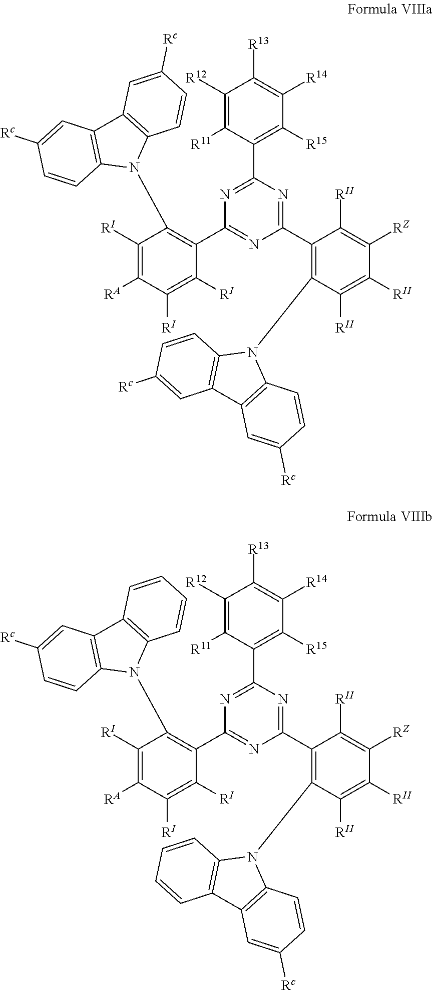

wherein R^{Z} is CN or CF₃ and
wherein R^{c} is at each occurrence independently from one another selected from the group consisting of: Me, ⁱPr, ^{t}Bu,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph,
carbazolyl, which is optionally substituted with one or more substituents independently from each other selected from the group consisting of Me, ⁱPr, ^{t}Bu, CN, CF₃, and Ph,
triazinyl, which is optionally substituted with one or more substituents independently from each other selected from the group consisting of Me, ⁱPr, ^{t}Bu, CN, CF₃, and Ph, and
N(Ph)₂.

In one embodiment of the invention, the organic molecule includes or consists of a structure according to Formula IIIa, IIIb, VIIIa, or VIIIb,
wherein R^{Z} is CN or CF₃ and
wherein R^{c} is at each occurrence independently from one another selected from the group consisting of: Me, ⁱPr, ^{t}Bu, CN, CF₃,
Ph, which is optionally substituted with one or more substituents independently from each other selected from the group consisting of Me, ⁱPr, ^{t}Bu, CN, CF₃, and Ph,
pyridinyl, which is optionally substituted with one or more substituents independently from each other selected from the group consisting of Me, ⁱPr, ^{t}Bu, CN, CF₃, and Ph,
pyrimidinyl, which is optionally substituted with one or more substituents independently from each other selected from the group consisting of Me, ⁱPr, ^{t}Bu, CN, CF₃, and Ph, and
triazinyl, which is optionally substituted with one or more substituents independently from each other selected from the group consisting of Me, ⁱPr, ^{t}Bu, CN, CF₃, and Ph.

In a preferred embodiment of the invention, the organic molecule includes or consists of a structure according to Formula IIIa, IIIb, VIIIa, or VIIIb, wherein R^{Z} is CN.

In a particularly preferred embodiment of the invention, the organic molecule includes or consists of a structure according any of the Formula IIIa, IIIb, VIIIa, or VIIIb, wherein R^{Z} is CN and wherein R^{I}, R^{II}, R¹¹, R¹², R¹³, R¹⁴, and R¹⁵ are at each occurrence hydrogen.

In one embodiment of the invention, the organic molecule includes or consists of a structure according to Formula IIIa, IIIb, VIIIa, or VIIIb, wherein R^{Z} is CF₃.

In one embodiment of the invention, the organic molecule includes or consists of a structure according to any of Formula IIIa, IIIb, VIIIa, or VIIIb, wherein R^{A} is represented by Formula BN-Ia.

In one embodiment of the invention, the organic molecule includes or consists of a structure according to any of Formula IIIa, IIIb, VIIIa, or VIIIb, wherein R^{A} is represented by Formula BN-Ib.

In one embodiment of the invention, the organic molecule includes or consists of a structure according to any of Formula IIIa, IIIb, VIIIa, or VIIIb, wherein R^{A} is represented by Formula BN-Ic.

As used above and herein, the terms “aryl” and “aromatic” may be understood in the broadest sense as any mono-, bi- or polycyclic aromatic moieties. Accordingly, an aryl group contains 6 to 60 aromatic ring atoms, and a heteroaryl group contains 5 to 60 aromatic ring atoms, of which at least one is a heteroatom. Notwithstanding, throughout the application the number of aromatic ring atoms may be given as subscripted number in the definition of certain substituents. In particular, the heteroaromatic ring includes one to three heteroatoms. Again, the terms “heteroaryl” and “heteroaromatic” may be understood in the broadest sense as any mono-, bi- or polycyclic hetero-aromatic moieties that include at least one heteroatom. The heteroatoms may at each occurrence be the same or different and be individually selected from the group consisting of N, O and S. Accordingly, the term “arylene” refers to a divalent substituent that bears two binding sites to other molecular structures and thereby serving as a linker structure. In case, a group in the exemplary embodiments is defined differently from the definitions given here, for example, the number of aromatic ring atoms or number of heteroatoms differs from the given definition, the definition in the exemplary embodiments is to be applied. According to the invention, a condensed (annulated) aromatic or heteroaromatic polycycle is built of two or more single aromatic or heteroaromatic cycles, which formed the polycycle via a condensation reaction.

In particular, as used throughout the present application, the term aryl group or heteroaryl group includes groups which can be bound via any position of the aromatic or heteroaromatic group, derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene; pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthoimidazole, phenanthroimidazole, pyridoimidazole, pyrazinoimidazole, quinoxalinoimidazole, oxazole, benzoxazole, naphthooxazole, anthroxazol, phenanthroxazol, isoxazole, 1,2-thiazole, 1,3-thiazole, benzothiazole, pyridazine, benzopyridazine, pyrimidine, benzopyrimidine, 1,3,5-triazine, quinoxaline, pyrazine, phenazine, naphthyridine, carboline, benzocarboline, phenanthroline, 1,2,3-triazole, 1,2,4-triazole, benzotriazole, 1,2,3-oxadiazole, 1,2,4-oxadiazole, 1,2,5-oxadiazole, 1,2,3,4-tetriazine, purine, pteridine, indolizine, benzothiadiazole, or combination(s) of the above mentioned groups.

As used throughout, the term cyclic group may be understood in the broadest sense as any mono-, bi- or polycyclic moieties.

As used above and herein, the term alkyl group may be understood in the broadest sense as any linear, branched, or cyclic alkyl substituent. In particular, examples of the term alkyl include the substituents methyl (Me), ethyl (Et), n-propyl (ⁿPr), i-propyl (ⁱPr), cyclopropyl, n-butyl (ⁿBu), i-butyl (^{t}Bu), s-butyl (^{s}Bu), t-butyl (^{t}Bu), cyclobutyl, 2-methylbutyl, n-pentyl, s-pentyl, t-pentyl, 2-pentyl, neo-pentyl, cyclopentyl, n-hexyl, s-hexyl, t-hexyl, 2-hexyl, 3-hexyl, neo-hexyl, cyclohexyl, 1-methylcyclopentyl, 2-methylpentyl, n-heptyl, 2-heptyl, 3-heptyl, 4-heptyl, cycloheptyl, 1-methylcyclohexyl, n-octyl, 2-ethylhexyl, cyclooctyl, 1-bicyclo[2,2,2]octyl, 2-bicyclo[2,2,2]-octyl, 2-(2,6-dimethyl)octyl, 3-(3,7-dimethyl)octyl, adamantyl, 2,2,2-trifluorethyl, 1,1-dimethyl-n-hex-1-yl, 1,1-dimethyl-n-hept-1-yl, 1,1-dimethyl-n-oct-1-yl, 1,1-dimethyl-n-dec-1-yl, 1,1-dimethyl-n-dodec-1-yl, 1,1-dimethyl-n-tetradec-1-yl, 1,1-dimethyl-n-hexadec-1-yl, 1,1-dimethyl-n-octadec-1-yl, 1,1-diethyl-n-hex-1-yl, 1,1-diethyl-n-hept-1-yl, 1,1-diethyl-n-oct-1-yl, 1,1-diethyl-n-dec-1-yl, 1,1-diethyl-n-dodec-1-yl, 1,1-diethyl-n-tetradec-1-yl, 1,1-diethyl-n-hexadec-1-yl, 1,1-diethyl-n-octadec-1-yl, 1-(n-propyl)-cyclohex-1-yl, 1-(n-butyl)-cyclohex-1-yl, 1-(n-hexyl)-cyclohex-1-yl, 1-(n-octyl)-cyclohex-1-yl and 1-(n-decyl)-cyclohex-1-yl.

As used above and herein, examples of the term alkenyl include linear, branched, and cyclic alkenyl substituents. The term alkenyl group, for example, includes the substituents ethenyl, propenyl, butenyl, pentenyl, cyclopentenyl, hexenyl, cyclohexenyl, heptenyl, cycloheptenyl, octenyl, cyclooctenyl or cyclooctadienyl.

As used above and herein, examples of the term alkynyl include linear, branched, and cyclic alkynyl substituents. The term alkynyl group, for example, includes ethynyl, propynyl, butynyl, pentynyl, hexynyl, heptynyl or octynyl.

As used above and herein, examples of the term alkoxy include linear, branched, and cyclic alkoxy substituents. The term alkoxy group, for example, includes methoxy, ethoxy, n-propoxy, i-propoxy, n-butoxy, i-butoxy, s-butoxy, t-butoxy and 2-methylbutoxy.

As used above and herein, examples of the term thioalkoxy include linear, branched, and cyclic thioalkoxy substituents, in which the O of, for example, the alkoxy groups is replaced by S.

As used above and herein, the terms “halogen” and “halo” may be understood in the broadest sense as being preferably fluorine, chlorine, bromine or iodine.

Whenever hydrogen (H) is mentioned herein, it could also be replaced by deuterium at each occurrence.

It is understood that when a molecular fragment is described as being a substituent or otherwise attached to another moiety, its name may be written as if it were a fragment (e.g. naphthyl, dibenzofuryl) or as if it were the whole molecule (e.g. naphthalene, dibenzofuran). As used herein, these different ways of designating a substituent or attached fragment are considered to be equivalent.

In one embodiment, the organic molecules according to the invention have an excited state lifetime of not more than 25 μs, of not more than 15 μs, in particular of not more than 10 μs, more preferably of not more than 8 μs or not more than 6 μs, and even more preferably of not more than 4 μs in a film of poly(methyl methacrylate) (PMMA) with 10% by weight of the organic molecule at room temperature.

In one embodiment of the invention, the organic molecules according to the invention represent thermally-activated delayed fluorescence (TADF) emitters, which exhibit a ΔE_{ST} value, which corresponds to the energy difference between the first excited singlet state (S1) and the first excited triplet state (T1), of less than 5000 cm⁻¹, preferably less than 3000 cm⁻¹, more preferably less than 1500 cm⁻¹, even more preferably less than 1000 cm⁻¹ or even less than 500 cm⁻¹.

In a further embodiment of the invention, the organic molecules according to the invention have an emission peak in the visible or nearest ultraviolet range, i.e., in the range of a wavelength of from 380 to 800 nm, with a full width at half maximum of less than 0.50 eV, preferably less than 0.48 eV, more preferably less than 0.45 eV, even more preferably less than 0.43 eV or even less than 0.40 eV in a film of poly(methyl methacrylate) (PMMA) with 10% by weight of the organic molecule at room temperature.

Orbital and excited state energies can be determined either by means of experimental methods or by calculations employing quantum-chemical methods, in particular, density functional theory calculations. The energy of the highest occupied molecular orbital E^{HOMO} is determined by methods known to the person skilled in the art from cyclic voltammetry measurements with an accuracy of 0.1 eV. The energy of the lowest unoccupied molecular orbital E^{LUMO} is calculated as E^{HOMO}+E^{gap}, wherein E^{gap} is determined as follows: For host compounds, the onset of the emission spectrum of a neat film with 10% by weight of the host in poly(methyl methacrylate) (PMMA) is used as E^{gap}, unless stated otherwise. For emitter molecules, E^{gap} is determined as the energy at which the excitation and emission spectra of a film with 10% by weight of the emitter in PMMA cross.

The energy of the first excited triplet state T1 is determined from the onset of the emission spectrum at low temperature, typically at 77 K. For host compounds, where the first excited singlet state and the lowest triplet state are energetically separated by >0.4 eV, the phosphorescence is usually visible in a steady-state spectrum in 2-Me-THF. The triplet energy can thus be determined as the onset of the phosphorescence spectrum. For TADF emitter molecules, the energy of the first excited triplet state T1 is determined from the onset of the delayed emission spectrum at 77 K, if not otherwise stated measured in a film of PMMA with 10% by weight of the emitter. For both, host and emitter compounds, the energy of the first excited singlet state S1 is determined from the onset of the emission spectrum (measured as follows: TADF emitters: concentration of 10% by weight in a film of PMMA; hosts: neat film).

The onset of an emission spectrum is determined by computing the intersection of the tangent to the emission spectrum with the x-axis. The tangent to the emission spectrum is set at the high-energy side of the emission band and at the point at half maximum of the maximum intensity of the emission spectrum.

A further aspect of the invention relates to a method for preparing the organic molecules (with an optional subsequent reaction) of the invention, wherein a substituted 2,4-dichloro-6-phenyltriazine is used as a reactant:

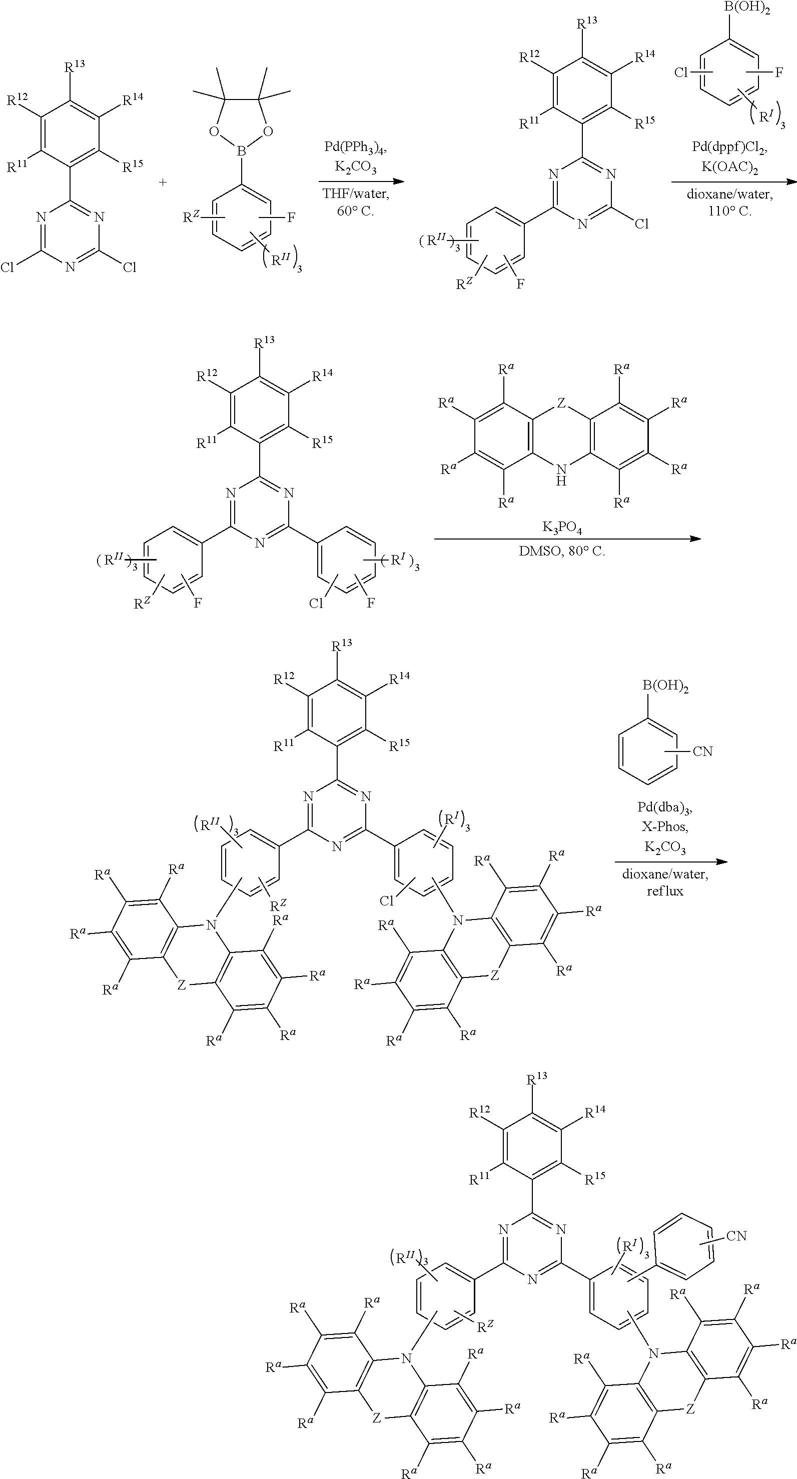

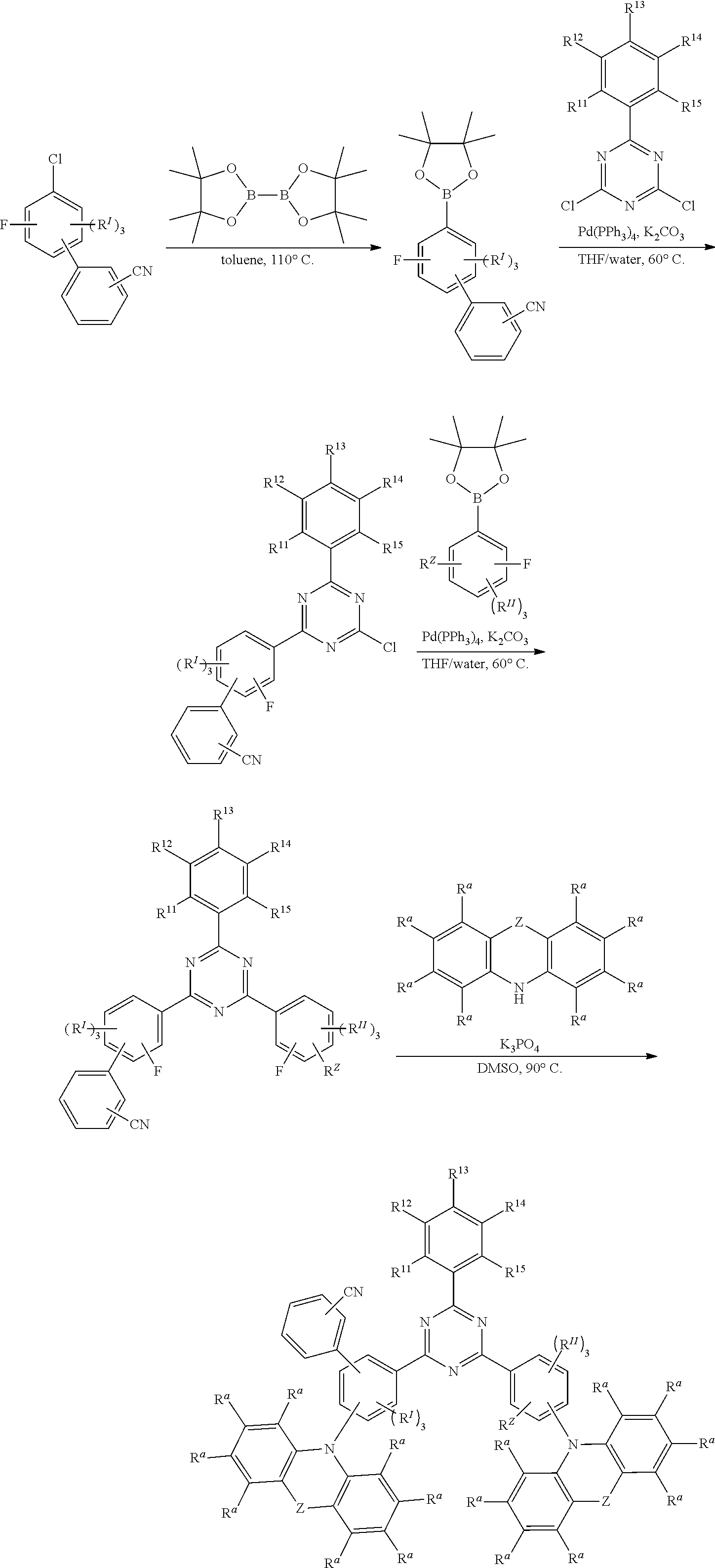

According to the invention, a boronic ester can be used instead of a boronic acid and vice versa.

For the reaction of a nitrogen heterocycle in a nucleophilic aromatic substitution with an aryl halide, preferably an aryl fluoride, typical conditions include the use of a base, such as tribasic potassium phosphate or sodium hydride, for example, in an aprotic polar solvent, such as dimethyl sulfoxide (DMSO) or N,N-dimethylformamide (DMF), for example.

An alternative synthesis route includes the introduction of a nitrogen heterocycle via copper- or palladium-catalyzed coupling to an aryl halide or aryl pseudohalide, preferably an aryl bromide, an aryl iodide, an aryl triflate or an aryl tosylate.

A further aspect of the invention relates to the use of an organic molecule according to the invention as a luminescent emitter or as an absorber, and/or as a host material and/or as an electron transport material, and/or as a hole injection material, and/or as a hole blocking material in an optoelectronic device.

The optoelectronic device may be understood in the broadest sense as any device based on organic materials that is suitable for emitting light in the visible or nearest ultraviolet (UV) range, i.e., in the range of a wavelength of from 380 to 800 nm. More preferably, the optoelectronic device may be able to emit light in the visible range, i.e., of from 400 to 800 nm.

In the context of such use, the optoelectronic device is more particularly selected from the group consisting of:
organic light-emitting diodes (OLEDs),
light-emitting electrochemical cells,
OLED sensors, especially in gas and vapor sensors not hermetically shielded to the outside,
organic diodes,
organic solar cells,
organic transistors,
organic field-effect transistors,
organic lasers and
down-conversion elements.

A light-emitting electrochemical cell consists of three layers, namely a cathode, an anode, and an active layer, which contains the organic molecule according to the invention.

In a preferred embodiment in the context of such use, the optoelectronic device is a device selected from the group consisting of an organic light emitting diode (OLED), a light emitting electrochemical cell (LEC), an organic laser, and a light-emitting transistor.

In one embodiment, the light-emitting layer of an organic light-emitting diode includes not only the organic molecules according to the invention but also a host material whose triplet (T1) and singlet (S1) energy levels are energetically higher than the triplet (T1) and singlet (S1) energy levels of the organic molecule.

A further aspect of the invention relates to a composition including or consisting of:
(a) the organic molecule of the invention, in particular in the form of an emitter and/or a host, and
(b) one or more emitter and/or host materials, which differ from the organic molecule of the invention, and
(c) optionally, one or more dyes and/or one or more solvents.

In a further embodiment of the invention, the composition has a photoluminescence quantum yield (PLQY) of more than 26%, preferably more than 40%, more preferably more than 60%, even more preferably more than 80% or even more than 90% at room temperature.

Compositions with at Least One Further Emitter

One embodiment of the invention relates to a composition including or consisting of:
(i) 1-50% by weight, preferably 5-40% by weight, in particular 10-30% by weight, of the organic molecule according to the invention;
(ii) 5-98% by weight, preferably 30-93.9% by weight, in particular 40-88% by weight, of one host compound H;
(iii) 1-30% by weight, in particular 1-20% by weight, preferably 1-5% by weight, of at least one further emitter molecule F with a structure differing from the structure of the molecules according to the invention; and
(iv) optionally 0-94% by weight, preferably 0.1-65% by weight, in particular 1-50% by weight, of at least one further host compound D with a structure differing from the structure of the molecules according to the invention; and
(v) optionally 0-94% by weight, preferably 0-65% by weight, in particular 0-50% by weight, of a solvent.

The components or the compositions are chosen such that the sum of the weight of the components add up to 100%.

In a further embodiment of the invention, the composition has an emission peak in the visible or nearest ultraviolet range, i.e., in the range of a wavelength of from 380 to 800 nm.

In one embodiment of the invention, the at least one further emitter molecule F is a purely organic emitter.

In one embodiment of the invention, the at least one further emitter molecule F is a purely organic TADF emitter. Purely organic TADF emitters are known from the state of the art, e.g. Wong and Zysman-Colman (“Purely Organic Thermally Activated Delayed Fluorescence Materials for Organic Light-Emitting Diodes.”, Adv. Mater. 2017 June; 29(22)).

In one embodiment of the invention, the at least one further emitter molecule F is a fluorescence emitter, in particular a blue, a green or a red fluorescence emitter.

In a further embodiment of the invention, the composition, containing the at least one further emitter molecule F shows an emission peak in the visible or nearest ultraviolet range, i.e., in the range of a wavelength of from 380 to 800 nm, with a full width at half maximum of less than 0.30 eV, in particular less than 0.25 eV, preferably less than 0.22 eV, more preferably less than 0.19 eV or even less than 0.17 eV at room temperature, with a lower limit of 0.05 eV.

### Light-Emitting Layer EML

In one embodiment, the light-emitting layer EML of an organic light-emitting diode of the invention includes (or essentially consists of) a composition including or consisting of:
(i) 1-50% by weight, preferably 5-40% by weight, in particular 10-30% by weight, of one or more organic molecules according to the invention;
(ii) 5-99% by weight, preferably 30-94.9% by weight, in particular 40-89% by weight, of at least one host compound H; and
(iii) optionally 0-94% by weight, preferably 0.1-65% by weight, in particular 1-50% by weight, of at least one further host compound D with a structure differing from the structure of the molecules according to the invention; and
(iv) optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

Preferably, energy can be transferred from the host compound H to the one or more organic molecules of the invention, in particular transferred from the first excited triplet state T1(H) of the host compound H to the first excited triplet state T1(E) of the one or more organic molecules according to the invention and/or from the first excited singlet state S1(H) of the host compound H to the first excited singlet state S1(E) of the one or more organic molecules according to the invention.

In one embodiment, the host compound H has a highest occupied molecular orbital HOMO(H) having an energy E^{HOMO}(H) in the range of from −5 eV to −6.5 eV and one organic molecule according to the invention E has a highest occupied molecular orbital HOMO(E) having an energy E^{HOMO}(E), wherein E^{HOMO}(H)>E^{HOMO}(E).

In a further embodiment, the host compound H has a lowest unoccupied molecular orbital LUMO(H) having an energy E^{LUMO}(H) and the one organic molecule according to the invention E has a lowest unoccupied molecular orbital LUMO(E) having an energy E^{LUMO}(E), wherein E^{LUMO}(H)>E^{LUMO}(E).

### Light-Emitting Layer EML Including at Least One Further Host Compound D

In a further embodiment, the light-emitting layer EML of an organic light-emitting diode of the invention includes (or essentially consists of) a composition including or consisting of:
(i) 1-50% by weight, preferably 5-40% by weight, in particular 10-30% by weight, of one organic molecule according to the invention;
(ii) 5-99% by weight, preferably 30-94.9% by weight, in particular 40-89% by weight, of one host compound H; and
(iii)0-94% by weight, preferably 0.1-65% by weight, in particular 1-50% by weight, of at least one further host compound D with a structure differing from the structure of the molecules according to the invention; and
(iv) optionally 0-94% by weight, preferably 0-65% by weight, in particular 0-50% by weight, of a solvent; and
(v) optionally 0-30% by weight, in particular 0-20% by weight, preferably 0-5% by weight, of at least one further emitter molecule F with a structure differing from the structure of the molecules according to the invention.

In one embodiment of the organic light-emitting diode of the invention, the host compound H has a highest occupied molecular orbital HOMO(H) having an energy E^{HOMO}(H) in the range of from −5 eV to −6.5 eV and the at least one further host compound D has a highest occupied molecular orbital HOMO(D) having an energy E^{HOMO}(D), wherein E^{HOMO}(H)>E^{HOMO}(D). The relation E^{HOMO}(H)>E^{HOMO}(D) favors an efficient hole transport.

In a further embodiment, the host compound H has a lowest unoccupied molecular orbital LUMO(H) having an energy E^{LUMO}(H) and the at least one further host compound D has a lowest unoccupied molecular orbital LUMO(D) having an energy E^{LUMO}(D), wherein E^{LUMO}(H)>E^{LUMO}(D). The relation E^{LUMO}(H)>E^{LUMO}(D) favors an efficient electron transport.

In one embodiment of the organic light-emitting diode of the invention, the host compound H has a highest occupied molecular orbital HOMO(H) having an energy E^{HOMO}(H) and a lowest unoccupied molecular orbital LUMO(H) having an energy E^{LUMO}(H), and
the at least one further host compound D has a highest occupied molecular orbital HOMO(D) having an energy E^{HOMO}(D) and a lowest unoccupied molecular orbital LUMO(D) having an energy E^{LUMO}(D),
the organic molecule E of the invention has a highest occupied molecular orbital HOMO(E) having an energy E^{HOMO}(E) and a lowest unoccupied molecular orbital LUMO(E) having an energy E^{LUMO}(E),
wherein
E^{HOMO}(H)>E^{HOMO}(D) and the difference between the energy level of the highest occupied molecular orbital HOMO(E) of the organic molecule according to the invention (E^{HOMO}(E)) and the energy level of the highest occupied molecular orbital HOMO(H) of the host compound H (E^{HOMO}(H)) is between −0.5 eV and 0.5 eV, more preferably between −0.3 eV and 0.3 eV, even more preferably between −0.2 eV and 0.2 eV or even between −0.1 eV and 0.1 eV; and
E^{LUMO}(H)>E^{LUMO}(D) and the difference between the energy level of the lowest unoccupied molecular orbital LUMO(E) of the organic molecule according to the invention (E^{LUMO}(E)) and the lowest unoccupied molecular orbital LUMO(D) of the at least one further host compound D (E^{LUMO}(D)) is between −0.5 eV and 0.5 eV, more preferably between −0.3 eV and 0.3 eV, even more preferably between −0.2 eV and 0.2 eV or even between −0.1 eV and 0.1 eV.

### Light-Emitting Layer EML Including at Least One Further Emitter Molecule F

In a further embodiment, the light-emitting layer EML includes (or (essentially) consists of) a composition including or consisting of:
(i) 1-50% by weight, preferably 5-40% by weight, in particular 10-30% by weight, of one organic molecule according to the invention;
(ii) 5-98% by weight, preferably 30-93.9% by weight, in particular 40-88% by weight, of one host compound H;
(iii) 1-30% by weight, in particular 1-20% by weight, preferably 1-5% by weight, of at least one further emitter molecule F with a structure differing from the structure of the molecules according to the invention; and
(iv) optionally 0-94% by weight, preferably 0.1-65% by weight, in particular 1-50% by weight, of at least one further host compound D with a structure differing from the structure of the molecules according to the invention; and
(v) optionally 0-94% by weight, preferably 0-65% by weight, in particular 0-50% by weight, of a solvent.

In a further embodiment, the light-emitting layer EML includes (or (essentially) consists of) a composition as described in Compositions with at least one further emitter, with the at least one further emitter molecule F as defined in Compositions with at least one further emitter, wherein the at least one further emitter molecule F is a blue fluorescence emitter.

In a further embodiment, the light-emitting layer EML includes (or (essentially) consists of) a composition as described in Compositions with at least one further emitter, with the at least one further emitter molecule F as defined in Compositions with at least one further emitter, wherein the at least one further emitter molecule F is a triplet-triplet annihilation (TTA) fluorescence emitter.

In a further embodiment, the light-emitting layer EML includes (or (essentially) consists of) a composition as described in Compositions with at least one further emitter, with the at least one further emitter molecule F as defined in Compositions with at least one further emitter, wherein the at least one further emitter molecule F is a green fluorescence emitter.

In a further embodiment, the light-emitting layer EML includes (or (essentially) consists of) a composition as described in Compositions with at least one further emitter, with the at least one further emitter molecule F as defined in Compositions with at least one further emitter, wherein the at least one further emitter molecule F is a red fluorescence emitter.

In one embodiment of the light-emitting layer EML including at least one further emitter molecule F, energy can be transferred from the one or more organic molecules of the invention E to the at least one further emitter molecule F, in particular transferred from the first excited singlet state S1(E) of one or more organic molecules of the invention E to the first excited singlet state S1(F) of the at least one further emitter molecule F.

In one embodiment, the first excited singlet state S1(H) of one host compound H of the light-emitting layer is higher in energy than the first excited singlet state S1(E) of the one or more organic molecules of the invention E: S1(H)>S1(E), and the first excited singlet state S1(H) of one host compound H is higher in energy than the first excited singlet state S1(F) of the at least one emitter molecule F: S1(H)>S1(F).

In one embodiment, the first excited triplet state T1(H) of one host compound H is higher in energy than the first excited triplet state T1(E) of the one or more organic molecules of the invention E: T1(H)>T1(E), and the first excited triplet state T1(H) of one host compound H is higher in energy than the first excited triplet state T1(F) of the at least one emitter molecule F: T1(H)>T1(F).

In one embodiment, the first excited singlet state S1(E) of the one or more organic molecules of the invention E is higher in energy than the first excited singlet state S1(F) of the at least one emitter molecule F: S1(E)>S1(F).

In one embodiment, the first excited triplet state T1(E) of the one or more organic molecules E of the invention is higher in energy than the first excited singlet state T1(F) of the at least one emitter molecule F: T1(E)>T1(F).

In one embodiment, the first excited triplet state T1(E) of the one or more organic molecules E of the invention is higher in energy than the first excited singlet state T1(F) of the at least one emitter molecule F: T1(E)>T1(F), wherein the absolute value of the energy difference between T1(E) and T1(F) is larger than 0.3 eV, preferably larger than 0.4 eV, or even larger than 0.5 eV.

In one embodiment, the host compound H has a highest occupied molecular orbital HOMO(H) having an energy E^{HOMO}(H) and a lowest unoccupied molecular orbital LUMO(H) having an energy E^{LUMO}(H), and
the one organic molecule according to the invention E has a highest occupied molecular orbital HOMO(E) having an energy E^{HOMO}(E) and a lowest unoccupied molecular orbital LUMO(E) having an energy E^{LUMO}(E),
the at least one further emitter molecule F has a highest occupied molecular orbital HOMO(F) having an energy E^{HOMO}(F) and a lowest unoccupied molecular orbital LUMO(E) having an energy E^{LUMO}(F),
wherein
E^{HOMO}(H)>E^{HOMO}(E) and the difference between the energy level of the highest occupied molecular orbital HOMO(F) of the at least one further emitter molecule (E^{HOMO}(F)) and the energy level of the highest occupied molecular orbital HOMO(H) of the host compound H (E^{HOMO}(H)) is between −0.5 eV and 0.5 eV, more preferably between −0.3 eV and 0.3 eV, even more preferably between −0.2 eV and 0.2 eV or even between −0.1 eV and 0.1 eV; and
E^{LUMO}(H)>E^{LUMO}(E) and the difference between the energy level of the lowest unoccupied molecular orbital LUMO(F) of the at least one further emitter molecule (E^{LUMO}(F)) and the lowest unoccupied molecular orbital LUMO(E) of the one organic molecule according to the invention (E^{LUMO}(E)) is between −0.5 eV and 0.5 eV, more preferably between −0.3 eV and 0.3 eV, even more preferably between −0.2 eV and 0.2 eV or even between −0.1 eV and 0.1 eV.

### Optoelectronic Devices

In a further aspect, the invention relates to an optoelectronic device including an organic molecule or a composition as described herein, more particularly in the form of a device selected from the group consisting of organic light-emitting diode (OLED), light-emitting electrochemical cell, OLED sensor, more particularly gas and vapour sensors not hermetically externally shielded, organic diode, organic solar cell, organic transistor, organic field-effect transistor, organic laser and down-conversion element.

In a preferred embodiment, the optoelectronic device is a device selected from the group consisting of an organic light emitting diode (OLED), a light emitting electrochemical cell (LEC), and a light-emitting transistor.

In one embodiment of the optoelectronic device of the invention, the organic molecule according to the invention is used as emission material in a light-emitting layer EML.

In one embodiment of the optoelectronic device of the invention, the light-emitting layer EML consists of the composition according to the invention described herein.

When the optoelectronic device is an OLED, it may, for example, exhibit the following layer structure:
1. substrate
2. anode layer, A
3. hole injection layer, HIL
4. hole transport layer, HTL
5. electron blocking layer, EBL
6. emitting layer, EML
7. hole blocking layer, HBL
8. electron transport layer, ETL
9. electron injection layer, EIL
10. cathode layer, C

wherein the OLED includes each layer only optionally, different layers may be merged and the OLED may include more than one layer of each layer type defined above.

Furthermore, the optoelectronic device may optionally include one or more protective layers protecting the device from damaging exposure to harmful species in the environment including, exemplarily moisture, vapor and/or gases.

In one embodiment of the invention, the optoelectronic device is an OLED, which exhibits the following inverted layer structure:
1. substrate
2. cathode layer, C
3. electron injection layer, EIL
4. electron transport layer, ETL
5. hole blocking layer, HBL
6. emitting layer, EML
7. electron blocking layer, EBL
8. hole transport layer, HTL
9. hole injection layer, HIL
10. anode layer, A

wherein the OLED with an inverted layer structure includes each layer only optionally, different layers may be merged together into, e.g., one or more layers, and the OLED may include more than one layer of each layer types defined above.

In one embodiment of the invention, the optoelectronic device is an OLED, which may exhibit stacked architecture. In this architecture, contrary to the typical arrangement, where the OLEDs are placed side by side, the individual units are stacked on top of each other. Blended light may be generated with OLEDs exhibiting a stacked architecture, in particular white light may be generated by stacking blue, green and red OLEDs. Furthermore, the OLED exhibiting a stacked architecture may optionally include a charge generation layer (CGL), which is typically located between two OLED subunits and typically consists of an n-doped layer and a p-doped layer with the n-doped layer of one CGL being typically located closer to the anode layer.

In one embodiment of the invention, the optoelectronic device is an OLED, which includes two or more emission layers between anode and cathode. In particular, this so-called tandem OLED includes three emission layers, wherein one emission layer emits red light, one emission layer emits green light and one emission layer emits blue light, and optionally may include further layers such as charge generation layers, blocking or transporting layers between the individual emission layers. In a further embodiment, the emission layers are adjacently stacked. In a further embodiment, the tandem OLED includes a charge generation layer between each two emission layers. In addition, adjacent emission layers or emission layers separated by a charge generation layer may be merged.

The substrate may be formed by any material or composition of materials. Most frequently, glass slides are used as substrates. Alternatively, thin metal layers (e.g., copper, gold, silver or aluminum films) or plastic films or slides may be used. This may allow a higher degree of flexibility. The anode layer A is mostly composed of materials allowing to obtain an (essentially) transparent film. As at least one of the two electrodes should be (essentially) transparent in order to allow light emission from the OLED, either the anode layer A or the cathode layer C is transparent. Preferably, the anode layer A includes a large content or even consists of transparent conductive oxides (TCOs). Such anode layer A may, for example, include indium tin oxide, aluminum zinc oxide, fluorine doped tin oxide, indium zinc oxide, PbO, SnO, zirconium oxide, molybdenum oxide, vanadium oxide, wolfram oxide, graphite, doped Si, doped Ge, doped GaAs, doped polyaniline, doped polypyrrol and/or doped polythiophene.

Preferably, the anode layer A (essentially) consists of indium tin oxide (ITO) (e.g., (InO3)0.9(SnO2)0.1). The roughness of the anode layer A caused by the transparent conductive oxides (TCOs) may be compensated by using a hole injection layer (HIL). Further, the HIL may facilitate the injection of quasi charge carriers (i.e., holes) in that the transport of the quasi charge carriers from the TCO to the hole transport layer (HTL) is facilitated. The hole injection layer (HIL) may include poly-3,4-ethylenedioxy thiophene (PEDOT), polystyrene sulfonate (PSS), MoO₂, V₂O₅, CuPC or CuI, in particular a mixture of PEDOT and PSS. The hole injection layer (HIL) may also prevent the diffusion of metals from the anode layer A into the hole transport layer (HTL). The HIL may include PEDOT:PSS (poly-3,4-ethylenedioxy thiophene: polystyrene sulfonate), PEDOT (poly-3,4-ethylenedioxy thiophene), mMTDATA (4,4′,4″-tris[phenyl(m-tolyl)amino]triphenylamine), Spiro-TAD (2,2′,7,7′-tetrakis(n,n-diphenylamino)-9,9′-spirobifluorene), DNTPD (N1,N1′-(biphenyl-4,4′-diyl)bis(N1-phenyl-N4,N4-di-m-tolylbenzene-1,4-diamine), NPB (N,N′-nis-(1-naphthalenyl)-N,N′-bis-phenyl-(1,1′-biphenyl)-4,4′-diamine), NPNPB (N,N′-diphenyl-N,N′-di-[4-(N,N-diphenyl-amino)phenyl]benzidine), MeO-TPD (N,N,N′,N′-tetrakis(4-methoxyphenyl)benzidine), HAT-CN (1,4,5,8,9,11-hexaazatriphenylen-hexacarbonitrle) and/or Spiro-NPD (N,N′-diphenyl-N,N′-bis-(1-naphthyl)-9,9′-spirobifluorene-2,7-diamine).

Adjacent to the anode layer A or hole injection layer (HIL) typically a hole transport layer (HTL) is located. Herein, any hole transport compound may be used. Exemplarily, electron-rich heteroaromatic compounds such as triarylamines and/or carbazoles may be used as hole transport compound. The HTL may decrease the energy barrier between the anode layer A and the light-emitting layer EML. The hole transport layer (HTL) may also be an electron blocking layer (EBL). Preferably, hole transport compounds bear comparably high energy levels of their triplet states T1. Exemplarily the hole transport layer (HTL) may include a star-shaped heterocycle such as tris(4-carbazoyl-9-ylphenyl)amine (TCTA), poly-TPD (poly(4-butylphenyl-diphenyl-amine)), [alpha]-NPD (poly(4-butylphenyl-diphenyl-amine)), TAPC (4,4′-cyclohexyliden-bis[N,N-bis(4-methylphenyl)benzenamine]), 2-TNATA (4,4′,4″-tris[2-naphthyl(phenyl)amino]triphenylamine), Spiro-TAD, DNTPD, NPB, NPNPB, MeO-TPD, HAT-CN and/or TrisPcz (9,9′-diphenyl-6-(9-phenyl-9H-carbazol-3-yl)-9H,9′H-3,3′-bicarbazole). In addition, the HTL may include a p-doped layer, which may be composed of an inorganic or organic dopant in an organic hole-transporting matrix. Transition metal oxides such as vanadium oxide, molybdenum oxide or tungsten oxide may be used as inorganic dopant. Tetrafluorotetracyanoquinodimethane (F4-TCNQ), copper-pentafluorobenzoate (Cu(I)pFBz) or transition metal complexes may be used as organic dopant.

The EBL may include mCP (1,3-bis(carbazol-9-yl)benzene), TCTA, 2-TNATA, mCBP (3,3-di(9H-carbazol-9-yl)biphenyl), tris-Pcz, CzSi (9-(4-tert-Butylphenyl)-3,6-bis(triphenylsilyl)-9H-carbazole), and/or DCB (N,N′-dicarbazolyl-1,4-dimethylbenzene).

Adjacent to the hole transport layer (HTL), typically, the light-emitting layer EML is located. The light-emitting layer EML includes at least one light emitting molecule. Particular, the EML includes at least one light emitting molecule according to the invention. Typically, the EML additionally includes one or more host material. Exemplarily, the host material is selected from CBP (4,4′-Bis-(N-carbazolyl)-biphenyl), mCP, mCBP Sif87 (dibenzo[b,d]thiophen-2-yltriphenylsilane), CzSi, Sif88 (dibenzo[b,d]thiophen-2-yl)diphenylsilane), DPEPO (bis[2-(diphenylphosphino)phenyl]ether oxide), 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole, 9-[3,5-bis(2-dibenzothiophenyl)phenyl]-9H-carbazole, T2T (2,4,6-tris(biphenyl-3-yl)-1,3,5-triazine), T3T (2,4,6-tris(triphenyl-3-yl)-1,3,5-triazine) and/or TST (2,4,6-tris(9,9′-spirobifluorene-2-yl)-1,3,5-triazine). The host material typically should be selected to exhibit first triplet (T1) and first singlet (S1) energy levels, which are energetically higher than the first triplet (T1) and first singlet (S1) energy levels of the organic molecule.

In one embodiment of the invention, the EML includes a so-called mixed-host system with at least one hole-dominant host and one electron-dominant host. In a particular embodiment, the EML includes exactly one light emitting molecule species according to the invention and a mixed-host system including T2T as the electron-dominant host and a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole as the hole-dominant host. In a further embodiment the EML includes 50-80% by weight, preferably 60-75% by weight of a host selected from CBP, mCP, mCBP, 9-[3-(dibenzofuran-2-yl)phenyl]-9H-carbazole, 9-[3-(dibenzofuran-2-yl)phenyl]-9H-carbazole, 9-[3-(dibenzothiophen-2-yl)phenyl]-9H-carbazole, 9-[3,5-bis(2-dibenzofuranyl)phenyl]-9H-carbazole and 9-[3,5-bis(2-dibenzothiophenyl)phenyl]-9H-carbazole; 10-45% by weight, preferably 15-30% by weight of T2T and 5-40% by weight, preferably 10-30% by weight of light emitting molecule according to the invention.

Adjacent to the light-emitting layer EML an electron transport layer (ETL) may be located. Herein, any electron transporter may be used. Exemplarily, electron-poor compounds such as, e.g., benzimidazoles, pyridines, triazoles, oxadiazoles (e.g., 1,3,4-oxadiazole), phosphinoxides and sulfone, may be used. An electron transporter may also be a star-shaped heterocycle such as 1,3,5-tri(1-phenyl-1H-benzo[d]imidazol-2-yl)phenyl (TPBi). The ETL may include NBphen (2,9-bis(naphthalen-2-yl)-4,7-diphenyl-1,10-phenanthroline), Alq3 (Aluminum-tris(8-hydroxyquinoline)), TSPO1 (diphenyl-4-triphenylsilylphenyl-phosphinoxide), BPyTP2 (2,7-di(2,2′-bipyridin-5-yl)triphenyle), Sif87 (dibenzo[b,d]thiophen-2-yltriphenylsilane), Sif88 (dibenzo[b,d]thiophen-2-yl)diphenylsilane), BmPyPhB (1,3-bis[3,5-di(pyridin-3-yl)phenyl]benzene) and/or BTB (4,4′-bis-[2-(4,6-diphenyl-1,3,5-triazinyl)]-1,1′-biphenyl). Optionally, the ETL may be doped with materials such as Liq. The electron transport layer (ETL) may also block holes or a hole blocking layer (HBL) is introduced.

The HBL may, for example, include BCP (2,9-dimethyl-4,7-diphenyl-1,10-phenanthroline=Bathocuproine), BAlq (bis(8-hydroxy-2-methylquinoline)-(4-phenylphenoxy)aluminum), NBphen (2,9-bis(naphthalen-2-yl)-4,7-diphenyl-1,10-phenanthroline), Alq3 (Aluminum-tris(8-hydroxyquinoline)), TSPO1 (diphenyl-4-triphenylsilylphenyl-phosphinoxide), T2T (2,4,6-tris(biphenyl-3-yl)-1,3,5-triazine), T3T (2,4,6-tris(triphenyl-3-yl)-1,3,5-triazine), TST (2,4,6-tris(9,9′-spirobifluorene-2-yl)-1,3,5-triazine), and/or TCB/TCP (1,3,5-tris(N-carbazolyl)benzol/1,3,5-tris(carbazol)-9-yl) benzene).

A cathode layer C may be located adjacent to the electron transport layer (ETL). For example, the cathode layer C may include or may consist of a metal (e.g., Al, Au, Ag, Pt, Cu, Zn, Ni, Fe, Pb, LiF, Ca, Ba, Mg, In, W, or Pd) or a metal alloy. For practical reasons, the cathode layer may also consist of (essentially) non-transparent metals such as Mg, Ca or Al. Alternatively or additionally, the cathode layer C may also include graphite and or carbon nanotubes (CNTs). Alternatively, the cathode layer C may also consist of nanoscalic silver wires.

An OLED may further, optionally, include a protection layer between the electron transport layer (ETL) and the cathode layer C (which may be designated as electron injection layer (EIL)). This layer may include lithium fluoride, cesium fluoride, silver, Liq (8-hydroxyquinolinolatolithium), Li₂O, BaF₂, MgO and/or NaF.

Optionally, also the electron transport layer (ETL) and/or a hole blocking layer (HBL) may include one or more host compounds.

In order to modify the emission spectrum and/or the absorption spectrum of the light-emitting layer EML further, the light-emitting layer EML may further include one or more additional emitter molecule F. Such an emitter molecule F may be any emitter molecule known in the art. Preferably such an emitter molecule F is a molecule with a structure differing from the structure of the molecules according to the invention. The emitter molecule F may optionally be a TADF emitter. Alternatively, the emitter molecule F may optionally be a fluorescent and/or phosphorescent emitter molecule which is able to shift the emission spectrum and/or the absorption spectrum of the light-emitting layer EML. For example, the triplet and/or singlet excitons may be transferred from the emitter molecule according to the invention to the emitter molecule F before relaxing to the ground state S0 by emitting light typically red-shifted in comparison to the light emitted by emitter molecule E. Optionally, the emitter molecule F may also provoke two-photon effects (i.e., the absorption of two photons of half the energy of the absorption maximum).

Optionally, an optoelectronic device (e.g., an OLED) may, for example, be an essentially white optoelectronic device. Exemplarily such white optoelectronic device may include at least one (deep) blue emitter molecule and one or more emitter molecules emitting green and/or red light. Then, there may also optionally be energy transmittance between two or more molecules as described above.

As used herein, if not defined more specifically in the particular context, the designation of the colors of emitted and/or absorbed light is as follows:
violet: wavelength range of >380-420 nm;
deep blue: wavelength range of >420-480 nm;
sky blue: wavelength range of >480-500 nm;
green: wavelength range of >500-560 nm;
yellow: wavelength range of >560-580 nm;
orange: wavelength range of >580-620 nm;
red: wavelength range of >620-800 nm.

With respect to emitter molecules, such colors refer to the emission maximum. Therefore, exemplarily, a deep blue emitter has an emission maximum in the range of from >420 to 480 nm, a sky-blue emitter has an emission maximum in the range of from >480 to 500 nm, a green emitter has an emission maximum in a range of from >500 to 560 nm, and a red emitter has an emission maximum in a range of from >620 to 800 nm.

A green emitter may preferably have an emission maximum between 500 and 560 nm, more preferably between 510 and 550 nm, and even more preferably between 520 and 540 nm.

A further embodiment of the present invention relates to an OLED, which emits light with CIEx and CIEy color coordinates close to the CIEx (=0.170) and CIEy (=0.797) color coordinates of the primary color green (CIEx=0.170 and CIEy=0.797) as defined by ITU-R Recommendation BT.2020 (Rec. 2020) and thus is suited for the use in Ultra High Definition (UHD) displays, e.g. UHD-TVs. In this context, the term “close to” refers to the ranges of CIEx and CIEy coordinates provided at the end of this paragraph. In commercial applications, typically top-emitting (top-electrode is transparent) devices are used, whereas test devices as used throughout the present application represent bottom-emitting devices (bottom-electrode and substrate are transparent). Accordingly, a further aspect of the present invention relates to an OLED, whose emission exhibits a CIEx color coordinate of between 0.06 and 0.34, preferably between 0.07 and 0.29, more preferably between 0.09 and 0.24 or even more preferably between 0.12 and 0.22 or even between 0.14 and 0.19 and/or a CIEy color coordinate of between 0.44 and 0.84, preferably between 0.55 and 0.83, more preferably between 0.65 and 0.82 or even more preferably between 0.70 and 0.81 or even between 0.75 and 0.8.

Accordingly, a further aspect of the present invention relates to an OLED, which exhibits an external quantum efficiency at 14500 cd/m² of more than 10%, more preferably of more than 13%, more preferably of more than 15%, even more preferably of more than 17% or even more than 20% and/or exhibits an emission maximum between 495 nm and 580 nm, preferably between 500 nm and 560 nm, more preferably between 510 nm and 550 nm, even more preferably between 515 nm and 540 nm and/or exhibits a LT97 value at 14500 cd/m² of more than 100 h, preferably more than 250 h, more preferably more than 500 h, even more preferably more than 750 h or even more than 1000 h.

A further aspect of the present invention relates to an OLED, which emits light at a distinct color point. According to the present invention, the OLED emits light with a narrow emission band (small full width at half maximum (FWHM)). In one aspect, the OLED according to the invention emits light with a FWHM of the main emission peak of less than 0.50 eV, preferably less than 0.48 eV, more preferably less than 0.45 eV, even more preferably less than 0.43 eV or even less than 0.40 eV.

In a further aspect, the invention relates to a method for producing an optoelectronic component. In this case an organic molecule of the invention is used.

The organic electroluminescent device, in particular the OLED according to the present invention can be fabricated by any means of vapor deposition and/or liquid processing. Accordingly, at least one layer is
prepared by means of a sublimation process,
prepared by means of an organic vapor phase deposition process,
prepared by means of a carrier gas sublimation process,
solution processed or printed.

The methods used to fabricate the organic electroluminescent device, in particular the OLED according to the present invention are known in the art. The different layers are individually and successively deposited on a suitable substrate by means of subsequent deposition processes. The individual layers may be deposited using the same or differing deposition methods.

Vapor deposition processes may include thermal (co)evaporation, chemical vapor deposition and physical vapor deposition. For active matrix OLED display, an AMOLED backplane is used as substrate. The individual layer may be processed from solutions or dispersions employing adequate solvents. Solution deposition process exemplarily include spin coating, dip coating and jet printing. Liquid processing may optionally be carried out in an inert atmosphere (e.g., in a nitrogen atmosphere) and the solvent may optionally be completely or partially removed by means known in the state of the art.

### EXAMPLES

### General Synthesis Scheme I

The general synthesis scheme I provides a synthesis scheme for organic molecules according to the invention.

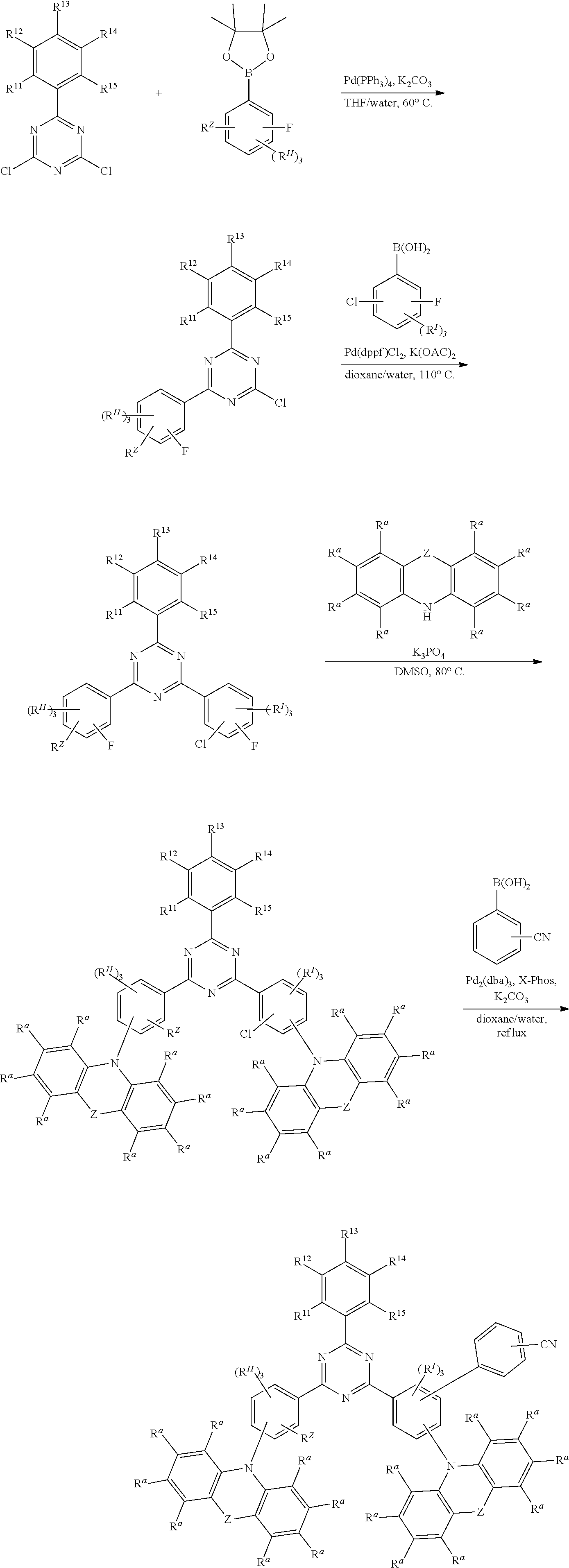

### General Synthesis Scheme II

The general synthesis scheme II provides an alternative synthesis scheme for organic molecules according to the invention.

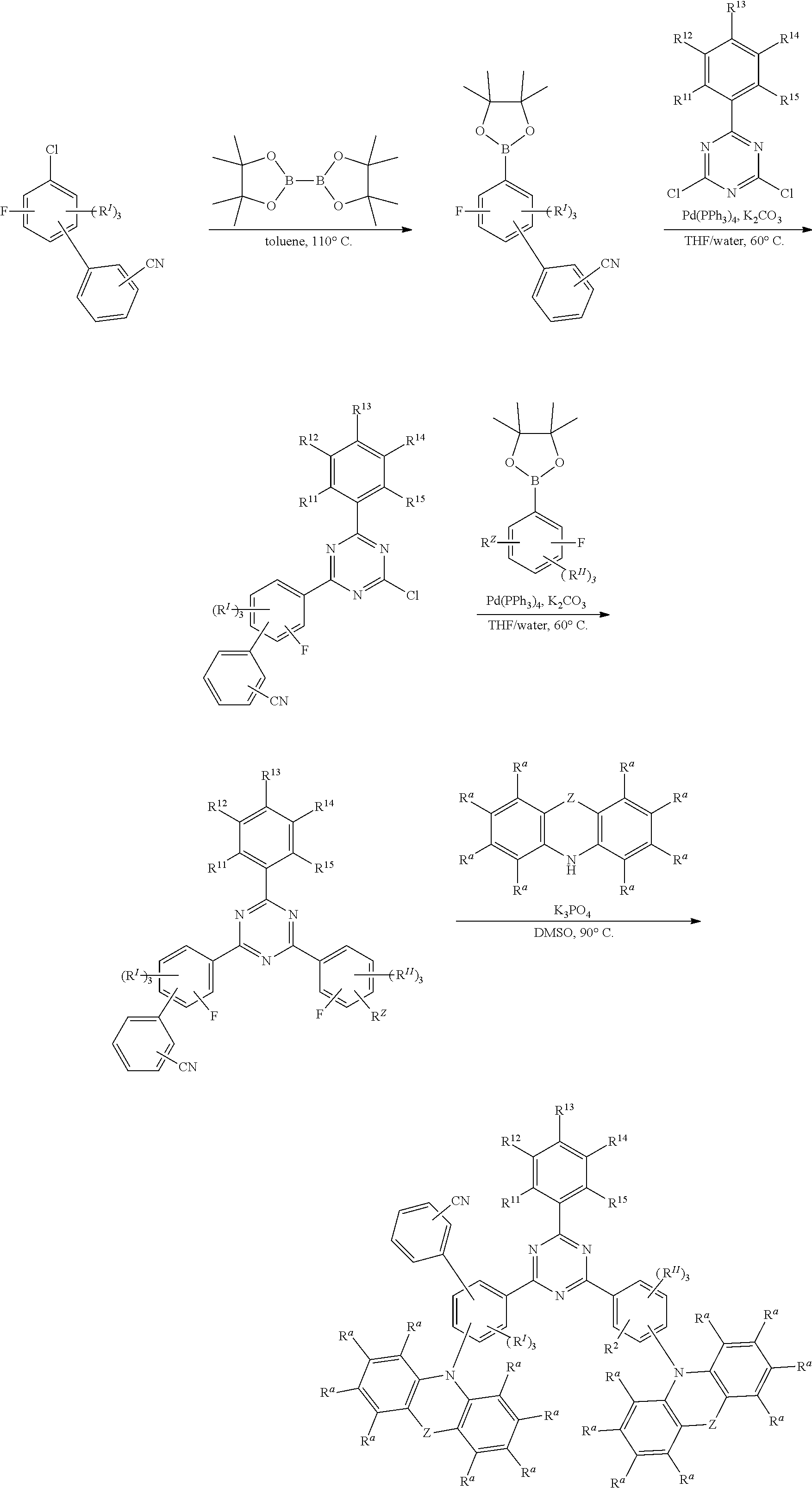

General Procedure for Synthesis AAV1-1

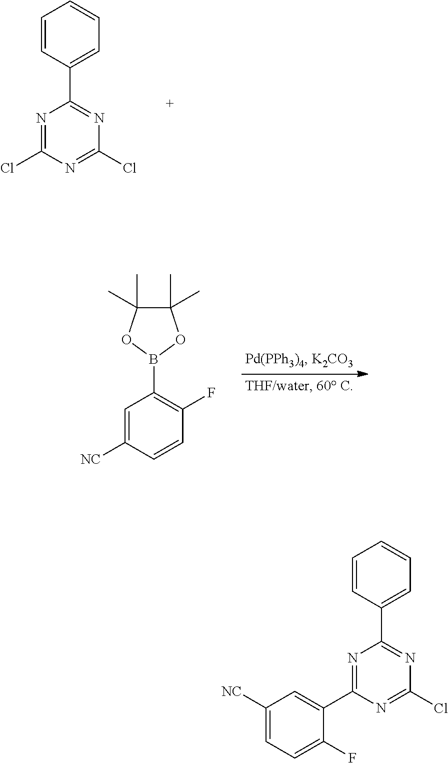 [SMILES: Clc1nc(Cl)nc(-c2ccccc2)n1 ; CC1(C)OB(c2cc(C#N)ccc2F)OC1(C)C ; N#Cc1ccc(F)c(-c2nc(Cl)nc(-c3ccccc3)n2)c1]

Under nitrogen atmosphere, a mixture of THF and water (ratio of 4:1) was added to 4-fluoro-3-(4,4,5,5-tetramethyl-1,3,2-dioxaborolan-2-yl)benzonitrile (1.00 equivalents, CAS 863868-29-5), 2,4-dichloro-6-phenyl-1,3,5-triazine (1.50 equivalents, CAS 1700-02-3), potassium carbonate (2.00 equivalents), and tetrakis(triphenylphosphine)palladium(0) (0.03 equivalents, CAS 14221-01-3), followed by nitrogen-sparging for 15 min. The reaction mixture was stirred at 60° C. until full conversion of the boronic pinacol ester was reached as judged by GC and TLC. After cooling to room temperature, the reaction mixture was extracted with ethyl acetate and brine. The organic extracts were concentrated under reduced pressure. The resulting crude product was heated to reflux in ethanol for 20 min, followed by hot filtration and washing of the solid with ethanol. Purification by MPLC using cyclohexane and dichloromethane (ratio of 1:1) yields the product as a solid.

### General Procedure for Synthesis AAV2-1

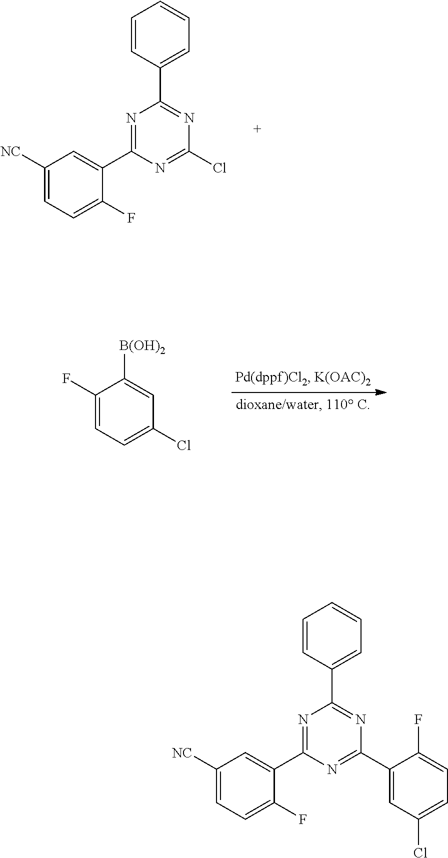 [SMILES: N#Cc1ccc(F)c(-c2nc(Cl)nc(-c3ccccc3)n2)c1 ; OB(O)c1cc(Cl)ccc1F ; N#Cc1ccc(F)c(-c2nc(-c3ccccc3)nc(-c3cc(Cl)ccc3F)n2)c1]

Under nitrogen atmosphere, a mixture of dioxane and water (ratio of 10:1; previously degassed by nitrogen-sparging for 15 min) was added to (5-chloro-2-fluorophenyl)boronic acid (1.00 equivalents, CAS 352535-83-2), 3-(4-chloro-6-phenyl-1,3,5-triazin-2-yl)-4-fluorobenzonitrile (1.10 equivalents, product of AAV1-1), potassium acetate (3.00 equivalents), and [1,1′-bis(diphenylphosphino)ferrocene]dichloropalladium(II) (0.03 equivalents, CAS 72287-26-4). The reaction mixture was stirred under reflux (heating plate set to 110° C.) for 4 h. After cooling to room temperature, water was added, followed by extraction with ethyl acetate. The combined organic layers were concentrated under reduced pressure. The resulting crude product was heated to reflux in ethanol for 3 h and, upon hot filtration, washed with ethanol. The product was obtained as a solid.

### General Procedure for Synthesis AAV2-2

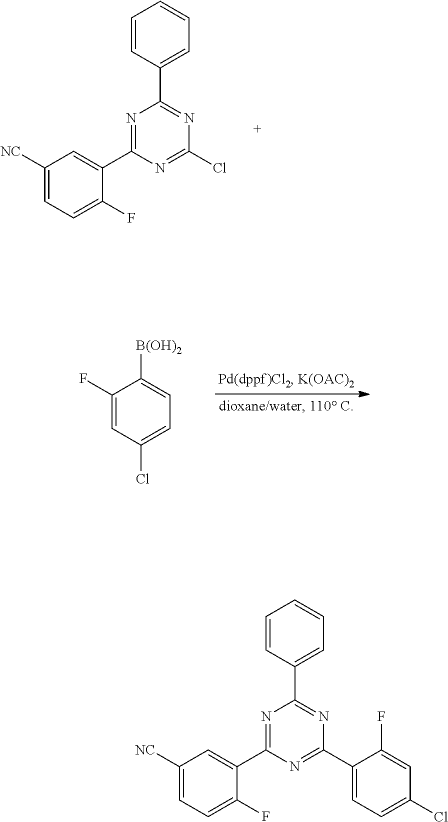 [SMILES: N#Cc1ccc(F)c(-c2nc(Cl)nc(-c3ccccc3)n2)c1 ; OB(O)c1ccc(Cl)cc1F ; N#Cc1ccc(F)c(-c2nc(-c3ccccc3)nc(-c3ccc(Cl)cc3F)n2)c1]

The reaction conditions were analogous to AAV2-1, but (4-chloro-2-fluorophenyl)boronic acid (1.00 equivalents, CAS 160591-91-3) was used as a reactant. After completion of the reaction (heated for 4 h) and cooling down to ambient temperature, the reaction mixture was poured into water. The resulting precipitate was filtered off and washed with water and cold ethanol. The crude product was heated to reflux in a mixture of toluene and cyclohexane (ratio of 3:1) for 1 h. Upon hot filtration, the product was washed with cold ethanol. It was obtained as a solid.

### General Procedure for Synthesis AAV3-1

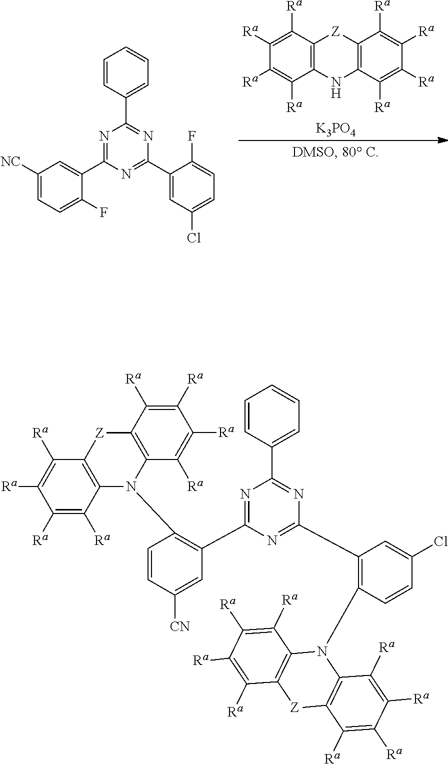

3-(4-(5-chloro-2-fluorophenyl)-6-phenyl-1,3,5-triazin-2-yl)-4-fluorobenzonitrile (1.00 equivalents, product of AAV2-1), the corresponding donor molecule D-H (2.20 equivalents), and tribasic potassium phosphate (3.00 equivalents) were suspended under nitrogen atmosphere in dry DMSO and stirred at 80° C. for 72 h. Subsequently, the reaction mixture was poured into a stirred mixture of water and ice. The resulting precipitate was filtered off and washed with water and n-hexane. The crude product was purified by MPLC using cyclohexane and dichloromethane (ratio of 1:1) and subsequently heated to reflux in ethanol for 2 h. Upon hot filtration, the product was washed with ethanol. It was obtained as a solid.

### General Procedure for Synthesis AAV3-2

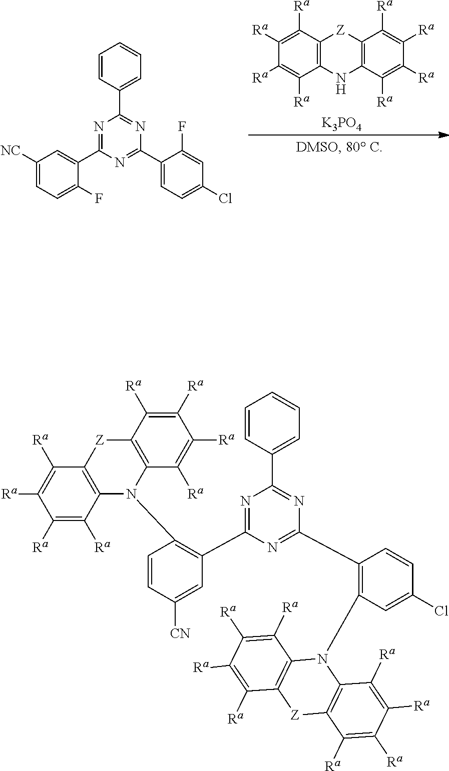

The reaction conditions were analogous to AAV3-1, but 3-(4-(4-chloro-2-fluorophenyl)-6-phenyl-1,3,5-triazin-2-yl)-4-fluorobenzonitrile (1.00 equivalents, product of AAV2-2) was used as a reactant. The filtration through the AlOₓ column was performed using toluene. The resulting crude product was heated to reflux in acetonitrile for 2 h. Upon hot filtration, the product was washed with acetonitrile. Recrystallization from a mixture of toluene and acetonitrile (ratio of 3:2) affords the product as a solid.

### General Procedure for Synthesis AAV4-1

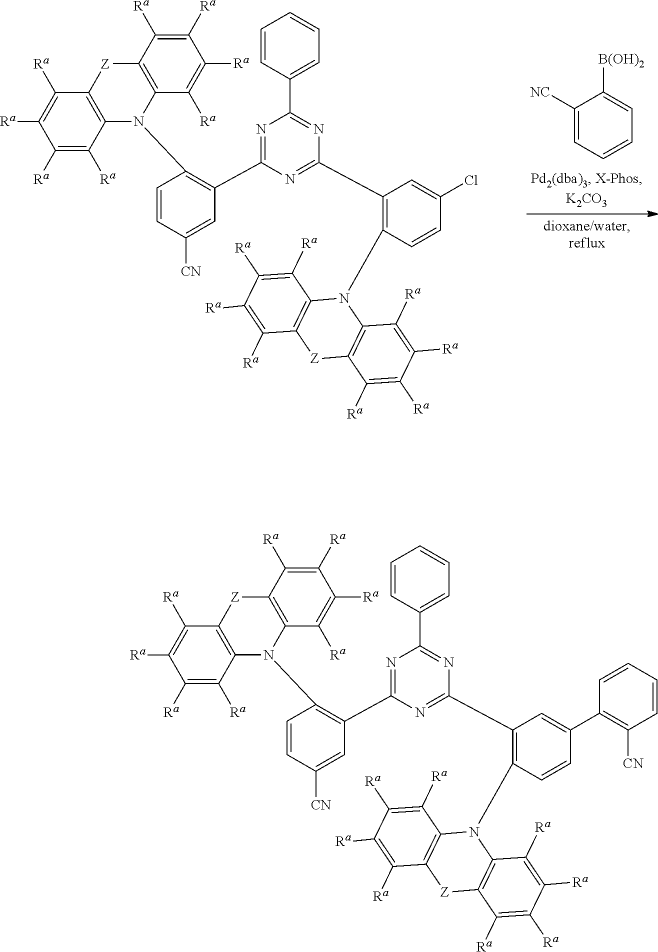

Under nitrogen atmosphere, a mixture of dioxane and water (ratio of 20:3) was added to the product of AAV3-1 (1.00 equivalents), (2-cyanophenyl)boronic acid (1.25 equivalents, CAS 150255-96-2), tris(dibenzylideneacetone)dipalladium(0) (0.04 equivalents, CAS 51364-51-3), 2-dicyclohexylphosphino-2′,4′,6′-triisopropylbiphenyl (X-Phos, 0.16 equivalents, CAS 564483-18-7), and potassium carbonate (2.50 equivalents), followed by nitrogen-sparging for 10 min. The reaction mixture was stirred under reflux for 2 h (reaction monitored by TLC), then cooled to ambient temperature, and poured into ice-cold water. The precipitate was filtered and washed with water. The crude product was recrystallized from n-hexane and ethyl acetate, followed by purification via MPLC using cyclohexane and dichloromethane in a ratio of 1:1. The product was obtained as a solid.

### General Procedure for Synthesis AAV4-2

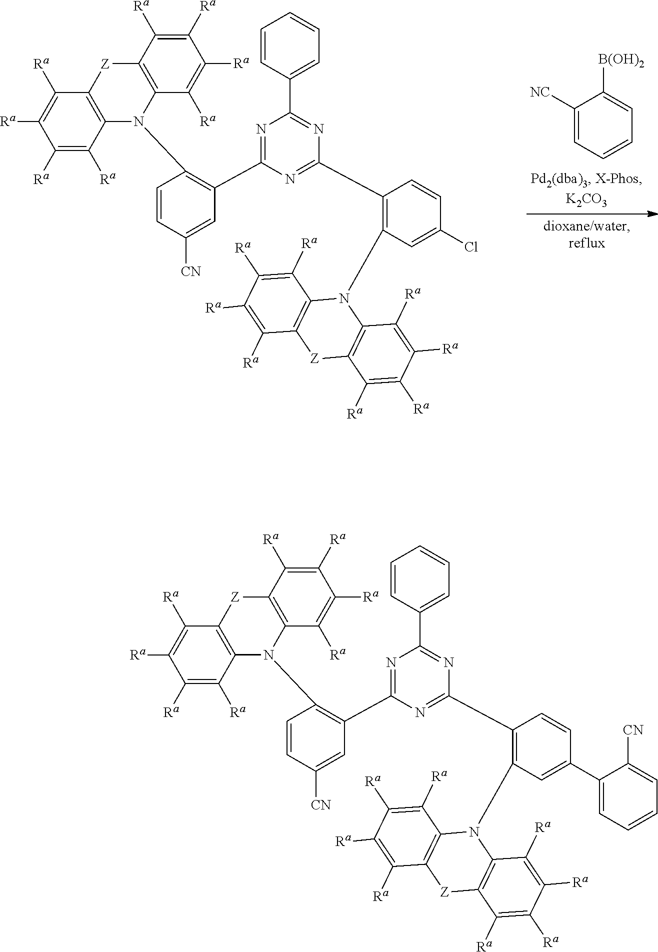

The reaction conditions were analogous to AAV4-1, but the product of AAV3-2 was used as a reactant.

### General Procedure for Synthesis AAV5-1

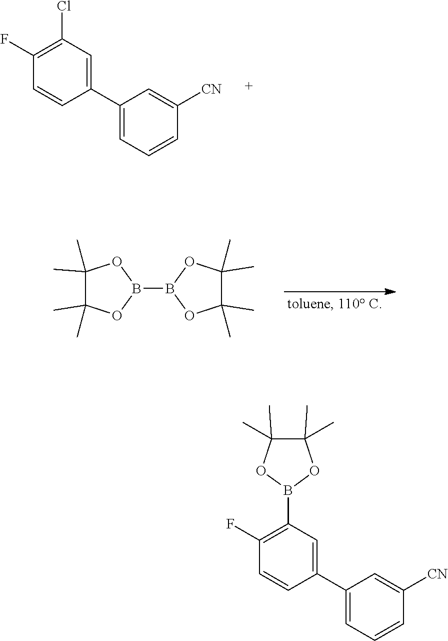

Under nitrogen atmosphere, dry toluene was added to 3′-chloro-4′-fluoro-[1,1′-biphenyl]-3-carbonitrile (1.00 equivalents) and 4,4,4′,4′,5,5,5′,5′-octamethyl-2,2′-bi(1,3,2-dioxaborolane) (1.30 equivalents, CAS 73183-34-3), followed by nitrogen-sparging for 15 min. The reaction mixture was stirred at 110° C. for 3 h (reaction monitored by TLC), then cooled to 70° C. Celite and charcoal were added and the suspension was stirred at 70° C. for 20 min, filtered and the filtrate was extracted with ethyl acetate and brine. The combined organic layers were concentrated under reduced pressure and the crude product was recrystallized from n-hexane. The product was obtained as a solid.

### General Procedure for Synthesis AAV6-1

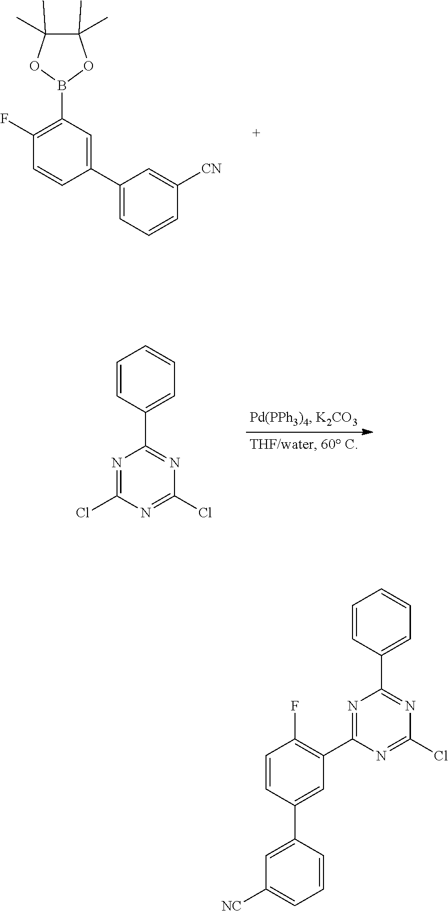 [SMILES: CC1(C)OB(c2cc(-c3cccc(C#N)c3)ccc2F)OC1(C)C ; Clc1nc(Cl)nc(-c2ccccc2)n1 ; N#Cc1cccc(-c2ccc(F)c(-c3nc(Cl)nc(-c4ccccc4)n3)c2)c1]

Under nitrogen atmosphere, a mixture of THF and water (ratio of 4:1) was added to 4′-fluoro-3′-(4,4,5,5-tetramethyl-1,3,2-dioxaborolan-2-yl)-[1,1′-biphenyl]-3-carbonitrile (1.00 equivalents, product of AAV5-1), 2,4-dichloro-6-phenyl-1,3,5-triazine (1.50 equivalents, CAS 1700-02-3), potassium carbonate (2.00 equivalents), and tetrakis(triphenylphosphine)palladium(0) (0.03 equivalents, CAS 14221-01-3), followed by nitrogen-sparging for 10 min. The reaction mixture was stirred at 60° C. for 16 h. After cooling to room temperature, the reaction mixture was extracted with ethyl acetate and brine. The organic extracts were concentrated under reduced pressure. Purification by MPLC using cyclohexane and dichloromethane (ration of 1:1) yields the product as a solid.

### General Procedure for Synthesis AAV7-1

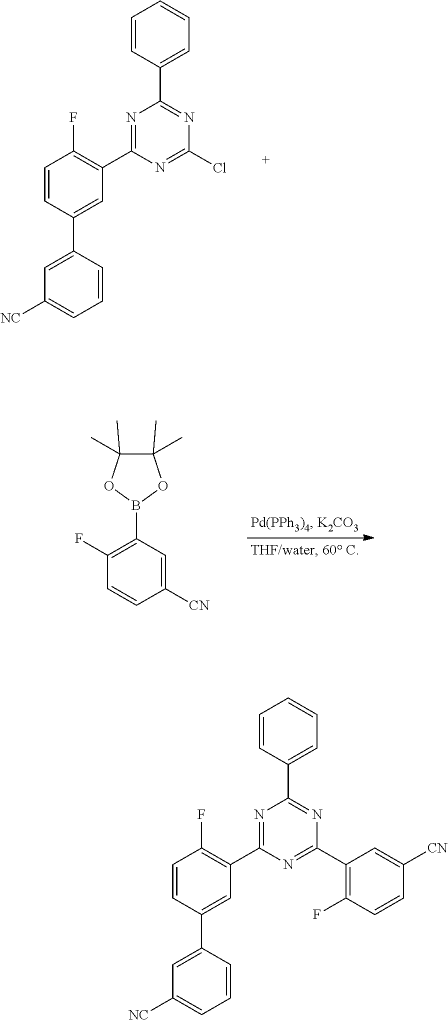 [SMILES: N#Cc1cccc(-c2ccc(F)c(-c3nc(Cl)nc(-c4ccccc4)n3)c2)c1 ; CC1(C)OB(c2cc(C#N)ccc2F)OC1(C)C ; N#Cc1cccc(-c2ccc(F)c(-c3nc(-c4ccccc4)nc(-c4cc(C#N)ccc4F)n3)c2)c1]

Under nitrogen atmosphere, a mixture of THF and water (ratio of 25:3) was added to 3′-(4-chloro-1-phenyl-1,3,5-triazin-2-yl)-4′-fluoro-[1,1′-biphenyl]-3-carbonitrile (1.00 equivalents, product of AAV6-1), 4-fluoro-3-(4,4,5,5-tetramethyl-1,3,2-dioxaborolan-2-yl)benzonitrile (1.20 equivalents, CAS 863868-29-5), potassium carbonate (2.00 equivalents), and tetrakis(triphenylphosphine)palladium(0) (0.03 equivalents, CAS 14221-01-3), followed by nitrogen-sparging for 15 min. The reaction mixture was stirred at 60° C. for 16 h. After cooling to room temperature, a mixture of water and THF (ratio of 1:1) was added. The precipitate was filtered off and dissolved in dichloromethane. After washing with water, dichloromethane was removed under reduced pressure yielding the product as a solid.

### General Procedure for Synthesis AAV8-1

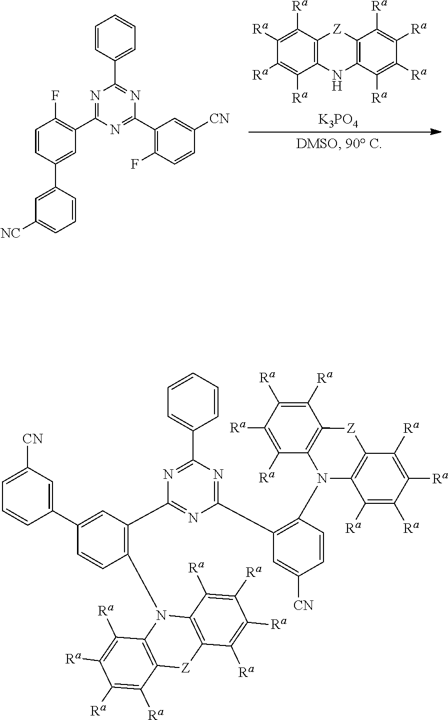

3′-(4-(5-cyano-2-fluorophenyl)-6-phenyl-1,3,5-triazin-2-yl)-4′-fluoro-[1,1′-biphenyl]-3-carbonitrile (1.00 equivalents, product of AAV7-1), the corresponding donor molecule D-H (2.20 equivalents), and tribasic potassium phosphate (3.00 equivalents) were suspended under nitrogen atmosphere in dry DMSO and stirred at 90° C. for 11 h. After cooling to room temperature, the reaction mixture was extracted with dichloromethane and water, followed by extraction of the organic layer with water. Dichloromethane was removed under reduced pressure affording the crude product. Filtration through a short pad of silica using dichloromethane was followed by concentration of the filtrate in vacuo. Acetonitrile was added and, upon treatment in an ultrasonic bath for 15 min and storing at −18° C., the formed precipitate was filtered off and washed with acetonitrile. The product was obtained as a solid.

In particular, the donor molecule D-H was a 3,6-substituted carbazole (e.g., 3,6-dimethylcarbazole, 3,6-diphenylcarbazole, 3,6-di-tert-butylcarbazole), a 2,7-substituted carbazole (e.g., 2,7-dimethylcarbazole, 2,7-diphenylcarbazole, 2,7-di-tert-butylcarbazole), a 1,8-substituted carbazole (e.g., 1,8-dimethylcarbazole, 1,8-diphenylcarbazole, 1,8-di-tert-butylcarbazole), a 1-substituted carbazole (e.g., 1-methylcarbazole, 1-phenylcarbazole, 1-tert-butylcarbazole), a 2-substituted carbazole (e.g., 2-methylcarbazole, 2-phenylcarbazole, 2-tert-butylcarbazole), or a 3-substituted carbazole (e.g., 3-methylcarbazole, 3-phenylcarbazole, 3-tert-butylcarbazole).

Exemplarily a halogen-substituted carbazole, particularly 3-bromocarbazole, can be used as D-H.

In a subsequent reaction a boronic acid ester functional group or boronic acid functional group may be exemplarily introduced at the position of the one or more halogen substituents, which was introduced via D-H, to yield the corresponding carbazol-3-ylboronic acid ester or carbazol-3-ylboronic acid, e.g., via the reaction with bis(pinacolato)diboron (CAS No. 73183-34-3). Subsequently, one or more substituents R^{a} may be introduced in place of the boronic acid ester group or the boronic acid group via a coupling reaction with the corresponding halogenated reactant R^{a}-Hal, preferably R^{a}—Cl and R^{a}—Br.

Alternatively, one or more substituents R^{a} may be introduced at the position of the one or more halogen substituents, which was introduced via D-H, via the reaction with a boronic acid of the substituent R^{a} [R^{a}—B(OH)₂] or a corresponding boronic acid ester.

HPLC-MS:

HPLC-MS analysis was performed on an HPLC by Agilent (1100 series) with MS-detector (Thermo LTQ XL).

Exemplary a typical HPLC method was as follows: a reverse phase column 4.6 mm×150 mm, particle size 3.5 μm from Agilent (ZORBAX Eclipse Plus 95 Å C18, 4.6×150 mm, 3.5 μm HPLC column) was used in the HPLC. The HPLC-MS measurements were performed at room temperature (rt) with the following gradients:

| Flow rate [ml/min] | time [min] | A[%] | B[%] | C[%] |
|---|---|---|---|---|
| 2.5 | 0 | 40 | 50 | 10 |
| 2.5 | 5 | 40 | 50 | 10 |
| 2.5 | 25 | 10 | 20 | 70 |
| 2.5 | 35 | 10 | 20 | 70 |
| 2.5 | 35.01 | 40 | 50 | 10 |
| 2.5 | 40.01 | 40 | 50 | 10 |
| 2.5 | 41.01 | 40 | 50 | 10 |

and using the following solvent mixtures:

| | | |
|---|---|---|
| Solvent A: | H2O (90%) | MeCN (10%) |
| Solvent B: | H2O (10%) | MeCN (90%) |
| Solvent C: | THF (50%) | MeCN (50%) |

An injection volume of 5 μL from a solution with a concentration of 0.5 mg/mL of the analyte was taken for the measurements.

Ionization of the probe was performed using an APCI (atmospheric pressure chemical ionization) source either in positive (APCI+) or negative (APCI−) ionization mode.

Cyclic Voltammetry

Cyclic voltammograms were measured from solutions having concentration of 10⁻³ mol/l of the organic molecules in dichloromethane or a suitable solvent and a suitable supporting electrolyte (e.g. 0.1 mol/L of tetrabutylammonium hexafluorophosphate). The measurements were conducted at room temperature under nitrogen atmosphere with a three-electrode assembly (Working and counter electrodes: Pt wire, reference electrode: Pt wire) and calibrated using FeCp₂/FeCp₂⁺ as internal standard. The HOMO data was corrected using ferrocene as internal standard against a saturated calomel electrode (SCE).

Density Functional Theory Calculation

Molecular structures were optimized employing the BP86 functional and the resolution of identity approach (RI). Excitation energies were calculated using the (BP86) optimized structures employing Time-Dependent DFT (TD-DFT) methods. Orbital and excited state energies were calculated with the B3LYP functional. Def2-SVP basis sets (and an m4-grid for numerical integration were used. The Turbomole program package was used for all calculations.

Photophysical Measurements

Sample pretreatment: Spin-coating

Apparatus: Spin150, SPS euro.

The sample concentration was 10 mg/ml, dissolved in a suitable solvent.

Program: 1) 3 s at 400 U/min; 2) 20 s at 1000 U/min at 1000 Upm/s. 3) 10 s at 4000 U/min at 1000 Upm/s. After coating, the films were tried at 70° C. for 1 min.

Photoluminescence spectroscopy and TCSPC (Time-correlated single-photon counting)

Steady-state emission spectroscopy was measured by a Horiba Scientific, Modell FluoroMax-4 equipped with a 150 W Xenon-Arc lamp, excitation- and emissions monochromators and a Hamamatsu R928 photomultiplier and a time-correlated single-photon counting option. Emissions and excitation spectra were corrected using standard correction fits.

Excited state lifetimes were determined employing the same system using the TCSPC method with FM-2013 equipment and a Horiba Yvon TCSPC hub.

Excitation sources:

NanoLED 370 (wavelength: 371 nm, puls duration: 1.1 ns)

NanoLED 290 (wavelength: 294 nm, puls duration: <1 ns)

SpectraLED 310 (wavelength: 314 nm)

SpectraLED 355 (wavelength: 355 nm).

Data analysis (exponential fit) was done using the software suite DataStation and DAS6 analysis software. The fit was specified using the chi-squared-test.

Photoluminescence Quantum Yield Measurements

For photoluminescence quantum yield (PLQY) measurements an Absolute PL Quantum Yield Measurement C9920-03G system (Hamamatsu Photonics) was used.

Quantum yields and CIE coordinates were determined using the software U6039-05 version 3.6.0.

Emission maxima were given in nm, quantum yields Φ in % and CIE coordinates as x,y values.

PLQY was determined using the following protocol:

Quality assurance: Anthracene in ethanol (known concentration) was used as reference

Excitation wavelength: the absorption maximum of the organic molecule was determined and the molecule was excited using this wavelength

Measurement

Quantum yields were measured for sample of solutions or films under nitrogen atmosphere. The yield was calculated using the equation:

$${Φ}_{PL} = \frac{{n}_{photon} , emited}{{n}_{photon} , absorbed} = \frac{\int \frac{λ}{hc} [ {Int}_{emitted}^{sample} ( λ ) - {Int}_{absored}^{sample} ( λ ) ] d λ}{\int \frac{λ}{hc} [ {Int}_{emitted}^{reference} ( λ ) - {Int}_{absored}^{reference} ( λ ) ] d λ}$$

wherein nₚₕₒₜₒₙ denotes the photon count and Int. denotes the intensity.

### Production and Characterization of Optoelectronic Devices

Optoelectronic devices, such as OLED devices, including an organic molecule according to the invention can be produced via vacuum-deposition methods. If a layer contains more than one compound, the weight-percentage of one or more compounds was given in %. The total weight-percentage values amount to 100%, thus if a value was not given, the fraction of this compound equals to the difference between the given values and 100%.

The not fully optimized OLEDs were characterized using standard methods and measuring electroluminescence spectra, the external quantum efficiency (in %) in dependency on the intensity, calculated using the light detected by the photodiode, and the current. The OLED device lifetime was extracted from the change of the luminance during operation at constant current density. The LT50 value corresponds to the time, where the measured luminance decreased to 50% of the initial luminance, analogously LT80 corresponds to the time point, at which the measured luminance decreased to 80% of the initial luminance, and LT 95 to the time point, at which the measured luminance decreased to 95% of the initial luminance etc.

Accelerated lifetime measurements were performed (e.g. applying increased current densities). For example, LT80 values at 500 cd/m² were determined using the following equation:

$$LT 80 ( 500 \frac{{cd}^{2}}{{m}^{2}} ) = LT 80 ( {L}_{0} ) {( \frac{{L}_{0}}{500 \frac{{cd}^{2}}{{m}^{2}}} )}^{1.6}$$

wherein L₀ denotes the initial luminance at the applied current density. The values correspond to the average of several pixels (typically two to eight), the standard deviation between these pixels was given. The figures show the data series for one OLED pixel.

### Example 1

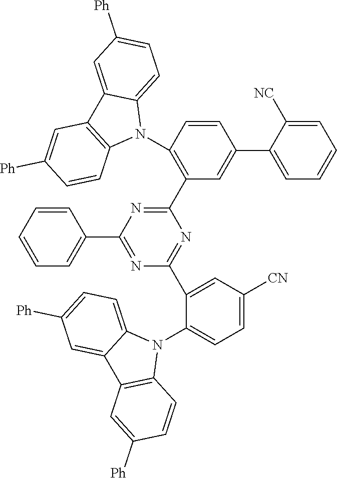 [SMILES: N#Cc1ccc(-n2c3ccc(-c4ccccc4)cc3c3cc(-c4ccccc4)ccc32)c(-c2nc(-c3ccccc3)nc(-c3cc(-c4ccccc4C#N)ccc3-n3c4ccc(-c5ccccc5)cc4c4cc(-c5ccccc5)ccc43)n2)c1]

Example 1 was synthesized according to AAV1-1 (yield 45%), AAV2-1 (yield 74%), AAV3-1 (yield 29%), and AAV4-1 (yield 29%).

MS (HPLC-MS), m/z (retention time): 1070.0 (5.79 min).

FIG. 1 depicts the emission spectrum of example 1 (10% by weight in PMMA). The emission maximum (□ₘₐₓ) was at 508 nm. The photoluminescence quantum yield (PLQY) was 70%, the full width at half maximum (FWHM) was 0.41 eV, and the emission lifetime was 8.9 μs. The resulting CIEx coordinate was determined at 0.28 and the CIEy coordinate at 0.53.

### Example 2

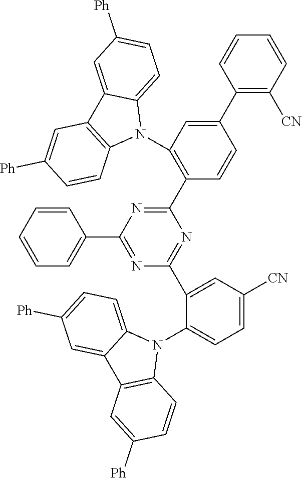 [SMILES: N#Cc1ccc(-n2c3ccc(-c4ccccc4)cc3c3cc(-c4ccccc4)ccc32)c(-c2nc(-c3ccccc3)nc(-c3ccc(-c4ccccc4C#N)cc3-n3c4ccc(-c5ccccc5)cc4c4cc(-c5ccccc5)ccc43)n2)c1]

Example 2 was synthesized according to AAV1-1 (yield 41%), AAV2-2 (yield 63%), AAV3-2 (yield 63%), and AAV4-2 (yield 44%).

MS (HPLC-MS), m/z (retention time): 1070.6 (5.80 min).

FIG. 2 depicts the emission spectrum of example 2 (10% by weight in PMMA). The emission maximum (□ₘₐₓ) was at 515 nm. The photoluminescence quantum yield (PLQY) was 66%, the full width at half maximum (FWHM) was 0.41 eV, and the emission lifetime was 9.2 μs. The resulting CIEₓ coordinate was determined at 0.31 and the CIE_{y} coordinate at 0.56.

### Example 3

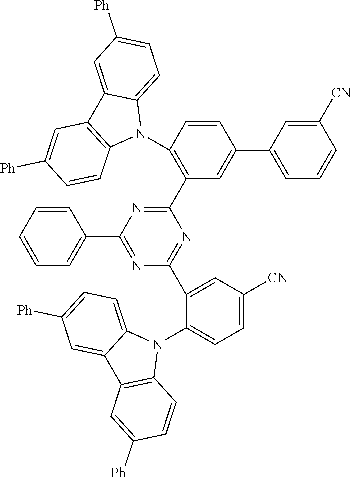 [SMILES: N#Cc1cccc(-c2ccc(-n3c4ccc(-c5ccccc5)cc4c4cc(-c5ccccc5)ccc43)c(-c3nc(-c4ccccc4)nc(-c4cc(C#N)ccc4-n4c5ccc(-c6ccccc6)cc5c5cc(-c6ccccc6)ccc54)n3)c2)c1]

Example 3 was synthesized according to AAV5-1 (yield 86%), AAV6-1 (yield 51%), AAV7-1 (yield 79%), and AAV8-1 (yield 10%).

MS (HPLC-MS), m/z (retention time): 1070.8 (5.92 min).

FIG. 3 depicts the emission spectrum of example 3 (10% by weight in PMMA). The emission maximum (□ₘₐₓ) was at 509 nm. The photoluminescence quantum yield (PLQY) was 71%, the full width at half maximum (FWHM) was 0.41 eV, and the emission lifetime was 10.2 μs. The resulting CIEₓ coordinate was determined at 0.28 and the CIE_{y} coordinate at 0.53.

### Example 4

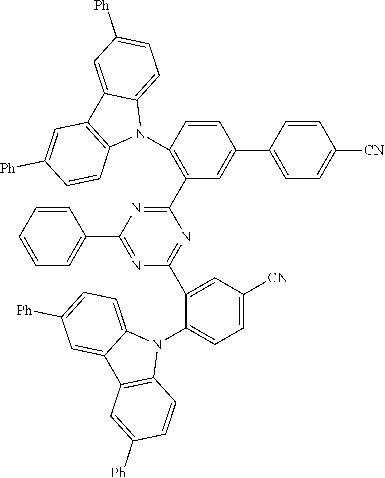 [SMILES: N#Cc1ccc(-c2ccc(-n3c4ccc(-c5ccccc5)cc4c4cc(-c5ccccc5)ccc43)c(-c3nc(-c4ccccc4)nc(-c4cc(C#N)ccc4-n4c5ccc(-c6ccccc6)cc5c5cc(-c6ccccc6)ccc54)n3)c2)cc1]

Example 4 was synthesized according to AAV1-1(yield 52%), AAV2-1(yield 74%), AAV3-1 (yield 59%), and AAV4-1 (yield 65%) replacing 2-cyanophenylboronic acid by 4-cyanophenylboronic acid (CAS 126747-14-6).

MS (HPLC-MS), m/z (retention time): 1072.1 (5.92 min).

FIG. 4 depicts the emission spectrum of example 4 (10% by weight in PMMA). The emission maximum (□ₘₐₓ) was at 509 nm. The photoluminescence quantum yield (PLQY) was 74%, the full width at half maximum (FWHM) was 0.41 eV, and the emission lifetime was 15.2 μs. The resulting CIEx coordinate was determined at 0.28 and the CIEy coordinate at 0.53.

### Example D1

Example 1 was tested in an optoelectronic device in the form of an OLED D1, which was fabricated with the following layer structure:

| Layer # | Thickness | | D1 |
|---|---|---|---|
| 10 | 100 | nm | Al |
| 9 | 2 | nm | Liq |
| 8 | 20 | nm | NBPhen |
| 7 | 10 | nm | MAT1 |
| 6 | 50 | nm | MAT2 (80%): |
| | | | Example 1 (20%) |
| 5 | 10 | nm | MAT2 |
| 4 | 10 | nm | TCTA |
| 3 | 50 | nm | NPB |
| 2 | 5 | nm | HAT-CN |
| 1 | 50 | nm | ITO |

| | |
|---|---|
| Substrate | Glass |

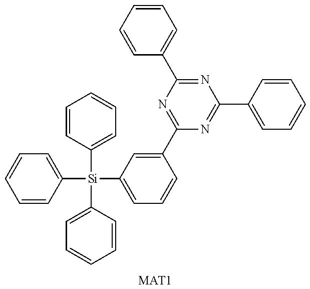

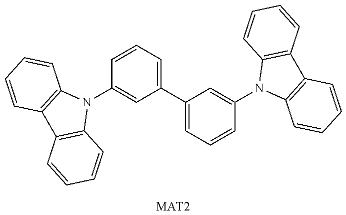

OLED D1 yielded an external quantum efficiency (EQE) at 1000 cd/m² of 18.4%. The emission maximum was at 512 nm with a FWHM of 76 nm at 7.0 V. The corresponding CIEx value was 0.28 and the CIEy value was 0.59. A LT95-value at 1200 cd/m² of 220 h was determined.

### Example D2

Example 3 was tested in the OLED D2, which was fabricated with the following layer structure:

| Layer # | Thickness | | D2 |
|---|---|---|---|
| 10 | 100 | nm | Al |
| 9 | 2 | nm | Liq |
| 8 | 20 | nm | NBPhen |
| 7 | 10 | nm | MAT1 |
| 6 | 50 | nm | MAT2 (85%):Example 3 (15%) |
| 5 | 10 | nm | MAT2 |
| 4 | 10 | nm | TCTA |
| 3 | 50 | nm | NPB |
| 2 | 5 | nm | HAT-CN |
| 1 | 50 | nm | ITO |
| Substrate | | | Glass |

OLED D2 yielded an external quantum efficiency (EQE) at 1000 cd/m² of 16.7%. The emission maximum was at 508 nm with a FWHM of 76 nm at 7.0 V. The corresponding CIEx value was 0.26 and the CIEy value was 0.58.

### Example D3

Example 3 was tested in the OLED D3, which was fabricated with the following layer structure:

| Layer # | Thickness | | D3 |
|---|---|---|---|
| 10 | 100 | nm | Al |
| 9 | 2 | nm | Liq |
| 8 | 20 | nm | NBPhen |
| 7 | 10 | nm | MAT1 |
| 6 | 50 | nm | MAT2 (84%): |
| | | | Example 3 (15%): |
| | | | MAT3 (1%) |
| 5 | 10 | nm | MAT2 |
| 4 | 10 | nm | TCTA |
| 3 | 50 | nm | NPB |
| 2 | 5 | nm | HAT-CN |
| 1 | 50 | nm | ITO |

| | |
|---|---|
| Substrate | Glass |

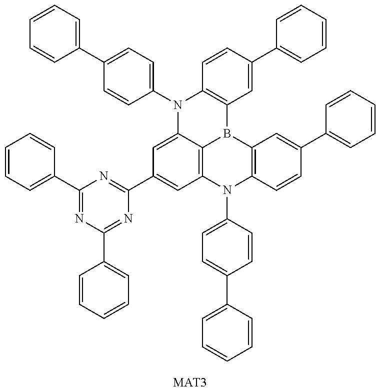

OLED D3 yielded an external quantum efficiency (EQE) at 1000 cd/m² of 18.3%. The emission maximum was at 532 nm with a FWHM of 36 nm at 5.5 V. The corresponding CIEx value was 0.31 and the CIEy value was 0.65.

### Example D4

Example 4 was tested in the OLED D4, which was fabricated with the following layer structure:

| Layer # | Thickness | | D4 |
|---|---|---|---|
| 10 | 100 | nm | Al |
| 9 | 2 | nm | Liq |
| 8 | 20 | nm | NBPhen |
| 7 | 10 | nm | MAT1 |
| 6 | 50 | nm | MAT2 (80%):Example 4 (20%) |
| 5 | 10 | nm | MAT2 |
| 4 | 10 | nm | TCTA |
| 3 | 50 | nm | NPB |
| 2 | 5 | nm | HAT-CN |
| 1 | 50 | nm | ITO |
| Substrate | | | Glass |

OLED D4 yielded an external quantum efficiency (EQE) at 1000 cd/m² of 19.3%. The emission maximum was at 514 nm with a FWHM of 78 nm at 6.4 V. The corresponding CIEx value was 0.28 and the CIEy value was 0.59. A LT95-value at 1200 cd/m² of 245 h was determined.

### Additional Examples of Organic Molecules of the Invention

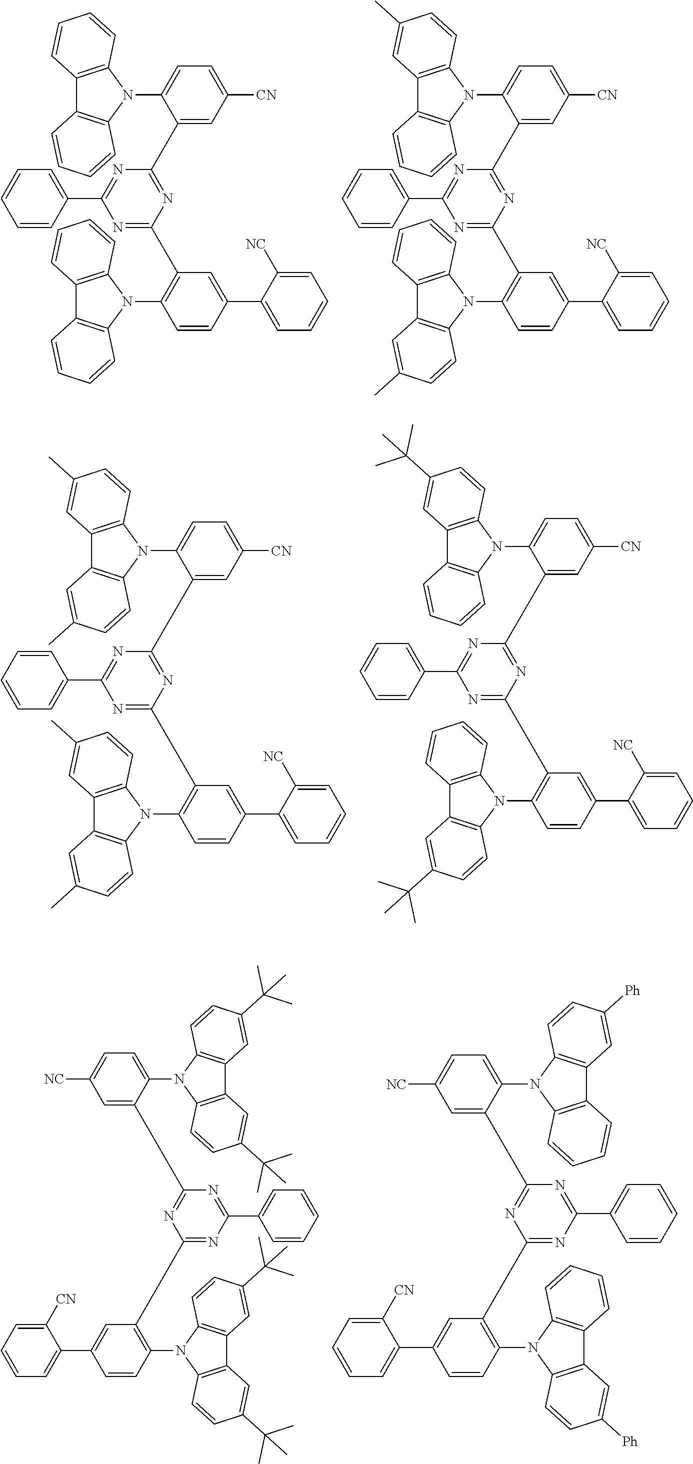

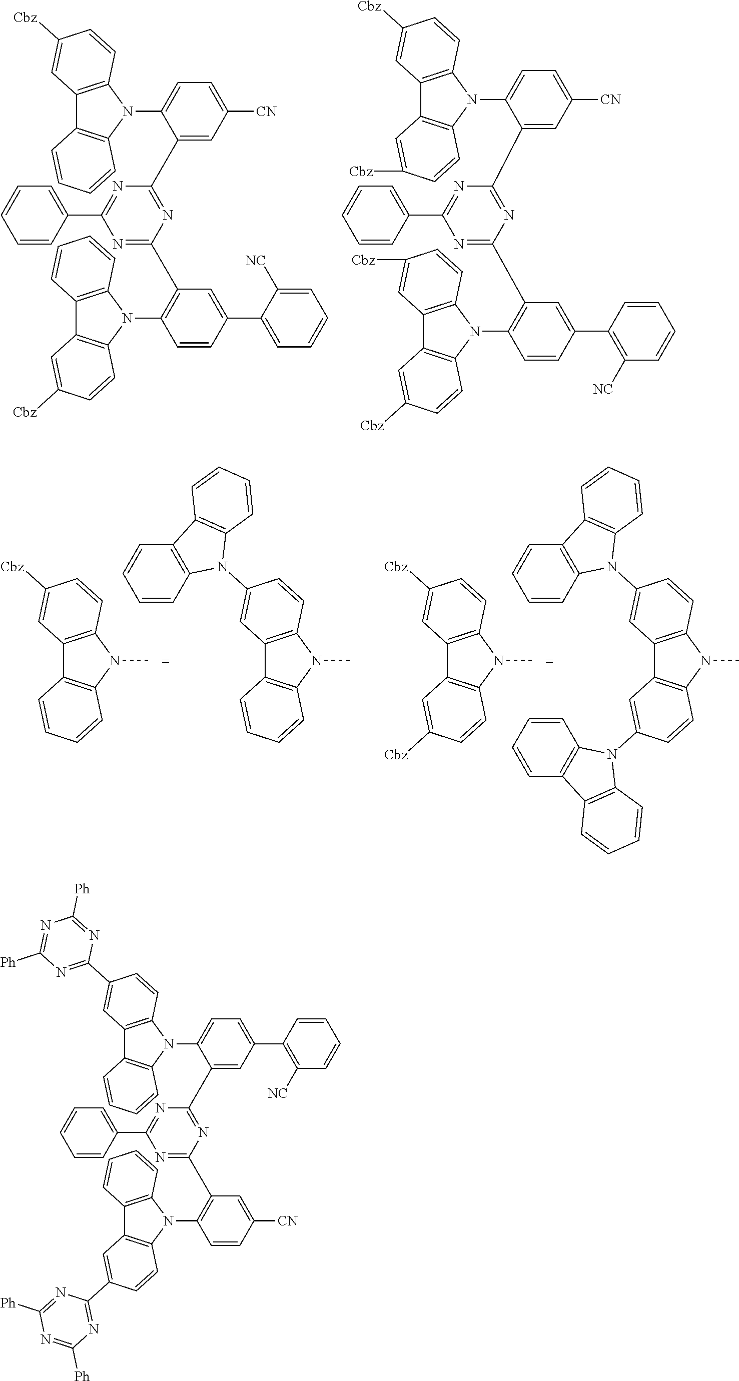

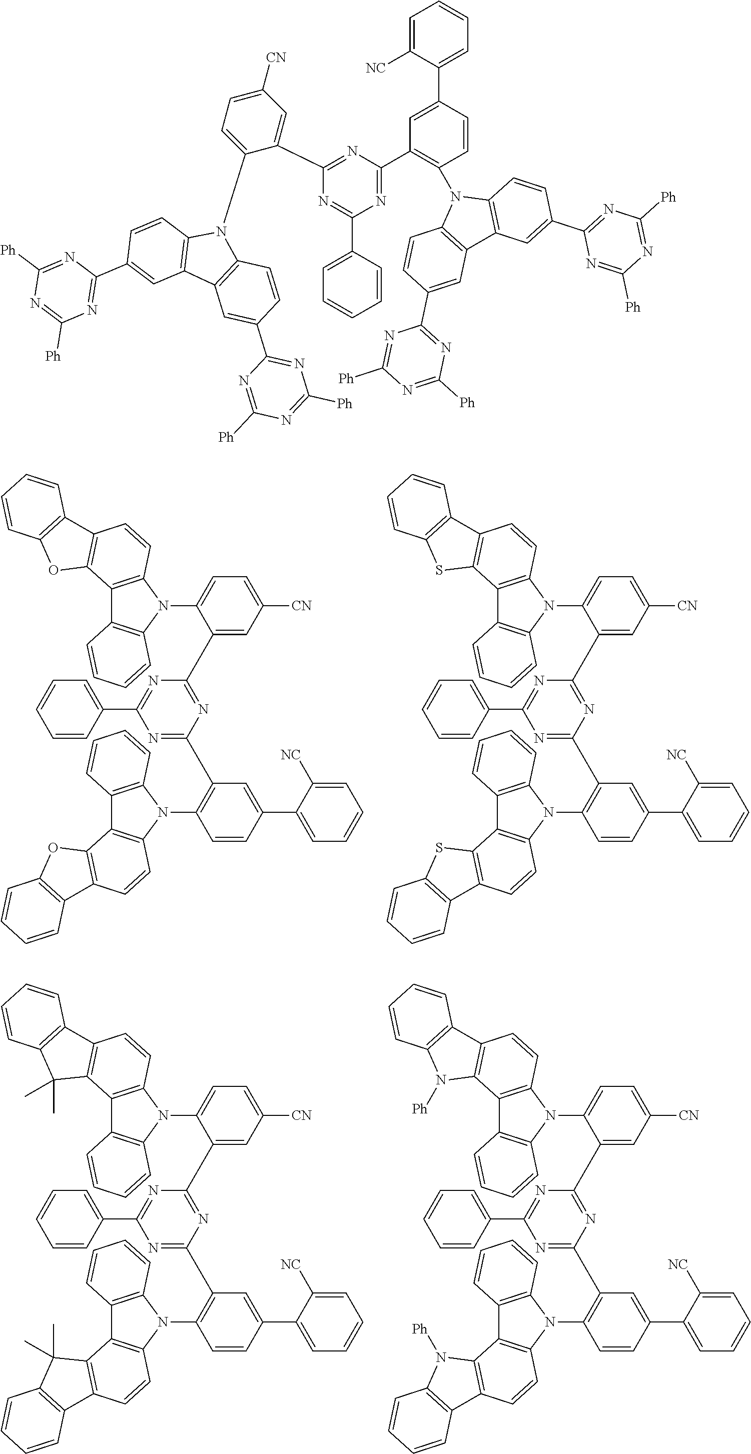

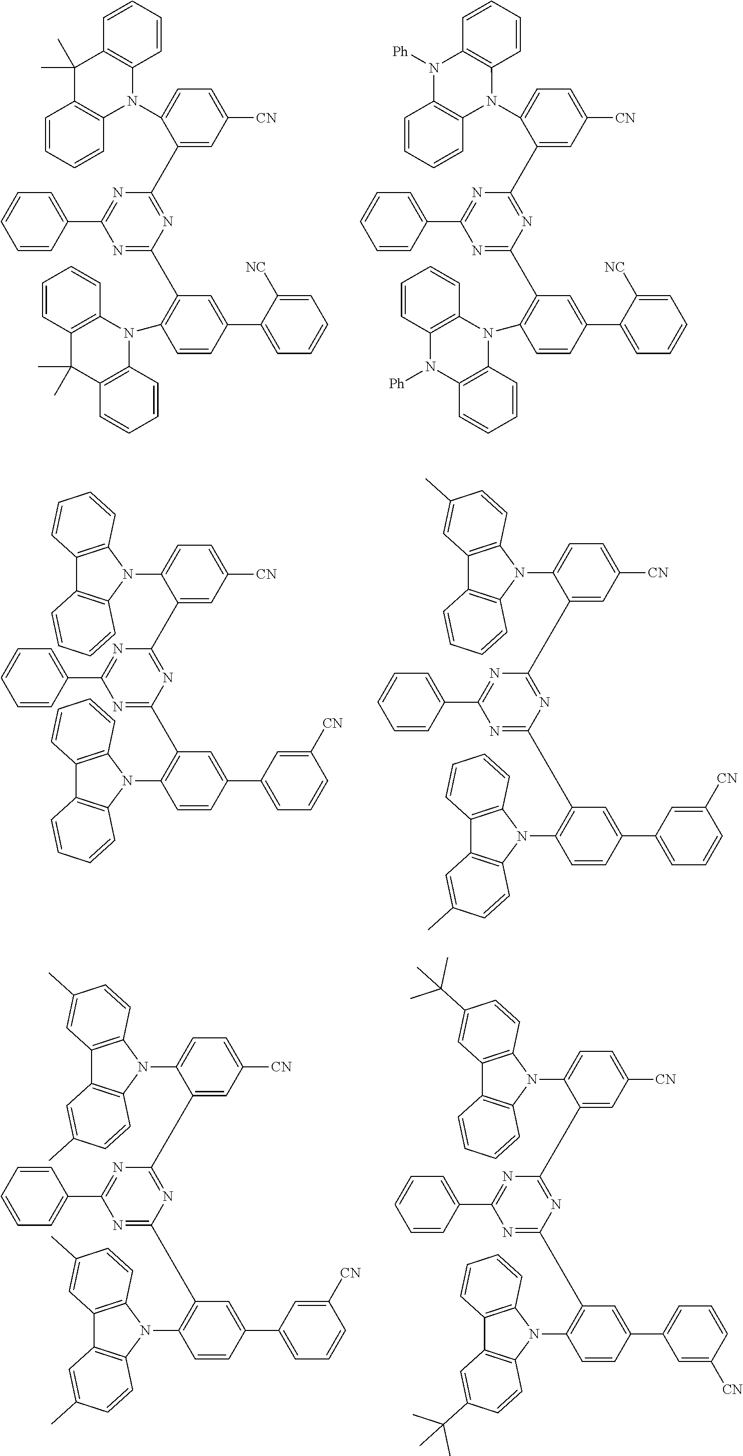

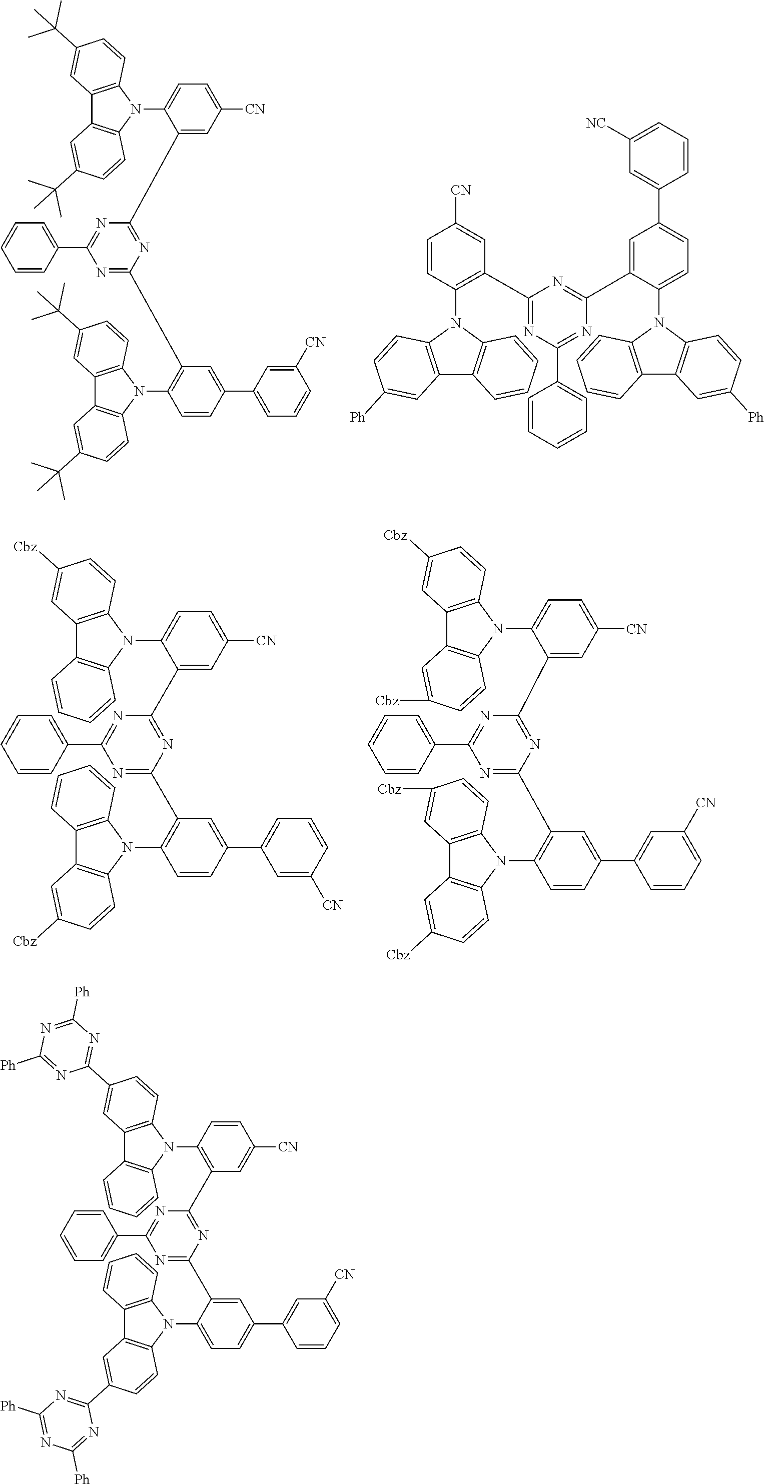

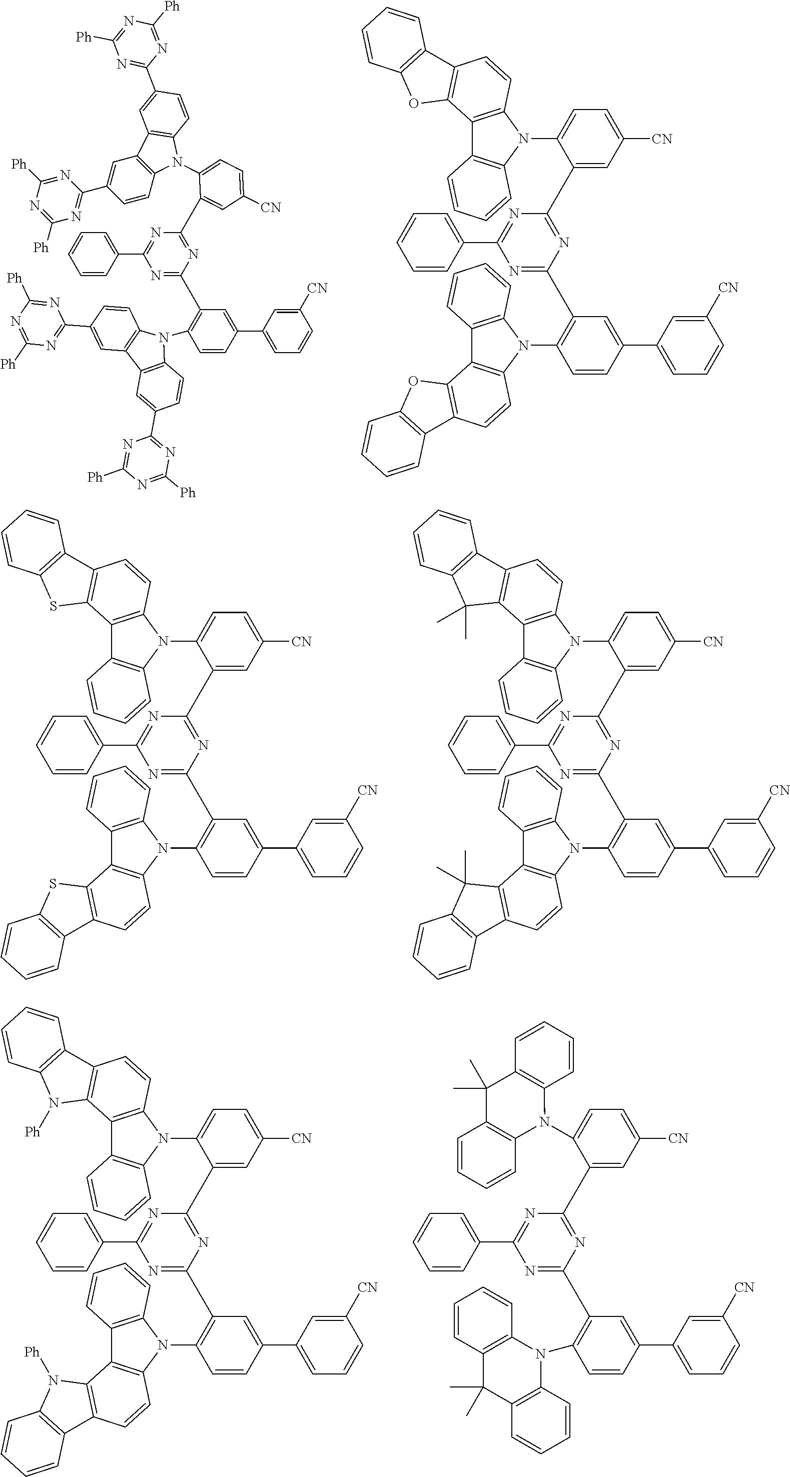

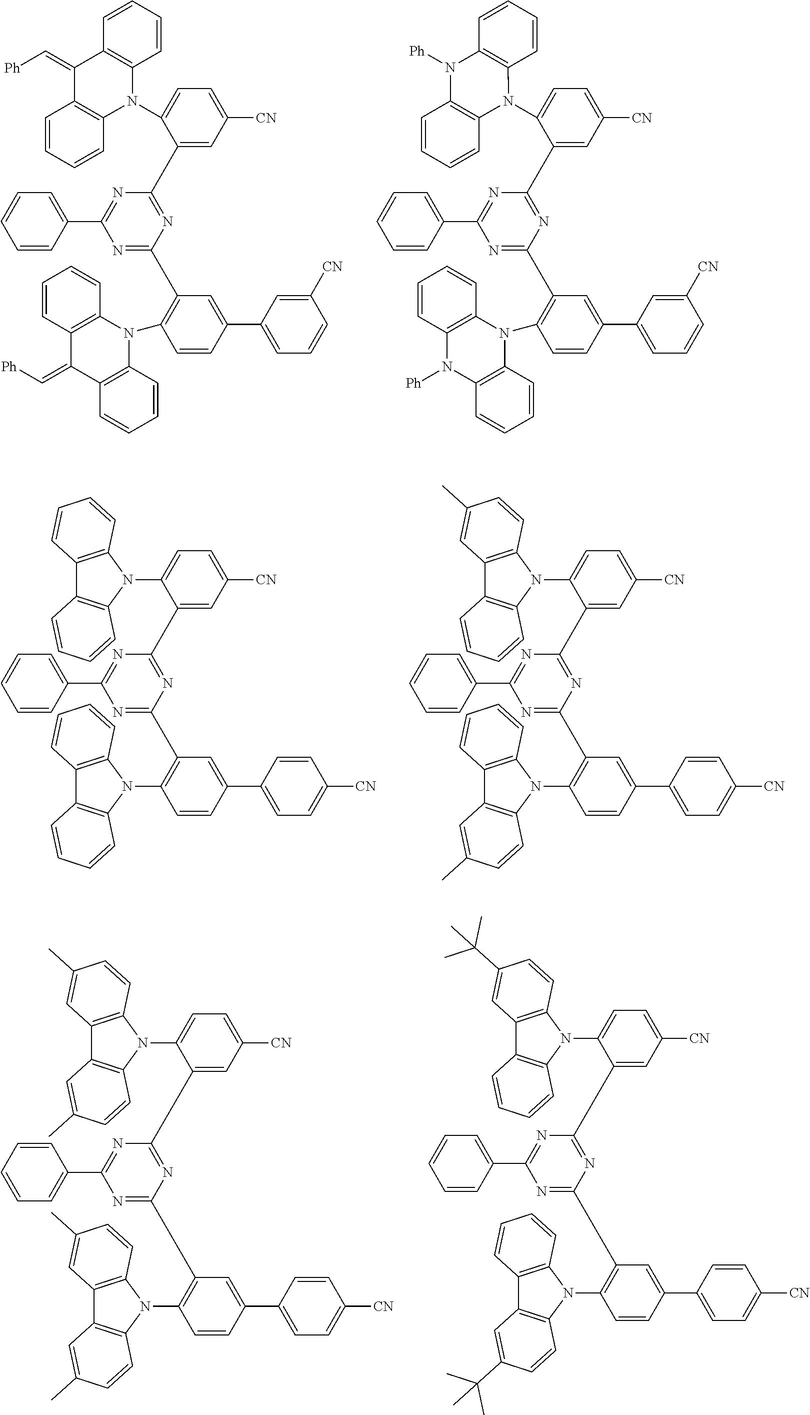

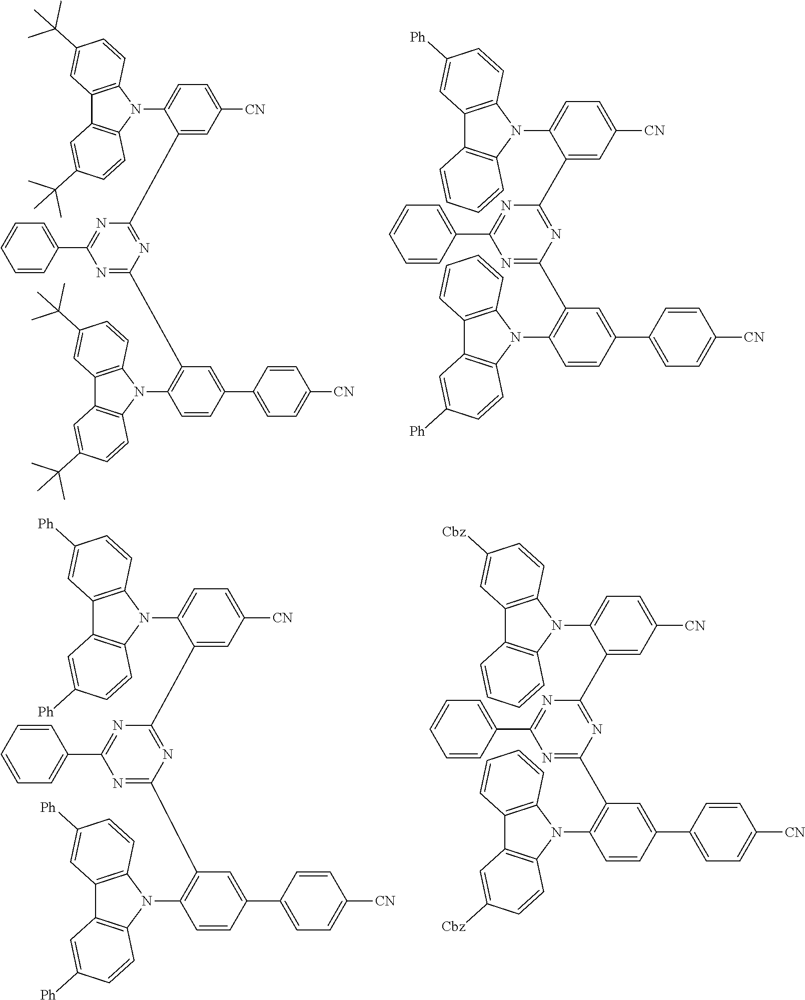

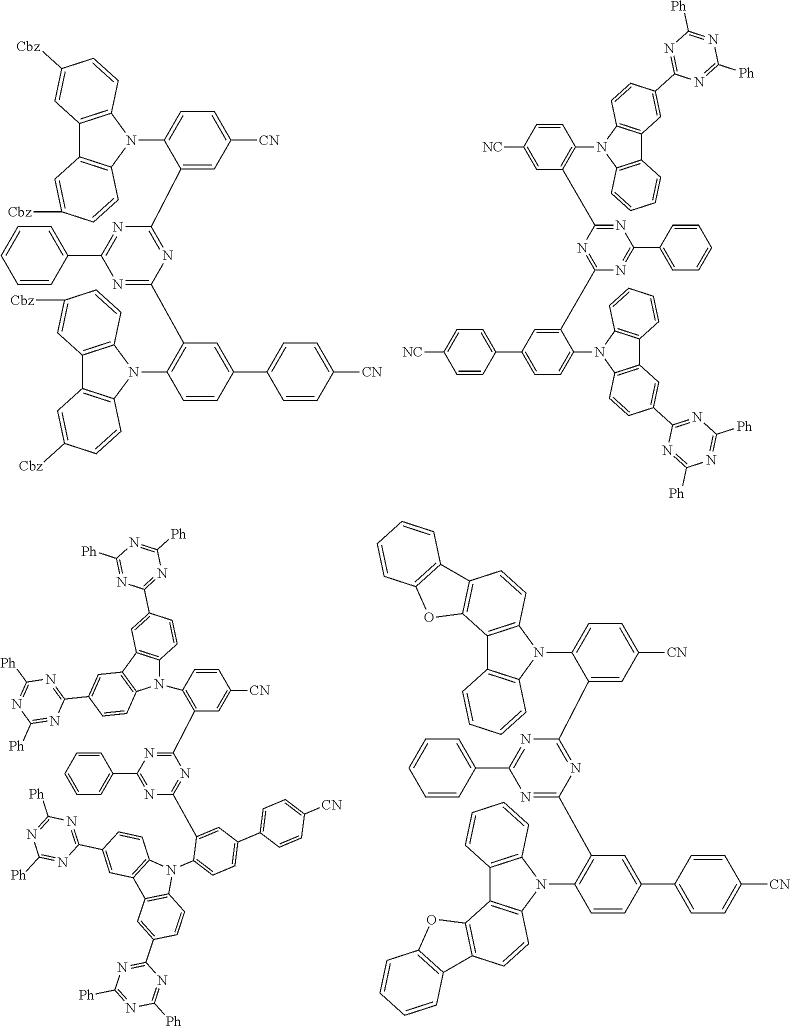

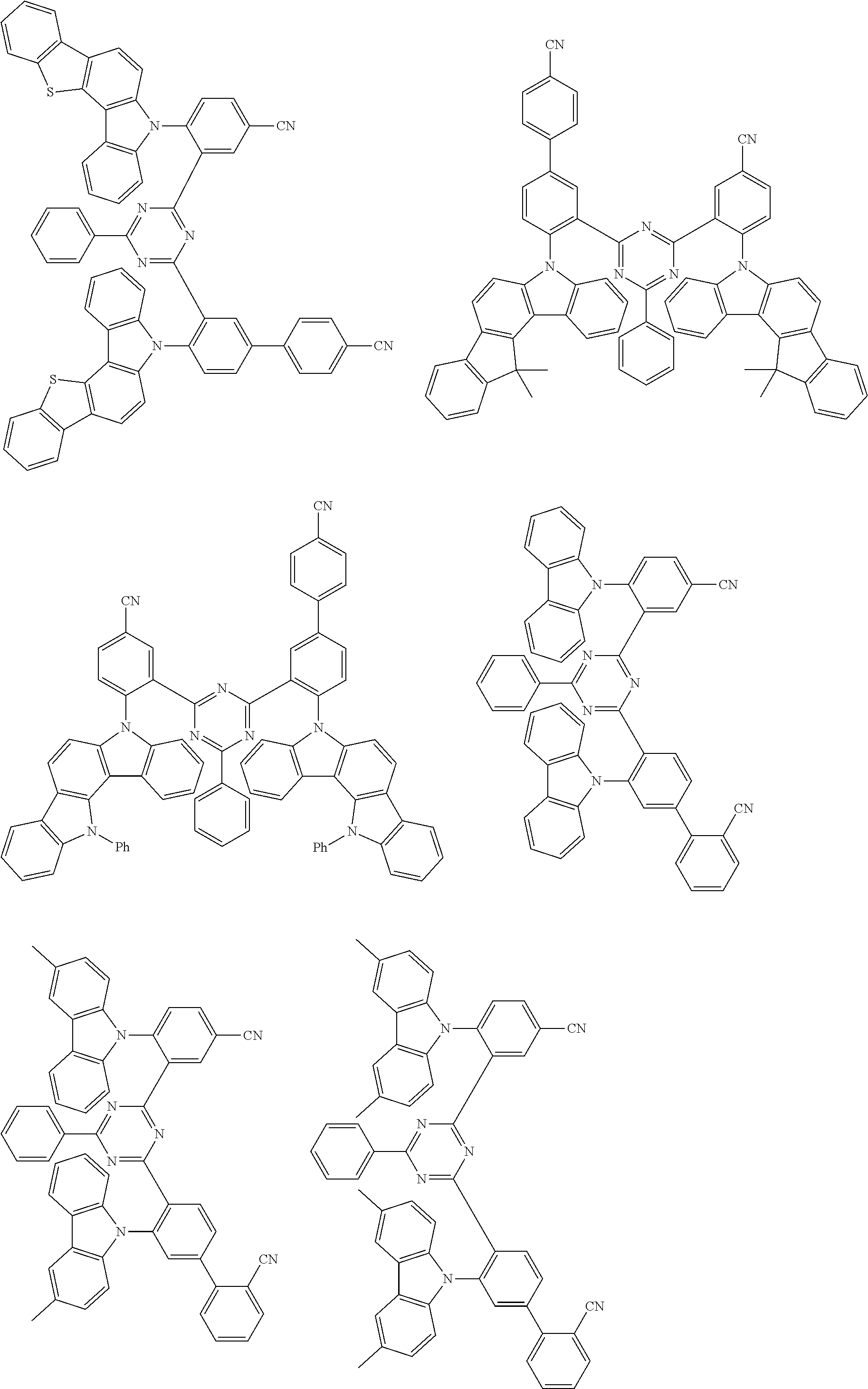

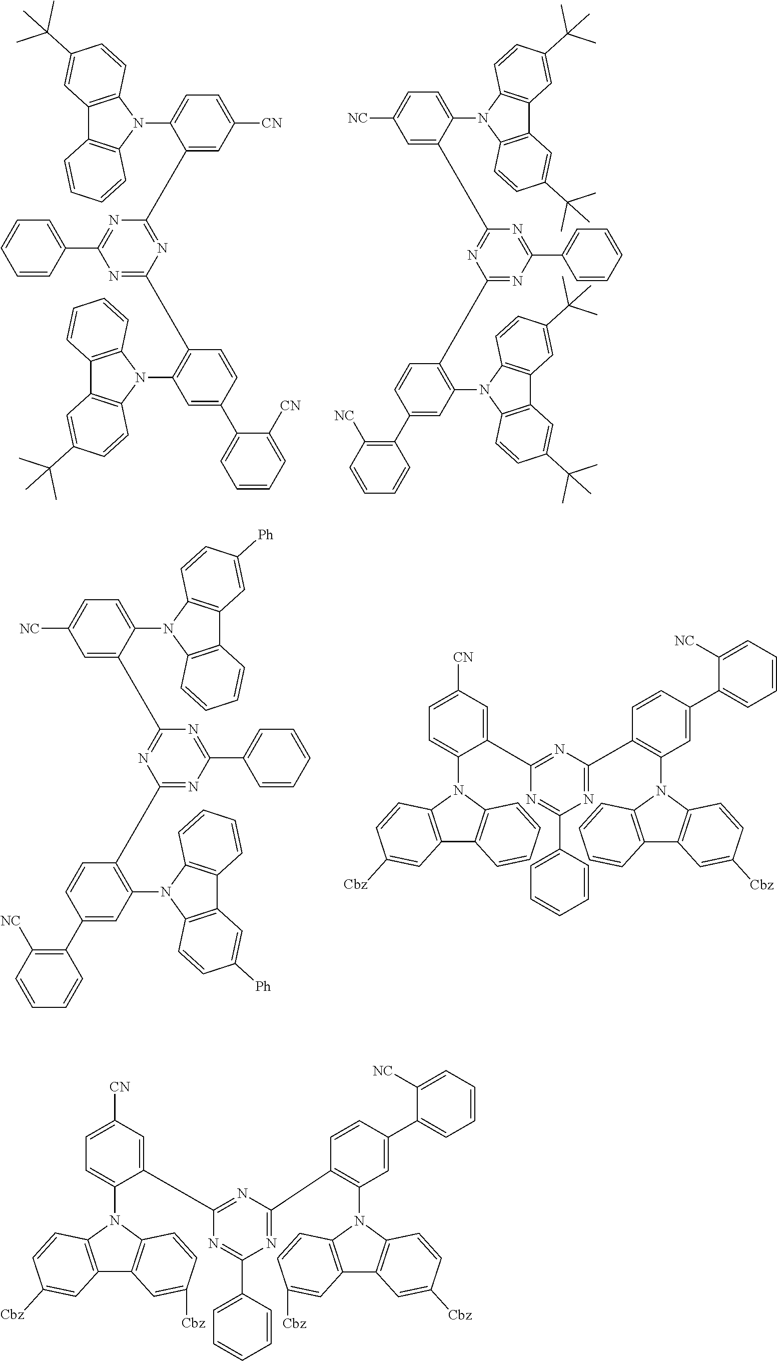

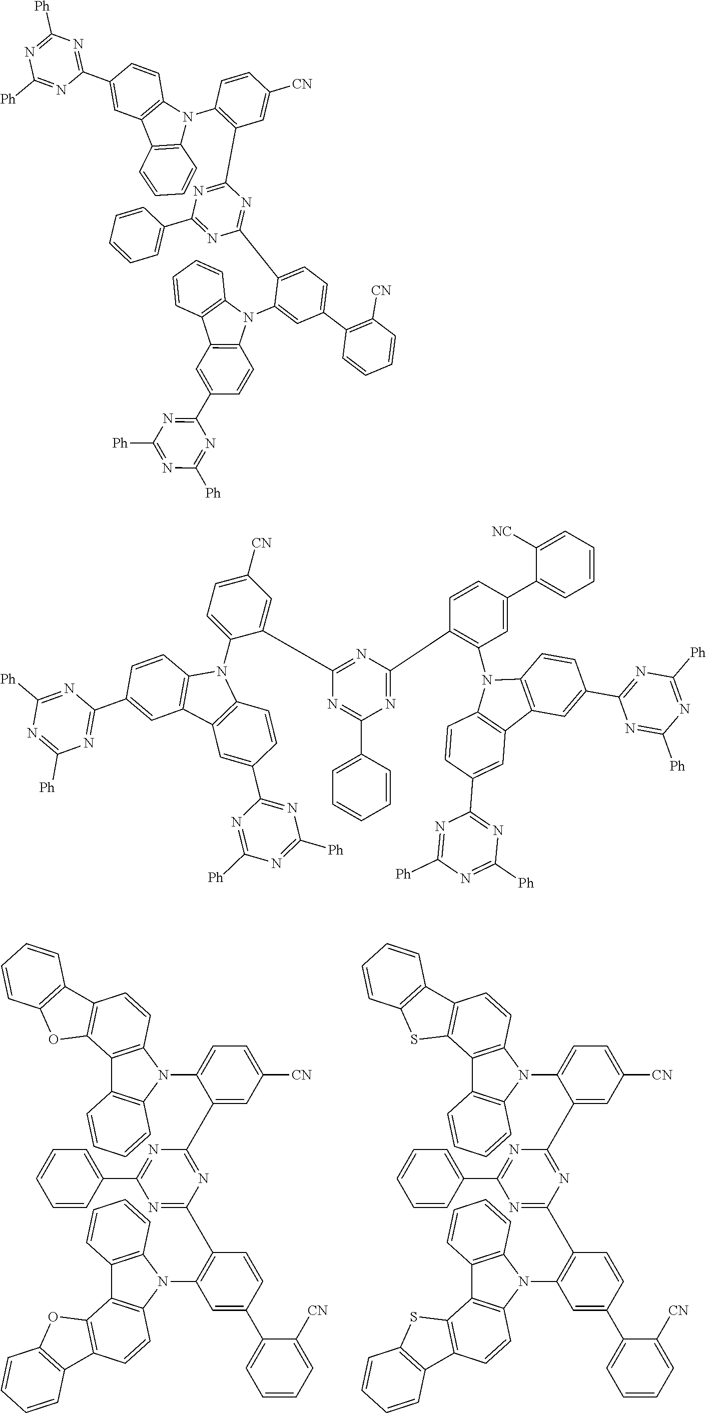

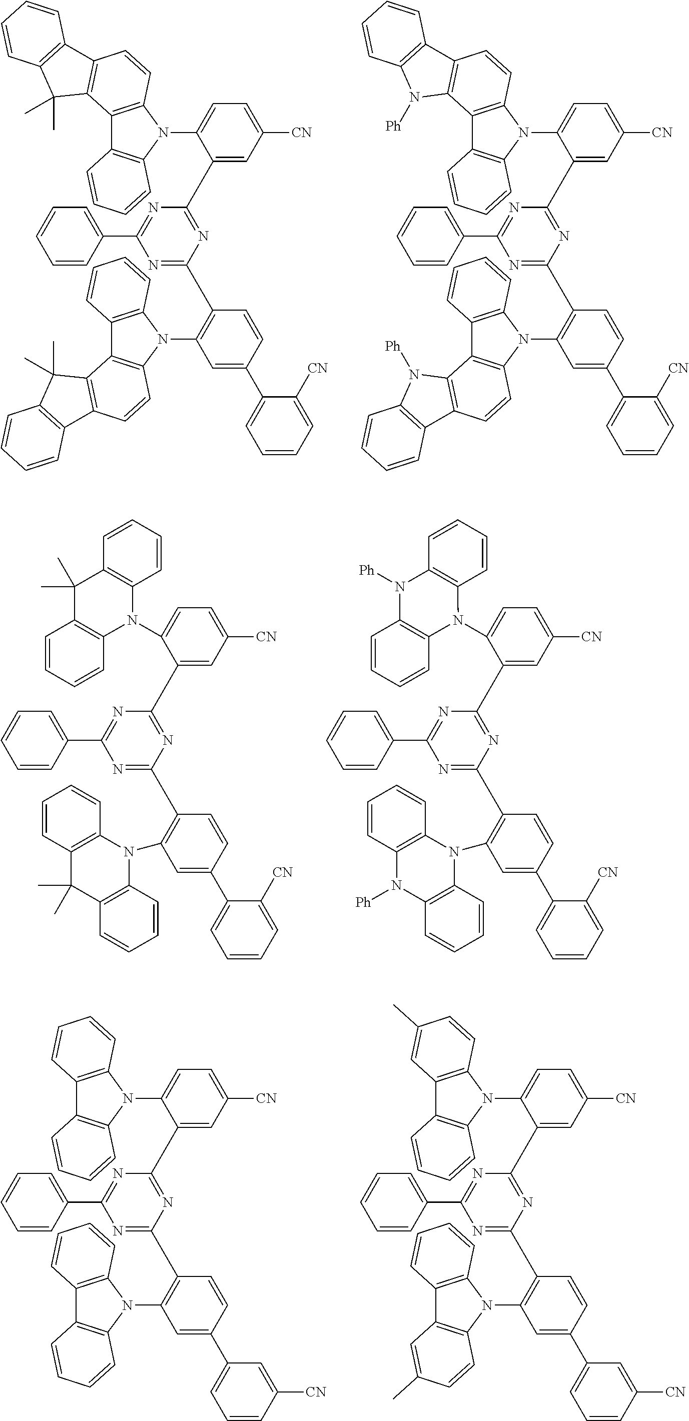

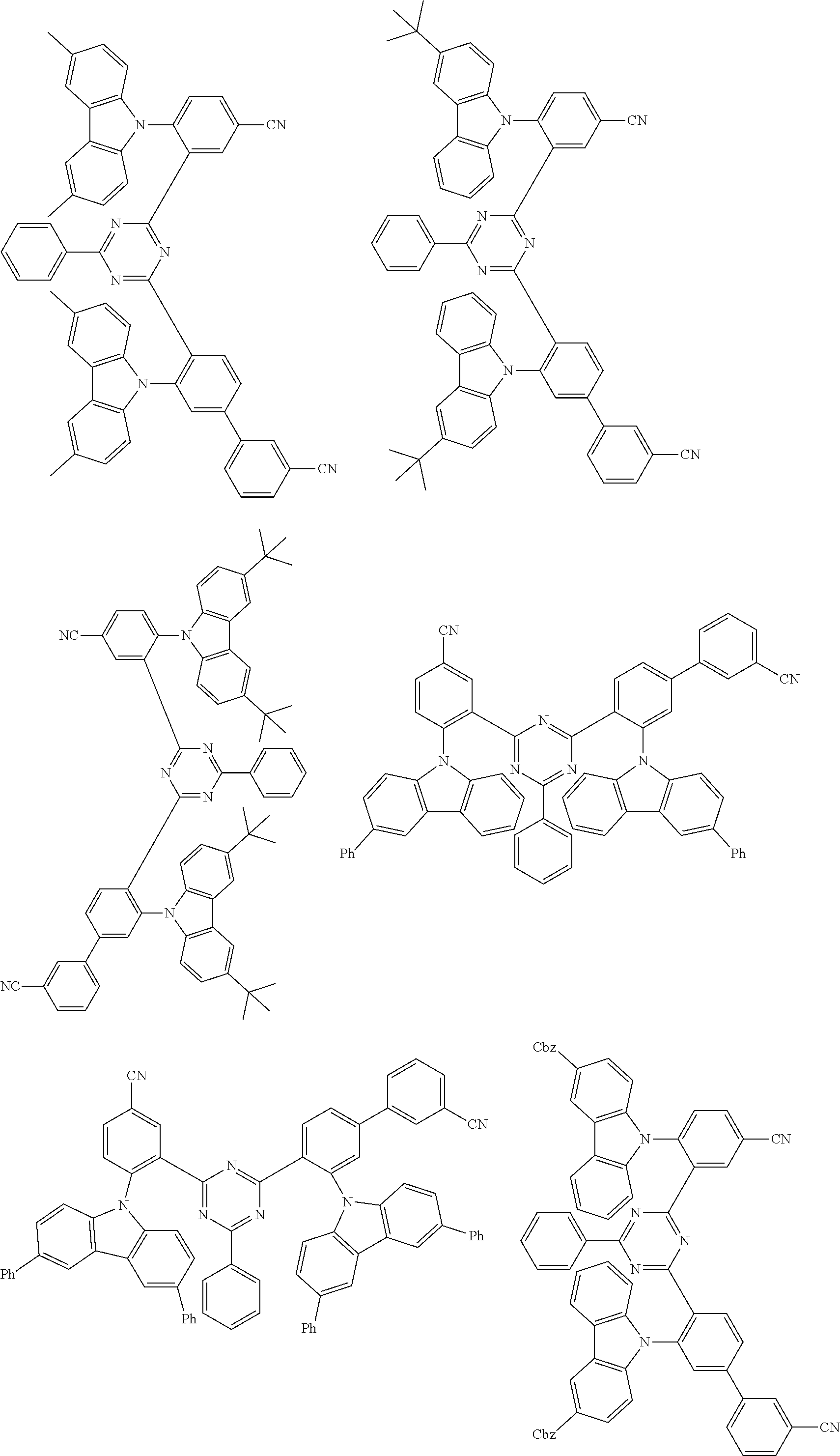

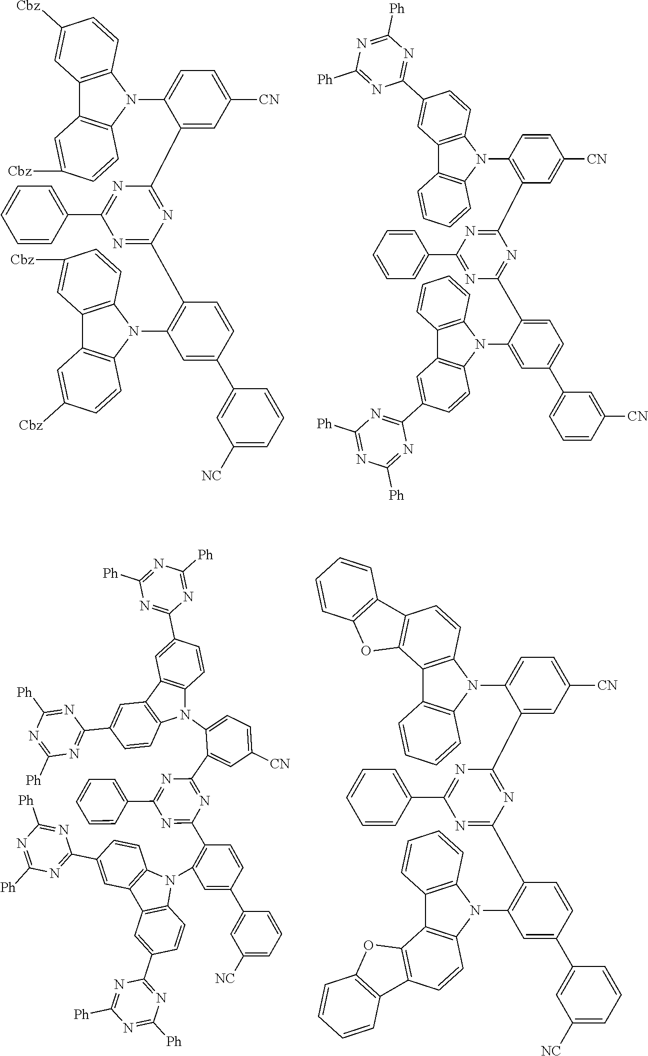

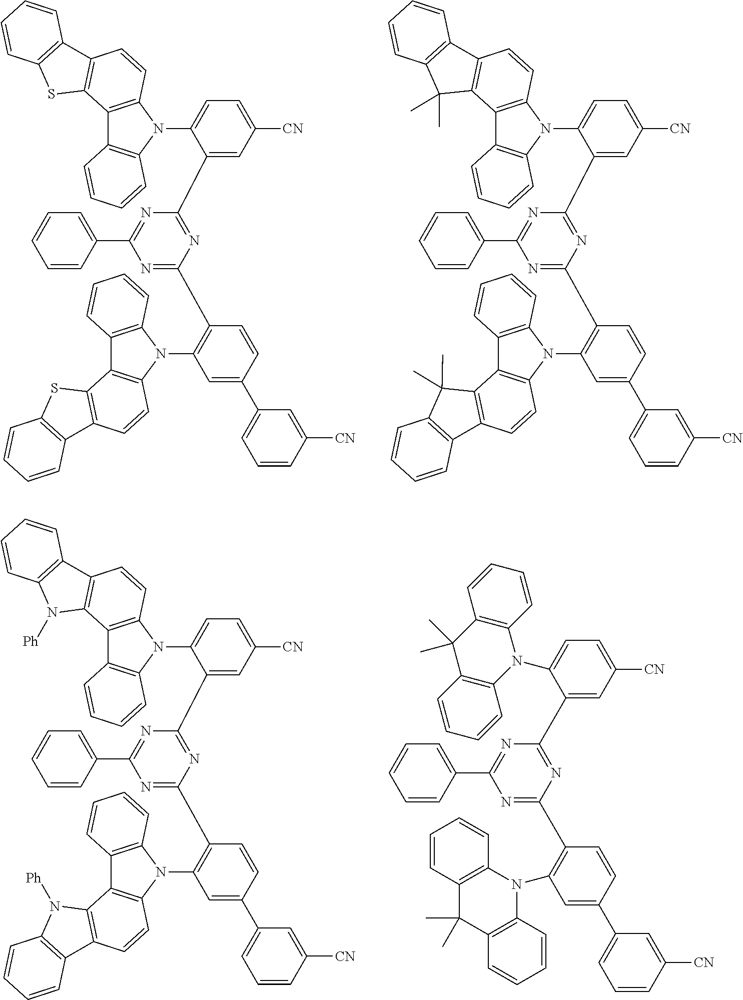

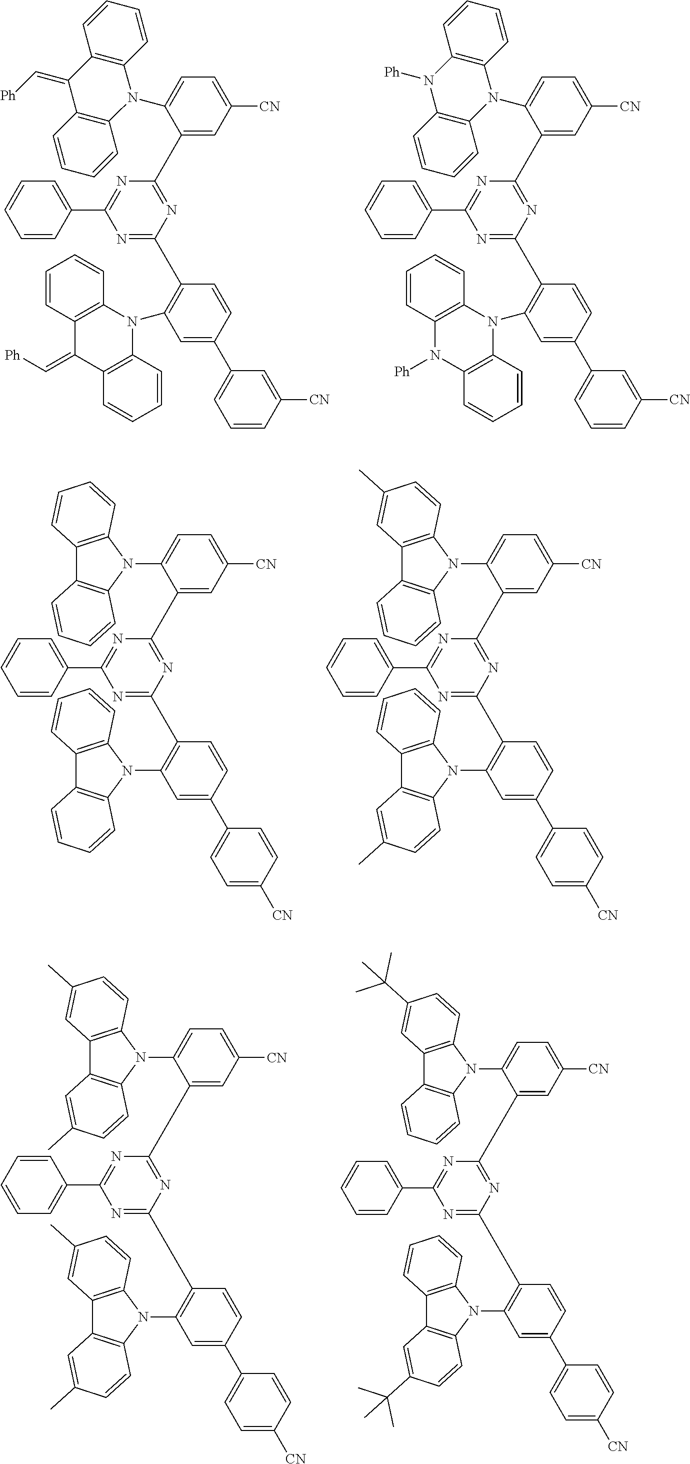

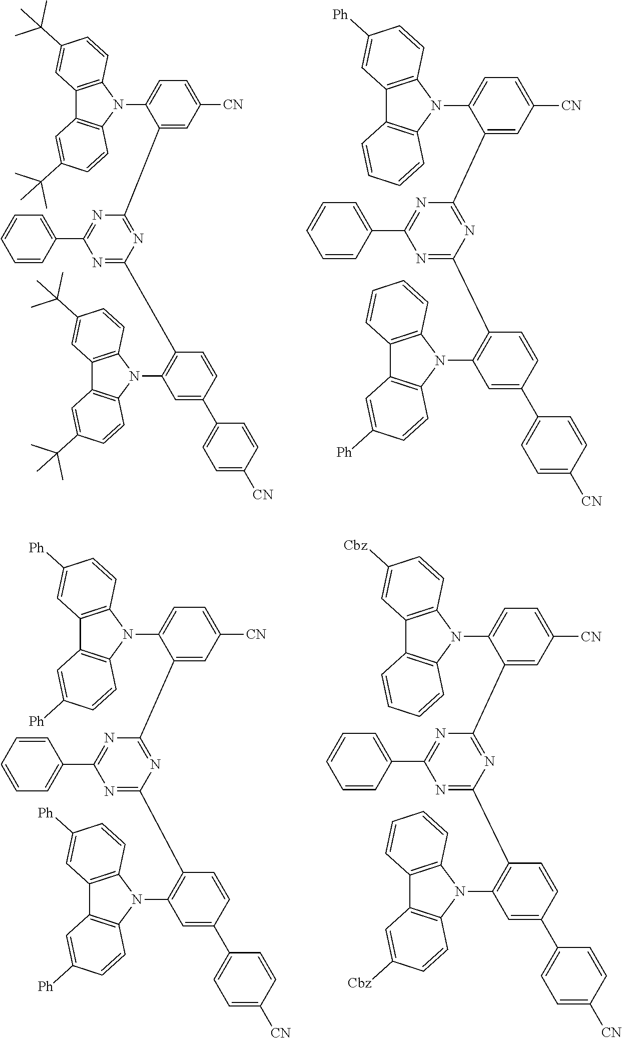

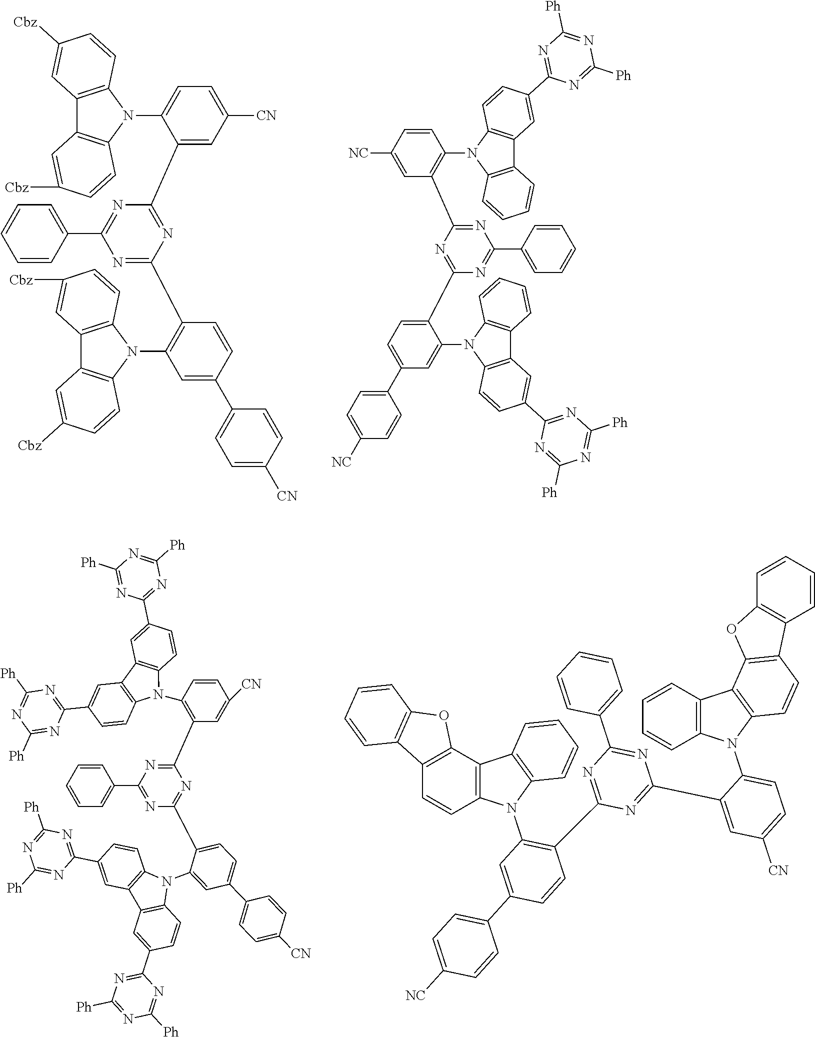

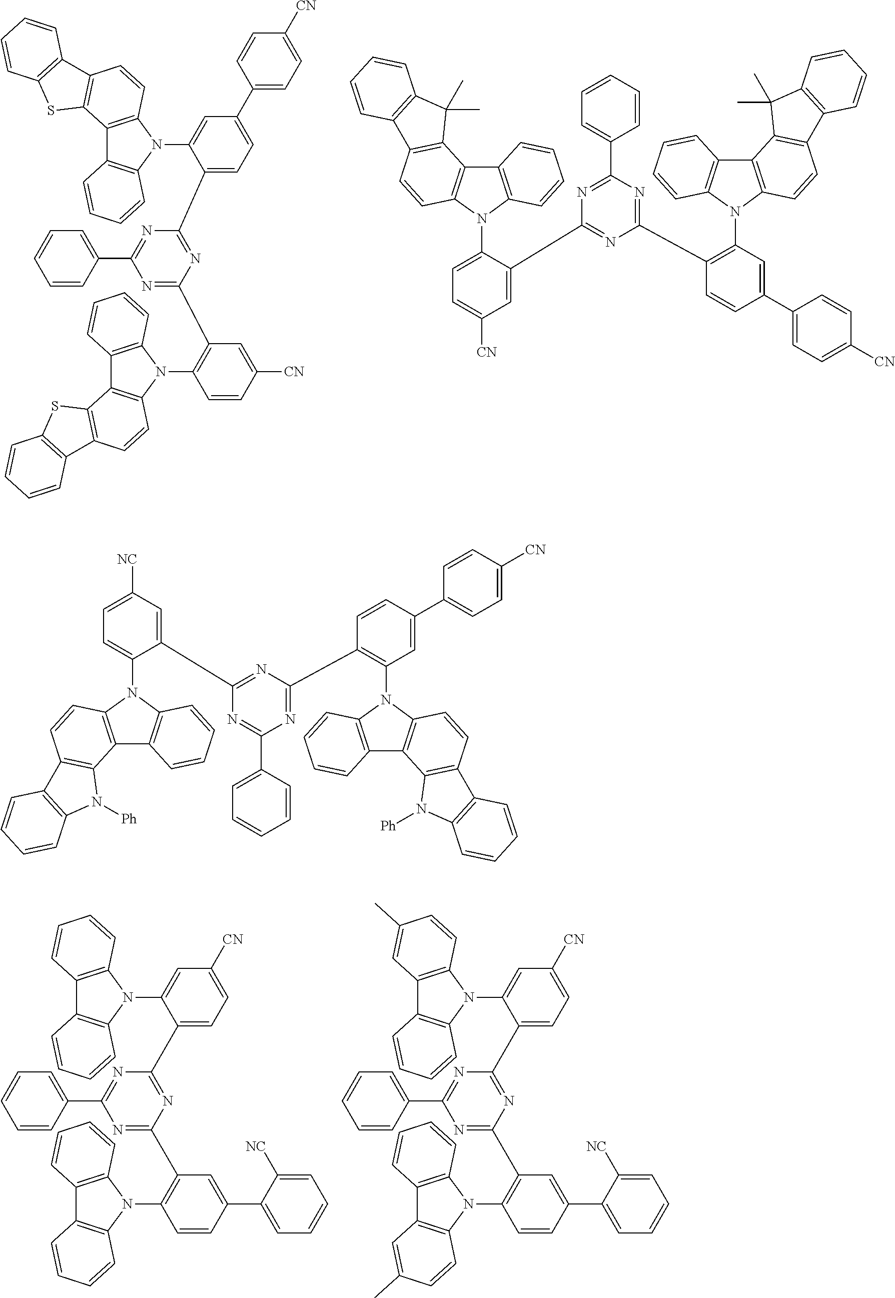

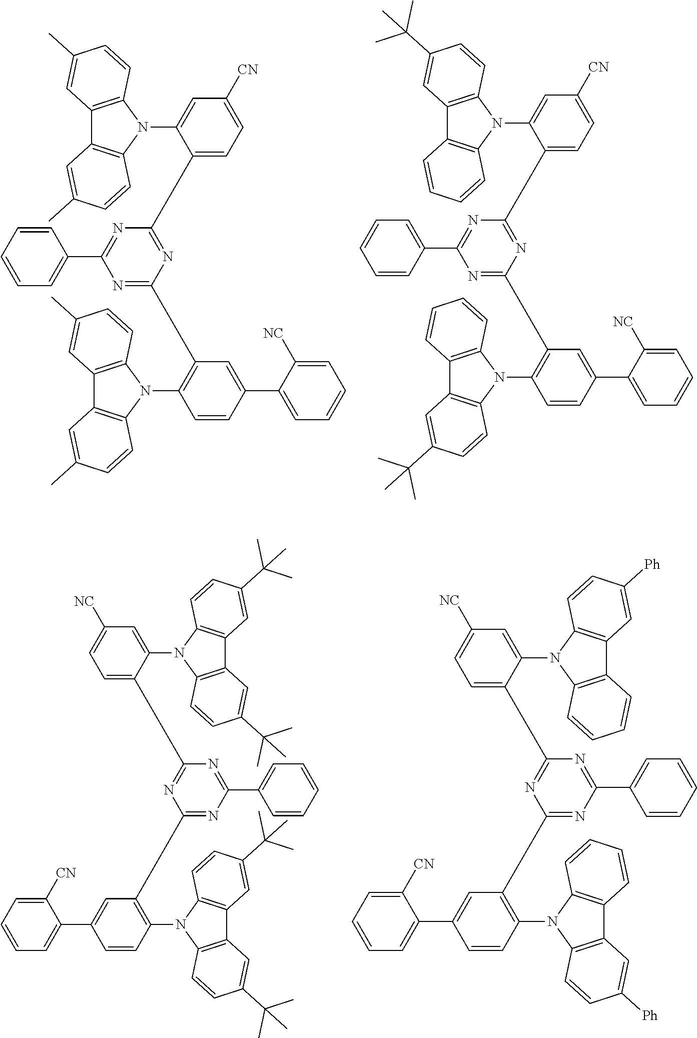 [SMILES: Cc1ccc2c(c1)c1cc(C)ccc1n2-c1ccc(-c2ccccc2C#N)cc1-c1nc(-c2ccccc2)nc(-c2ccc(C#N)cc2-n2c3ccc(C)cc3c3cc(C)ccc32)n1 ; CC(C)(C)c1ccc2c(c1)c1ccccc1n2-c1ccc(-c2ccccc2C#N)cc1-c1nc(-c2ccccc2)nc(-c2ccc(C#N)cc2-n2c3ccccc3c3cc(C(C)(C)C)ccc32)n1 ; CC(C)(C)c1ccc2c(c1)c1cc(C(C)(C)C)ccc1n2-c1ccc(-c2ccccc2C#N)cc1-c1nc(-c2ccccc2)nc(-c2ccc(C#N)cc2-n2c3ccc(C(C)(C)C)cc3c3cc(C(C)(C)C)ccc32)n1 ; N#Cc1ccc(-c2nc(-c3ccccc3)nc(-c3cc(-c4ccccc4C#N)ccc3-n3c4ccccc4c4cc(-c5ccccc5)ccc43)n2)c(-n2c3ccccc3c3cc(-c4ccccc4)ccc32)c1]

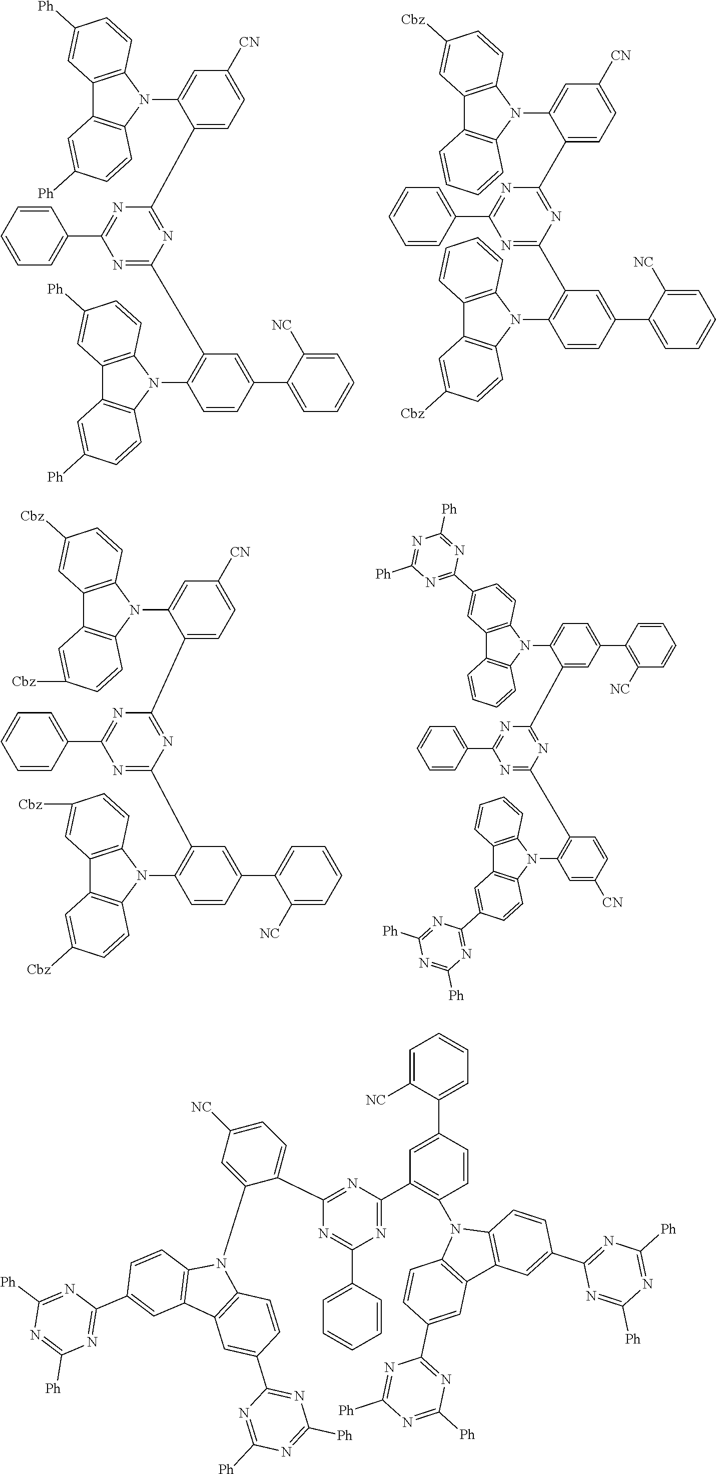

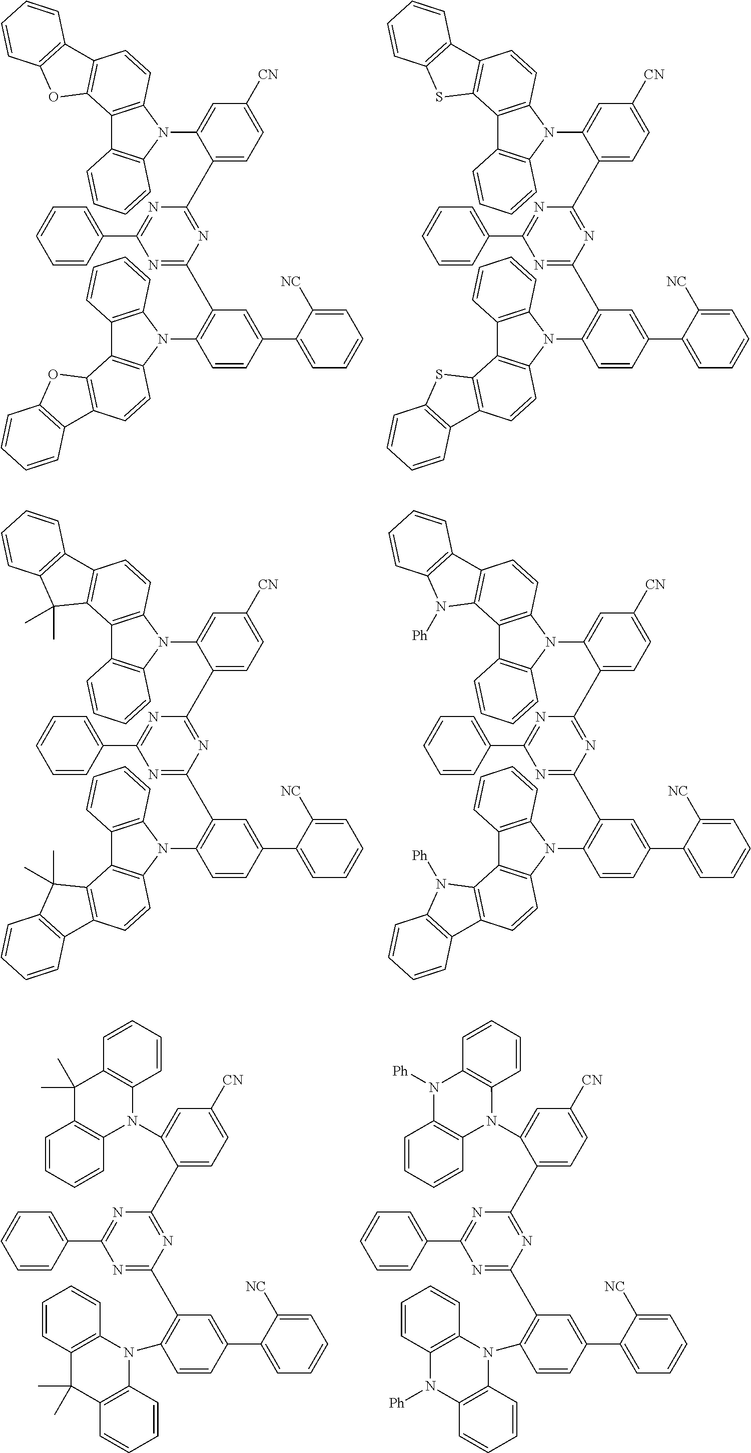

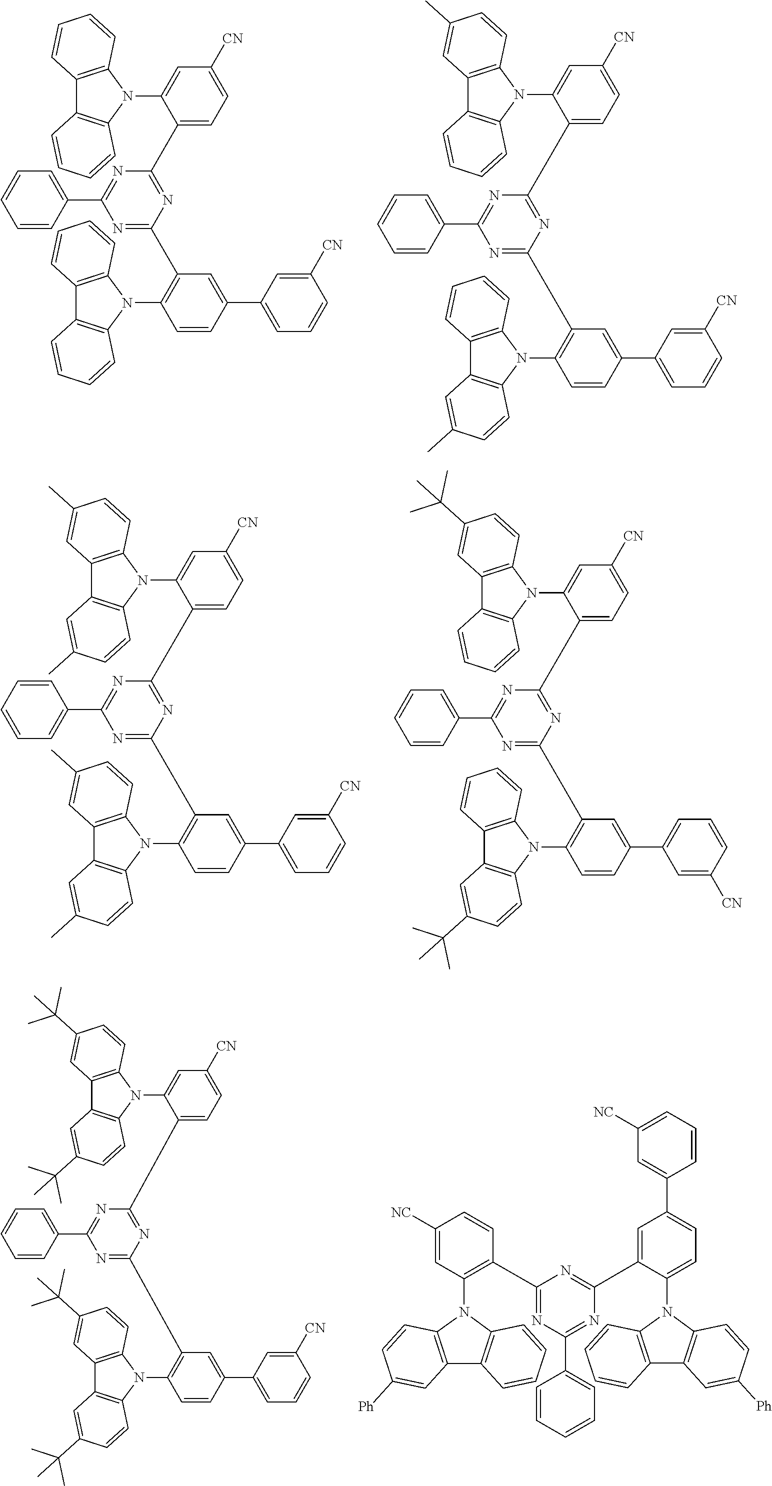

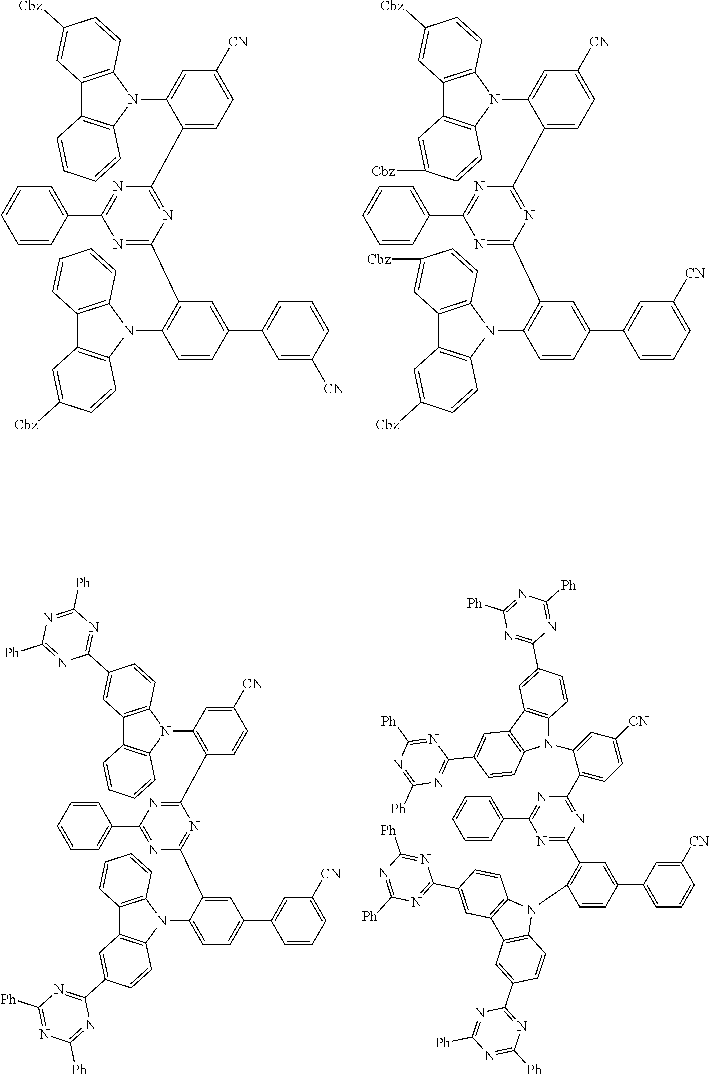

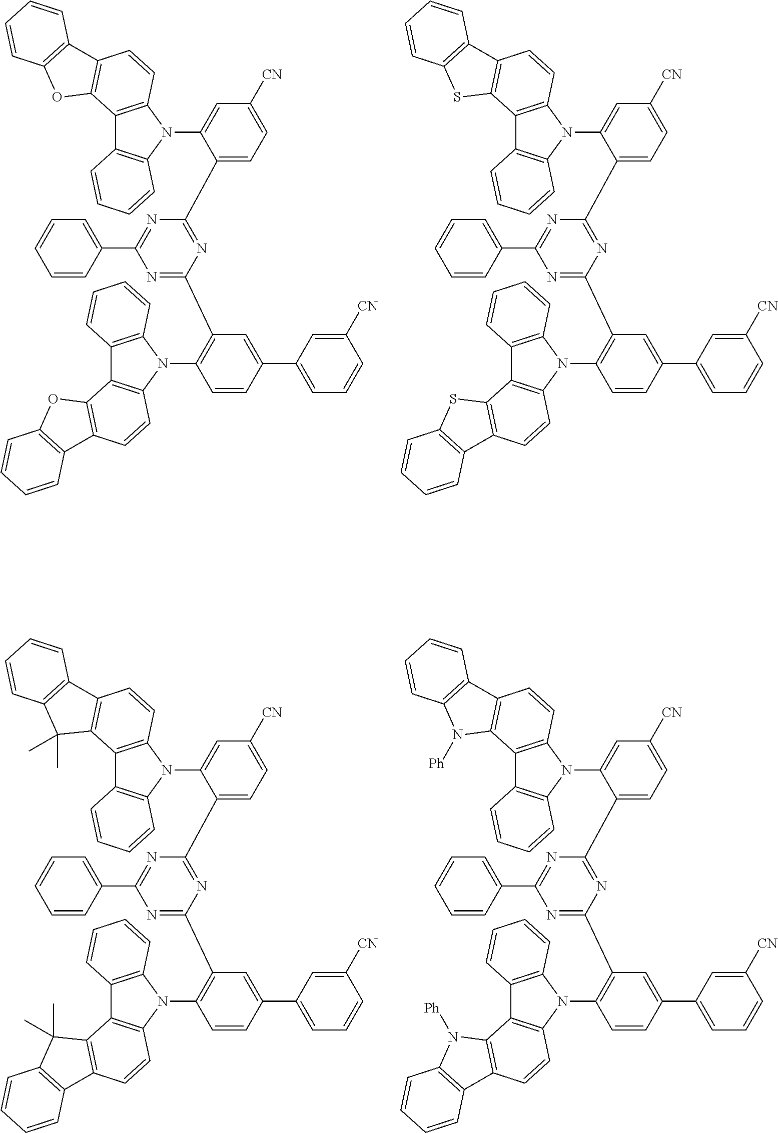

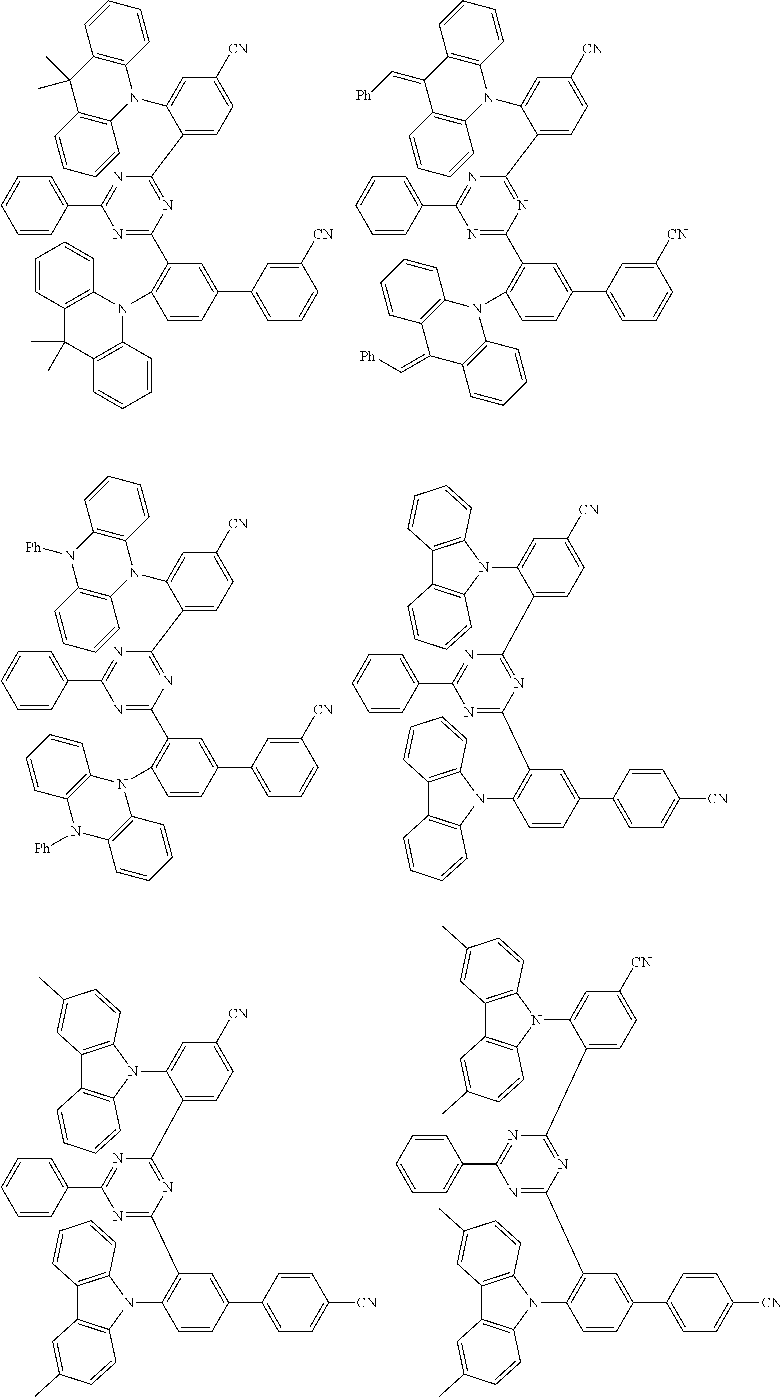

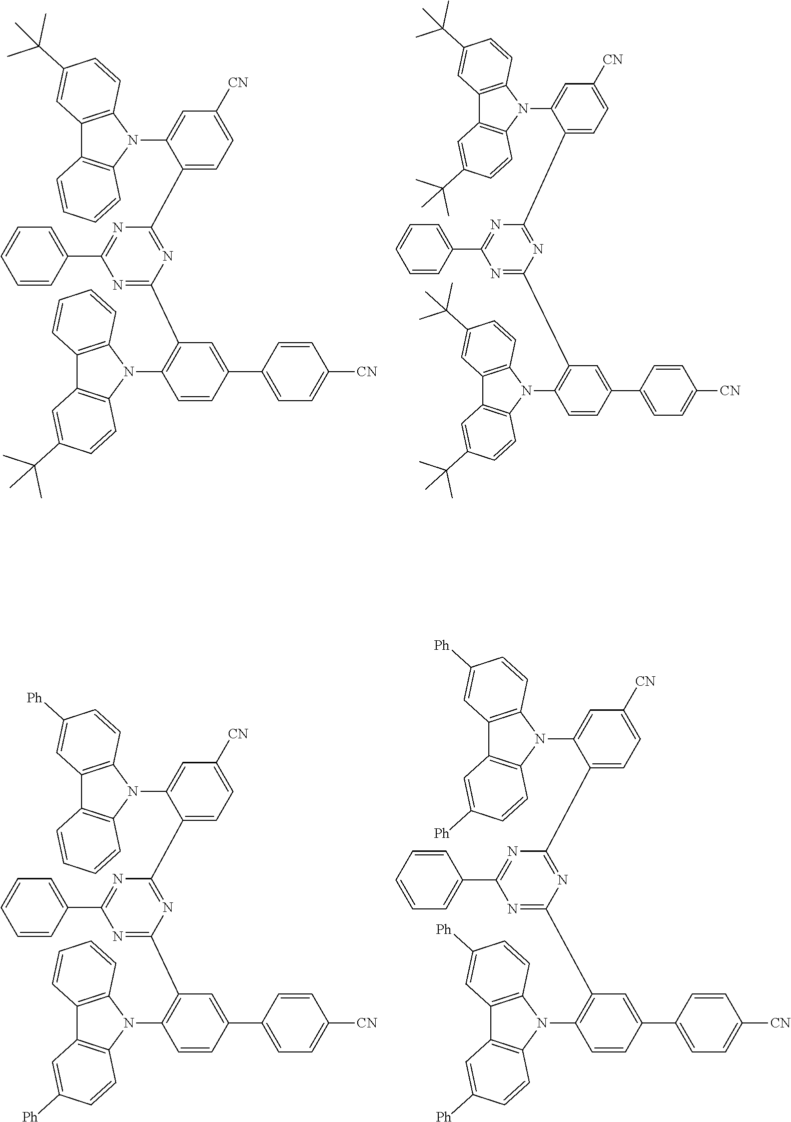

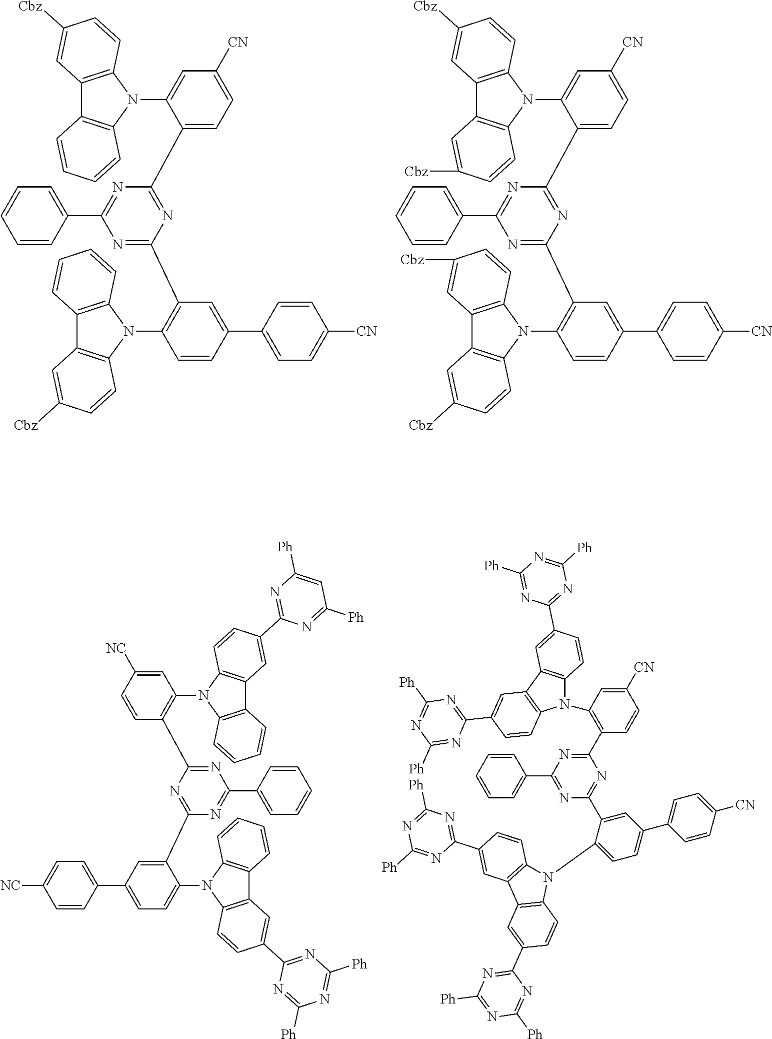

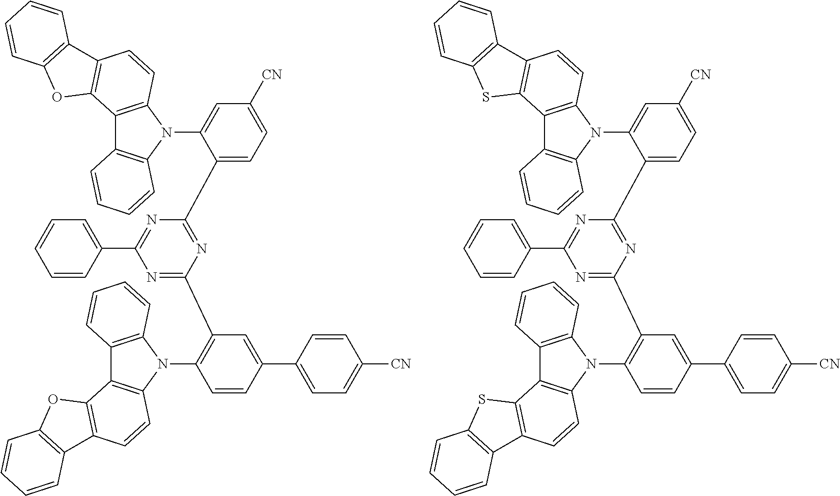 [SMILES: N#Cc1ccc(-c2ccc(-n3c4ccccc4c4c5oc6ccccc6c5ccc43)c(-c3nc(-c4ccccc4)nc(-c4ccc(C#N)cc4-n4c5ccccc5c5c6oc7ccccc7c6ccc54)n3)c2)cc1 ; N#Cc1ccc(-c2ccc(-n3c4ccccc4c4c5sc6ccccc6c5ccc43)c(-c3nc(-c4ccccc4)nc(-c4ccc(C#N)cc4-n4c5ccccc5c5c6sc7ccccc7c6ccc54)n3)c2)cc1]

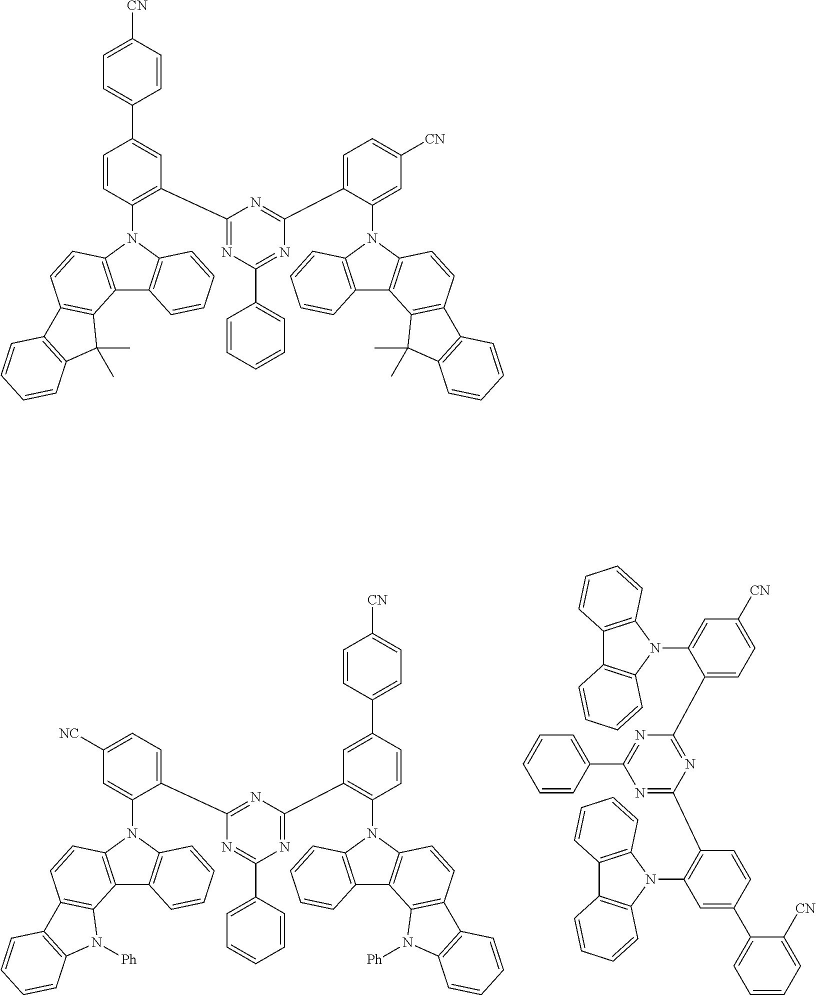 [SMILES: CC1(C)c2ccccc2-c2ccc3c(c21)c1ccccc1n3-c1ccc(-c2ccc(C#N)cc2)cc1-c1nc(-c2ccccc2)nc(-c2ccc(C#N)cc2-n2c3ccccc3c3c4c(ccc32)-c2ccccc2C4(C)C)n1 ; N#Cc1ccc(-c2ccc(-n3c4ccccc4c4c3ccc3c5ccccc5n(-c5ccccc5)c34)c(-c3nc(-c4ccccc4)nc(-c4ccc(C#N)cc4-n4c5ccccc5c5c4ccc4c6ccccc6n(-c6ccccc6)c45)n3)c2)cc1 ; N#Cc1ccc(-c2nc(-c3ccccc3)nc(-c3ccc(-c4ccccc4C#N)cc3-n3c4ccccc4c4ccccc43)n2)c(-n2c3ccccc3c3ccccc32)c1]

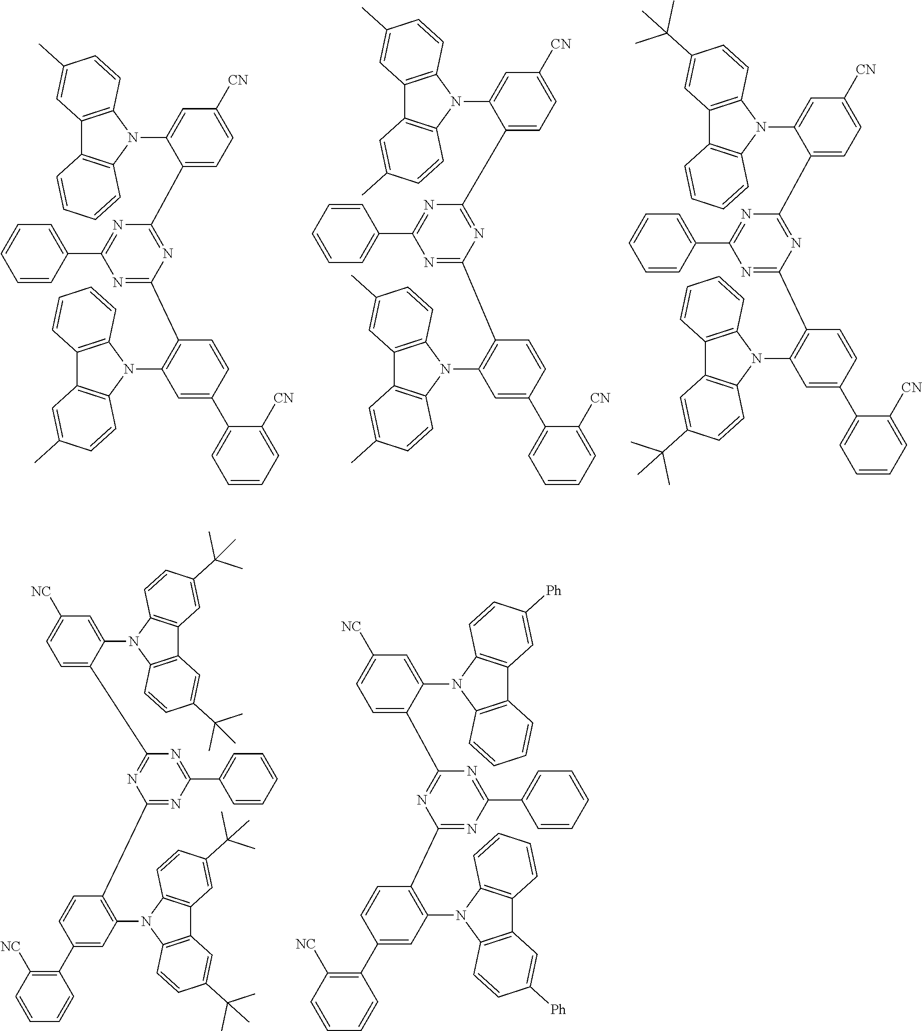

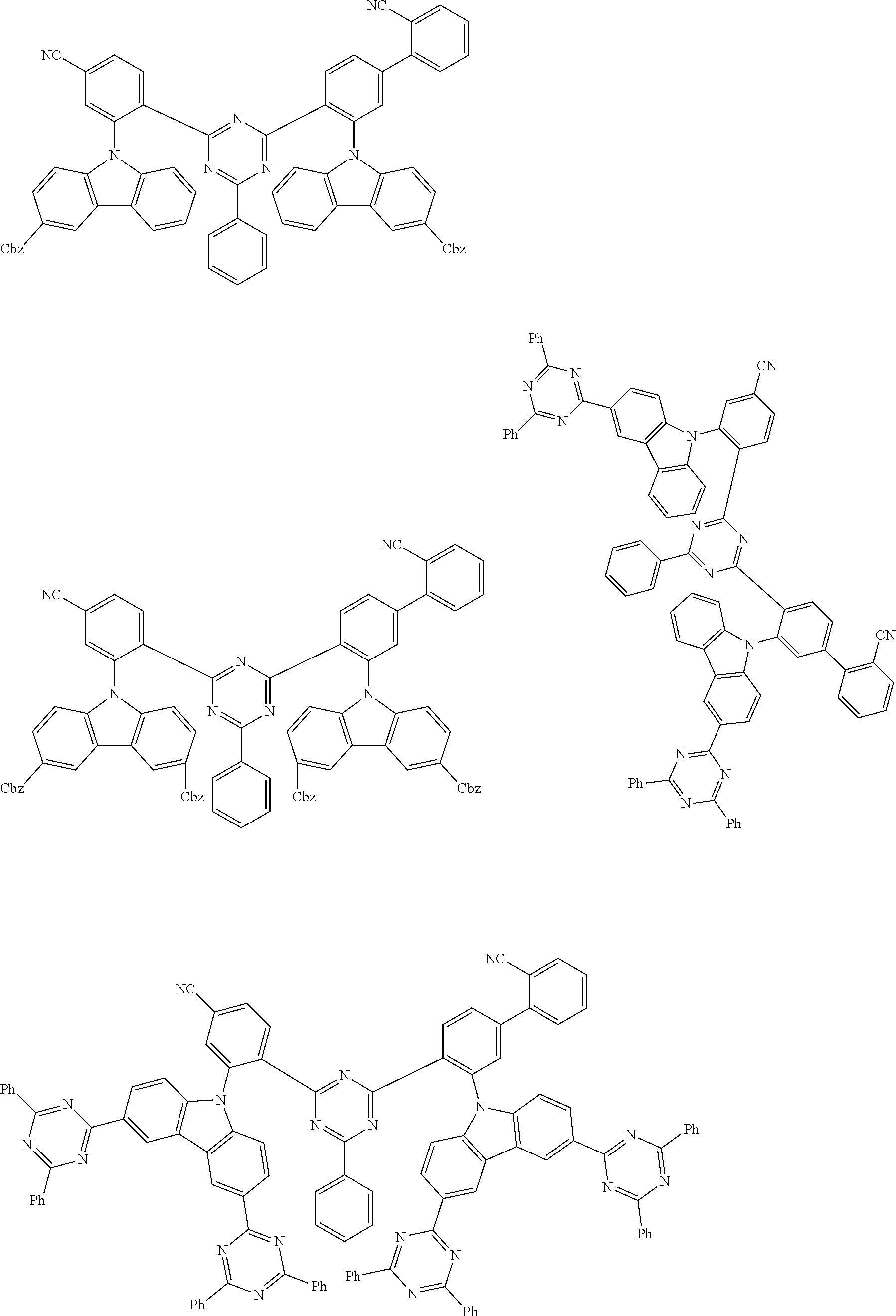

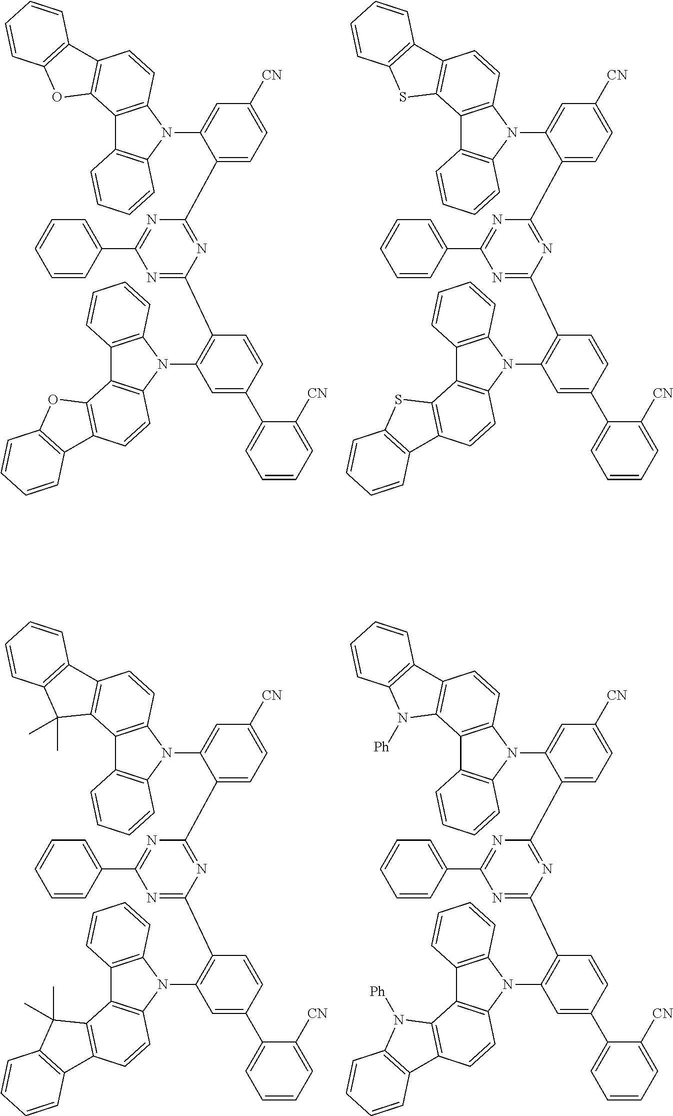

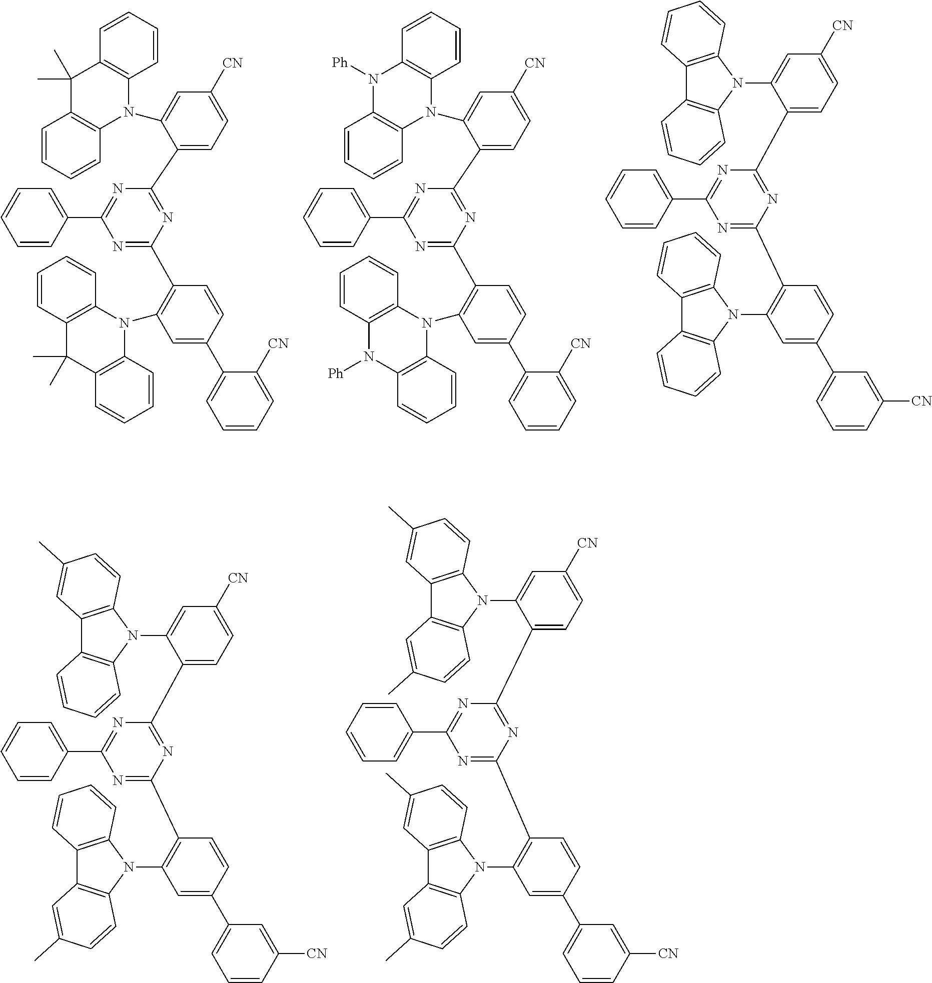

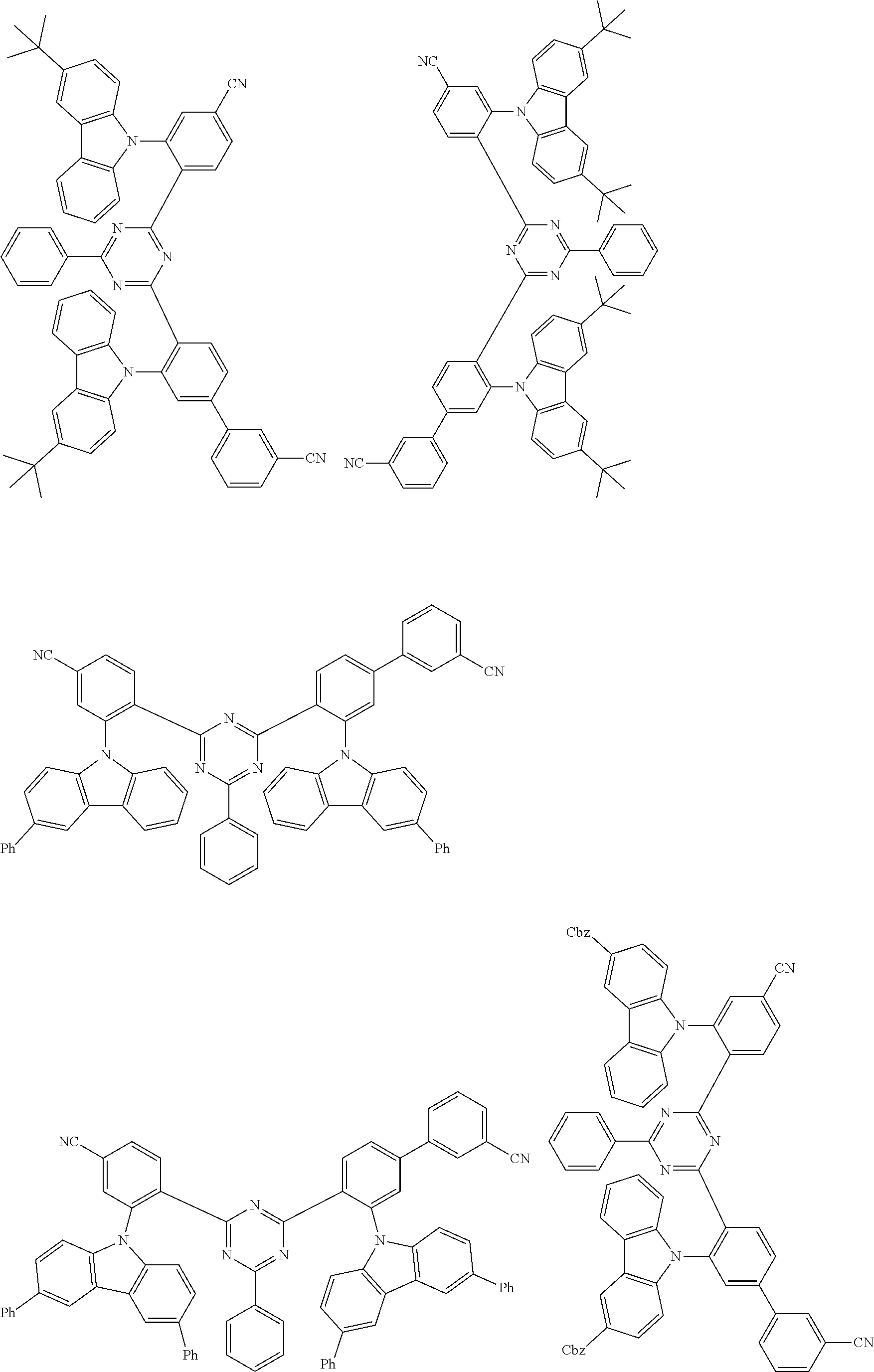

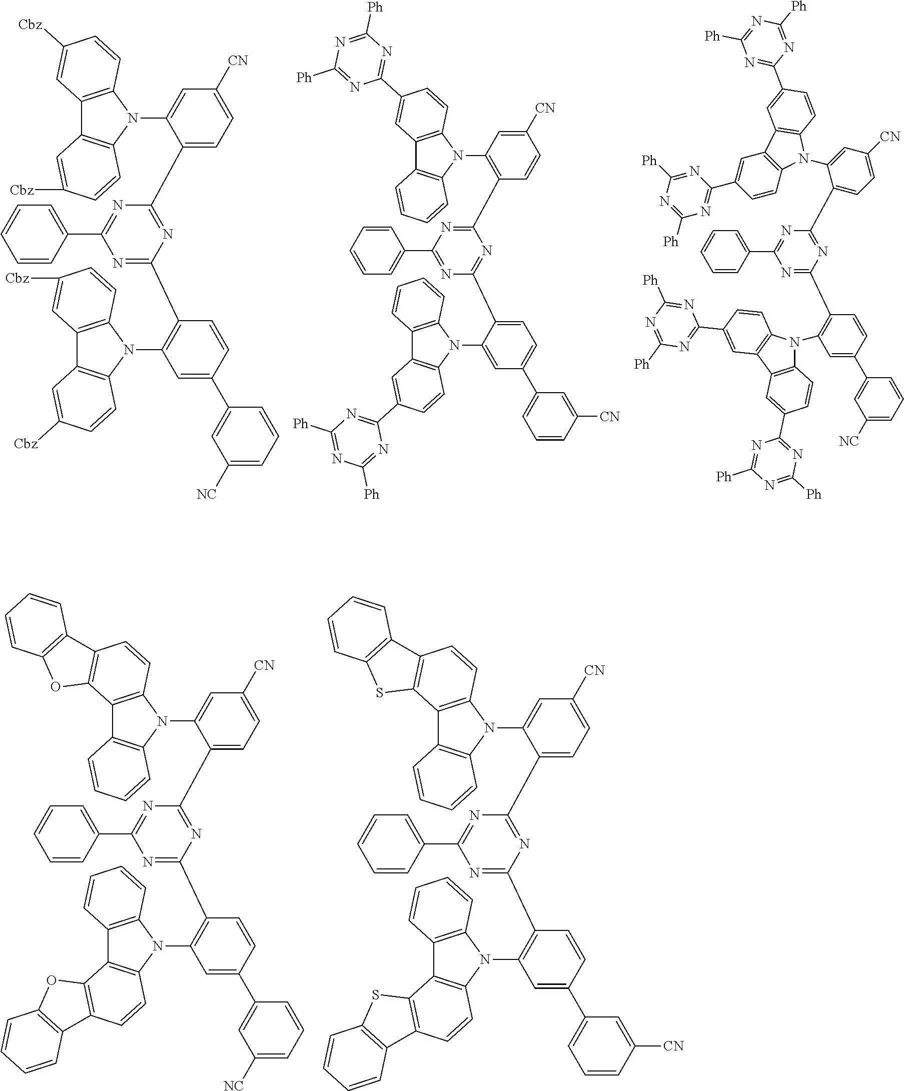

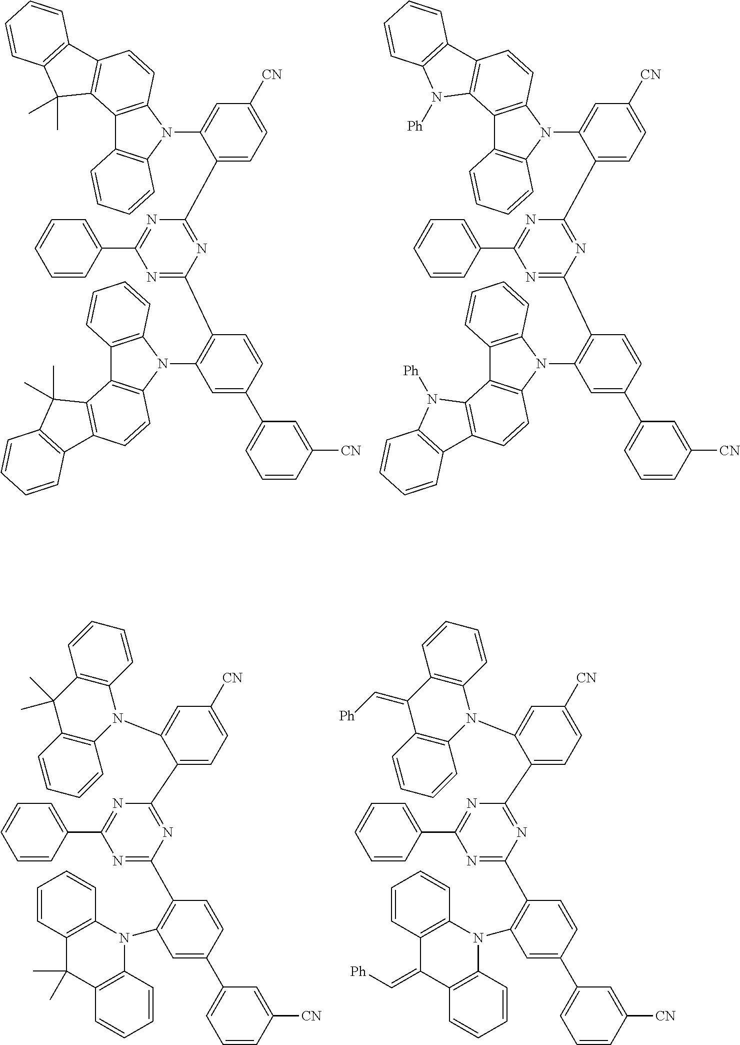

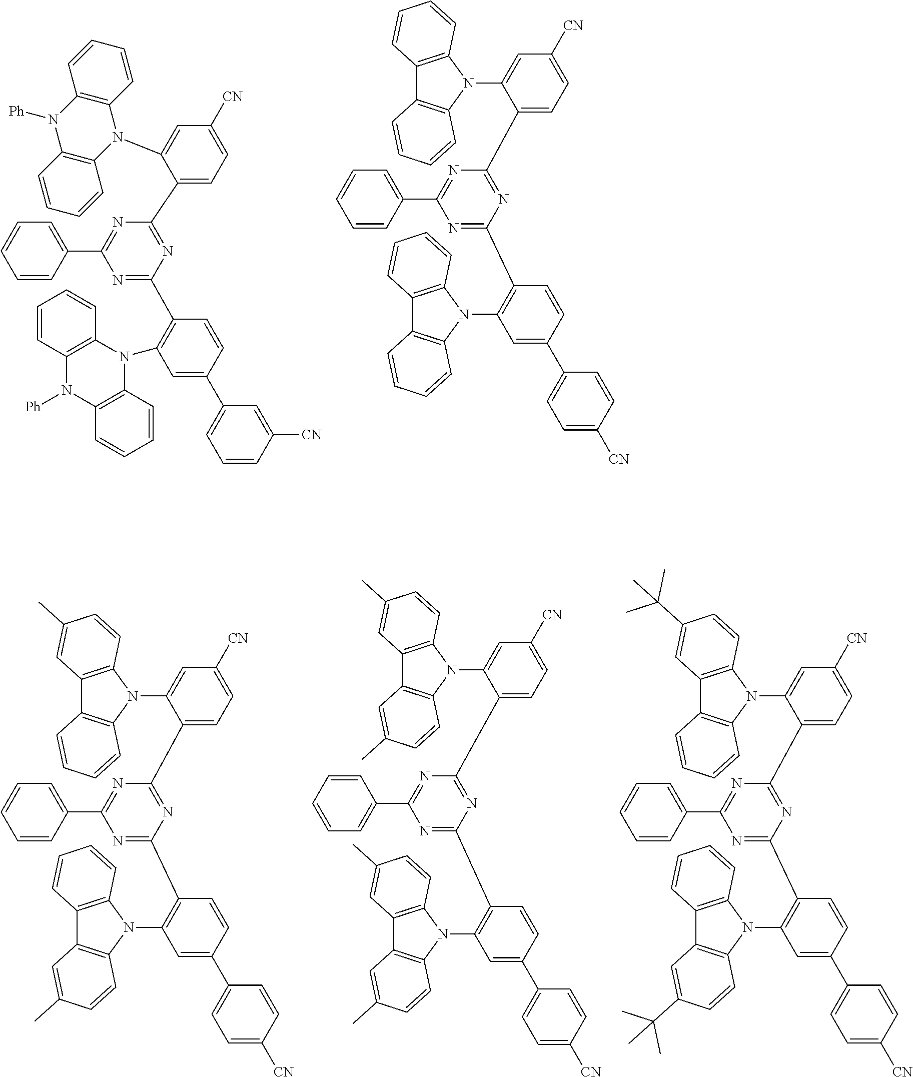

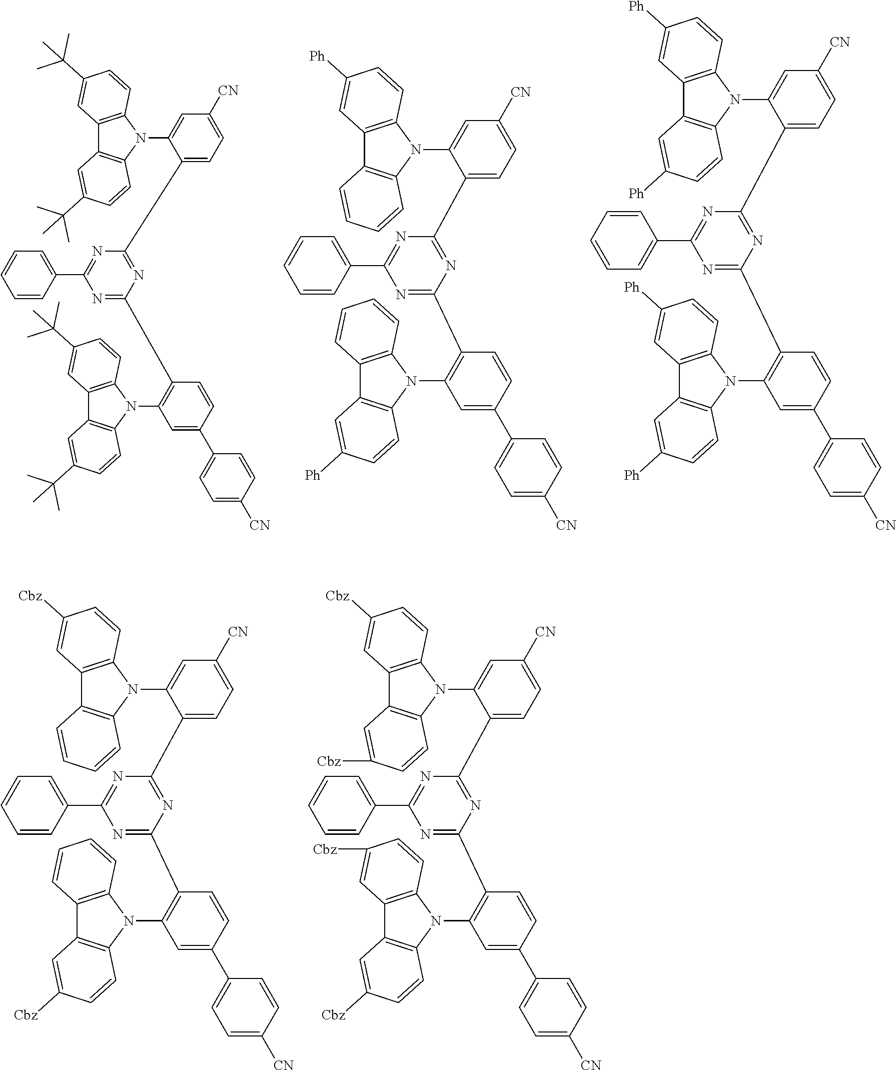

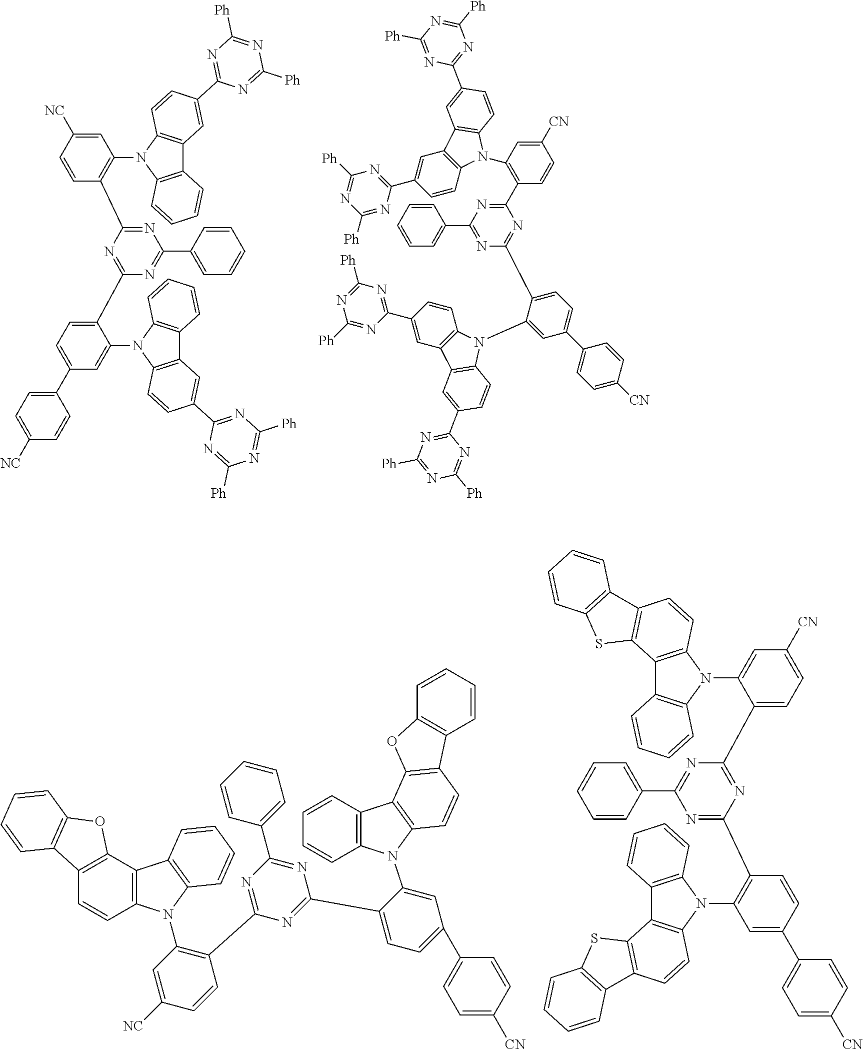

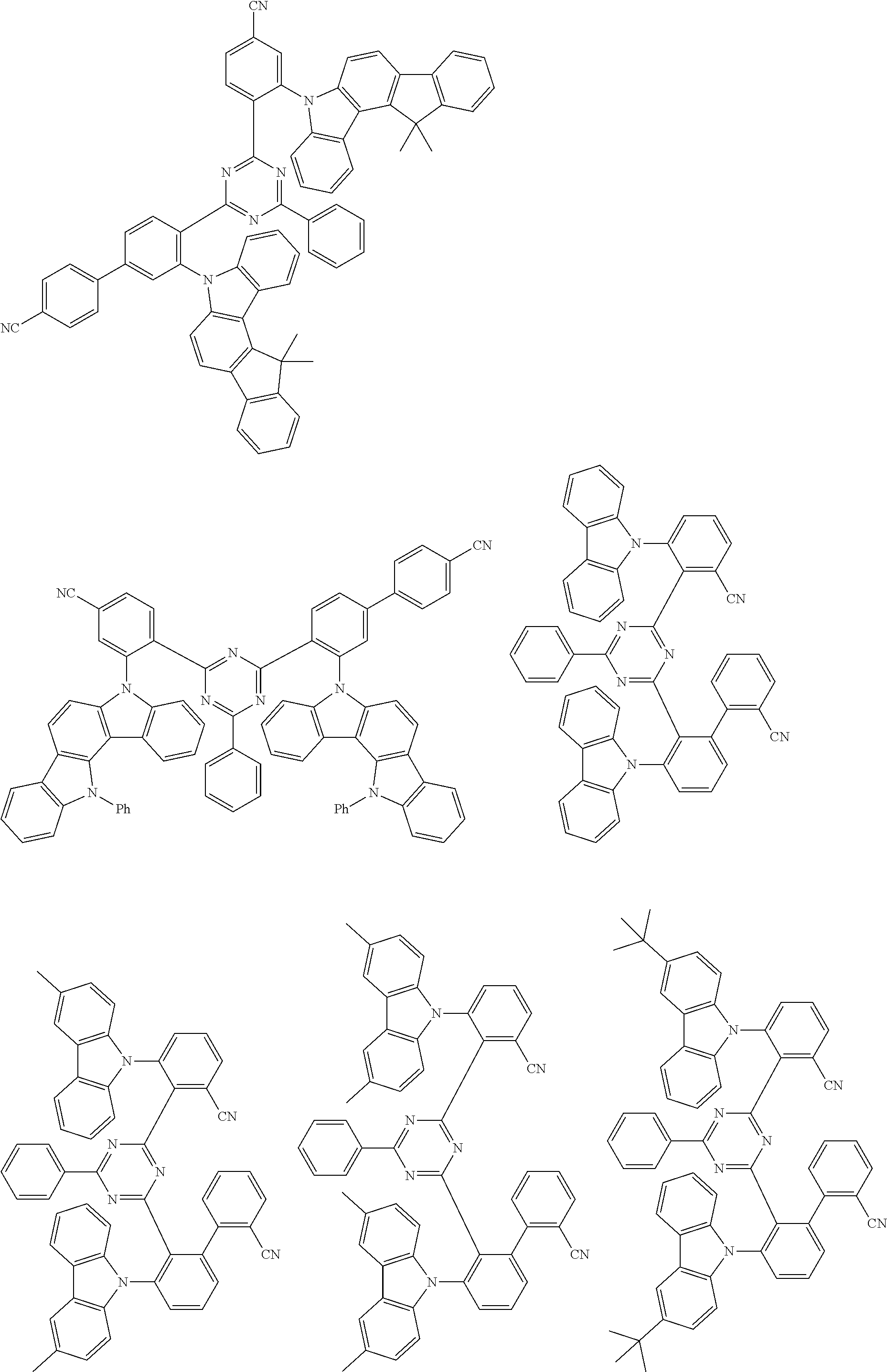

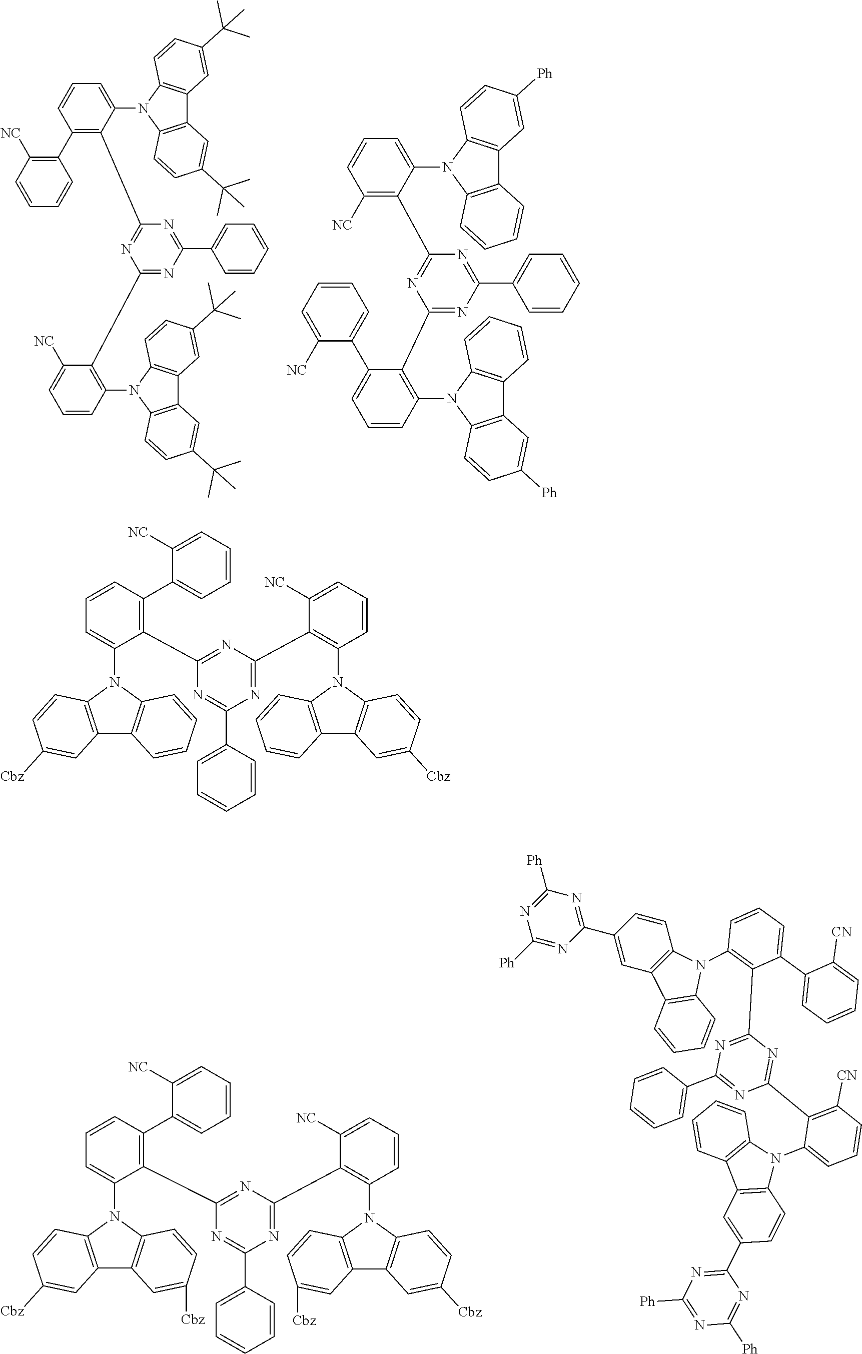

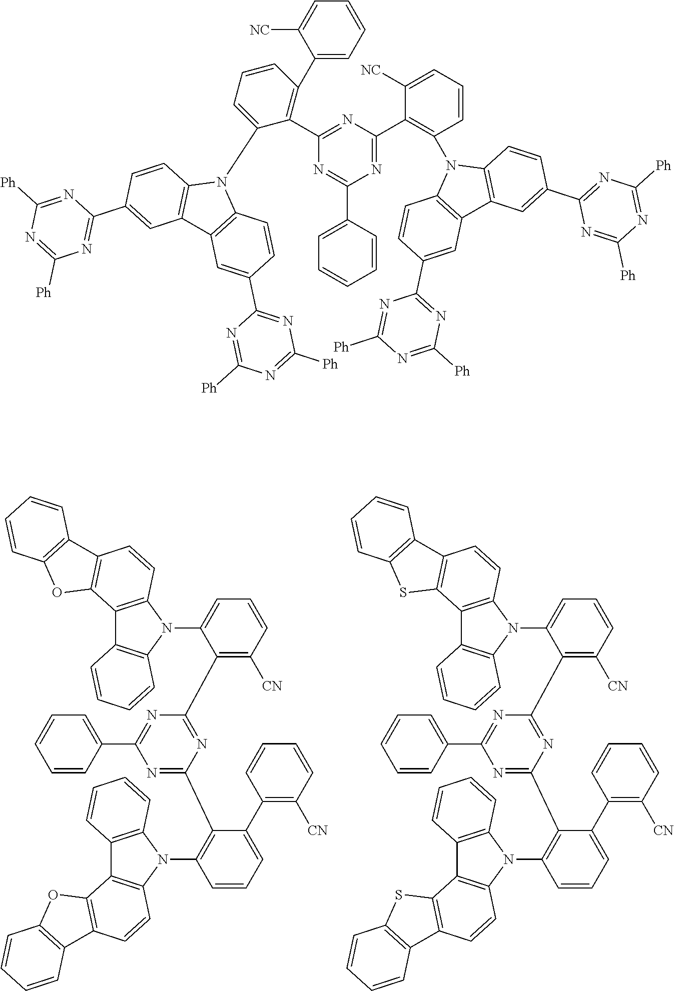 [SMILES: N#Cc1ccccc1-c1cccc(-n2c3ccc(-c4nc(-c5ccccc5)nc(-c5ccccc5)n4)cc3c3cc(-c4nc(-c5ccccc5)nc(-c5ccccc5)n4)ccc32)c1-c1nc(-c2ccccc2)nc(-c2c(C#N)cccc2-n2c3ccc(-c4nc(-c5ccccc5)nc(-c5ccccc5)n4)cc3c3cc(-c4nc(-c5ccccc5)nc(-c5ccccc5)n4)ccc32)n1 ; N#Cc1ccccc1-c1cccc(-n2c3ccccc3c3c4oc5ccccc5c4ccc32)c1-c1nc(-c2ccccc2)nc(-c2c(C#N)cccc2-n2c3ccccc3c3c4oc5ccccc5c4ccc32)n1 ; N#Cc1ccccc1-c1cccc(-n2c3ccccc3c3c4sc5ccccc5c4ccc32)c1-c1nc(-c2ccccc2)nc(-c2c(C#N)cccc2-n2c3ccccc3c3c4sc5ccccc5c4ccc32)n1]

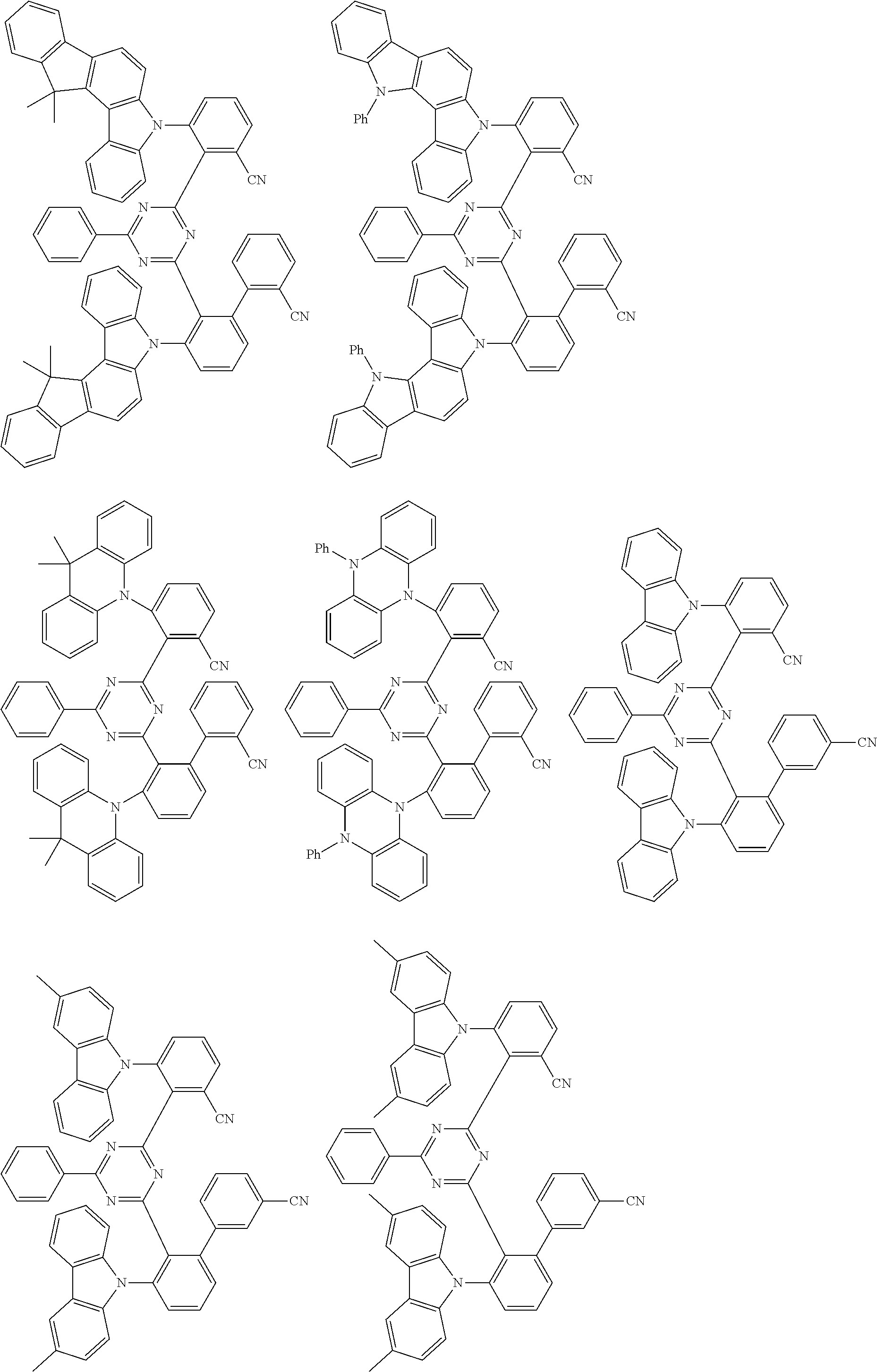

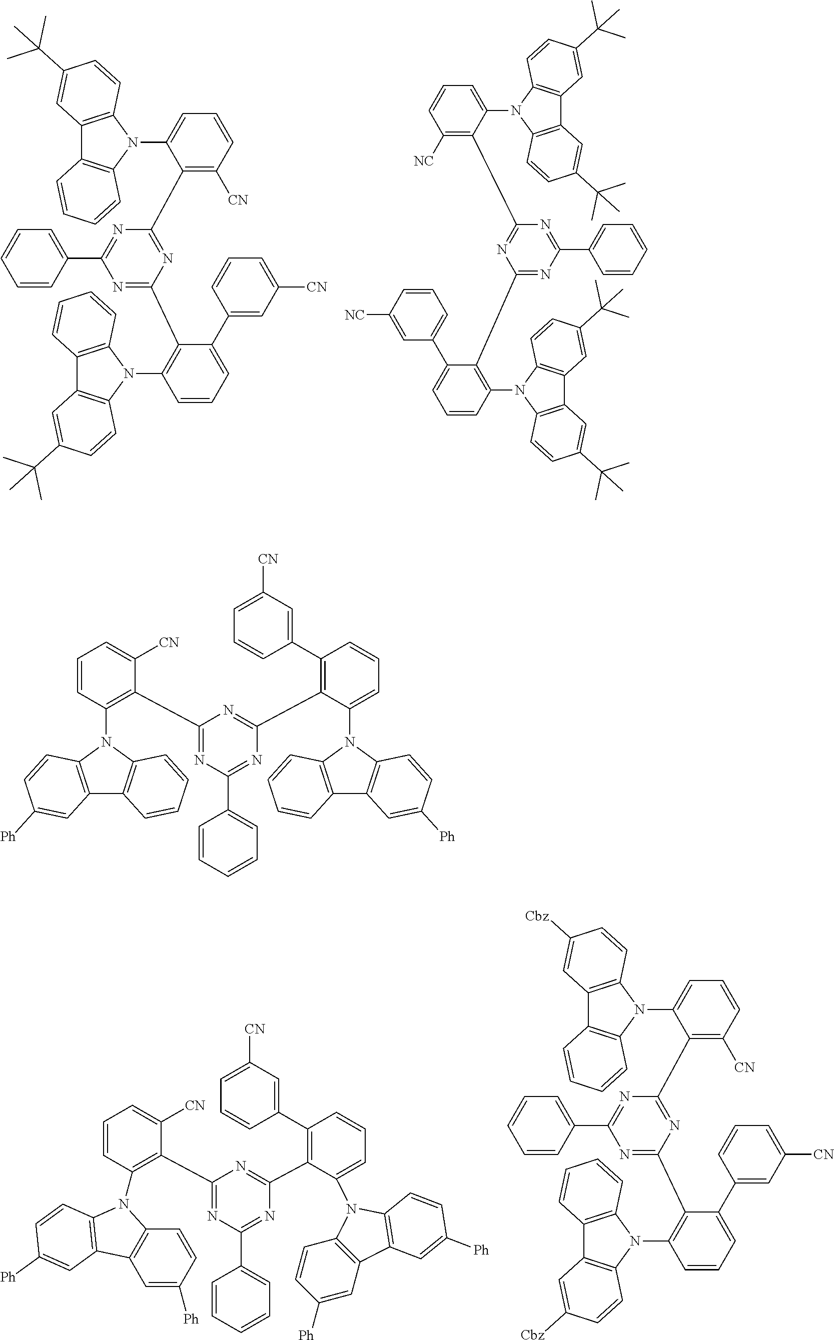

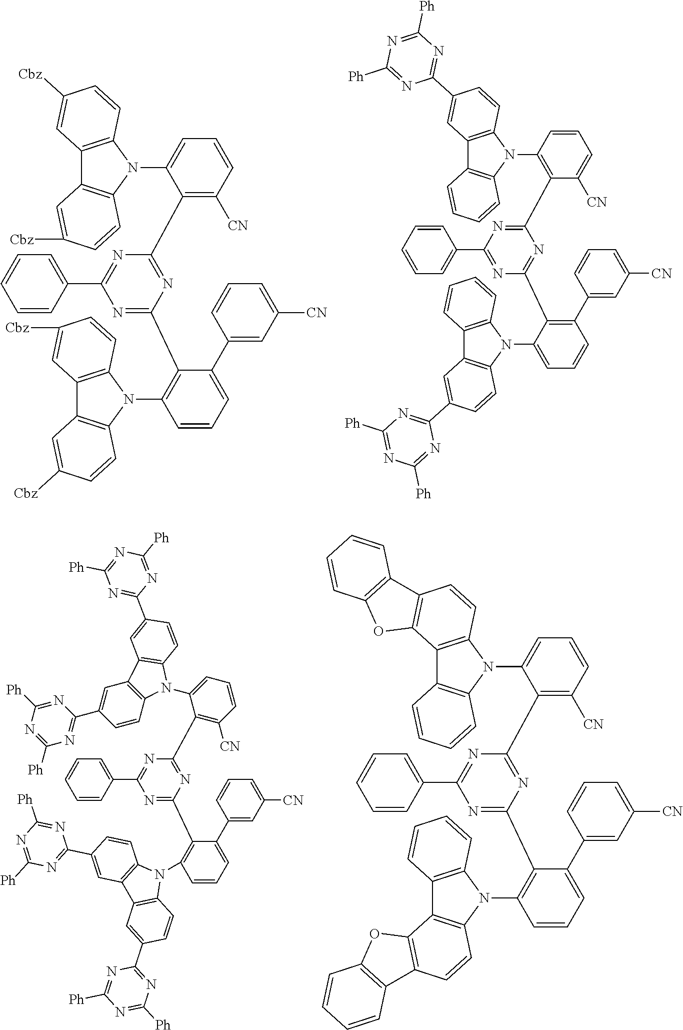

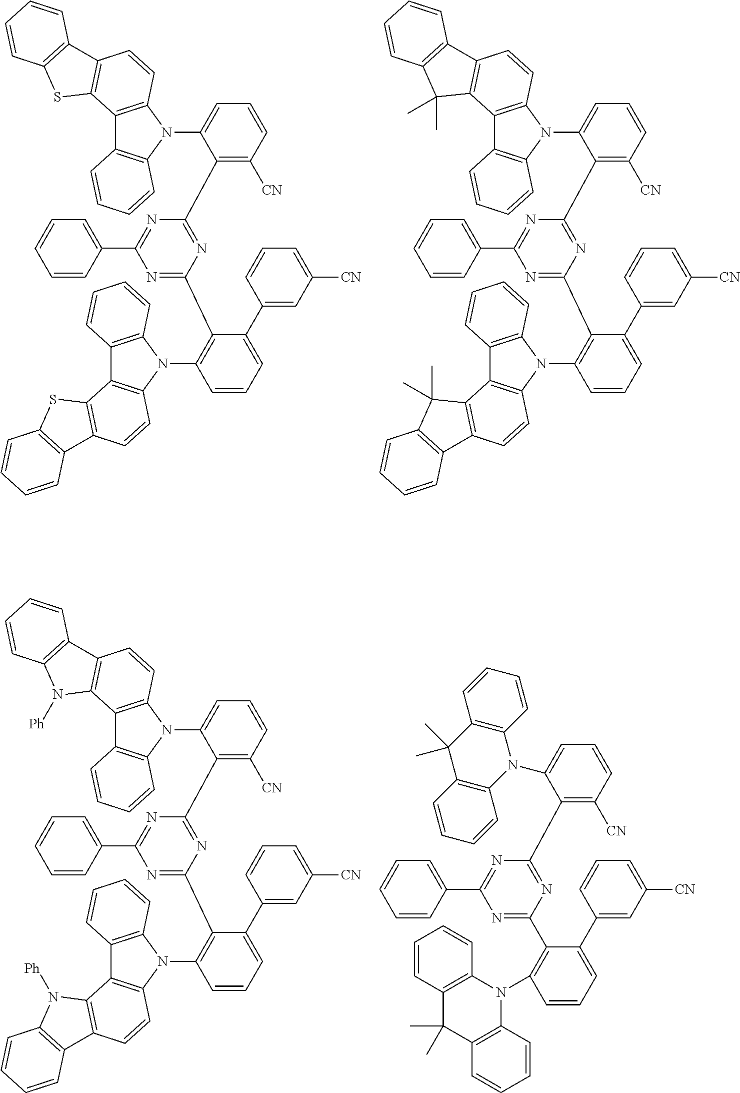

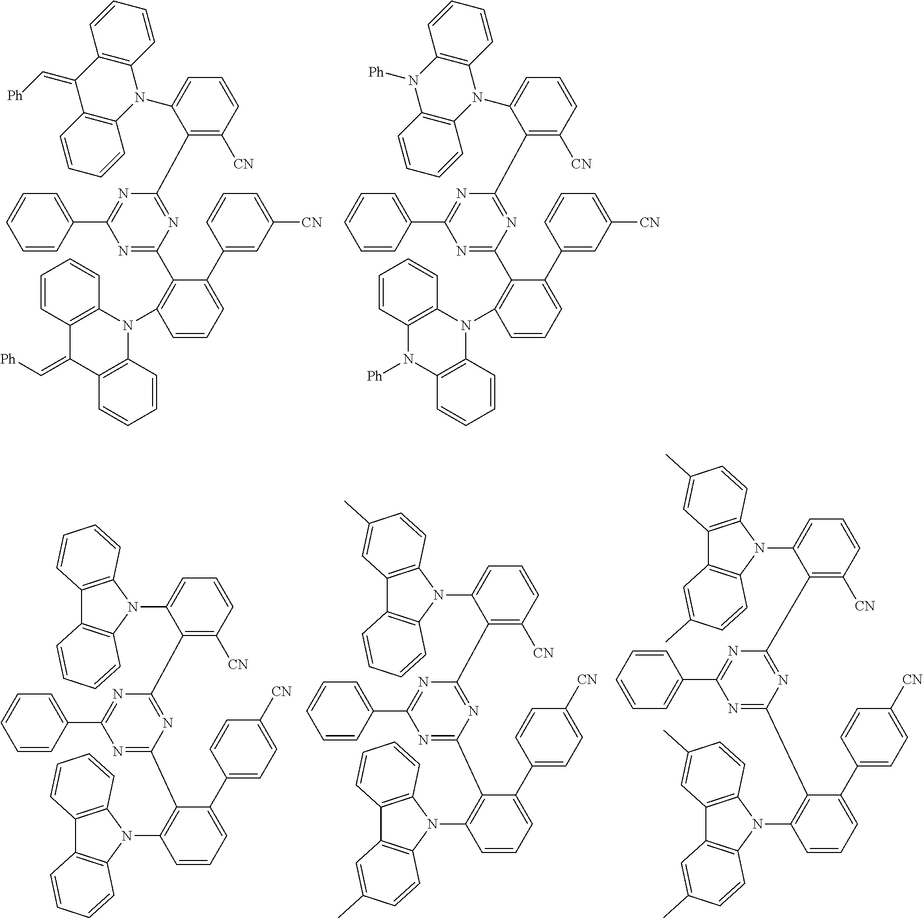

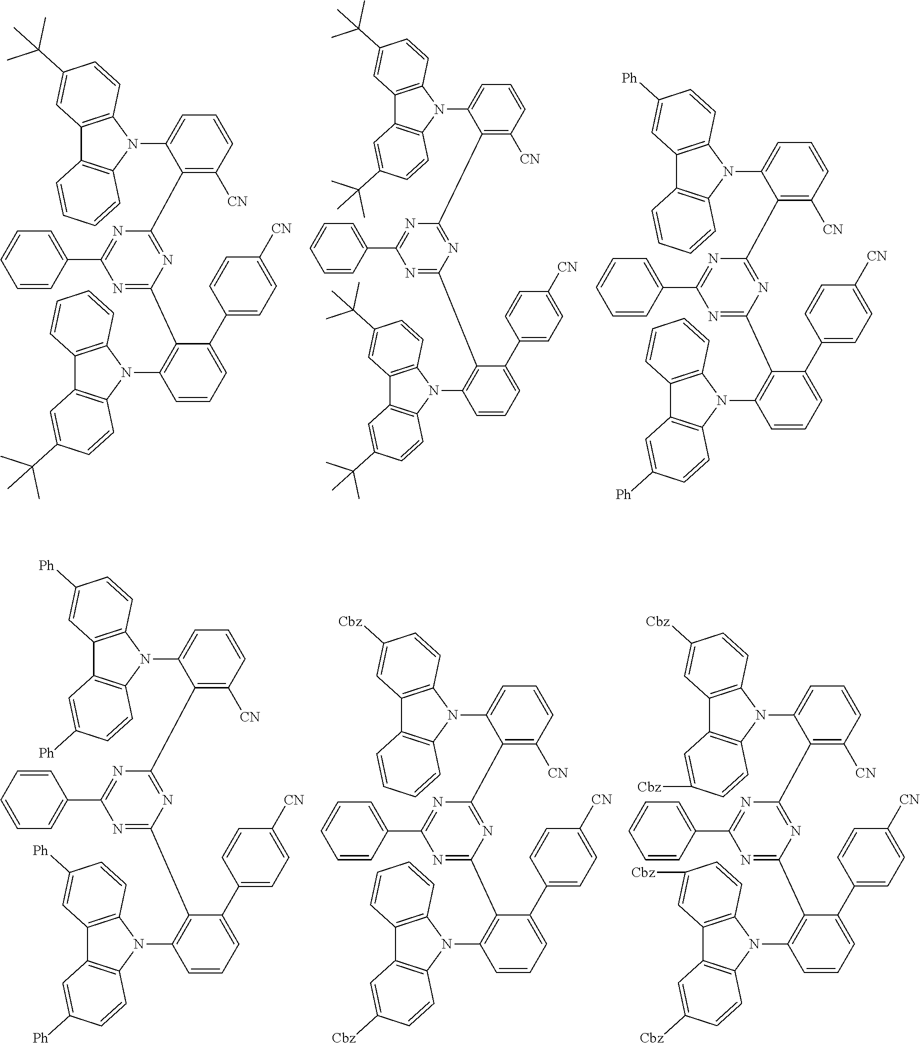

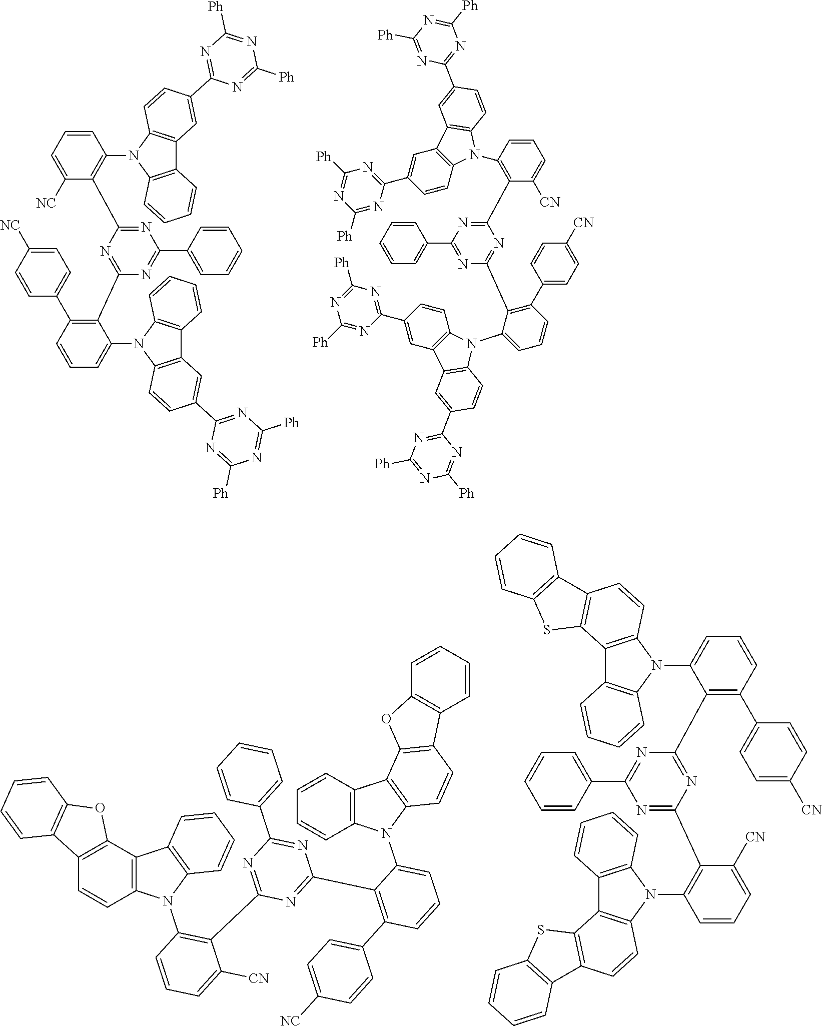

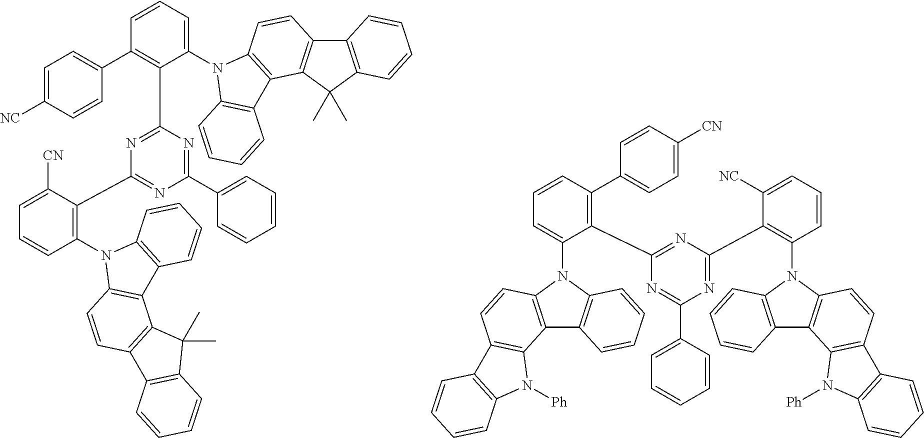 [SMILES: CC1(C)c2ccccc2-c2ccc3c(c21)c1ccccc1n3-c1cccc(C#N)c1-c1nc(-c2ccccc2)nc(-c2c(-c3ccc(C#N)cc3)cccc2-n2c3ccccc3c3c4c(ccc32)-c2ccccc2C4(C)C)n1 ; N#Cc1ccc(-c2cccc(-n3c4ccccc4c4c3ccc3c5ccccc5n(-c5ccccc5)c34)c2-c2nc(-c3ccccc3)nc(-c3c(C#N)cccc3-n3c4ccccc4c4c3ccc3c5ccccc5n(-c5ccccc5)c34)n2)cc1]

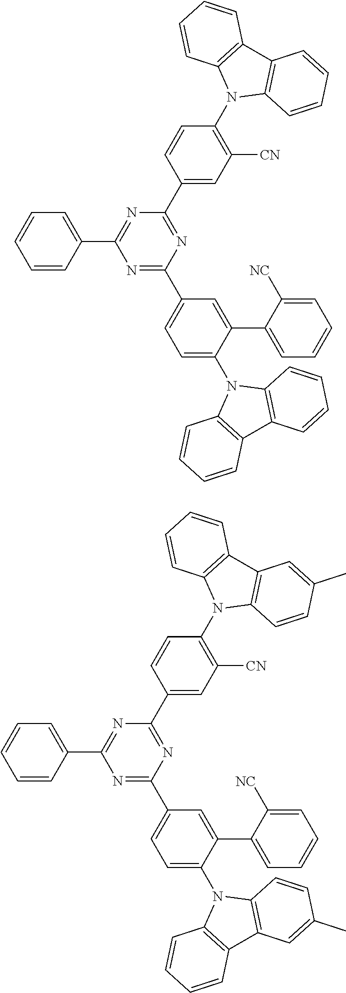

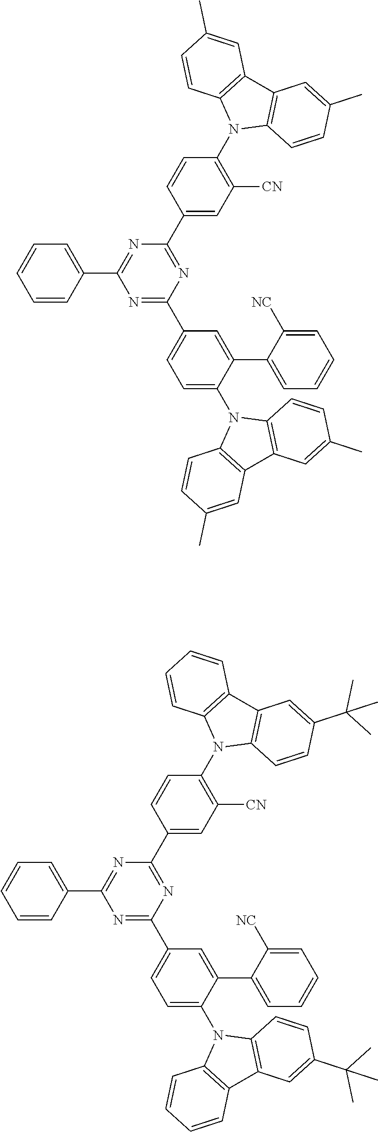 [SMILES: Cc1ccc2c(c1)c1cc(C)ccc1n2-c1ccc(-c2nc(-c3ccccc3)nc(-c3ccc(-n4c5ccc(C)cc5c5cc(C)ccc54)c(-c4ccccc4C#N)c3)n2)cc1C#N ; CC(C)(C)c1ccc2c(c1)c1ccccc1n2-c1ccc(-c2nc(-c3ccccc3)nc(-c3ccc(-n4c5ccccc5c5cc(C(C)(C)C)ccc54)c(-c4ccccc4C#N)c3)n2)cc1C#N]

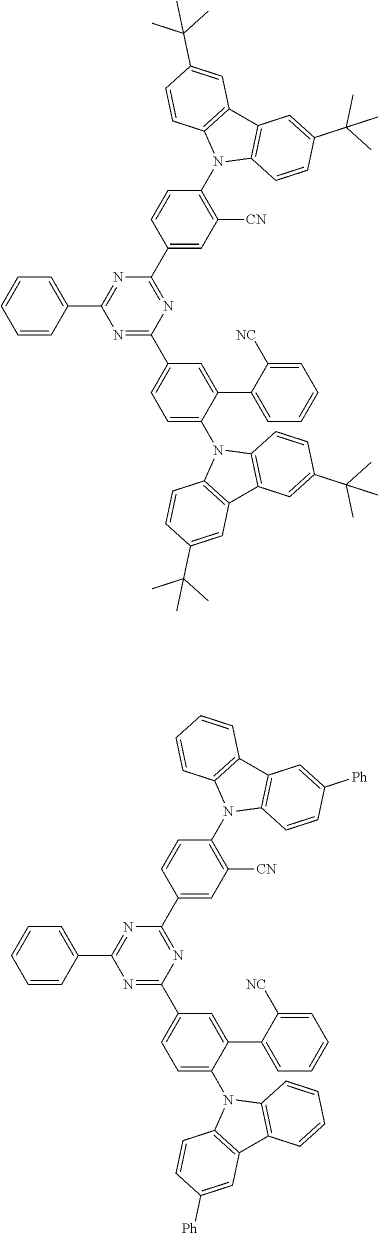 [SMILES: CC(C)(C)c1ccc2c(c1)c1cc(C(C)(C)C)ccc1n2-c1ccc(-c2nc(-c3ccccc3)nc(-c3ccc(-n4c5ccc(C(C)(C)C)cc5c5cc(C(C)(C)C)ccc54)c(-c4ccccc4C#N)c3)n2)cc1C#N ; N#Cc1ccccc1-c1cc(-c2nc(-c3ccccc3)nc(-c3ccc(-n4c5ccccc5c5cc(-c6ccccc6)ccc54)c(C#N)c3)n2)ccc1-n1c2ccccc2c2cc(-c3ccccc3)ccc21]

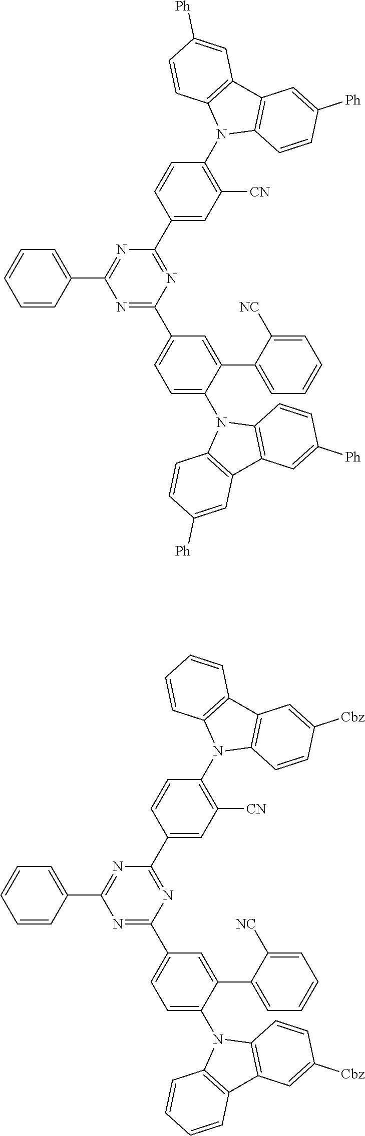 [SMILES: N#Cc1ccccc1-c1cc(-c2nc(-c3ccccc3)nc(-c3ccc(-n4c5ccc(-c6ccccc6)cc5c5cc(-c6ccccc6)ccc54)c(C#N)c3)n2)ccc1-n1c2ccc(-c3ccccc3)cc2c2cc(-c3ccccc3)ccc21 ; N#Cc1ccccc1-c1cc(-c2nc(-c3ccccc3)nc(-c3ccc(-n4c5ccccc5c5cc(C(=O)OCc6ccccc6)ccc54)c(C#N)c3)n2)ccc1-n1c2ccccc2c2cc(C(=O)OCc3ccccc3)ccc21]

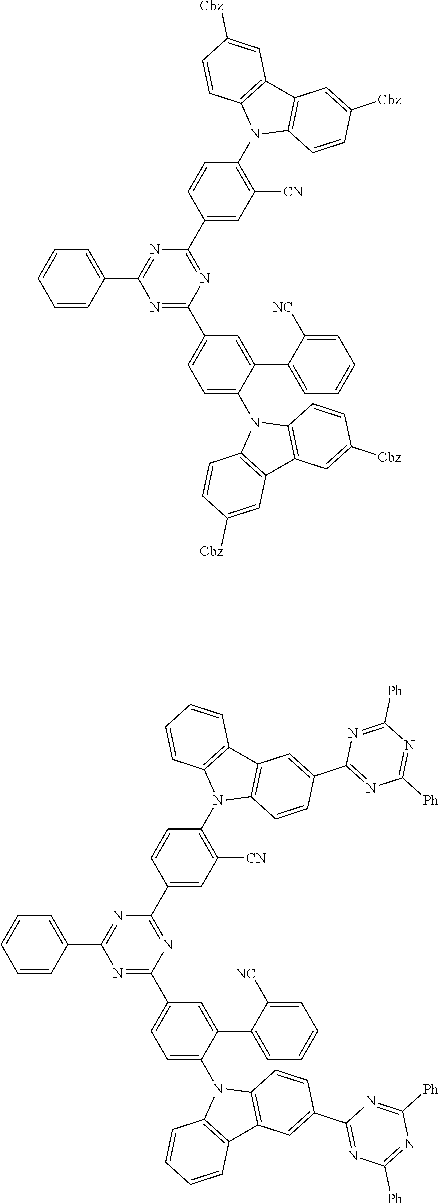 [SMILES: N#Cc1ccccc1-c1cc(-c2nc(-c3ccccc3)nc(-c3ccc(-n4c5ccc(C(=O)OCc6ccccc6)cc5c5cc(C(=O)OCc6ccccc6)ccc54)c(C#N)c3)n2)ccc1-n1c2ccc(C(=O)OCc3ccccc3)cc2c2cc(C(=O)OCc3ccccc3)ccc21 ; N#Cc1ccccc1-c1cc(-c2nc(-c3ccccc3)nc(-c3ccc(-n4c5ccccc5c5cc(-c6nc(-c7ccccc7)nc(-c7ccccc7)n6)ccc54)c(C#N)c3)n2)ccc1-n1c2ccccc2c2cc(-c3nc(-c4ccccc4)nc(-c4ccccc4)n3)ccc21]

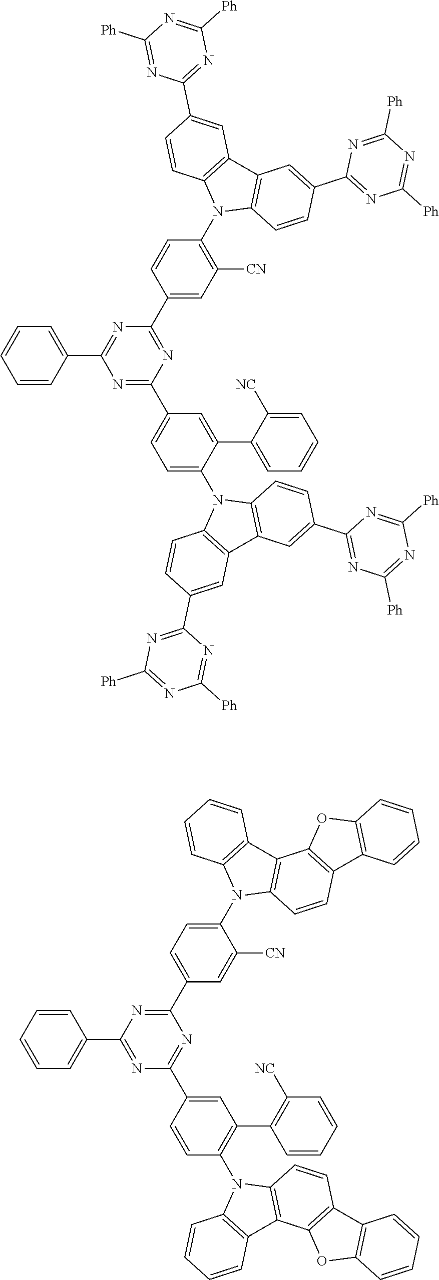 [SMILES: N#Cc1ccccc1-c1cc(-c2nc(-c3ccccc3)nc(-c3ccc(-n4c5ccc(-c6nc(-c7ccccc7)nc(-c7ccccc7)n6)cc5c5cc(-c6nc(-c7ccccc7)nc(-c7ccccc7)n6)ccc54)c(C#N)c3)n2)ccc1-n1c2ccc(-c3nc(-c4ccccc4)nc(-c4ccccc4)n3)cc2c2cc(-c3nc(-c4ccccc4)nc(-c4ccccc4)n3)ccc21 ; N#Cc1ccccc1-c1cc(-c2nc(-c3ccccc3)nc(-c3ccc(-n4c5ccccc5c5c6oc7ccccc7c6ccc54)c(C#N)c3)n2)ccc1-n1c2ccccc2c2c3oc4ccccc4c3ccc21]

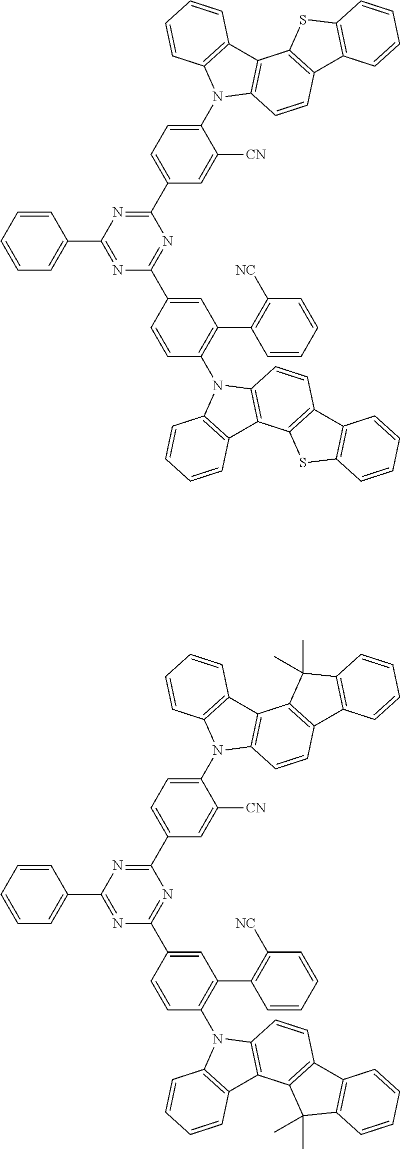 [SMILES: N#Cc1ccccc1-c1cc(-c2nc(-c3ccccc3)nc(-c3ccc(-n4c5ccccc5c5c6sc7ccccc7c6ccc54)c(C#N)c3)n2)ccc1-n1c2ccccc2c2c3sc4ccccc4c3ccc21 ; CC1(C)c2ccccc2-c2ccc3c(c21)c1ccccc1n3-c1ccc(-c2nc(-c3ccccc3)nc(-c3ccc(-n4c5ccccc5c5c6c(ccc54)-c4ccccc4C6(C)C)c(-c4ccccc4C#N)c3)n2)cc1C#N]

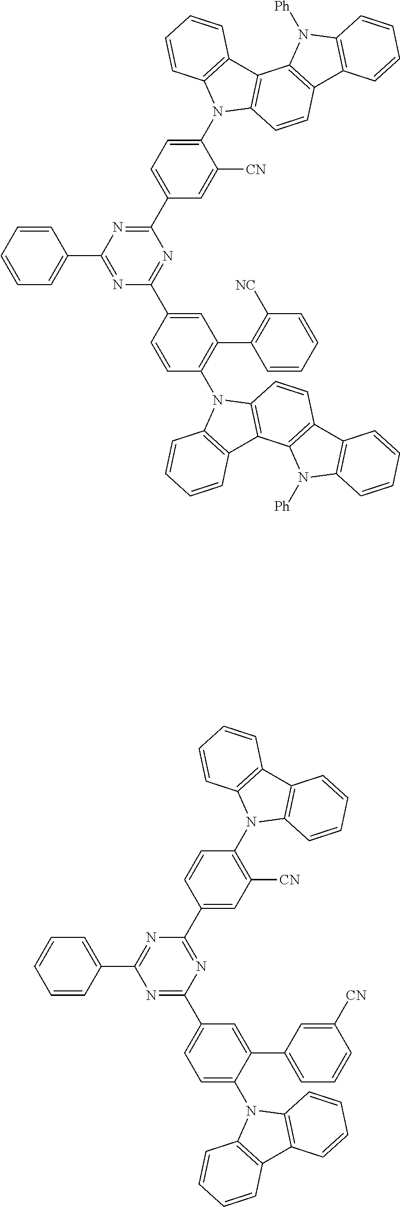 [SMILES: N#Cc1ccccc1-c1cc(-c2nc(-c3ccccc3)nc(-c3ccc(-n4c5ccccc5c5c4ccc4c6ccccc6n(-c6ccccc6)c45)c(C#N)c3)n2)ccc1-n1c2ccccc2c2c1ccc1c3ccccc3n(-c3ccccc3)c12 ; N#Cc1cccc(-c2cc(-c3nc(-c4ccccc4)nc(-c4ccc(-n5c6ccccc6c6ccccc65)c(C#N)c4)n3)ccc2-n2c3ccccc3c3ccccc32)c1]

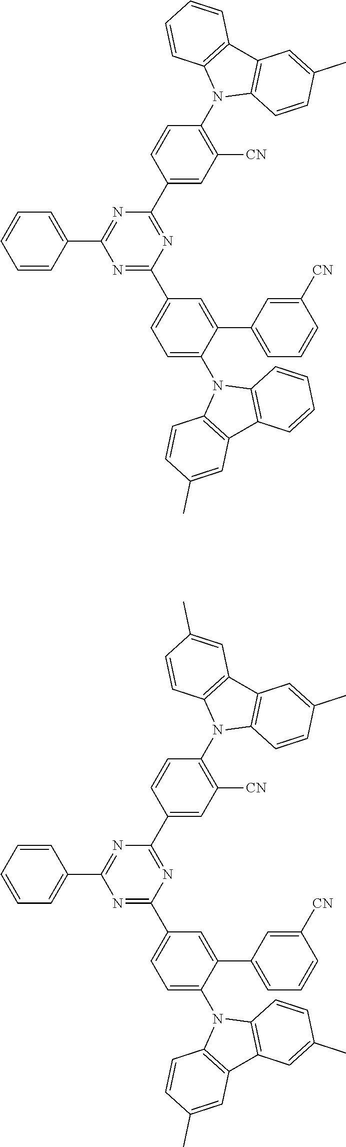 [SMILES: Cc1ccc2c(c1)c1ccccc1n2-c1ccc(-c2nc(-c3ccccc3)nc(-c3ccc(-n4c5ccccc5c5cc(C)ccc54)c(-c4cccc(C#N)c4)c3)n2)cc1C#N ; Cc1ccc2c(c1)c1cc(C)ccc1n2-c1ccc(-c2nc(-c3ccccc3)nc(-c3ccc(-n4c5ccc(C)cc5c5cc(C)ccc54)c(-c4cccc(C#N)c4)c3)n2)cc1C#N]

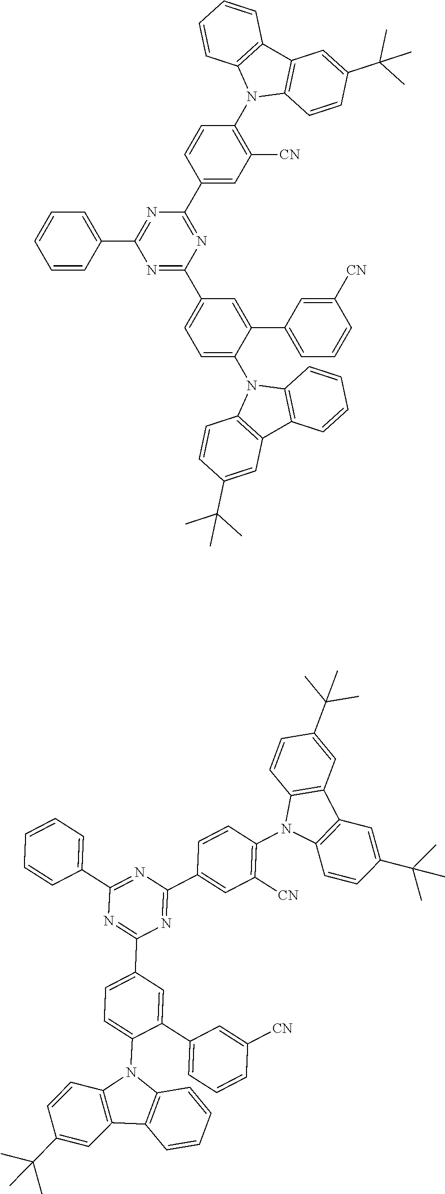

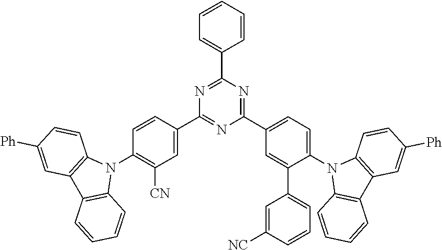 [SMILES: N#Cc1cccc(-c2cc(-c3nc(-c4ccccc4)nc(-c4ccc(-n5c6ccccc6c6cc(-c7ccccc7)ccc65)c(C#N)c4)n3)ccc2-n2c3ccccc3c3cc(-c4ccccc4)ccc32)c1]

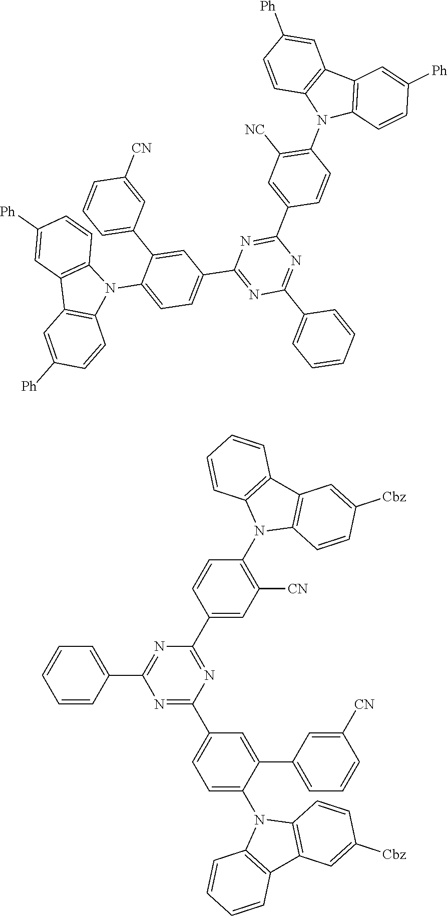 [SMILES: N#Cc1cccc(-c2cc(-c3nc(-c4ccccc4)nc(-c4ccc(-n5c6ccc(-c7ccccc7)cc6c6cc(-c7ccccc7)ccc65)c(C#N)c4)n3)ccc2-n2c3ccc(-c4ccccc4)cc3c3cc(-c4ccccc4)ccc32)c1 ; N#Cc1cccc(-c2cc(-c3nc(-c4ccccc4)nc(-c4ccc(-n5c6ccccc6c6cc(C(=O)OCc7ccccc7)ccc65)c(C#N)c4)n3)ccc2-n2c3ccccc3c3cc(C(=O)OCc4ccccc4)ccc32)c1]

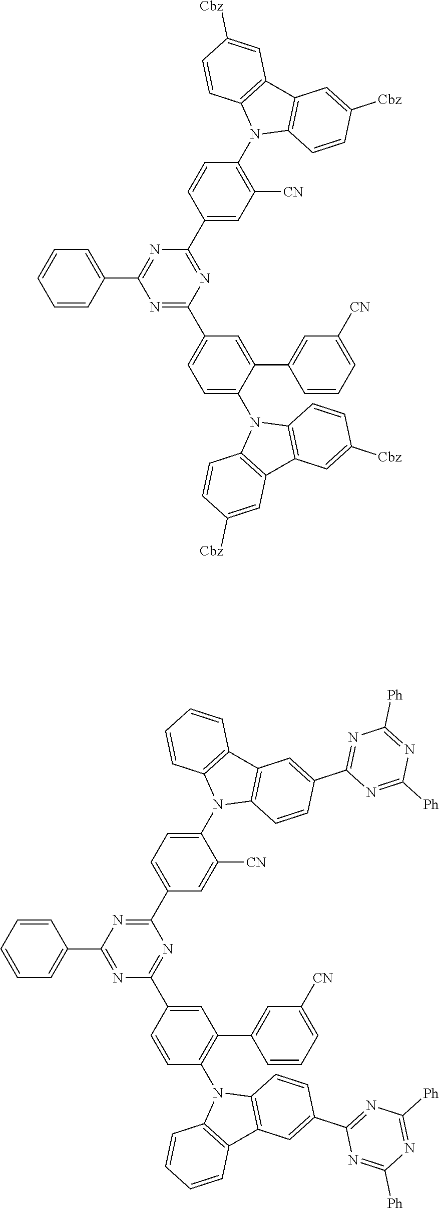 [SMILES: N#Cc1cccc(-c2cc(-c3nc(-c4ccccc4)nc(-c4ccc(-n5c6ccc(C(=O)OCc7ccccc7)cc6c6cc(C(=O)OCc7ccccc7)ccc65)c(C#N)c4)n3)ccc2-n2c3ccc(C(=O)OCc4ccccc4)cc3c3cc(C(=O)OCc4ccccc4)ccc32)c1 ; N#Cc1cccc(-c2cc(-c3nc(-c4ccccc4)nc(-c4ccc(-n5c6ccccc6c6cc(-c7nc(-c8ccccc8)nc(-c8ccccc8)n7)ccc65)c(C#N)c4)n3)ccc2-n2c3ccccc3c3cc(-c4nc(-c5ccccc5)nc(-c5ccccc5)n4)ccc32)c1]

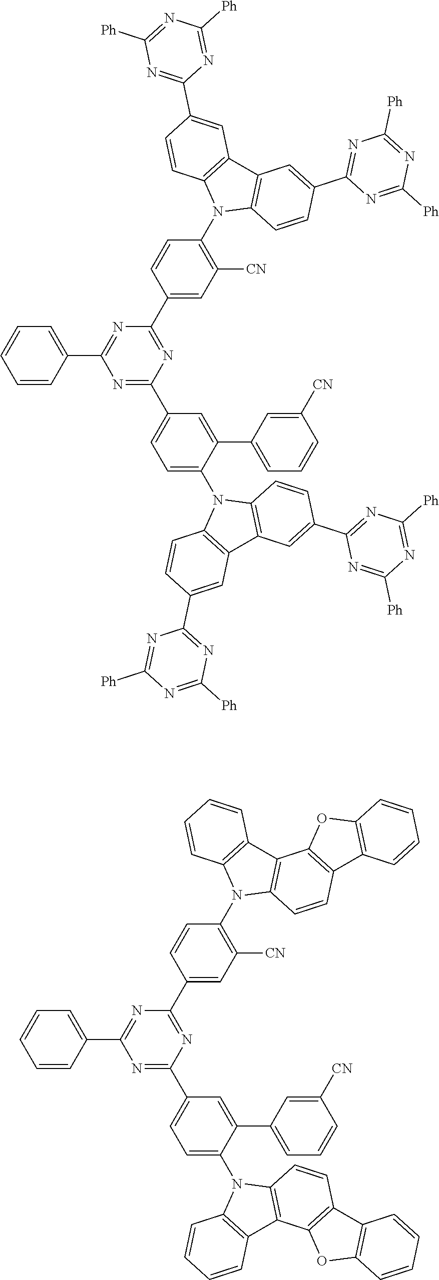 [SMILES: N#Cc1cccc(-c2cc(-c3nc(-c4ccccc4)nc(-c4ccc(-n5c6ccc(-c7nc(-c8ccccc8)nc(-c8ccccc8)n7)cc6c6cc(-c7nc(-c8ccccc8)nc(-c8ccccc8)n7)ccc65)c(C#N)c4)n3)ccc2-n2c3ccc(-c4nc(-c5ccccc5)nc(-c5ccccc5)n4)cc3c3cc(-c4nc(-c5ccccc5)nc(-c5ccccc5)n4)ccc32)c1 ; N#Cc1cccc(-c2cc(-c3nc(-c4ccccc4)nc(-c4ccc(-n5c6ccccc6c6c7oc8ccccc8c7ccc65)c(C#N)c4)n3)ccc2-n2c3ccccc3c3c4oc5ccccc5c4ccc32)c1]

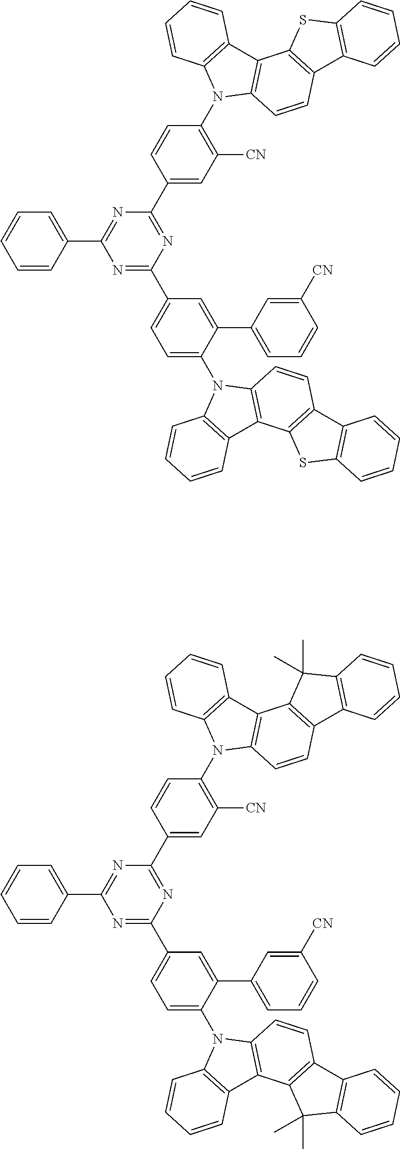 [SMILES: N#Cc1cccc(-c2cc(-c3nc(-c4ccccc4)nc(-c4ccc(-n5c6ccccc6c6c7sc8ccccc8c7ccc65)c(C#N)c4)n3)ccc2-n2c3ccccc3c3c4sc5ccccc5c4ccc32)c1 ; CC1(C)c2ccccc2-c2ccc3c(c21)c1ccccc1n3-c1ccc(-c2nc(-c3ccccc3)nc(-c3ccc(-n4c5ccccc5c5c6c(ccc54)-c4ccccc4C6(C)C)c(-c4cccc(C#N)c4)c3)n2)cc1C#N]

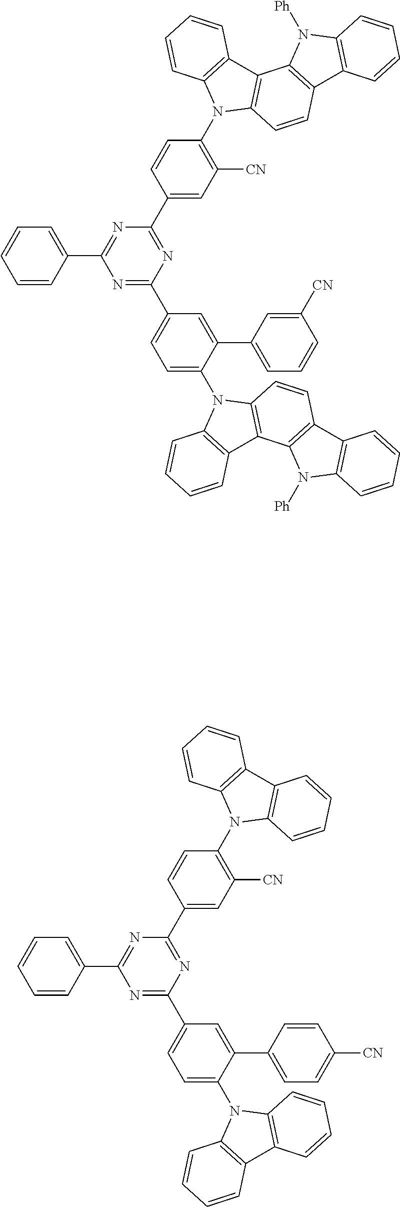 [SMILES: N#Cc1cccc(-c2cc(-c3nc(-c4ccccc4)nc(-c4ccc(-n5c6ccccc6c6c5ccc5c7ccccc7n(-c7ccccc7)c56)c(C#N)c4)n3)ccc2-n2c3ccccc3c3c2ccc2c4ccccc4n(-c4ccccc4)c23)c1 ; N#Cc1ccc(-c2cc(-c3nc(-c4ccccc4)nc(-c4ccc(-n5c6ccccc6c6ccccc65)c(C#N)c4)n3)ccc2-n2c3ccccc3c3ccccc32)cc1]

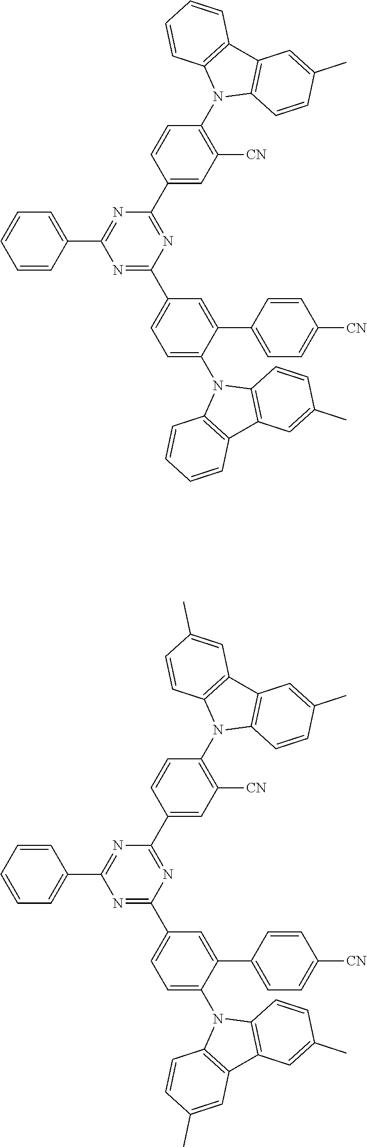 [SMILES: Cc1ccc2c(c1)c1ccccc1n2-c1ccc(-c2nc(-c3ccccc3)nc(-c3ccc(-n4c5ccccc5c5cc(C)ccc54)c(-c4ccc(C#N)cc4)c3)n2)cc1C#N ; Cc1ccc2c(c1)c1cc(C)ccc1n2-c1ccc(-c2nc(-c3ccccc3)nc(-c3ccc(-n4c5ccc(C)cc5c5cc(C)ccc54)c(-c4ccc(C#N)cc4)c3)n2)cc1C#N]

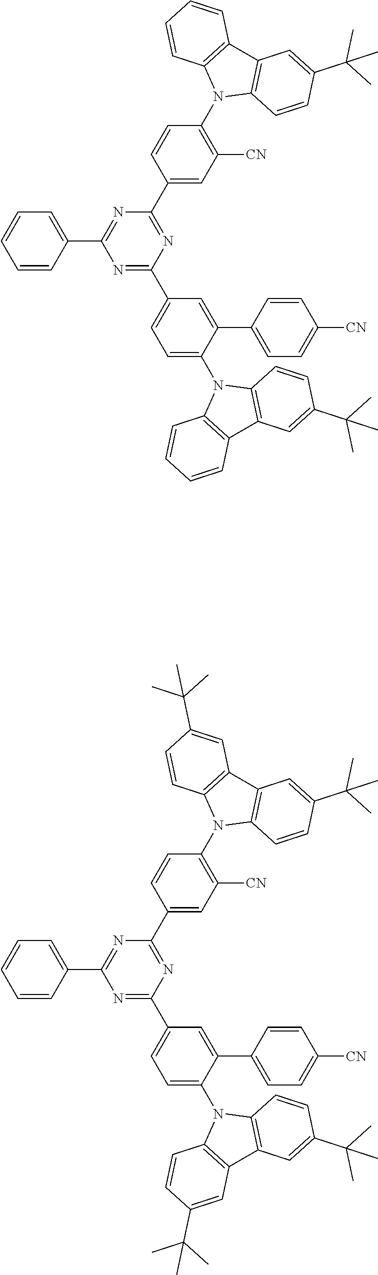 [SMILES: CC(C)(C)c1ccc2c(c1)c1ccccc1n2-c1ccc(-c2nc(-c3ccccc3)nc(-c3ccc(-n4c5ccccc5c5cc(C(C)(C)C)ccc54)c(-c4ccc(C#N)cc4)c3)n2)cc1C#N ; CC(C)(C)c1ccc2c(c1)c1cc(C(C)(C)C)ccc1n2-c1ccc(-c2nc(-c3ccccc3)nc(-c3ccc(-n4c5ccc(C(C)(C)C)cc5c5cc(C(C)(C)C)ccc54)c(-c4ccc(C#N)cc4)c3)n2)cc1C#N]

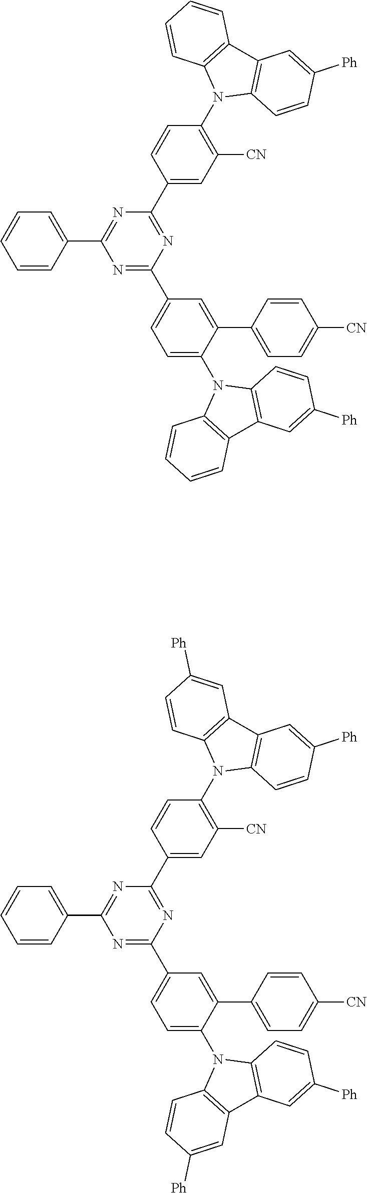 [SMILES: N#Cc1ccc(-c2cc(-c3nc(-c4ccccc4)nc(-c4ccc(-n5c6ccccc6c6cc(-c7ccccc7)ccc65)c(C#N)c4)n3)ccc2-n2c3ccccc3c3cc(-c4ccccc4)ccc32)cc1 ; N#Cc1ccc(-c2cc(-c3nc(-c4ccccc4)nc(-c4ccc(-n5c6ccc(-c7ccccc7)cc6c6cc(-c7ccccc7)ccc65)c(C#N)c4)n3)ccc2-n2c3ccc(-c4ccccc4)cc3c3cc(-c4ccccc4)ccc32)cc1]

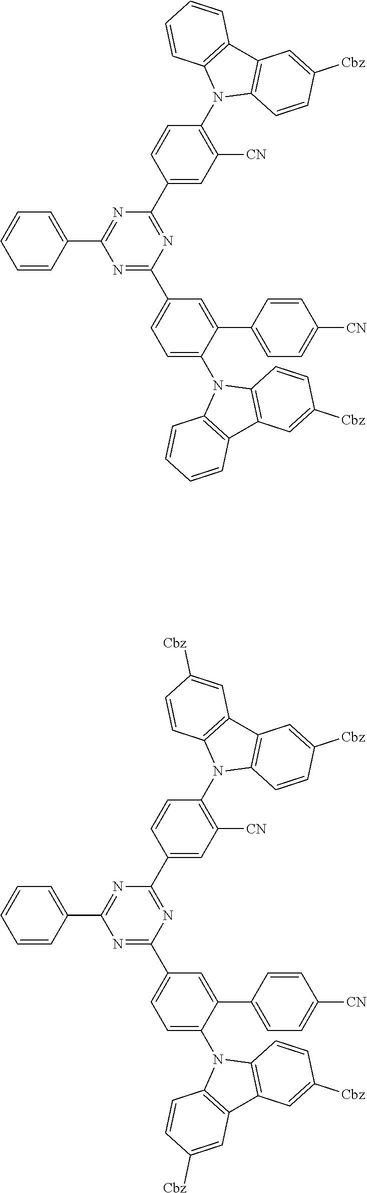 [SMILES: N#Cc1ccc(-c2cc(-c3nc(-c4ccccc4)nc(-c4ccc(-n5c6ccccc6c6cc(C(=O)OCc7ccccc7)ccc65)c(C#N)c4)n3)ccc2-n2c3ccccc3c3cc(C(=O)OCc4ccccc4)ccc32)cc1 ; N#Cc1ccc(-c2cc(-c3nc(-c4ccccc4)nc(-c4ccc(-n5c6ccc(C(=O)OCc7ccccc7)cc6c6cc(C(=O)OCc7ccccc7)ccc65)c(C#N)c4)n3)ccc2-n2c3ccc(C(=O)OCc4ccccc4)cc3c3cc(C(=O)OCc4ccccc4)ccc32)cc1]

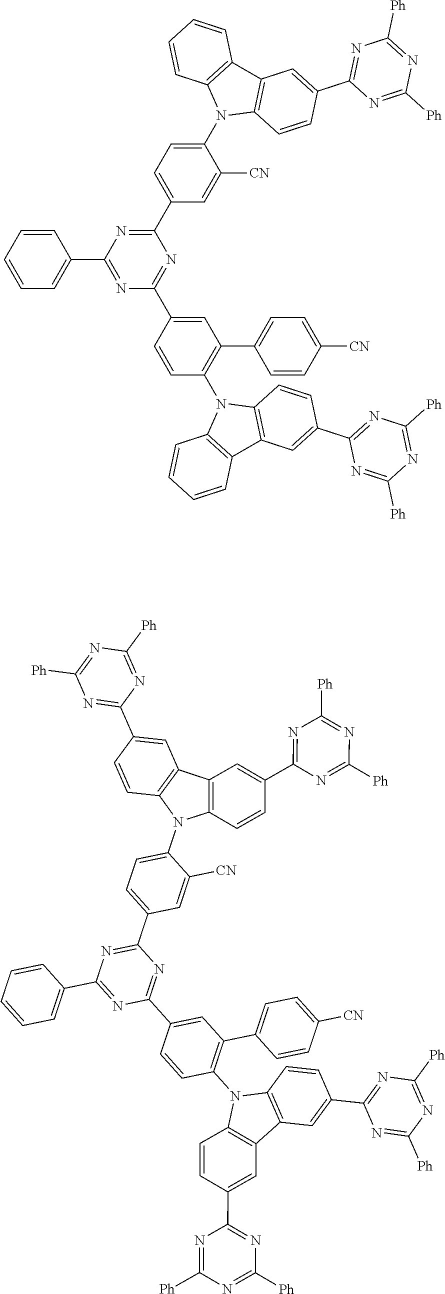 [SMILES: N#Cc1ccc(-c2cc(-c3nc(-c4ccccc4)nc(-c4ccc(-n5c6ccccc6c6cc(-c7nc(-c8ccccc8)nc(-c8ccccc8)n7)ccc65)c(C#N)c4)n3)ccc2-n2c3ccccc3c3cc(-c4nc(-c5ccccc5)nc(-c5ccccc5)n4)ccc32)cc1 ; N#Cc1ccc(-c2cc(-c3nc(-c4ccccc4)nc(-c4ccc(-n5c6ccc(-c7nc(-c8ccccc8)nc(-c8ccccc8)n7)cc6c6cc(-c7nc(-c8ccccc8)nc(-c8ccccc8)n7)ccc65)c(C#N)c4)n3)ccc2-n2c3ccc(-c4nc(-c5ccccc5)nc(-c5ccccc5)n4)cc3c3cc(-c4nc(-c5ccccc5)nc(-c5ccccc5)n4)ccc32)cc1]

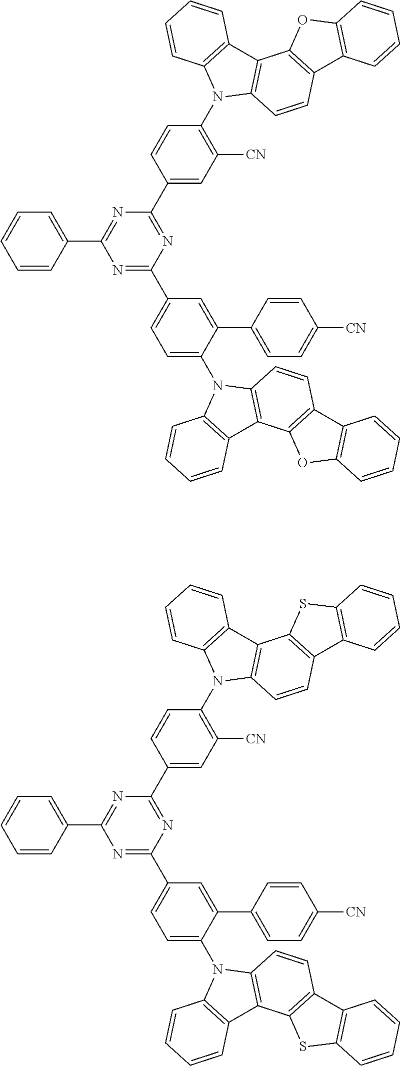 [SMILES: N#Cc1ccc(-c2cc(-c3nc(-c4ccccc4)nc(-c4ccc(-n5c6ccccc6c6c7oc8ccccc8c7ccc65)c(C#N)c4)n3)ccc2-n2c3ccccc3c3c4oc5ccccc5c4ccc32)cc1 ; N#Cc1ccc(-c2cc(-c3nc(-c4ccccc4)nc(-c4ccc(-n5c6ccccc6c6c7sc8ccccc8c7ccc65)c(C#N)c4)n3)ccc2-n2c3ccccc3c3c4sc5ccccc5c4ccc32)cc1]

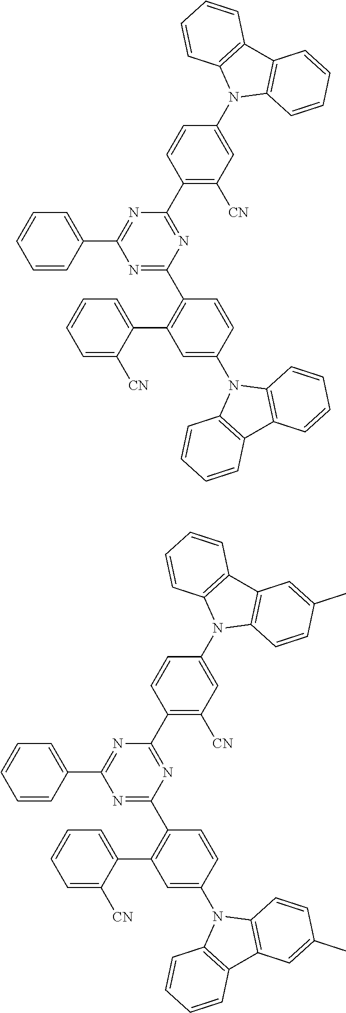 [SMILES: N#Cc1cc(-n2c3ccccc3c3ccccc32)ccc1-c1nc(-c2ccccc2)nc(-c2ccc(-n3c4ccccc4c4ccccc43)cc2-c2ccccc2C#N)n1 ; Cc1ccc2c(c1)c1ccccc1n2-c1ccc(-c2nc(-c3ccccc3)nc(-c3ccc(-n4c5ccccc5c5cc(C)ccc54)cc3-c3ccccc3C#N)n2)c(C#N)c1]

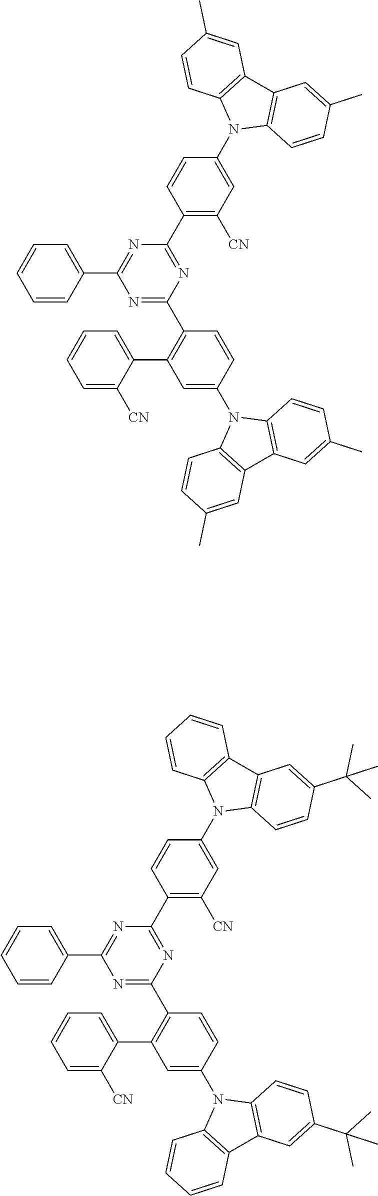 [SMILES: Cc1ccc2c(c1)c1cc(C)ccc1n2-c1ccc(-c2nc(-c3ccccc3)nc(-c3ccc(-n4c5ccc(C)cc5c5cc(C)ccc54)cc3-c3ccccc3C#N)n2)c(C#N)c1 ; CC(C)(C)c1ccc2c(c1)c1ccccc1n2-c1ccc(-c2nc(-c3ccccc3)nc(-c3ccc(-n4c5ccccc5c5cc(C(C)(C)C)ccc54)cc3-c3ccccc3C#N)n2)c(C#N)c1]

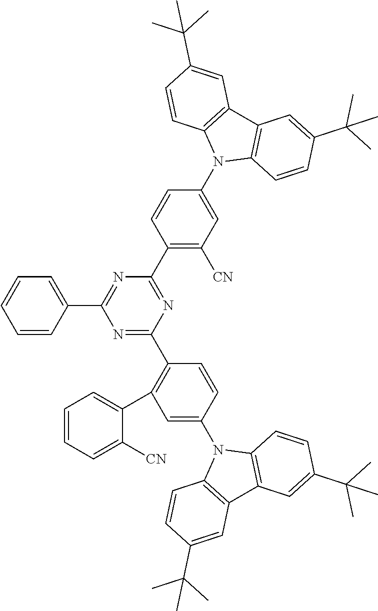 [SMILES: CC(C)(C)c1ccc2c(c1)c1cc(C(C)(C)C)ccc1n2-c1ccc(-c2nc(-c3ccccc3)nc(-c3ccc(-n4c5ccc(C(C)(C)C)cc5c5cc(C(C)(C)C)ccc54)cc3-c3ccccc3C#N)n2)c(C#N)c1]

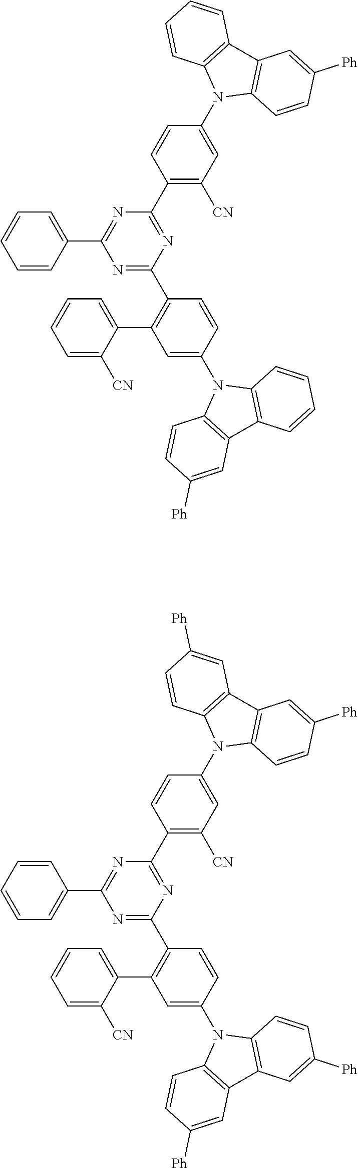 [SMILES: N#Cc1cc(-n2c3ccccc3c3cc(-c4ccccc4)ccc32)ccc1-c1nc(-c2ccccc2)nc(-c2ccc(-n3c4ccccc4c4cc(-c5ccccc5)ccc43)cc2-c2ccccc2C#N)n1 ; N#Cc1cc(-n2c3ccc(-c4ccccc4)cc3c3cc(-c4ccccc4)ccc32)ccc1-c1nc(-c2ccccc2)nc(-c2ccc(-n3c4ccc(-c5ccccc5)cc4c4cc(-c5ccccc5)ccc43)cc2-c2ccccc2C#N)n1]

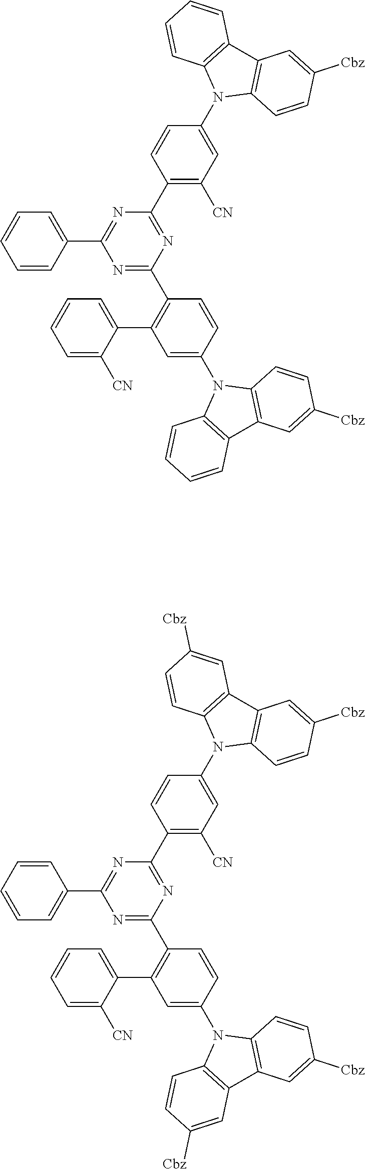 [SMILES: N#Cc1cc(-n2c3ccccc3c3cc(C(=O)OCc4ccccc4)ccc32)ccc1-c1nc(-c2ccccc2)nc(-c2ccc(-n3c4ccccc4c4cc(C(=O)OCc5ccccc5)ccc43)cc2-c2ccccc2C#N)n1 ; N#Cc1cc(-n2c3ccc(C(=O)OCc4ccccc4)cc3c3cc(C(=O)OCc4ccccc4)ccc32)ccc1-c1nc(-c2ccccc2)nc(-c2ccc(-n3c4ccc(C(=O)OCc5ccccc5)cc4c4cc(C(=O)OCc5ccccc5)ccc43)cc2-c2ccccc2C#N)n1]

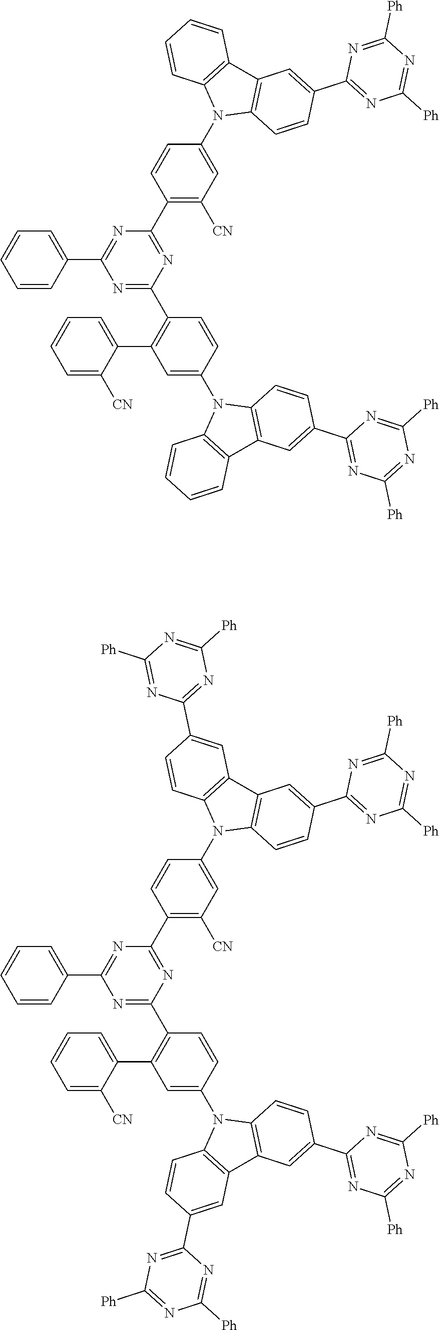 [SMILES: N#Cc1cc(-n2c3ccccc3c3cc(-c4nc(-c5ccccc5)nc(-c5ccccc5)n4)ccc32)ccc1-c1nc(-c2ccccc2)nc(-c2ccc(-n3c4ccccc4c4cc(-c5nc(-c6ccccc6)nc(-c6ccccc6)n5)ccc43)cc2-c2ccccc2C#N)n1 ; N#Cc1cc(-n2c3ccc(-c4nc(-c5ccccc5)nc(-c5ccccc5)n4)cc3c3cc(-c4nc(-c5ccccc5)nc(-c5ccccc5)n4)ccc32)ccc1-c1nc(-c2ccccc2)nc(-c2ccc(-n3c4ccc(-c5nc(-c6ccccc6)nc(-c6ccccc6)n5)cc4c4cc(-c5nc(-c6ccccc6)nc(-c6ccccc6)n5)ccc43)cc2-c2ccccc2C#N)n1]

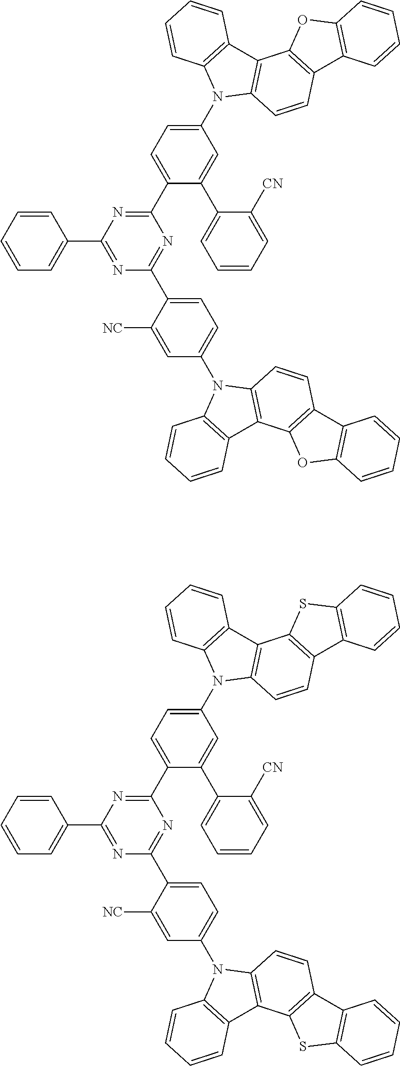 [SMILES: N#Cc1cc(-n2c3ccccc3c3c4oc5ccccc5c4ccc32)ccc1-c1nc(-c2ccccc2)nc(-c2ccc(-n3c4ccccc4c4c5oc6ccccc6c5ccc43)cc2-c2ccccc2C#N)n1 ; N#Cc1cc(-n2c3ccccc3c3c4sc5ccccc5c4ccc32)ccc1-c1nc(-c2ccccc2)nc(-c2ccc(-n3c4ccccc4c4c5sc6ccccc6c5ccc43)cc2-c2ccccc2C#N)n1]

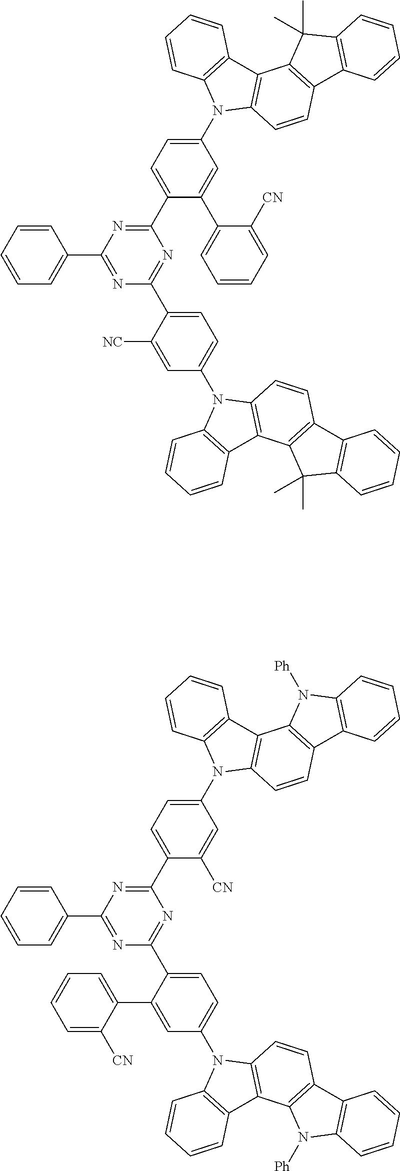 [SMILES: CC1(C)c2ccccc2-c2ccc3c(c21)c1ccccc1n3-c1ccc(-c2nc(-c3ccccc3)nc(-c3ccc(-n4c5ccccc5c5c6c(ccc54)-c4ccccc4C6(C)C)cc3-c3ccccc3C#N)n2)c(C#N)c1 ; N#Cc1cc(-n2c3ccccc3c3c2ccc2c4ccccc4n(-c4ccccc4)c23)ccc1-c1nc(-c2ccccc2)nc(-c2ccc(-n3c4ccccc4c4c3ccc3c5ccccc5n(-c5ccccc5)c34)cc2-c2ccccc2C#N)n1]

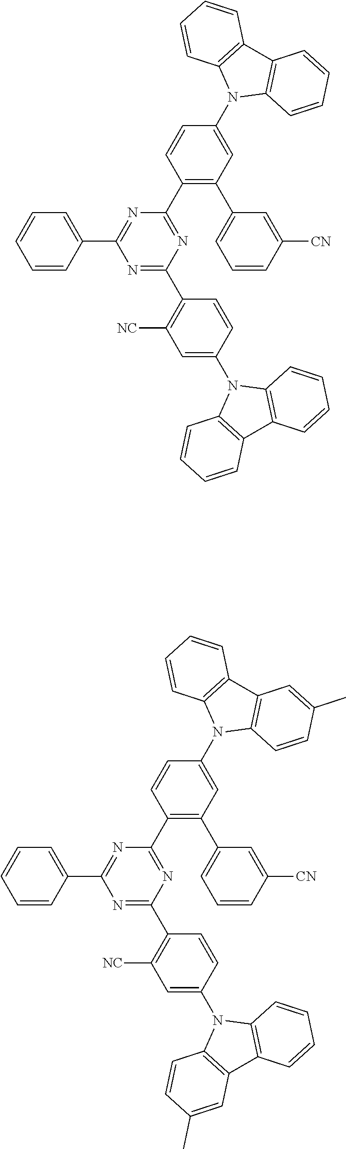 [SMILES: N#Cc1cccc(-c2cc(-n3c4ccccc4c4ccccc43)ccc2-c2nc(-c3ccccc3)nc(-c3ccc(-n4c5ccccc5c5ccccc54)cc3C#N)n2)c1 ; Cc1ccc2c(c1)c1ccccc1n2-c1ccc(-c2nc(-c3ccccc3)nc(-c3ccc(-n4c5ccccc5c5cc(C)ccc54)cc3-c3cccc(C#N)c3)n2)c(C#N)c1]

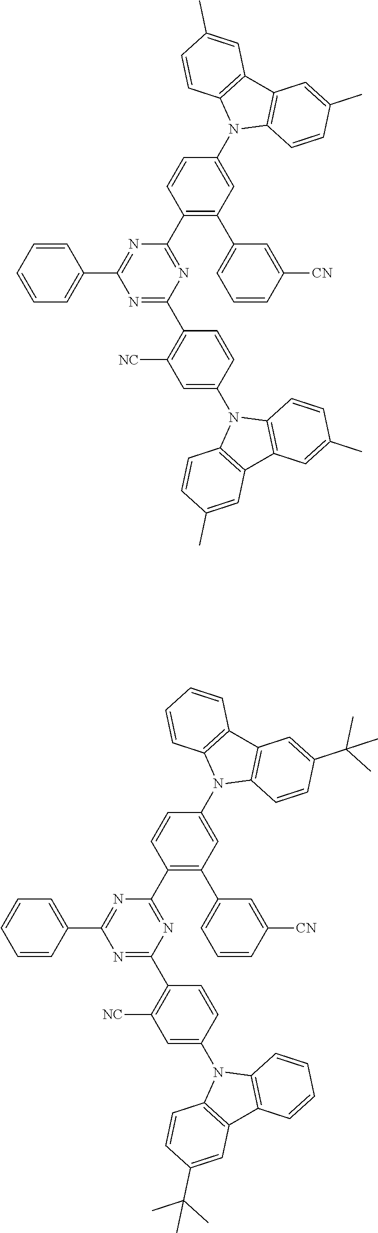 [SMILES: Cc1ccc2c(c1)c1cc(C)ccc1n2-c1ccc(-c2nc(-c3ccccc3)nc(-c3ccc(-n4c5ccc(C)cc5c5cc(C)ccc54)cc3-c3cccc(C#N)c3)n2)c(C#N)c1 ; CC(C)(C)c1ccc2c(c1)c1ccccc1n2-c1ccc(-c2nc(-c3ccccc3)nc(-c3ccc(-n4c5ccccc5c5cc(C(C)(C)C)ccc54)cc3-c3cccc(C#N)c3)n2)c(C#N)c1]

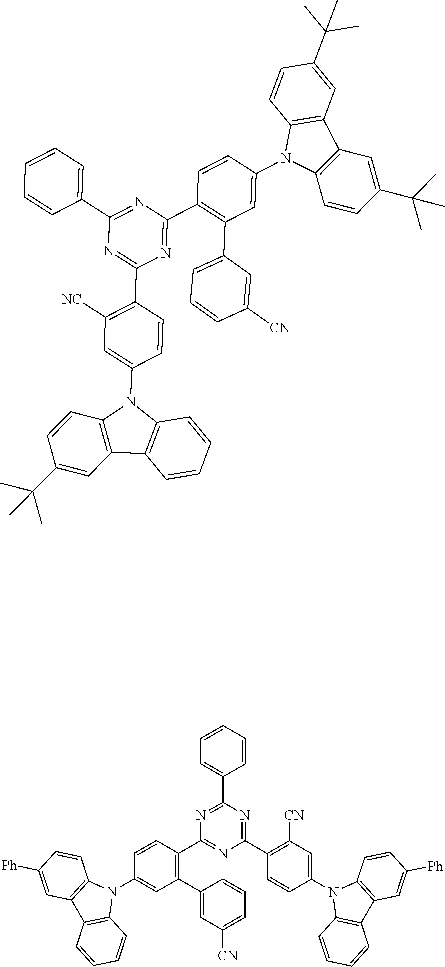 [SMILES: CC(C)(C)c1ccc2c(c1)c1ccccc1n2-c1ccc(-c2nc(-c3ccccc3)nc(-c3ccc(-n4c5ccc(C(C)(C)C)cc5c5cc(C(C)(C)C)ccc54)cc3-c3cccc(C#N)c3)n2)c(C#N)c1 ; N#Cc1cccc(-c2cc(-n3c4ccccc4c4cc(-c5ccccc5)ccc43)ccc2-c2nc(-c3ccccc3)nc(-c3ccc(-n4c5ccccc5c5cc(-c6ccccc6)ccc54)cc3C#N)n2)c1]

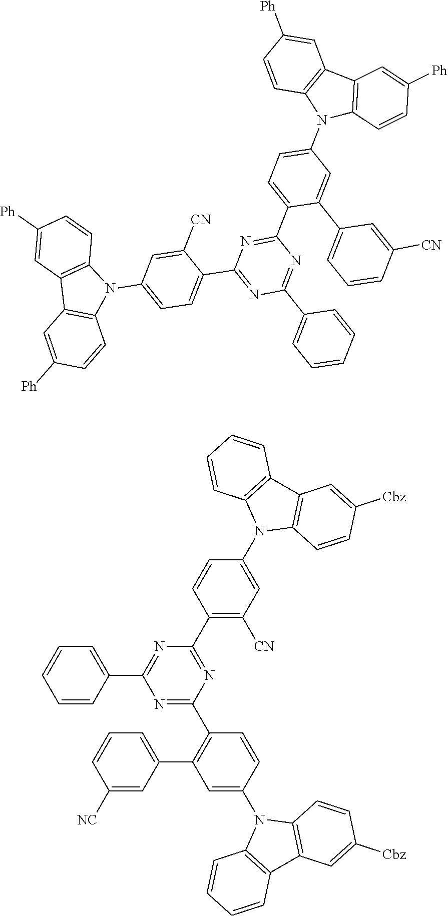 [SMILES: N#Cc1cccc(-c2cc(-n3c4ccc(-c5ccccc5)cc4c4cc(-c5ccccc5)ccc43)ccc2-c2nc(-c3ccccc3)nc(-c3ccc(-n4c5ccc(-c6ccccc6)cc5c5cc(-c6ccccc6)ccc54)cc3C#N)n2)c1 ; N#Cc1cccc(-c2cc(-n3c4ccccc4c4cc(C(=O)OCc5ccccc5)ccc43)ccc2-c2nc(-c3ccccc3)nc(-c3ccc(-n4c5ccccc5c5cc(C(=O)OCc6ccccc6)ccc54)cc3C#N)n2)c1]

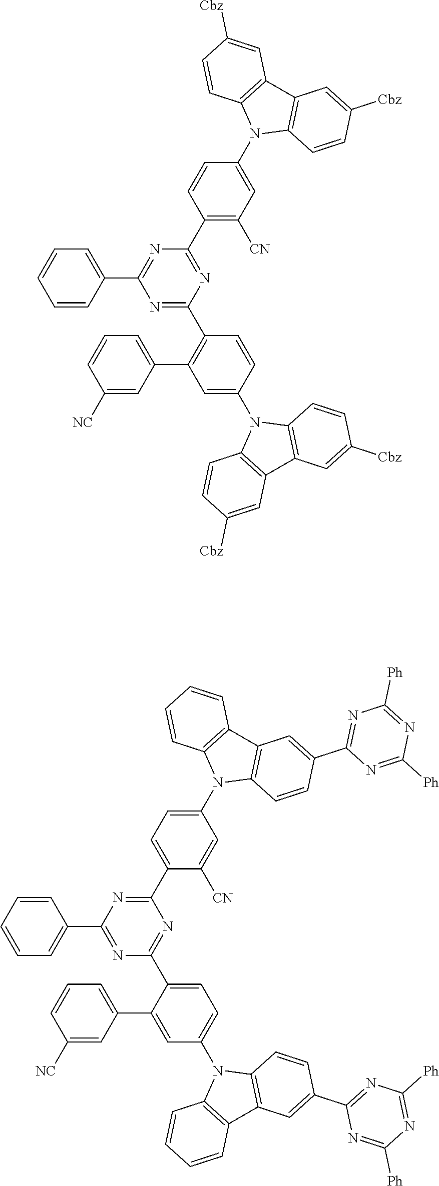 [SMILES: N#Cc1cccc(-c2cc(-n3c4ccc(C(=O)OCc5ccccc5)cc4c4cc(C(=O)OCc5ccccc5)ccc43)ccc2-c2nc(-c3ccccc3)nc(-c3ccc(-n4c5ccc(C(=O)OCc6ccccc6)cc5c5cc(C(=O)OCc6ccccc6)ccc54)cc3C#N)n2)c1 ; N#Cc1cccc(-c2cc(-n3c4ccccc4c4cc(-c5nc(-c6ccccc6)nc(-c6ccccc6)n5)ccc43)ccc2-c2nc(-c3ccccc3)nc(-c3ccc(-n4c5ccccc5c5cc(-c6nc(-c7ccccc7)nc(-c7ccccc7)n6)ccc54)cc3C#N)n2)c1]

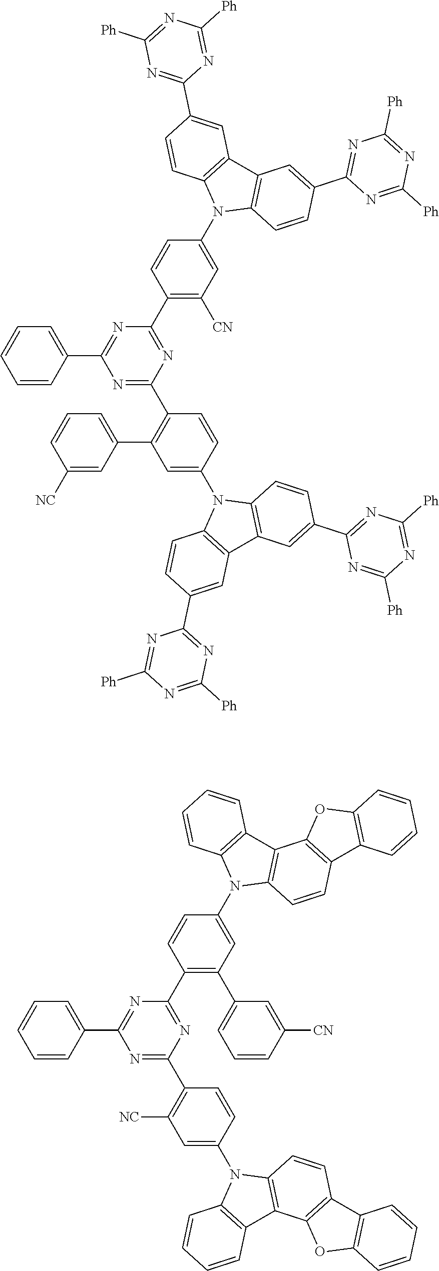 [SMILES: N#Cc1cccc(-c2cc(-n3c4ccc(-c5nc(-c6ccccc6)nc(-c6ccccc6)n5)cc4c4cc(-c5nc(-c6ccccc6)nc(-c6ccccc6)n5)ccc43)ccc2-c2nc(-c3ccccc3)nc(-c3ccc(-n4c5ccc(-c6nc(-c7ccccc7)nc(-c7ccccc7)n6)cc5c5cc(-c6nc(-c7ccccc7)nc(-c7ccccc7)n6)ccc54)cc3C#N)n2)c1 ; N#Cc1cccc(-c2cc(-n3c4ccccc4c4c5oc6ccccc6c5ccc43)ccc2-c2nc(-c3ccccc3)nc(-c3ccc(-n4c5ccccc5c5c6oc7ccccc7c6ccc54)cc3C#N)n2)c1]

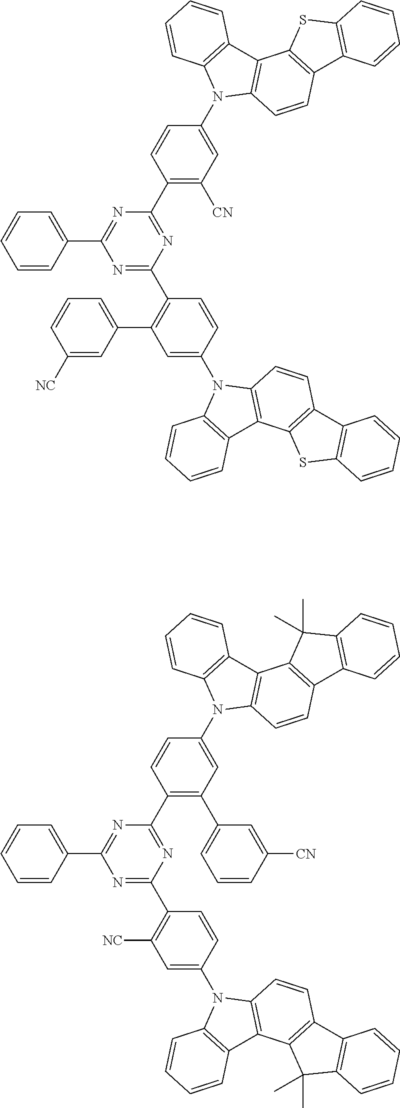 [SMILES: N#Cc1cccc(-c2cc(-n3c4ccccc4c4c5sc6ccccc6c5ccc43)ccc2-c2nc(-c3ccccc3)nc(-c3ccc(-n4c5ccccc5c5c6sc7ccccc7c6ccc54)cc3C#N)n2)c1 ; CC1(C)c2ccccc2-c2ccc3c(c21)c1ccccc1n3-c1ccc(-c2nc(-c3ccccc3)nc(-c3ccc(-n4c5ccccc5c5c6c(ccc54)-c4ccccc4C6(C)C)cc3-c3cccc(C#N)c3)n2)c(C#N)c1]

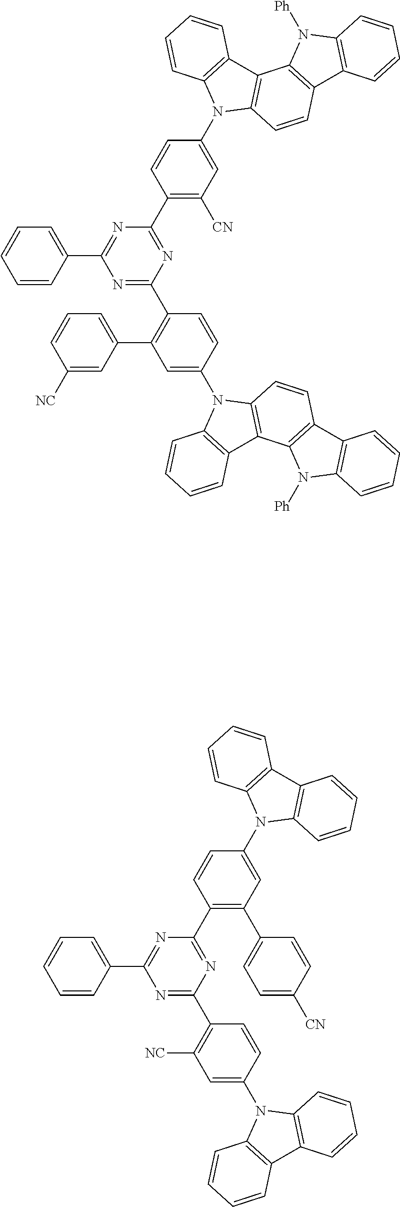 [SMILES: N#Cc1cccc(-c2cc(-n3c4ccccc4c4c3ccc3c5ccccc5n(-c5ccccc5)c34)ccc2-c2nc(-c3ccccc3)nc(-c3ccc(-n4c5ccccc5c5c4ccc4c6ccccc6n(-c6ccccc6)c45)cc3C#N)n2)c1 ; N#Cc1ccc(-c2cc(-n3c4ccccc4c4ccccc43)ccc2-c2nc(-c3ccccc3)nc(-c3ccc(-n4c5ccccc5c5ccccc54)cc3C#N)n2)cc1]

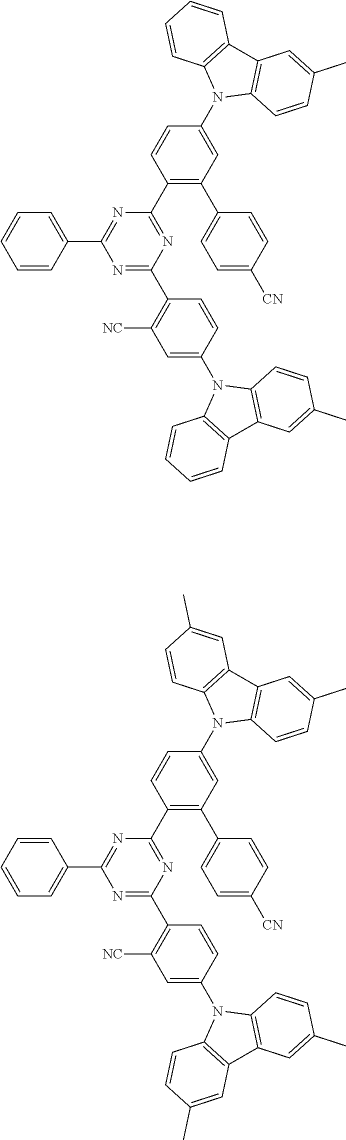 [SMILES: Cc1ccc2c(c1)c1ccccc1n2-c1ccc(-c2nc(-c3ccccc3)nc(-c3ccc(-n4c5ccccc5c5cc(C)ccc54)cc3-c3ccc(C#N)cc3)n2)c(C#N)c1 ; Cc1ccc2c(c1)c1cc(C)ccc1n2-c1ccc(-c2nc(-c3ccccc3)nc(-c3ccc(-n4c5ccc(C)cc5c5cc(C)ccc54)cc3-c3ccc(C#N)cc3)n2)c(C#N)c1]

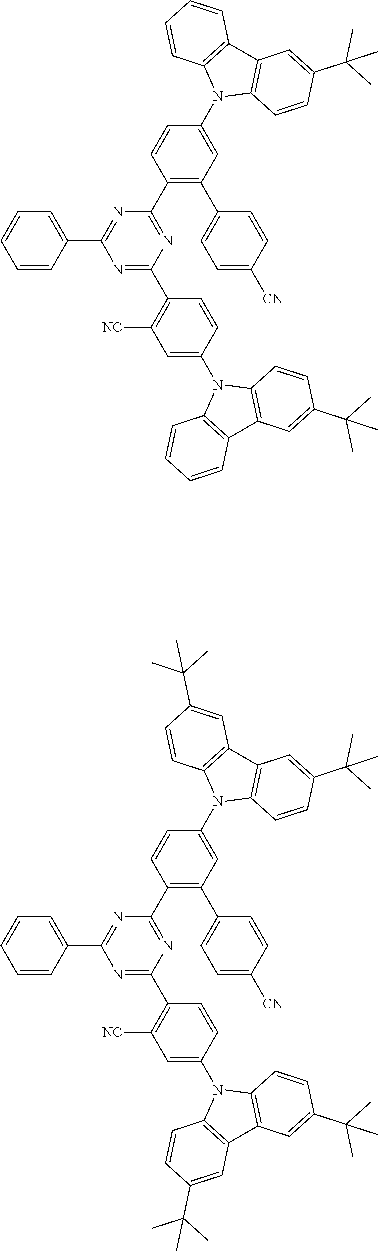 [SMILES: CC(C)(C)c1ccc2c(c1)c1ccccc1n2-c1ccc(-c2nc(-c3ccccc3)nc(-c3ccc(-n4c5ccccc5c5cc(C(C)(C)C)ccc54)cc3-c3ccc(C#N)cc3)n2)c(C#N)c1 ; CC(C)(C)c1ccc2c(c1)c1cc(C(C)(C)C)ccc1n2-c1ccc(-c2nc(-c3ccccc3)nc(-c3ccc(-n4c5ccc(C(C)(C)C)cc5c5cc(C(C)(C)C)ccc54)cc3-c3ccc(C#N)cc3)n2)c(C#N)c1]

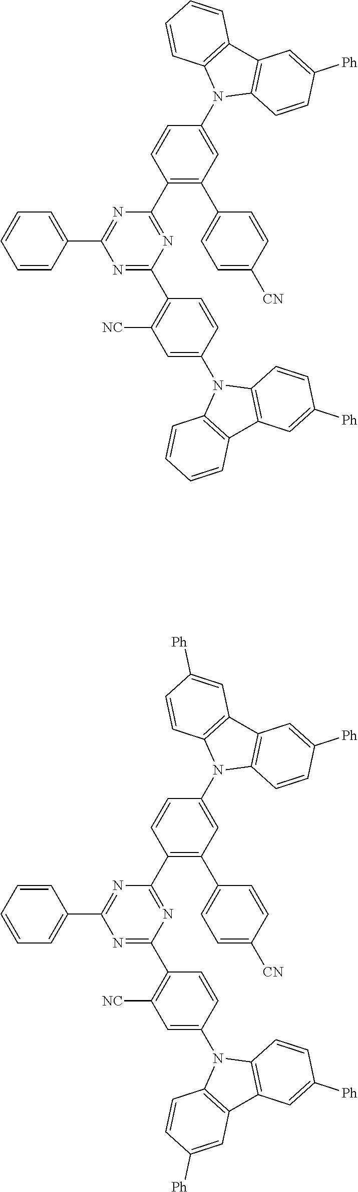 [SMILES: N#Cc1ccc(-c2cc(-n3c4ccccc4c4cc(-c5ccccc5)ccc43)ccc2-c2nc(-c3ccccc3)nc(-c3ccc(-n4c5ccccc5c5cc(-c6ccccc6)ccc54)cc3C#N)n2)cc1 ; N#Cc1ccc(-c2cc(-n3c4ccc(-c5ccccc5)cc4c4cc(-c5ccccc5)ccc43)ccc2-c2nc(-c3ccccc3)nc(-c3ccc(-n4c5ccc(-c6ccccc6)cc5c5cc(-c6ccccc6)ccc54)cc3C#N)n2)cc1]

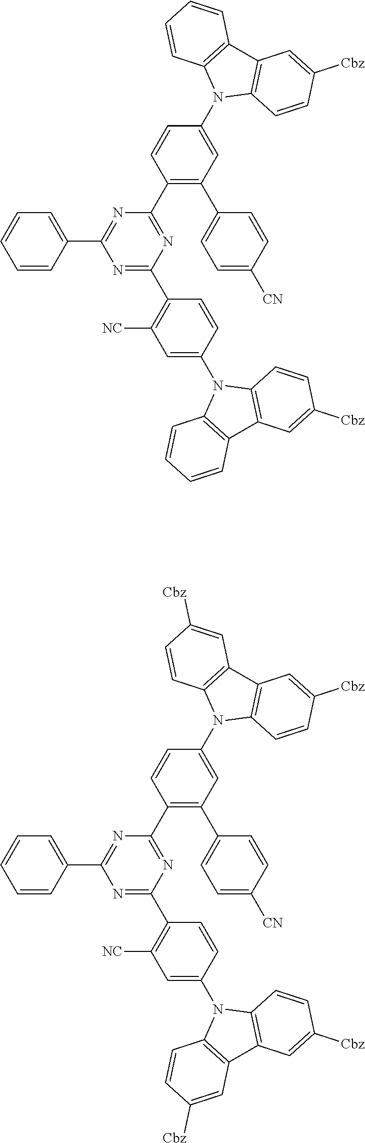 [SMILES: N#Cc1ccc(-c2cc(-n3c4ccccc4c4cc(C(=O)OCc5ccccc5)ccc43)ccc2-c2nc(-c3ccccc3)nc(-c3ccc(-n4c5ccccc5c5cc(C(=O)OCc6ccccc6)ccc54)cc3C#N)n2)cc1 ; N#Cc1ccc(-c2cc(-n3c4ccc(C(=O)OCc5ccccc5)cc4c4cc(C(=O)OCc5ccccc5)ccc43)ccc2-c2nc(-c3ccccc3)nc(-c3ccc(-n4c5ccc(C(=O)OCc6ccccc6)cc5c5cc(C(=O)OCc6ccccc6)ccc54)cc3C#N)n2)cc1]

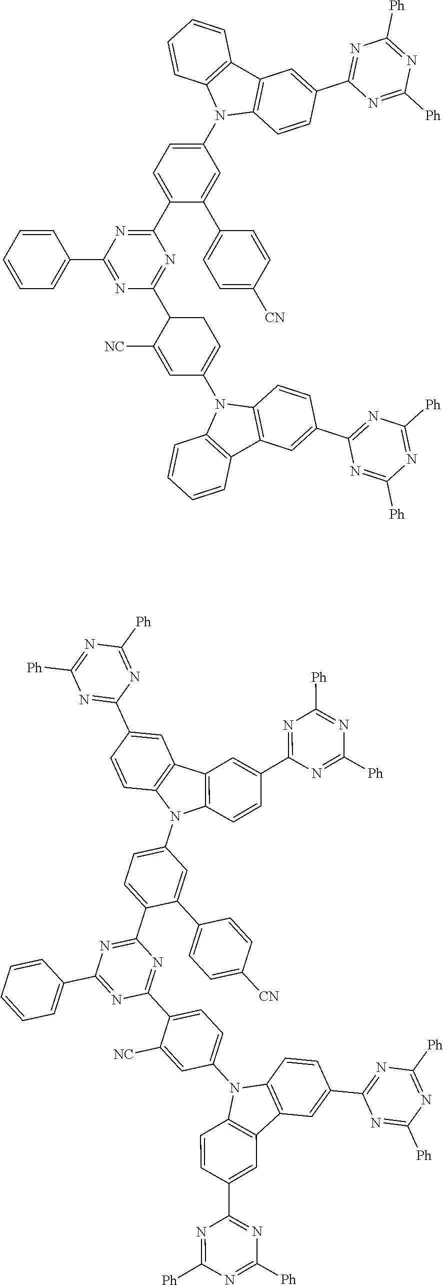 [SMILES: N#CC1=CC(n2c3ccccc3c3cc(-c4nc(-c5ccccc5)nc(-c5ccccc5)n4)ccc32)=CCC1c1nc(-c2ccccc2)nc(-c2ccc(-n3c4ccccc4c4cc(-c5nc(-c6ccccc6)nc(-c6ccccc6)n5)ccc43)cc2-c2ccc(C#N)cc2)n1 ; N#Cc1ccc(-c2cc(-n3c4ccc(-c5nc(-c6ccccc6)nc(-c6ccccc6)n5)cc4c4cc(-c5nc(-c6ccccc6)nc(-c6ccccc6)n5)ccc43)ccc2-c2nc(-c3ccccc3)nc(-c3ccc(-n4c5ccc(-c6nc(-c7ccccc7)nc(-c7ccccc7)n6)cc5c5cc(-c6nc(-c7ccccc7)nc(-c7ccccc7)n6)ccc54)cc3C#N)n2)cc1]

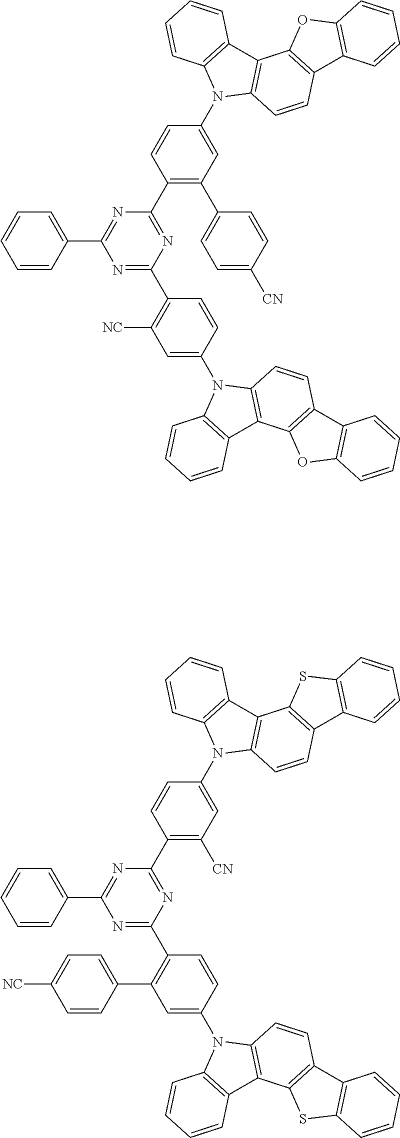 [SMILES: N#Cc1ccc(-c2cc(-n3c4ccccc4c4c5oc6ccccc6c5ccc43)ccc2-c2nc(-c3ccccc3)nc(-c3ccc(-n4c5ccccc5c5c6oc7ccccc7c6ccc54)cc3C#N)n2)cc1 ; N#Cc1ccc(-c2cc(-n3c4ccccc4c4c5sc6ccccc6c5ccc43)ccc2-c2nc(-c3ccccc3)nc(-c3ccc(-n4c5ccccc5c5c6sc7ccccc7c6ccc54)cc3C#N)n2)cc1]

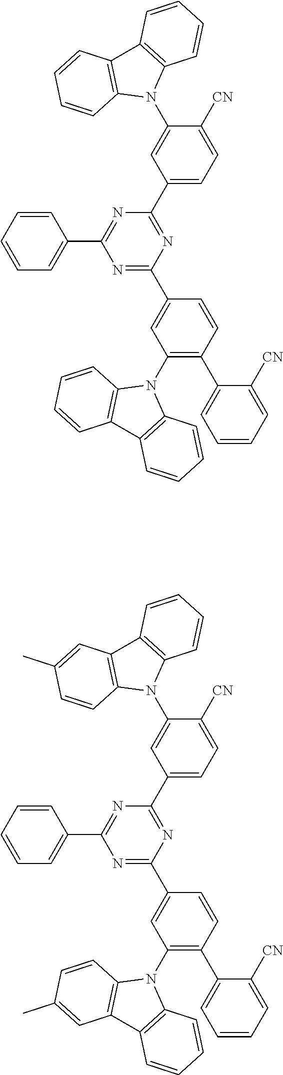 [SMILES: N#Cc1ccccc1-c1ccc(-c2nc(-c3ccccc3)nc(-c3ccc(C#N)c(-n4c5ccccc5c5ccccc54)c3)n2)cc1-n1c2ccccc2c2ccccc21 ; Cc1ccc2c(c1)c1ccccc1n2-c1cc(-c2nc(-c3ccccc3)nc(-c3ccc(-c4ccccc4C#N)c(-n4c5ccccc5c5cc(C)ccc54)c3)n2)ccc1C#N]

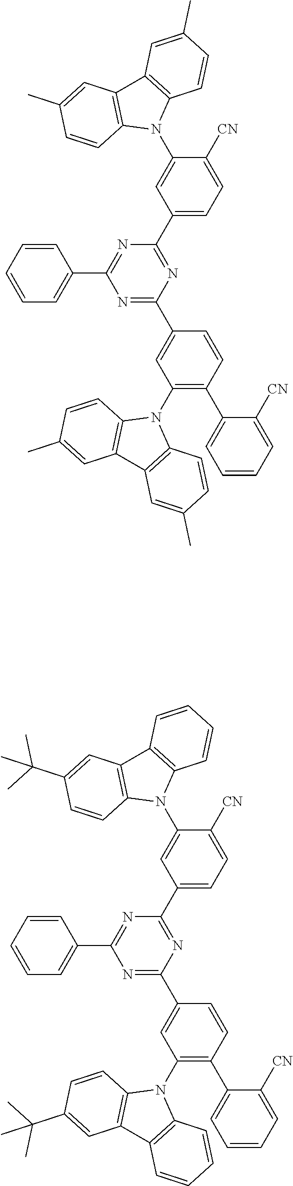 [SMILES: Cc1ccc2c(c1)c1cc(C)ccc1n2-c1cc(-c2nc(-c3ccccc3)nc(-c3ccc(-c4ccccc4C#N)c(-n4c5ccc(C)cc5c5cc(C)ccc54)c3)n2)ccc1C#N ; CC(C)(C)c1ccc2c(c1)c1ccccc1n2-c1cc(-c2nc(-c3ccccc3)nc(-c3ccc(-c4ccccc4C#N)c(-n4c5ccccc5c5cc(C(C)(C)C)ccc54)c3)n2)ccc1C#N]

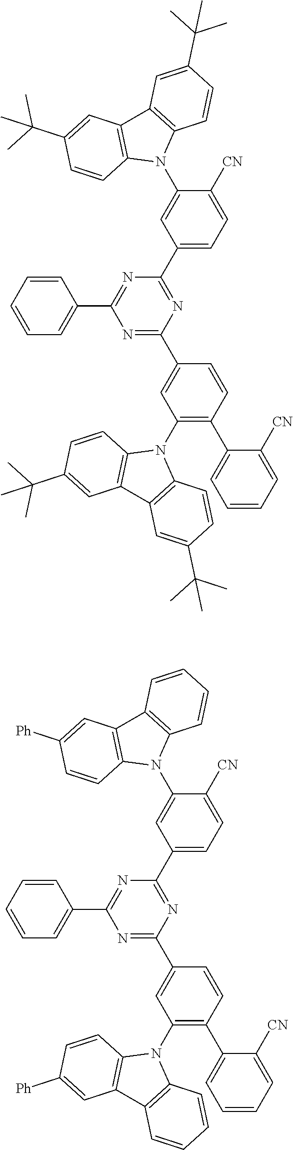 [SMILES: CC(C)(C)c1ccc2c(c1)c1cc(C(C)(C)C)ccc1n2-c1cc(-c2nc(-c3ccccc3)nc(-c3ccc(-c4ccccc4C#N)c(-n4c5ccc(C(C)(C)C)cc5c5cc(C(C)(C)C)ccc54)c3)n2)ccc1C#N ; N#Cc1ccccc1-c1ccc(-c2nc(-c3ccccc3)nc(-c3ccc(C#N)c(-n4c5ccccc5c5cc(-c6ccccc6)ccc54)c3)n2)cc1-n1c2ccccc2c2cc(-c3ccccc3)ccc21]

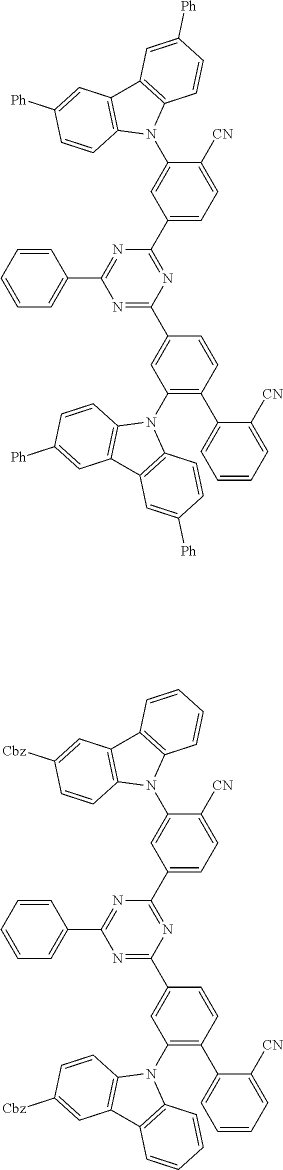 [SMILES: N#Cc1ccccc1-c1ccc(-c2nc(-c3ccccc3)nc(-c3ccc(C#N)c(-n4c5ccc(-c6ccccc6)cc5c5cc(-c6ccccc6)ccc54)c3)n2)cc1-n1c2ccc(-c3ccccc3)cc2c2cc(-c3ccccc3)ccc21 ; N#Cc1ccccc1-c1ccc(-c2nc(-c3ccccc3)nc(-c3ccc(C#N)c(-n4c5ccccc5c5cc(C(=O)OCc6ccccc6)ccc54)c3)n2)cc1-n1c2ccccc2c2cc(C(=O)OCc3ccccc3)ccc21]

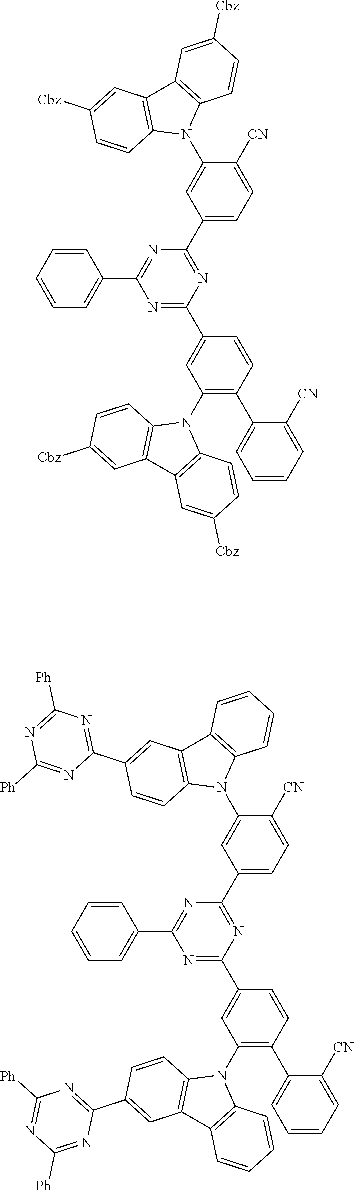 [SMILES: N#Cc1ccccc1-c1ccc(-c2nc(-c3ccccc3)nc(-c3ccc(C#N)c(-n4c5ccc(C(=O)OCc6ccccc6)cc5c5cc(C(=O)OCc6ccccc6)ccc54)c3)n2)cc1-n1c2ccc(C(=O)OCc3ccccc3)cc2c2cc(C(=O)OCc3ccccc3)ccc21 ; N#Cc1ccccc1-c1ccc(-c2nc(-c3ccccc3)nc(-c3ccc(C#N)c(-n4c5ccccc5c5cc(-c6nc(-c7ccccc7)nc(-c7ccccc7)n6)ccc54)c3)n2)cc1-n1c2ccccc2c2cc(-c3nc(-c4ccccc4)nc(-c4ccccc4)n3)ccc21]

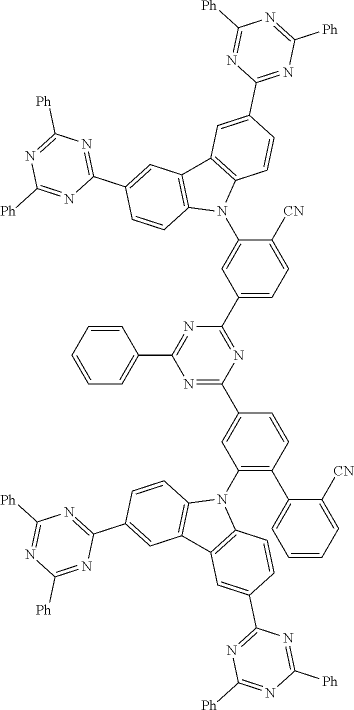 [SMILES: N#Cc1ccccc1-c1ccc(-c2nc(-c3ccccc3)nc(-c3ccc(C#N)c(-n4c5ccc(-c6nc(-c7ccccc7)nc(-c7ccccc7)n6)cc5c5cc(-c6nc(-c7ccccc7)nc(-c7ccccc7)n6)ccc54)c3)n2)cc1-n1c2ccc(-c3nc(-c4ccccc4)nc(-c4ccccc4)n3)cc2c2cc(-c3nc(-c4ccccc4)nc(-c4ccccc4)n3)ccc21]

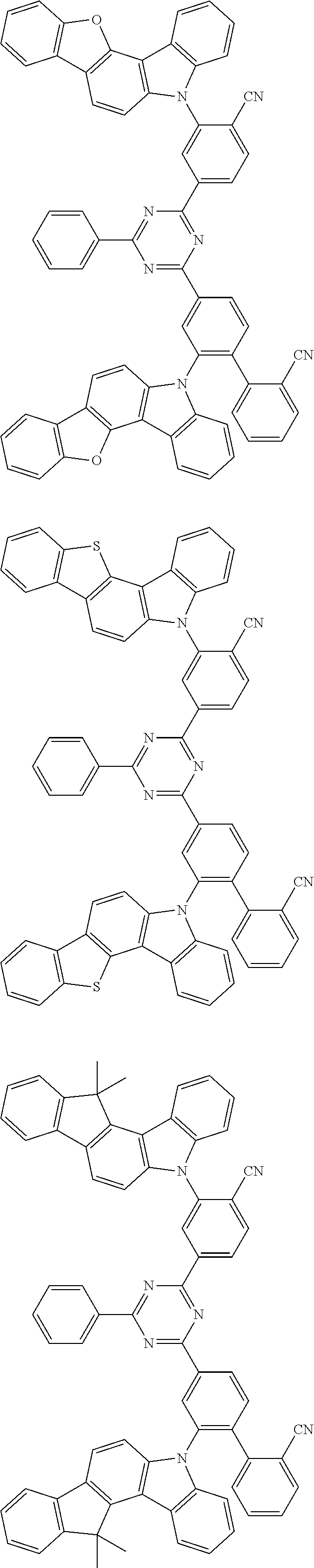 [SMILES: N#Cc1ccccc1-c1ccc(-c2nc(-c3ccccc3)nc(-c3ccc(C#N)c(-n4c5ccccc5c5c6oc7ccccc7c6ccc54)c3)n2)cc1-n1c2ccccc2c2c3oc4ccccc4c3ccc21 ; N#Cc1ccccc1-c1ccc(-c2nc(-c3ccccc3)nc(-c3ccc(C#N)c(-n4c5ccccc5c5c6sc7ccccc7c6ccc54)c3)n2)cc1-n1c2ccccc2c2c3sc4ccccc4c3ccc21 ; CC1(C)c2ccccc2-c2ccc3c(c21)c1ccccc1n3-c1cc(-c2nc(-c3ccccc3)nc(-c3ccc(-c4ccccc4C#N)c(-n4c5ccccc5c5c6c(ccc54)-c4ccccc4C6(C)C)c3)n2)ccc1C#N]

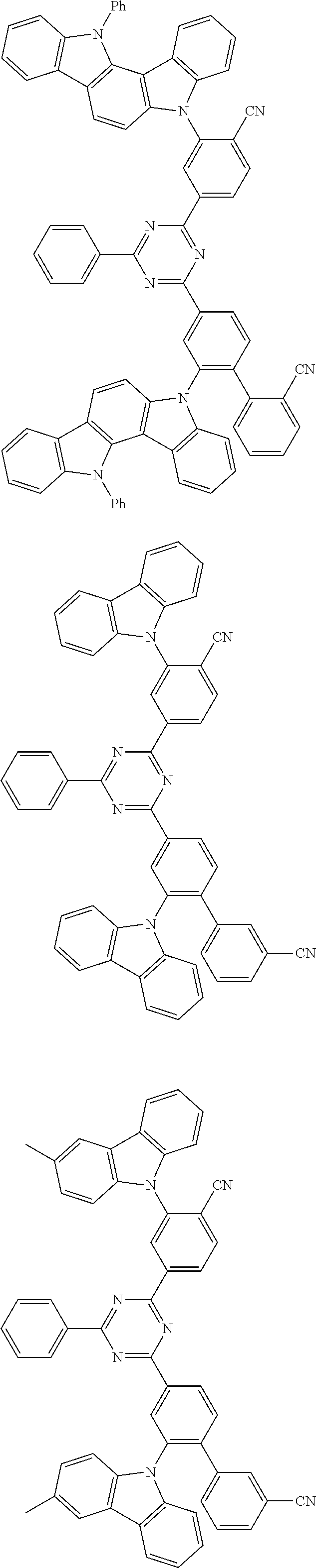 [SMILES: N#Cc1ccccc1-c1ccc(-c2nc(-c3ccccc3)nc(-c3ccc(C#N)c(-n4c5ccccc5c5c4ccc4c6ccccc6n(-c6ccccc6)c45)c3)n2)cc1-n1c2ccccc2c2c1ccc1c3ccccc3n(-c3ccccc3)c12 ; N#Cc1cccc(-c2ccc(-c3nc(-c4ccccc4)nc(-c4ccc(C#N)c(-n5c6ccccc6c6ccccc65)c4)n3)cc2-n2c3ccccc3c3ccccc32)c1 ; Cc1ccc2c(c1)c1ccccc1n2-c1cc(-c2nc(-c3ccccc3)nc(-c3ccc(-c4cccc(C#N)c4)c(-n4c5ccccc5c5cc(C)ccc54)c3)n2)ccc1C#N]

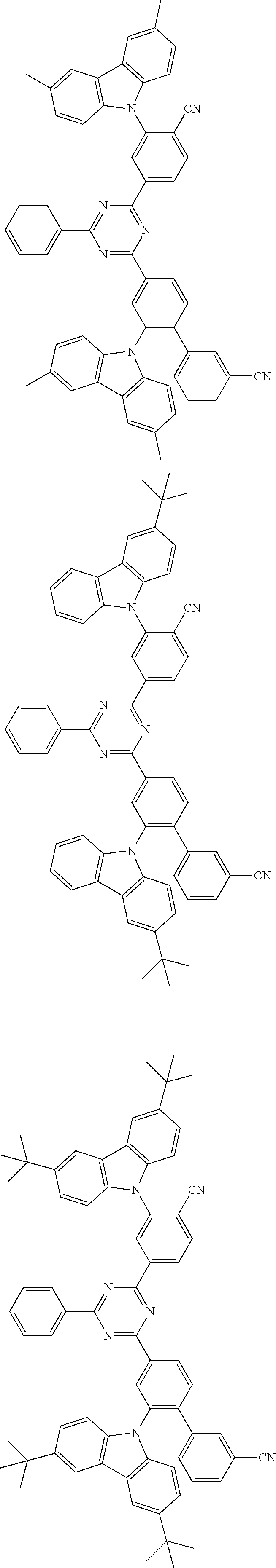

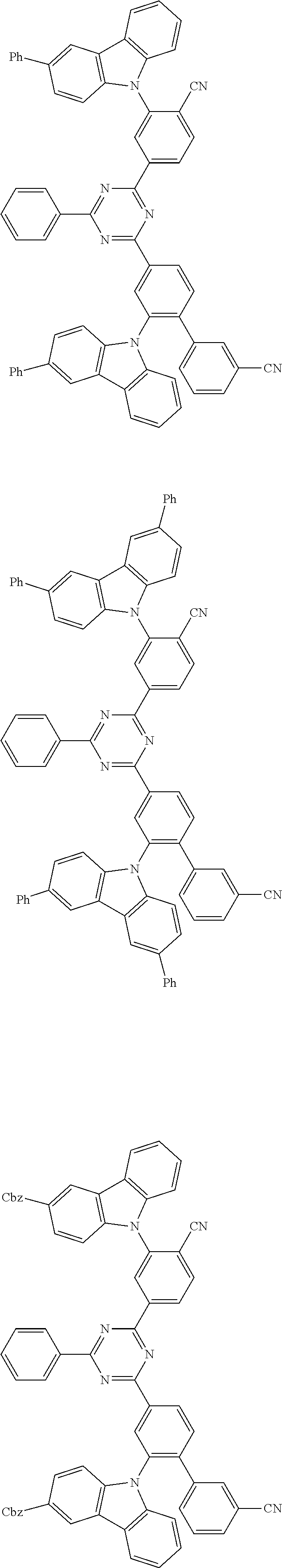 [SMILES: N#Cc1cccc(-c2ccc(-c3nc(-c4ccccc4)nc(-c4ccc(C#N)c(-n5c6ccccc6c6cc(-c7ccccc7)ccc65)c4)n3)cc2-n2c3ccccc3c3cc(-c4ccccc4)ccc32)c1 ; N#Cc1cccc(-c2ccc(-c3nc(-c4ccccc4)nc(-c4ccc(C#N)c(-n5c6ccc(-c7ccccc7)cc6c6cc(-c7ccccc7)ccc65)c4)n3)cc2-n2c3ccc(-c4ccccc4)cc3c3cc(-c4ccccc4)ccc32)c1 ; N#Cc1cccc(-c2ccc(-c3nc(-c4ccccc4)nc(-c4ccc(C#N)c(-n5c6ccccc6c6cc(C(=O)OCc7ccccc7)ccc65)c4)n3)cc2-n2c3ccccc3c3cc(C(=O)OCc4ccccc4)ccc32)c1]

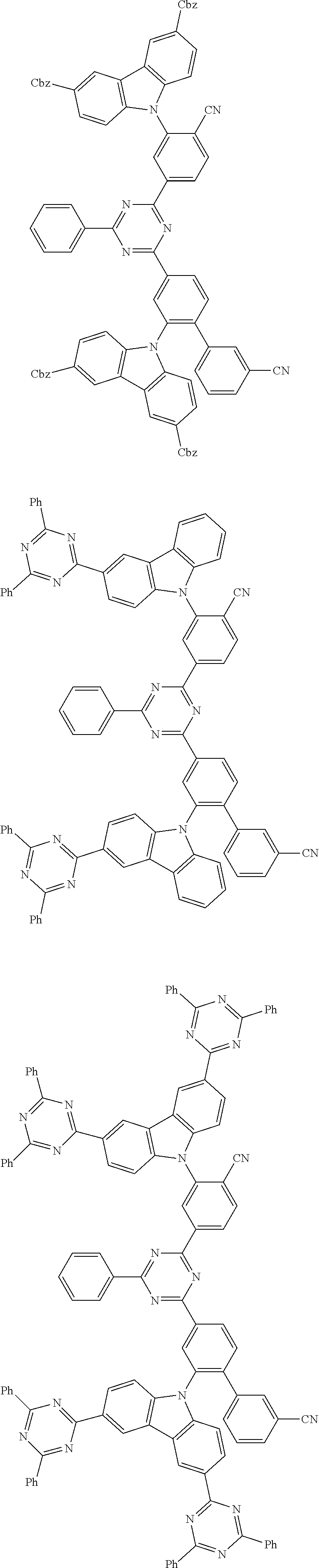

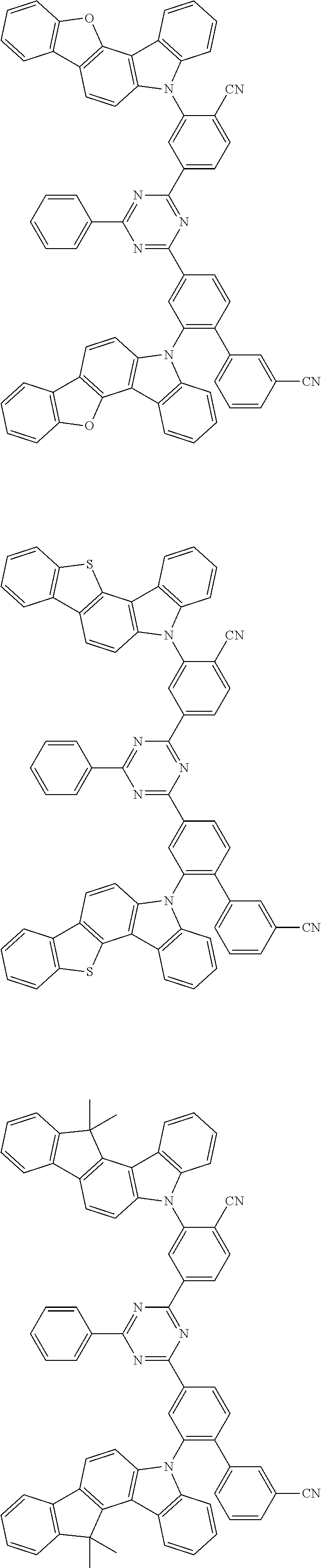 [SMILES: N#Cc1cccc(-c2ccc(-c3nc(-c4ccccc4)nc(-c4ccc(C#N)c(-n5c6ccccc6c6c7oc8ccccc8c7ccc65)c4)n3)cc2-n2c3ccccc3c3c4oc5ccccc5c4ccc32)c1 ; N#Cc1cccc(-c2ccc(-c3nc(-c4ccccc4)nc(-c4ccc(C#N)c(-n5c6ccccc6c6c7sc8ccccc8c7ccc65)c4)n3)cc2-n2c3ccccc3c3c4sc5ccccc5c4ccc32)c1 ; CC1(C)c2ccccc2-c2ccc3c(c21)c1ccccc1n3-c1cc(-c2nc(-c3ccccc3)nc(-c3ccc(-c4cccc(C#N)c4)c(-n4c5ccccc5c5c6c(ccc54)-c4ccccc4C6(C)C)c3)n2)ccc1C#N]

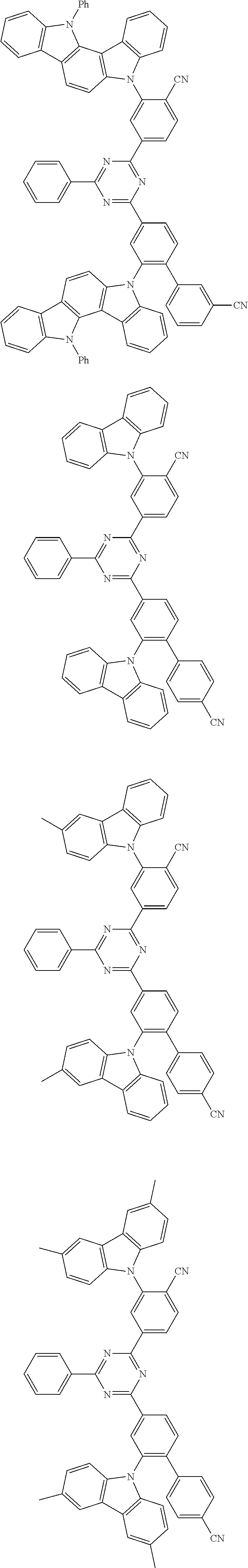 [SMILES: N#Cc1cccc(-c2ccc(-c3nc(-c4ccccc4)nc(-c4ccc(C#N)c(-n5c6ccccc6c6c5ccc5c7ccccc7n(-c7ccccc7)c56)c4)n3)cc2-n2c3ccccc3c3c2ccc2c4ccccc4n(-c4ccccc4)c23)c1 ; N#Cc1ccc(-c2ccc(-c3nc(-c4ccccc4)nc(-c4ccc(C#N)c(-n5c6ccccc6c6ccccc65)c4)n3)cc2-n2c3ccccc3c3ccccc32)cc1 ; Cc1ccc2c(c1)c1ccccc1n2-c1cc(-c2nc(-c3ccccc3)nc(-c3ccc(-c4ccc(C#N)cc4)c(-n4c5ccccc5c5cc(C)ccc54)c3)n2)ccc1C#N ; Cc1ccc2c(c1)c1cc(C)ccc1n2-c1cc(-c2nc(-c3ccccc3)nc(-c3ccc(-c4ccc(C#N)cc4)c(-n4c5ccc(C)cc5c5cc(C)ccc54)c3)n2)ccc1C#N]

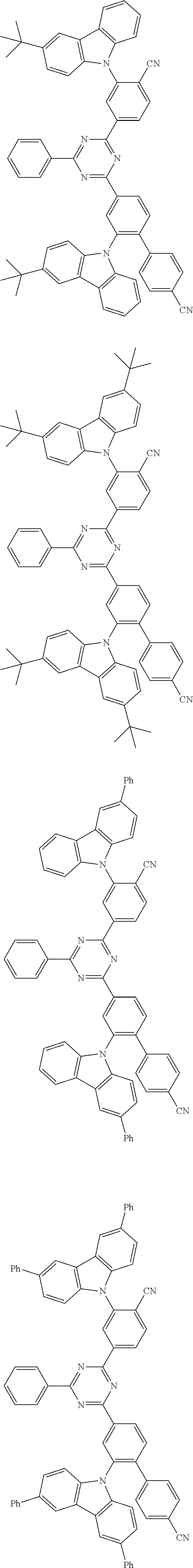

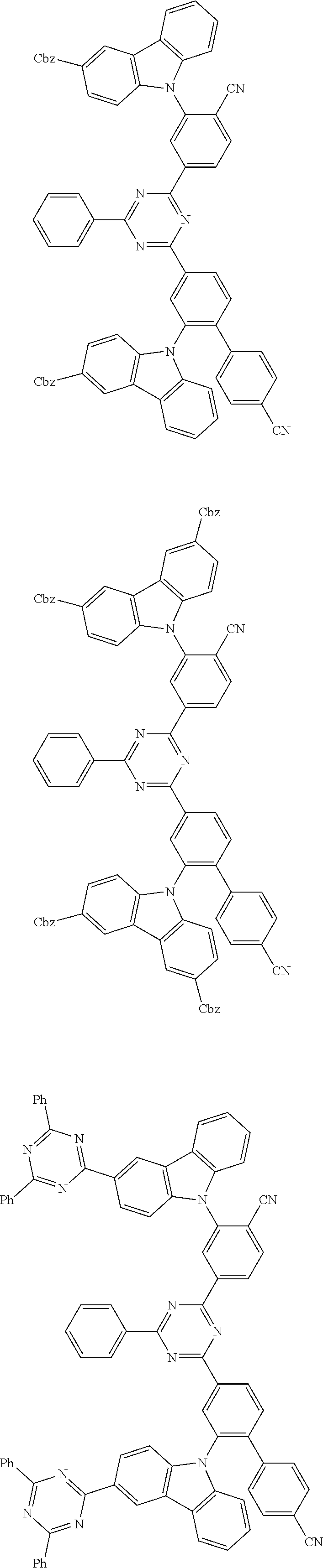 [SMILES: N#Cc1ccc(-c2ccc(-c3nc(-c4ccccc4)nc(-c4ccc(C#N)c(-n5c6ccccc6c6cc(C(=O)OCc7ccccc7)ccc65)c4)n3)cc2-n2c3ccccc3c3cc(C(=O)OCc4ccccc4)ccc32)cc1 ; N#Cc1ccc(-c2ccc(-c3nc(-c4ccccc4)nc(-c4ccc(C#N)c(-n5c6ccc(C(=O)OCc7ccccc7)cc6c6cc(C(=O)OCc7ccccc7)ccc65)c4)n3)cc2-n2c3ccc(C(=O)OCc4ccccc4)cc3c3cc(C(=O)OCc4ccccc4)ccc32)cc1 ; N#Cc1ccc(-c2ccc(-c3nc(-c4ccccc4)nc(-c4ccc(C#N)c(-n5c6ccccc6c6cc(-c7nc(-c8ccccc8)nc(-c8ccccc8)n7)ccc65)c4)n3)cc2-n2c3ccccc3c3cc(-c4nc(-c5ccccc5)nc(-c5ccccc5)n4)ccc32)cc1]

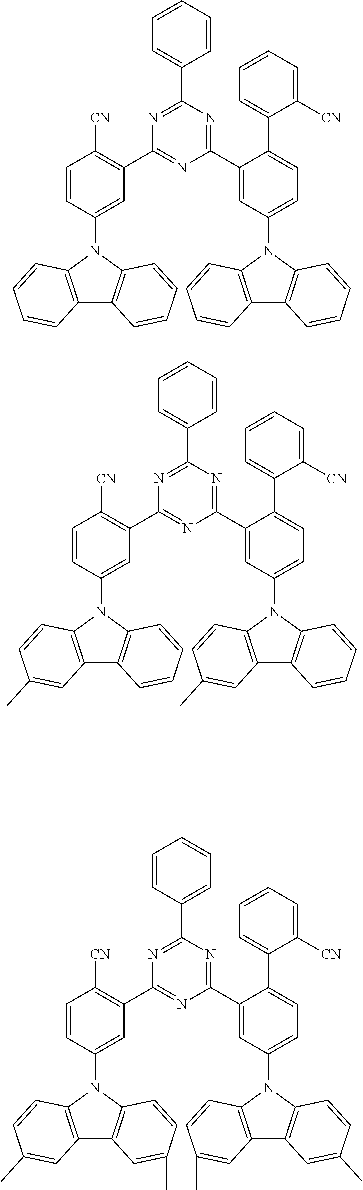

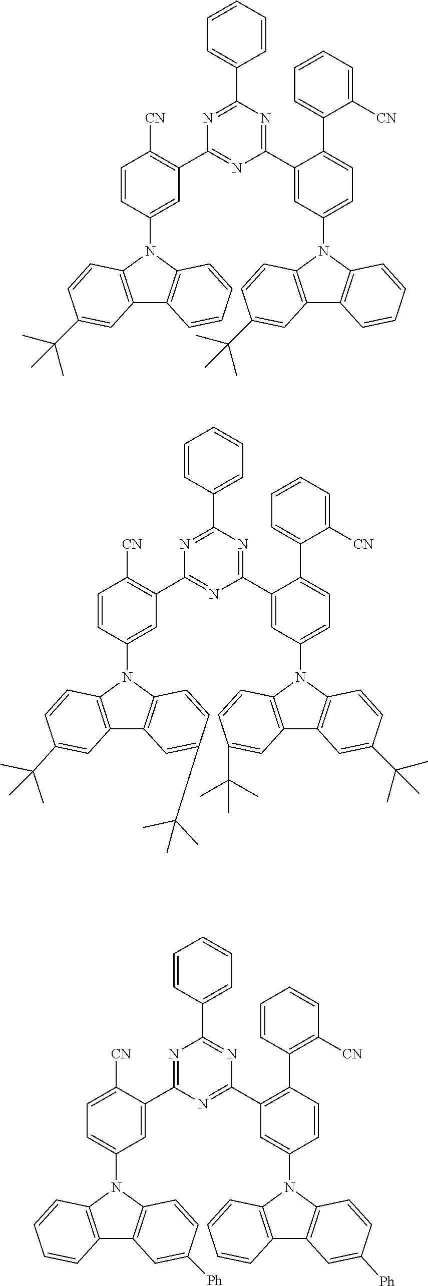 [SMILES: CC(C)(C)c1ccc2c(c1)c1ccccc1n2-c1ccc(C#N)c(-c2nc(-c3ccccc3)nc(-c3cc(-n4c5ccccc5c5cc(C(C)(C)C)ccc54)ccc3-c3ccccc3C#N)n2)c1 ; CC(C)(C)c1ccc2c(c1)c1cc(C(C)(C)C)ccc1n2-c1ccc(C#N)c(-c2nc(-c3ccccc3)nc(-c3cc(-n4c5ccc(C(C)(C)C)cc5c5cc(C(C)(C)C)ccc54)ccc3-c3ccccc3C#N)n2)c1 ; N#Cc1ccc(-n2c3ccccc3c3cc(-c4ccccc4)ccc32)cc1-c1nc(-c2ccccc2)nc(-c2cc(-n3c4ccccc4c4cc(-c5ccccc5)ccc43)ccc2-c2ccccc2C#N)n1]

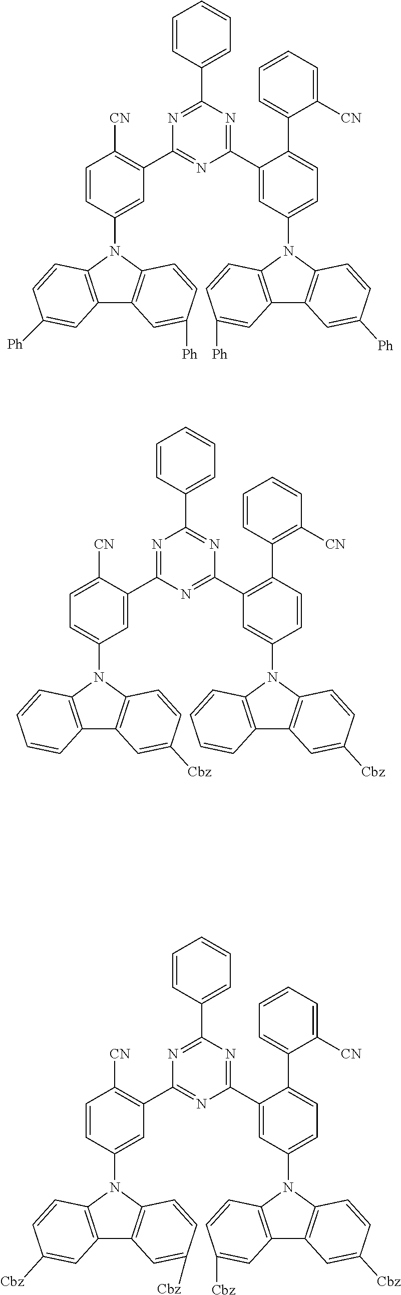 [SMILES: N#Cc1ccc(-n2c3ccc(-c4ccccc4)cc3c3cc(-c4ccccc4)ccc32)cc1-c1nc(-c2ccccc2)nc(-c2cc(-n3c4ccc(-c5ccccc5)cc4c4cc(-c5ccccc5)ccc43)ccc2-c2ccccc2C#N)n1 ; N#Cc1ccc(-n2c3ccccc3c3cc(C(=O)OCc4ccccc4)ccc32)cc1-c1nc(-c2ccccc2)nc(-c2cc(-n3c4ccccc4c4cc(C(=O)OCc5ccccc5)ccc43)ccc2-c2ccccc2C#N)n1 ; N#Cc1ccc(-n2c3ccc(C(=O)OCc4ccccc4)cc3c3cc(C(=O)OCc4ccccc4)ccc32)cc1-c1nc(-c2ccccc2)nc(-c2cc(-n3c4ccc(C(=O)OCc5ccccc5)cc4c4cc(C(=O)OCc5ccccc5)ccc43)ccc2-c2ccccc2C#N)n1]

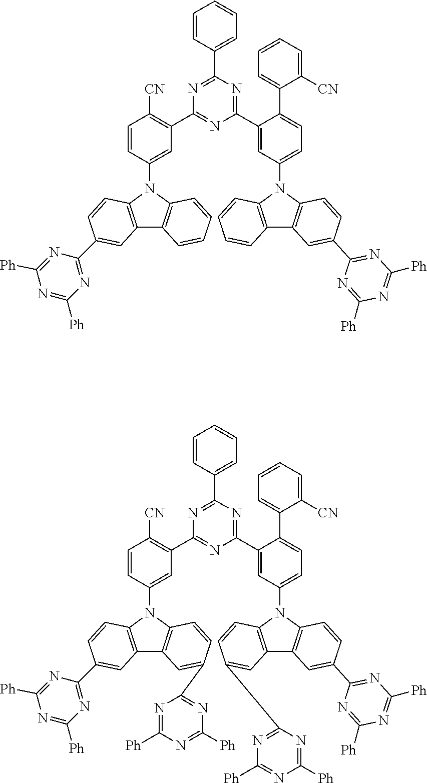 [SMILES: N#Cc1ccc(-n2c3ccccc3c3cc(-c4nc(-c5ccccc5)nc(-c5ccccc5)n4)ccc32)cc1-c1nc(-c2ccccc2)nc(-c2cc(-n3c4ccccc4c4cc(-c5nc(-c6ccccc6)nc(-c6ccccc6)n5)ccc43)ccc2-c2ccccc2C#N)n1 ; N#Cc1ccc(-n2c3ccc(-c4nc(-c5ccccc5)nc(-c5ccccc5)n4)cc3c3cc(-c4nc(-c5ccccc5)nc(-c5ccccc5)n4)ccc32)cc1-c1nc(-c2ccccc2)nc(-c2cc(-n3c4ccc(-c5nc(-c6ccccc6)nc(-c6ccccc6)n5)cc4c4cc(-c5nc(-c6ccccc6)nc(-c6ccccc6)n5)ccc43)ccc2-c2ccccc2C#N)n1]

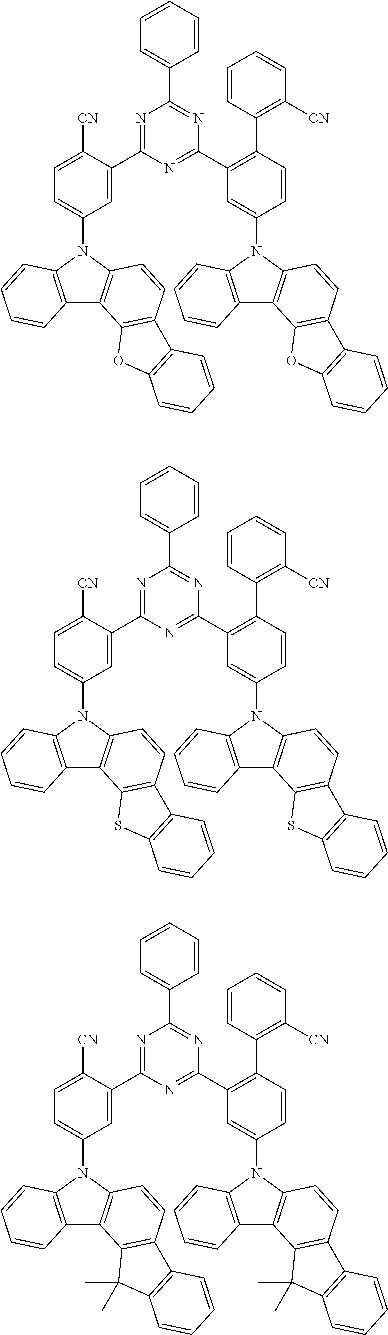 [SMILES: N#Cc1ccc(-n2c3ccccc3c3c4oc5ccccc5c4ccc32)cc1-c1nc(-c2ccccc2)nc(-c2cc(-n3c4ccccc4c4c5oc6ccccc6c5ccc43)ccc2-c2ccccc2C#N)n1 ; N#Cc1ccc(-n2c3ccccc3c3c4sc5ccccc5c4ccc32)cc1-c1nc(-c2ccccc2)nc(-c2cc(-n3c4ccccc4c4c5sc6ccccc6c5ccc43)ccc2-c2ccccc2C#N)n1 ; CC1(C)c2ccccc2-c2ccc3c(c21)c1ccccc1n3-c1ccc(C#N)c(-c2nc(-c3ccccc3)nc(-c3cc(-n4c5ccccc5c5c6c(ccc54)-c4ccccc4C6(C)C)ccc3-c3ccccc3C#N)n2)c1]

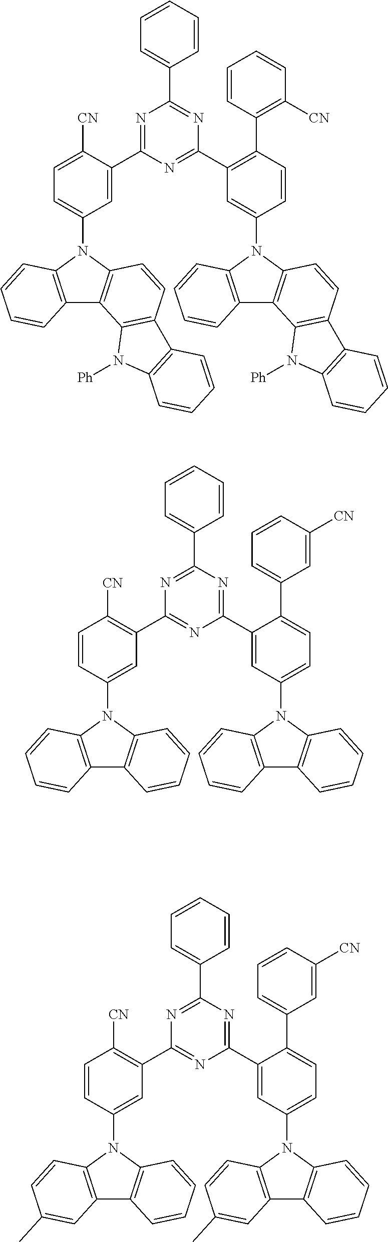 [SMILES: N#Cc1ccc(-n2c3ccccc3c3c2ccc2c4ccccc4n(-c4ccccc4)c23)cc1-c1nc(-c2ccccc2)nc(-c2cc(-n3c4ccccc4c4c3ccc3c5ccccc5n(-c5ccccc5)c34)ccc2-c2ccccc2C#N)n1 ; N#Cc1cccc(-c2ccc(-n3c4ccccc4c4ccccc43)cc2-c2nc(-c3ccccc3)nc(-c3cc(-n4c5ccccc5c5ccccc54)ccc3C#N)n2)c1 ; Cc1ccc2c(c1)c1ccccc1n2-c1ccc(C#N)c(-c2nc(-c3ccccc3)nc(-c3cc(-n4c5ccccc5c5cc(C)ccc54)ccc3-c3cccc(C#N)c3)n2)c1]

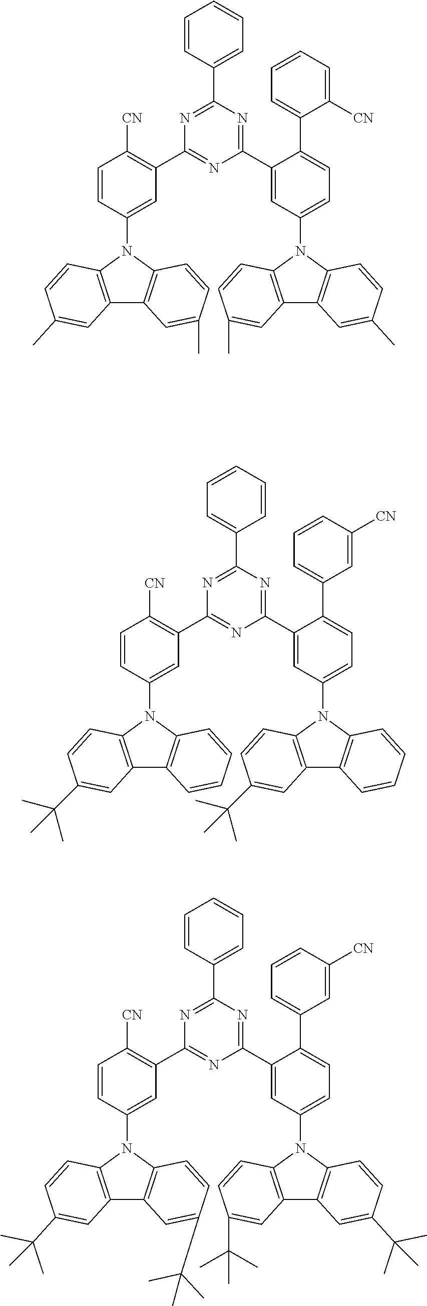

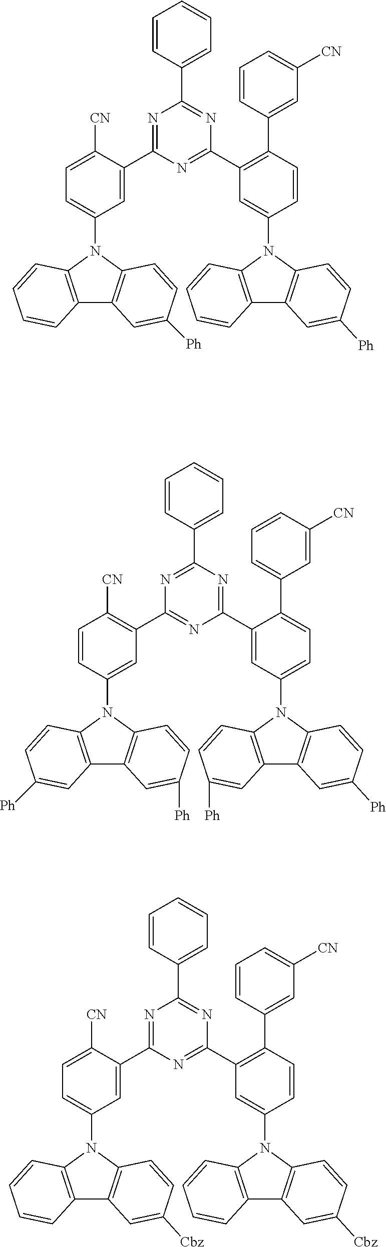 [SMILES: N#Cc1cccc(-c2ccc(-n3c4ccccc4c4cc(-c5ccccc5)ccc43)cc2-c2nc(-c3ccccc3)nc(-c3cc(-n4c5ccccc5c5cc(-c6ccccc6)ccc54)ccc3C#N)n2)c1 ; N#Cc1cccc(-c2ccc(-n3c4ccc(-c5ccccc5)cc4c4cc(-c5ccccc5)ccc43)cc2-c2nc(-c3ccccc3)nc(-c3cc(-n4c5ccc(-c6ccccc6)cc5c5cc(-c6ccccc6)ccc54)ccc3C#N)n2)c1 ; N#Cc1cccc(-c2ccc(-n3c4ccccc4c4cc(C(=O)OCc5ccccc5)ccc43)cc2-c2nc(-c3ccccc3)nc(-c3cc(-n4c5ccccc5c5cc(C(=O)OCc6ccccc6)ccc54)ccc3C#N)n2)c1]

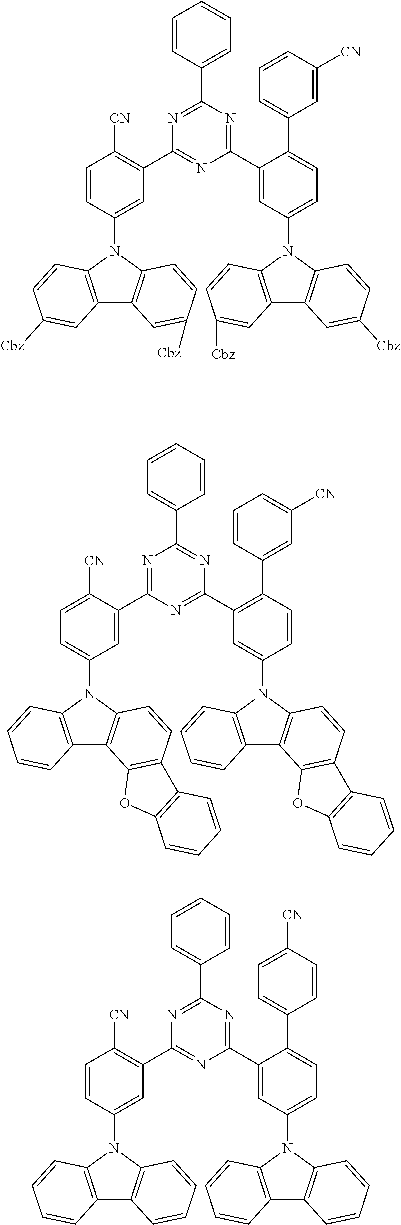

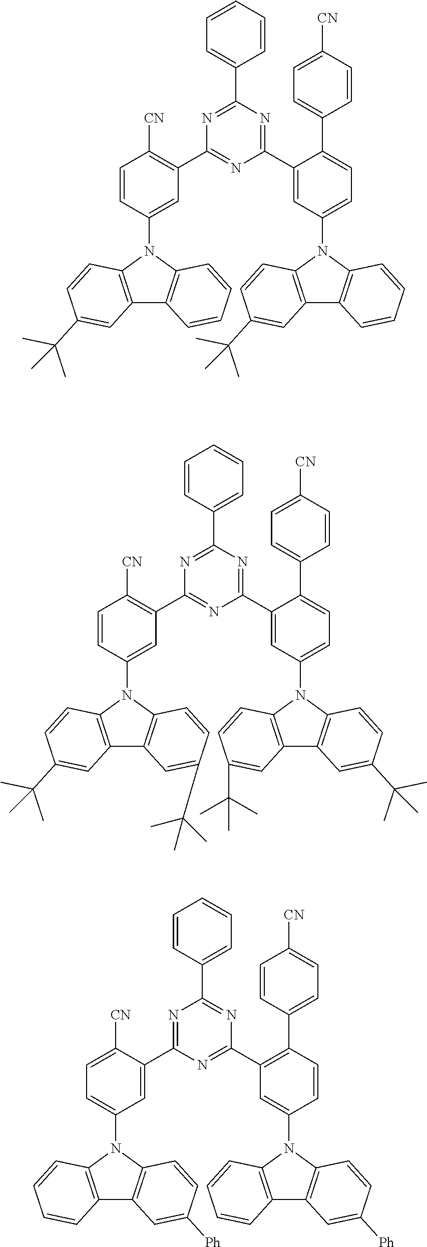 [SMILES: CC(C)(C)c1ccc2c(c1)c1ccccc1n2-c1ccc(C#N)c(-c2nc(-c3ccccc3)nc(-c3cc(-n4c5ccccc5c5cc(C(C)(C)C)ccc54)ccc3-c3ccc(C#N)cc3)n2)c1 ; CC(C)(C)c1ccc2c(c1)c1cc(C(C)(C)C)ccc1n2-c1ccc(C#N)c(-c2nc(-c3ccccc3)nc(-c3cc(-n4c5ccc(C(C)(C)C)cc5c5cc(C(C)(C)C)ccc54)ccc3-c3ccc(C#N)cc3)n2)c1 ; N#Cc1ccc(-c2ccc(-n3c4ccccc4c4cc(-c5ccccc5)ccc43)cc2-c2nc(-c3ccccc3)nc(-c3cc(-n4c5ccccc5c5cc(-c6ccccc6)ccc54)ccc3C#N)n2)cc1]

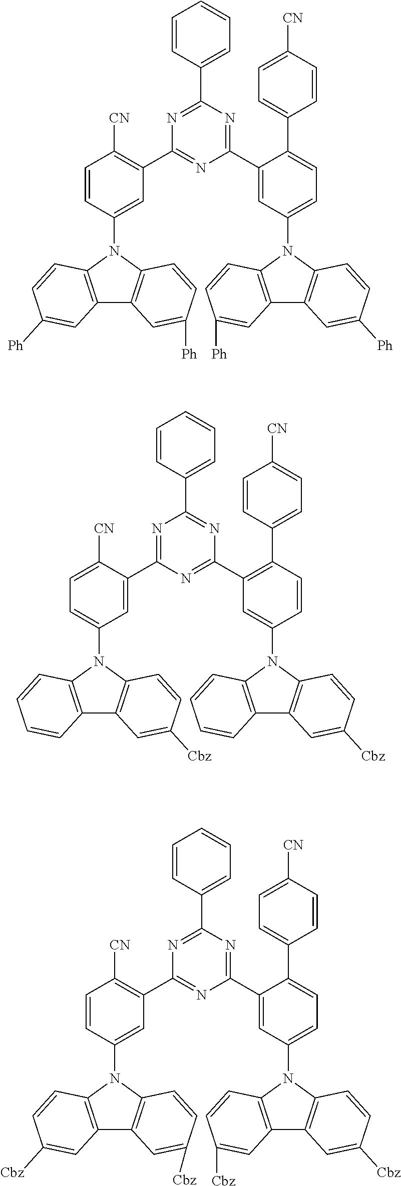 [SMILES: N#Cc1ccc(-c2ccc(-n3c4ccc(-c5ccccc5)cc4c4cc(-c5ccccc5)ccc43)cc2-c2nc(-c3ccccc3)nc(-c3cc(-n4c5ccc(-c6ccccc6)cc5c5cc(-c6ccccc6)ccc54)ccc3C#N)n2)cc1 ; N#Cc1ccc(-c2ccc(-n3c4ccccc4c4cc(C(=O)OCc5ccccc5)ccc43)cc2-c2nc(-c3ccccc3)nc(-c3cc(-n4c5ccccc5c5cc(C(=O)OCc6ccccc6)ccc54)ccc3C#N)n2)cc1 ; N#Cc1ccc(-c2ccc(-n3c4ccc(C(=O)OCc5ccccc5)cc4c4cc(C(=O)OCc5ccccc5)ccc43)cc2-c2nc(-c3ccccc3)nc(-c3cc(-n4c5ccc(C(=O)OCc6ccccc6)cc5c5cc(C(=O)OCc6ccccc6)ccc54)ccc3C#N)n2)cc1]

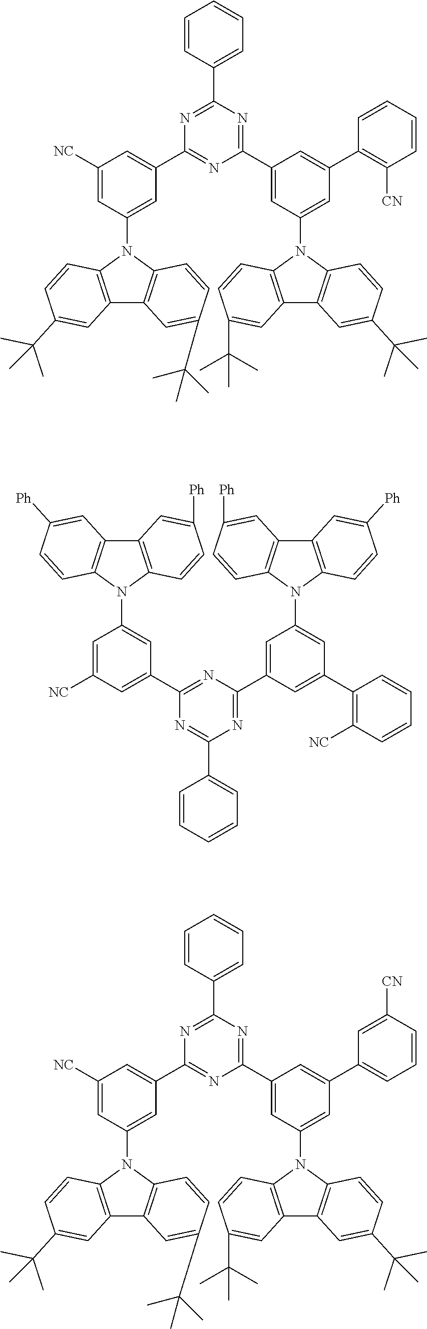

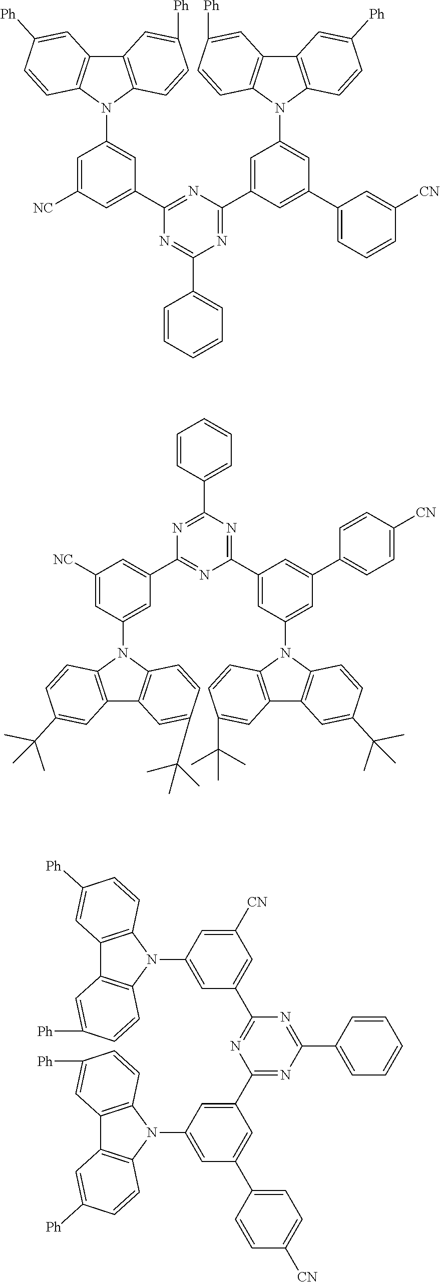 [SMILES: N#Cc1cccc(-c2cc(-c3nc(-c4ccccc4)nc(-c4cc(C#N)cc(-n5c6ccc(-c7ccccc7)cc6c6cc(-c7ccccc7)ccc65)c4)n3)cc(-n3c4ccc(-c5ccccc5)cc4c4cc(-c5ccccc5)ccc43)c2)c1 ; CC(C)(C)c1ccc2c(c1)c1cc(C(C)(C)C)ccc1n2-c1cc(C#N)cc(-c2nc(-c3ccccc3)nc(-c3cc(-c4ccc(C#N)cc4)cc(-n4c5ccc(C(C)(C)C)cc5c5cc(C(C)(C)C)ccc54)c3)n2)c1 ; N#Cc1ccc(-c2cc(-c3nc(-c4ccccc4)nc(-c4cc(C#N)cc(-n5c6ccc(-c7ccccc7)cc6c6cc(-c7ccccc7)ccc65)c4)n3)cc(-n3c4ccc(-c5ccccc5)cc4c4cc(-c5ccccc5)ccc43)c2)cc1]

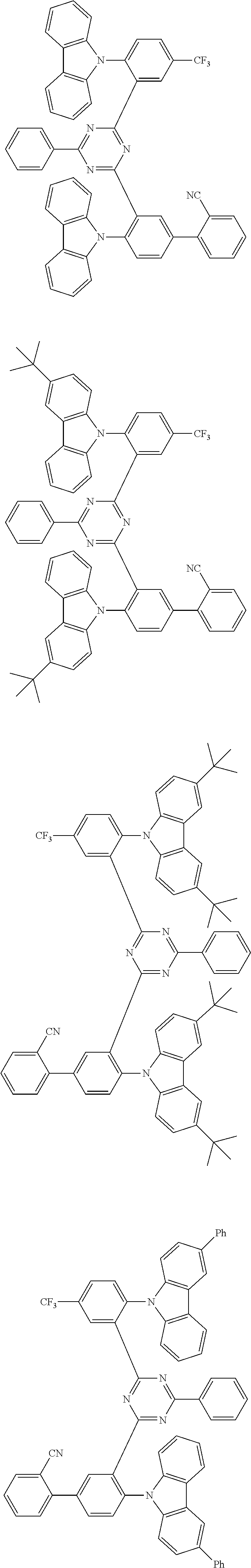

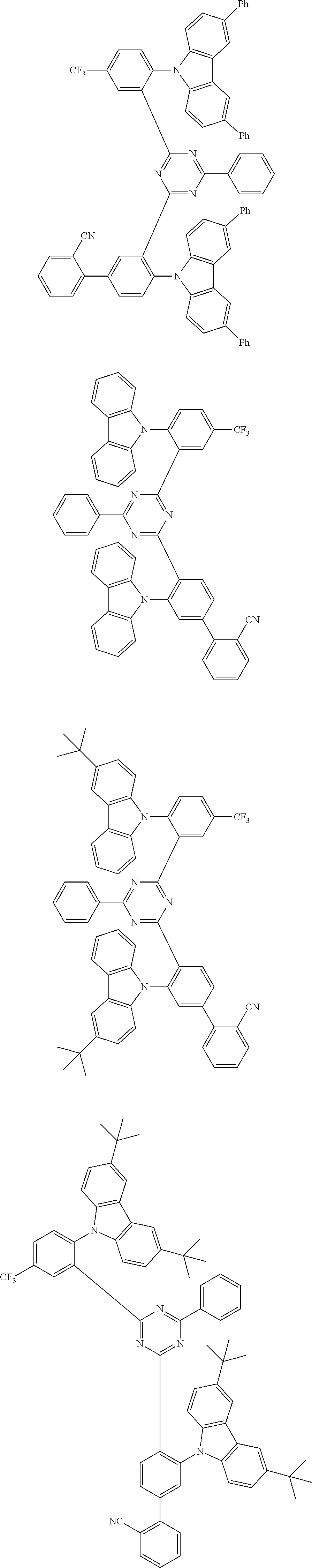

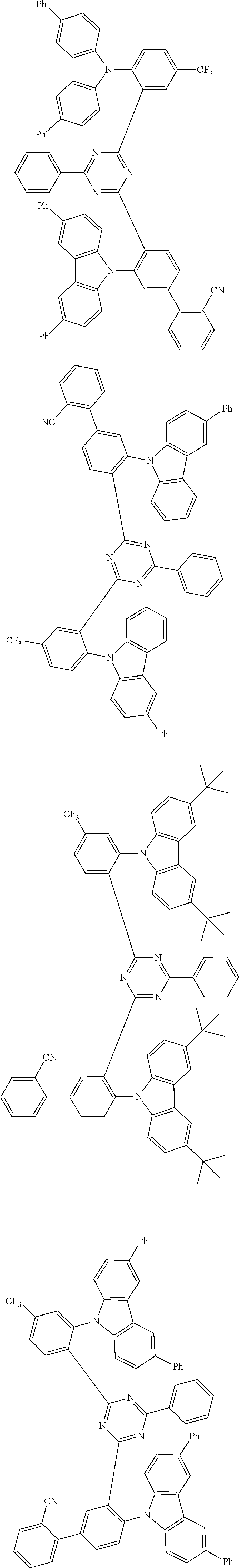

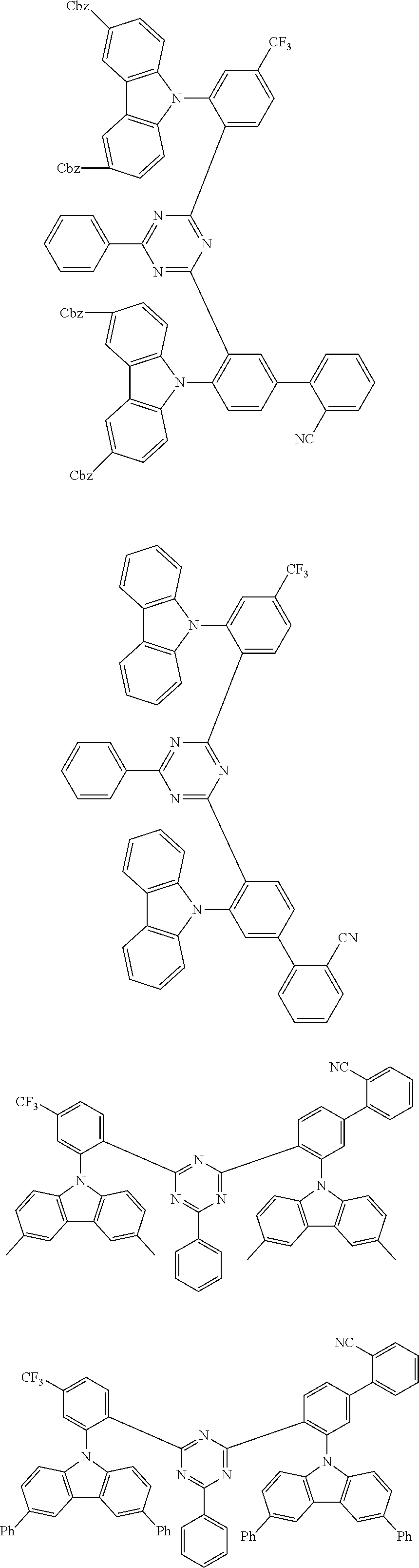

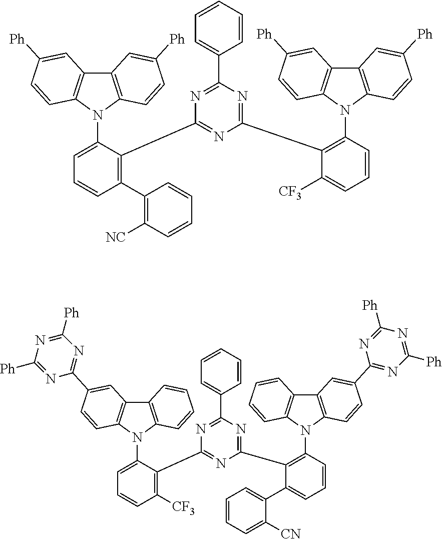 [SMILES: N#Cc1ccccc1-c1cccc(-n2c3ccc(-c4ccccc4)cc3c3cc(-c4ccccc4)ccc32)c1-c1nc(-c2ccccc2)nc(-c2c(-n3c4ccc(-c5ccccc5)cc4c4cc(-c5ccccc5)ccc43)cccc2C(F)(F)F)n1 ; N#Cc1ccccc1-c1cccc(-n2c3ccccc3c3cc(-c4nc(-c5ccccc5)nc(-c5ccccc5)n4)ccc32)c1-c1nc(-c2ccccc2)nc(-c2c(-n3c4ccccc4c4cc(-c5nc(-c6ccccc6)nc(-c6ccccc6)n5)ccc43)cccc2C(F)(F)F)n1]

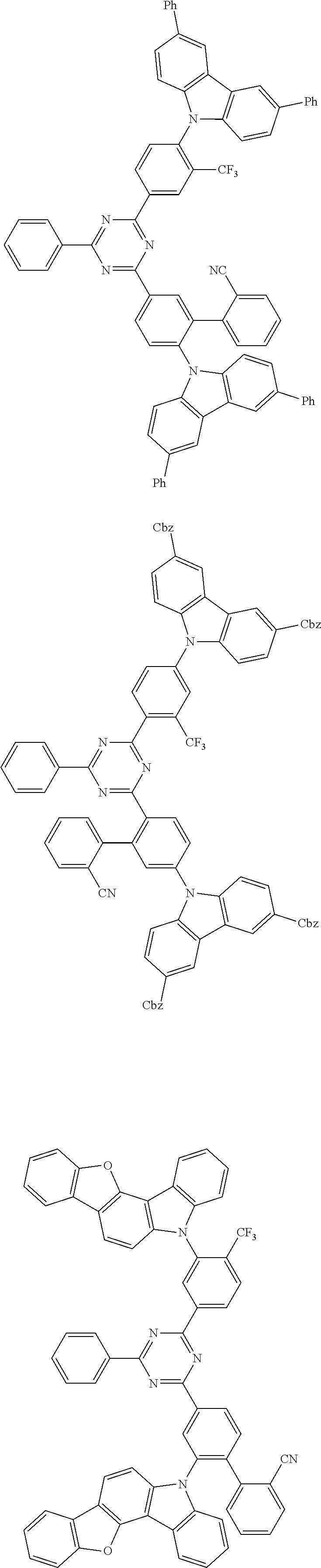 [SMILES: N#Cc1ccccc1-c1cc(-c2nc(-c3ccccc3)nc(-c3ccc(-n4c5ccc(-c6ccccc6)cc5c5cc(-c6ccccc6)ccc54)c(C(F)(F)F)c3)n2)ccc1-n1c2ccc(-c3ccccc3)cc2c2cc(-c3ccccc3)ccc21 ; N#Cc1ccccc1-c1cc(-n2c3ccc(C(=O)OCc4ccccc4)cc3c3cc(C(=O)OCc4ccccc4)ccc32)ccc1-c1nc(-c2ccccc2)nc(-c2ccc(-n3c4ccc(C(=O)OCc5ccccc5)cc4c4cc(C(=O)OCc5ccccc5)ccc43)cc2C(F)(F)F)n1 ; N#Cc1ccccc1-c1ccc(-c2nc(-c3ccccc3)nc(-c3ccc(C(F)(F)F)c(-n4c5ccccc5c5c6oc7ccccc7c6ccc54)c3)n2)cc1-n1c2ccccc2c2c3oc4ccccc4c3ccc21]

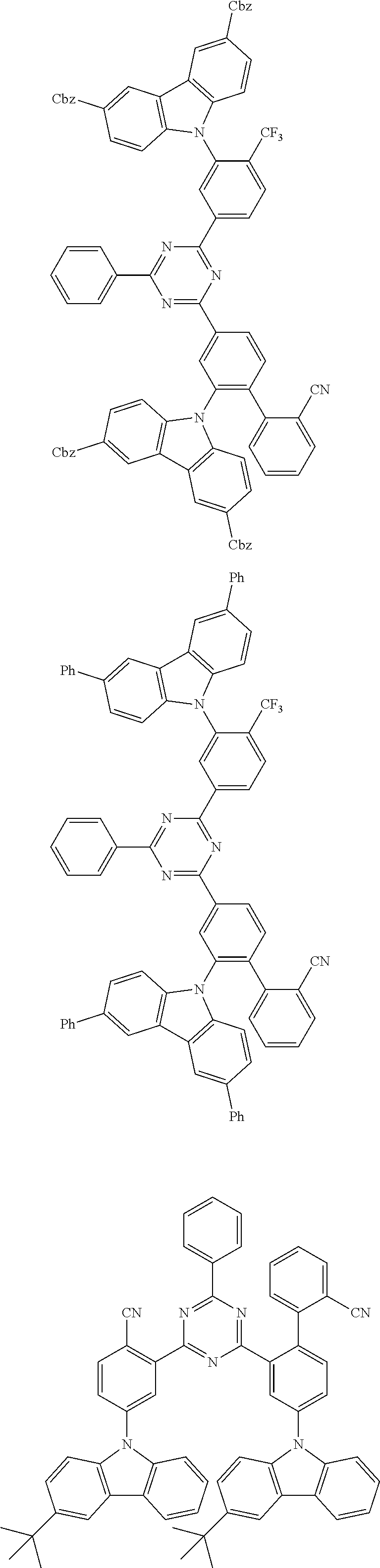

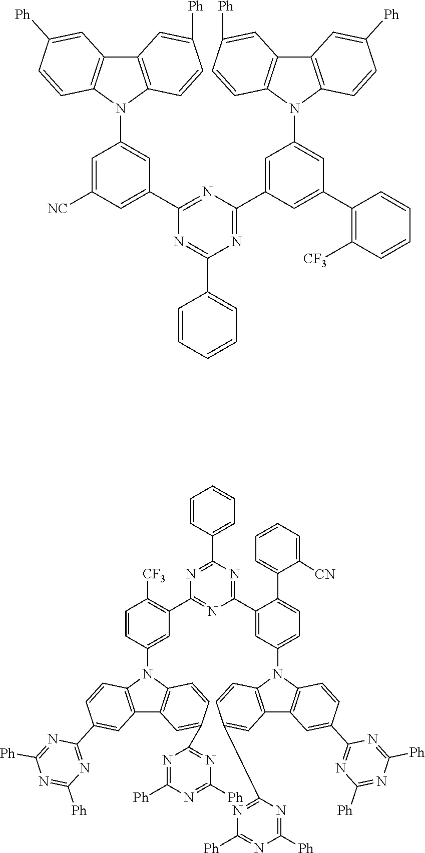 [SMILES: N#Cc1cc(-c2nc(-c3ccccc3)nc(-c3cc(-c4ccccc4C(F)(F)F)cc(-n4c5ccc(-c6ccccc6)cc5c5cc(-c6ccccc6)ccc54)c3)n2)cc(-n2c3ccc(-c4ccccc4)cc3c3cc(-c4ccccc4)ccc32)c1 ; N#Cc1ccccc1-c1ccc(-n2c3ccc(-c4nc(-c5ccccc5)nc(-c5ccccc5)n4)cc3c3cc(-c4nc(-c5ccccc5)nc(-c5ccccc5)n4)ccc32)cc1-c1nc(-c2ccccc2)nc(-c2cc(-n3c4ccc(-c5nc(-c6ccccc6)nc(-c6ccccc6)n5)cc4c4cc(-c5nc(-c6ccccc6)nc(-c6ccccc6)n5)ccc43)ccc2C(F)(F)F)n1]

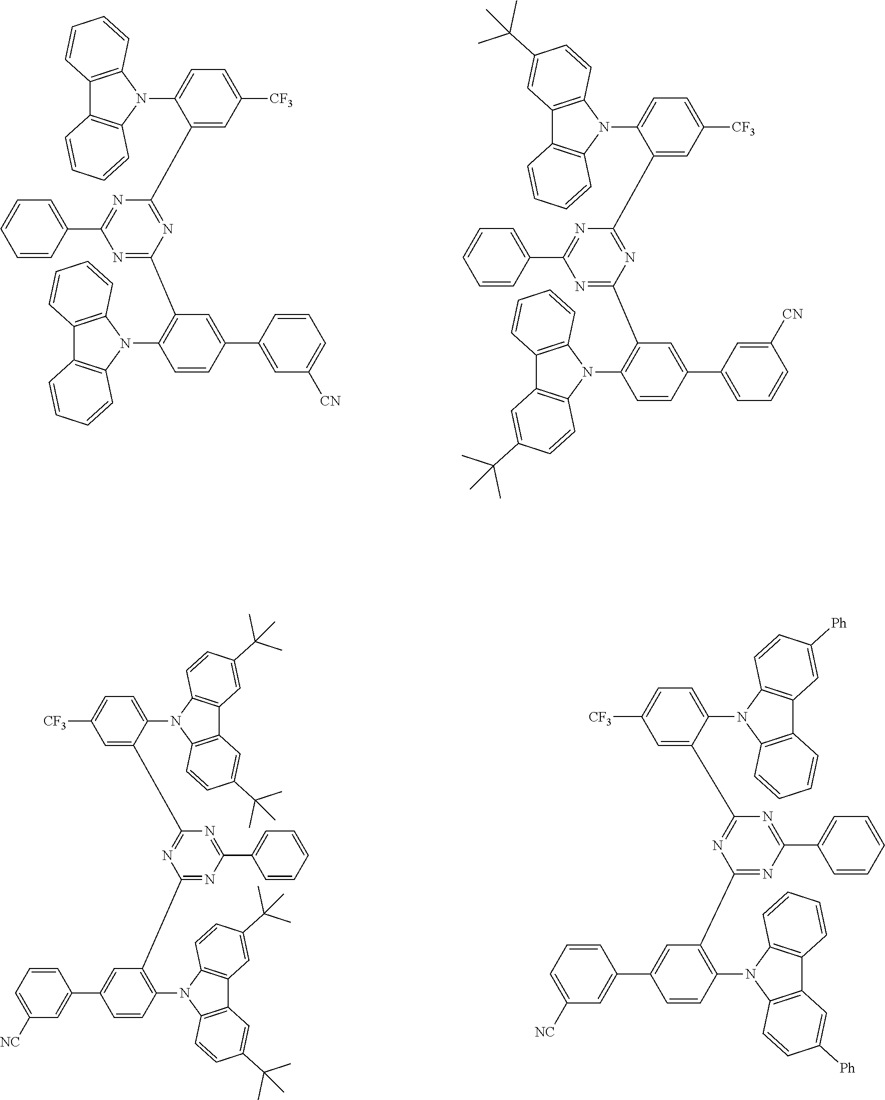

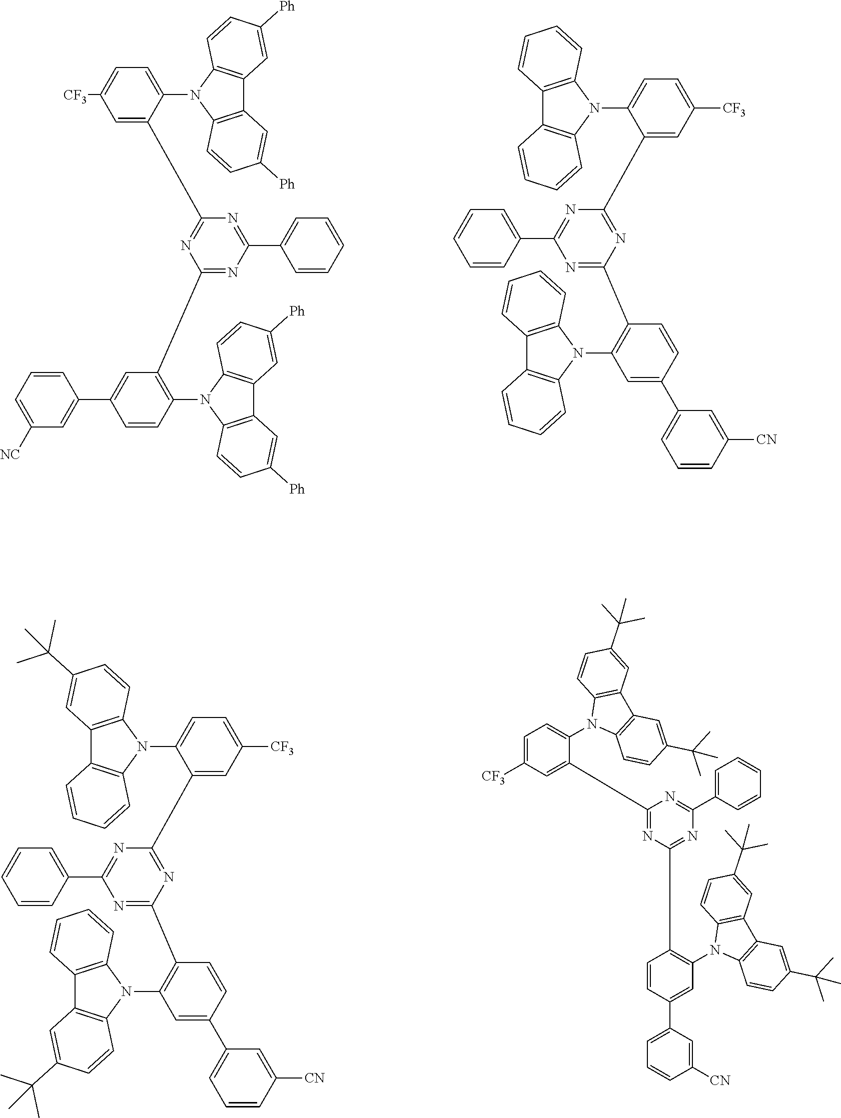

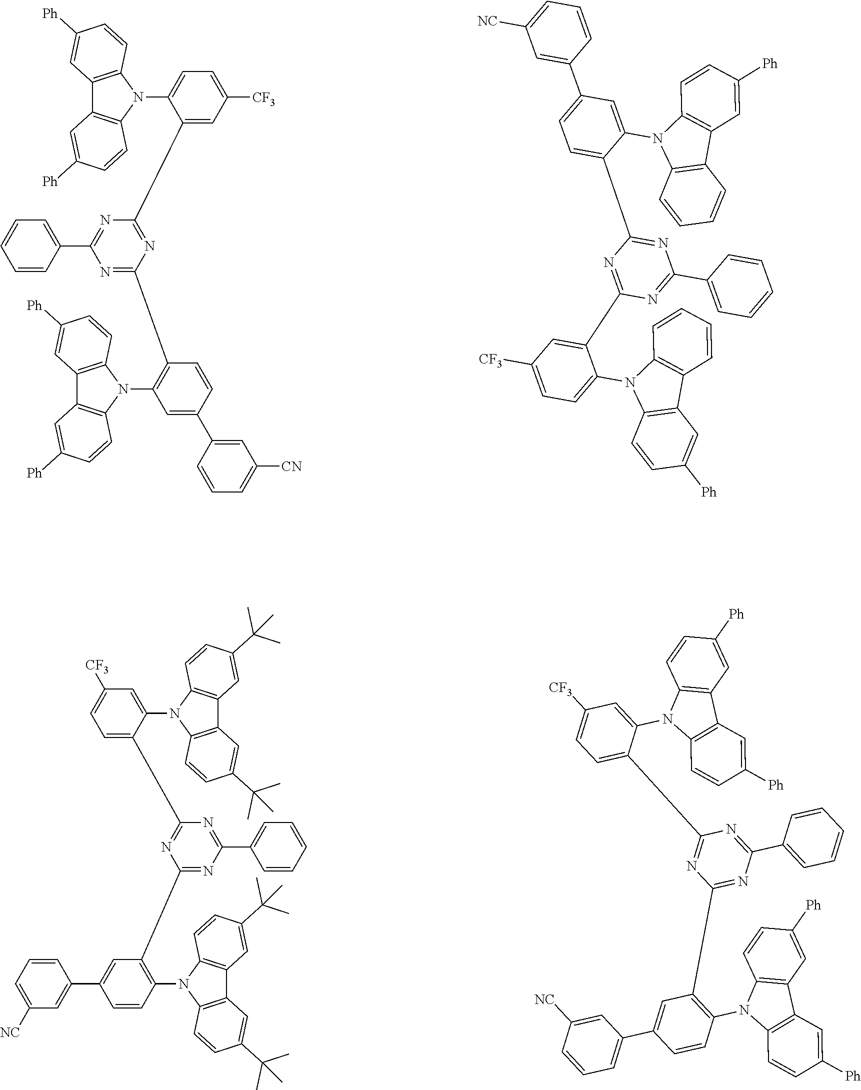

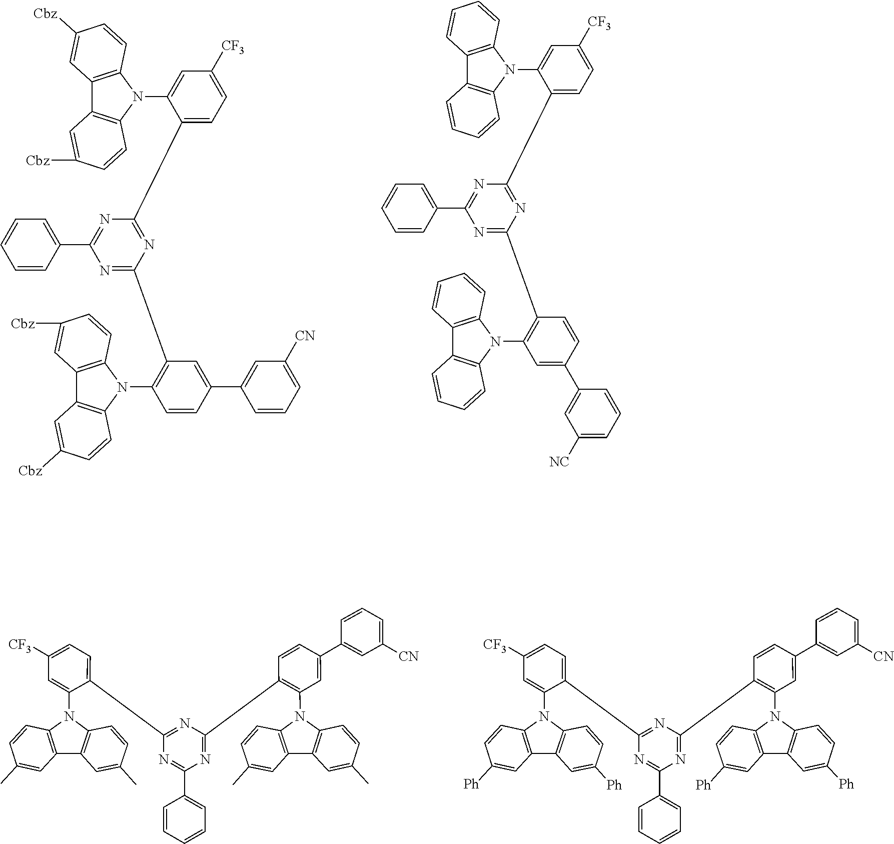

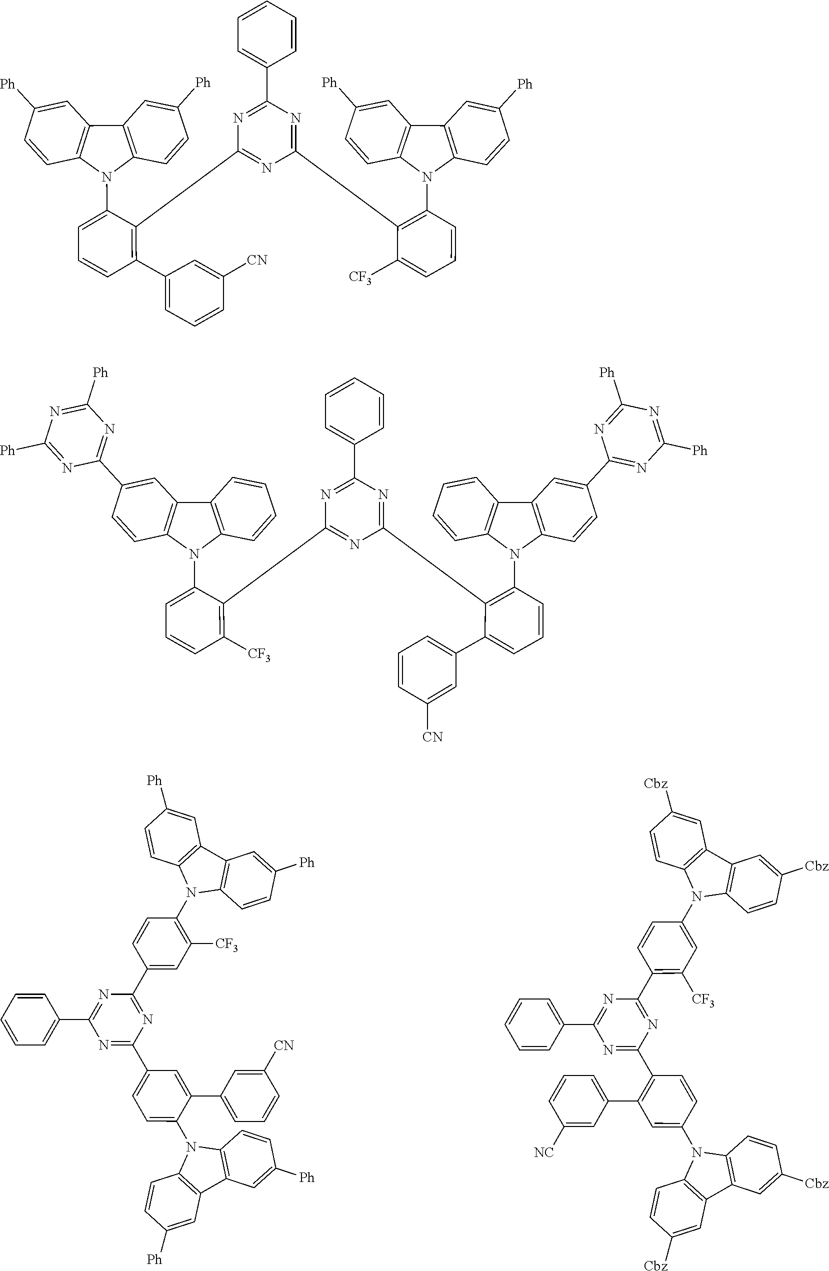

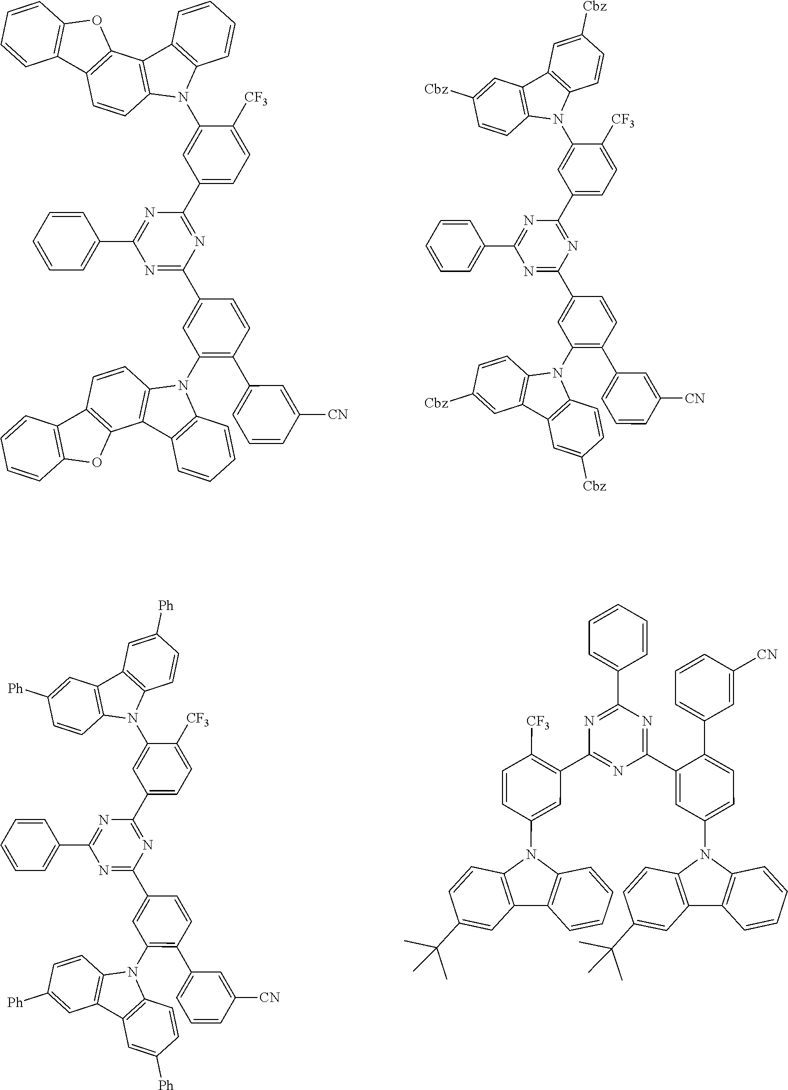

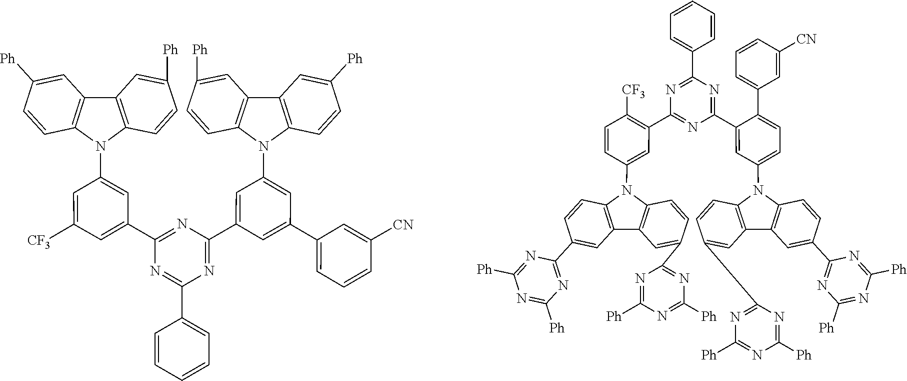 [SMILES: N#Cc1cccc(-c2cc(-c3nc(-c4ccccc4)nc(-c4cc(-n5c6ccc(-c7ccccc7)cc6c6cc(-c7ccccc7)ccc65)cc(C(F)(F)F)c4)n3)cc(-n3c4ccc(-c5ccccc5)cc4c4cc(-c5ccccc5)ccc43)c2)c1 ; N#Cc1cccc(-c2ccc(-n3c4ccc(-c5nc(-c6ccccc6)nc(-c6ccccc6)n5)cc4c4cc(-c5nc(-c6ccccc6)nc(-c6ccccc6)n5)ccc43)cc2-c2nc(-c3ccccc3)nc(-c3cc(-n4c5ccc(-c6nc(-c7ccccc7)nc(-c7ccccc7)n6)cc5c5cc(-c6nc(-c7ccccc7)nc(-c7ccccc7)n6)ccc54)ccc3C(F)(F)F)n2)c1]

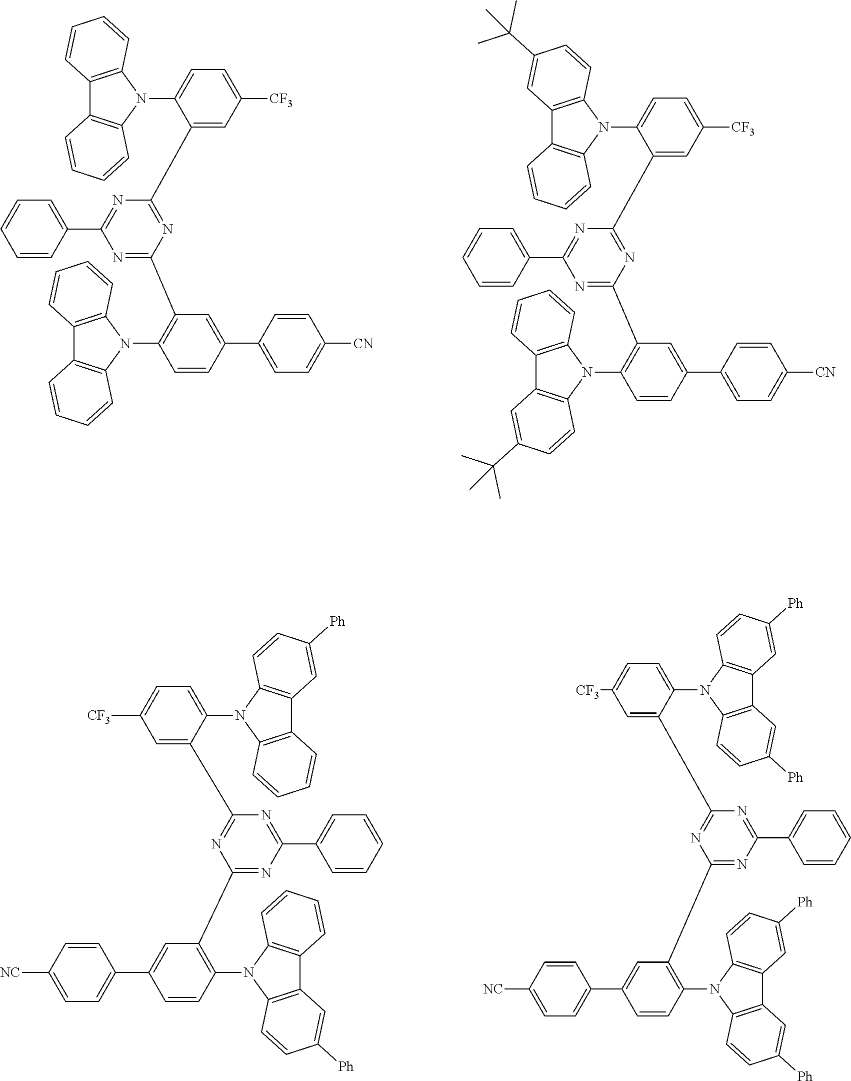

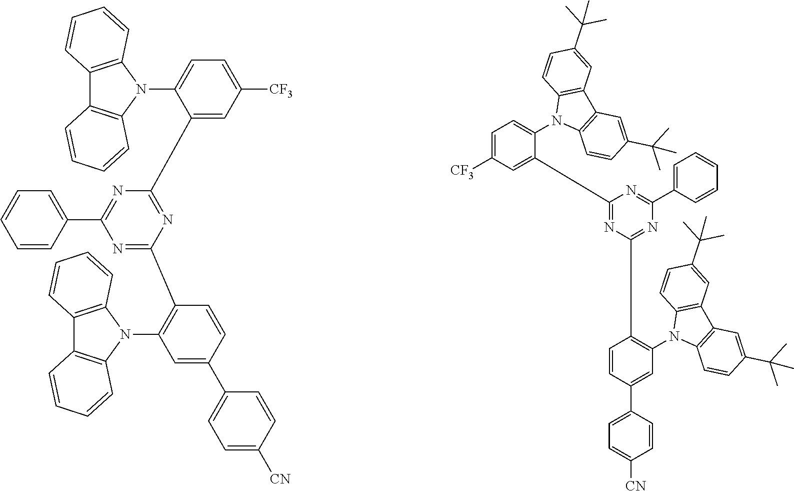 [SMILES: N#Cc1ccc(-c2ccc(-c3nc(-c4ccccc4)nc(-c4cc(C(F)(F)F)ccc4-n4c5ccccc5c5ccccc54)n3)c(-n3c4ccccc4c4ccccc43)c2)cc1 ; CC(C)(C)c1ccc2c(c1)c1cc(C(C)(C)C)ccc1n2-c1ccc(C(F)(F)F)cc1-c1nc(-c2ccccc2)nc(-c2ccc(-c3ccc(C#N)cc3)cc2-n2c3ccc(C(C)(C)C)cc3c3cc(C(C)(C)C)ccc32)n1]

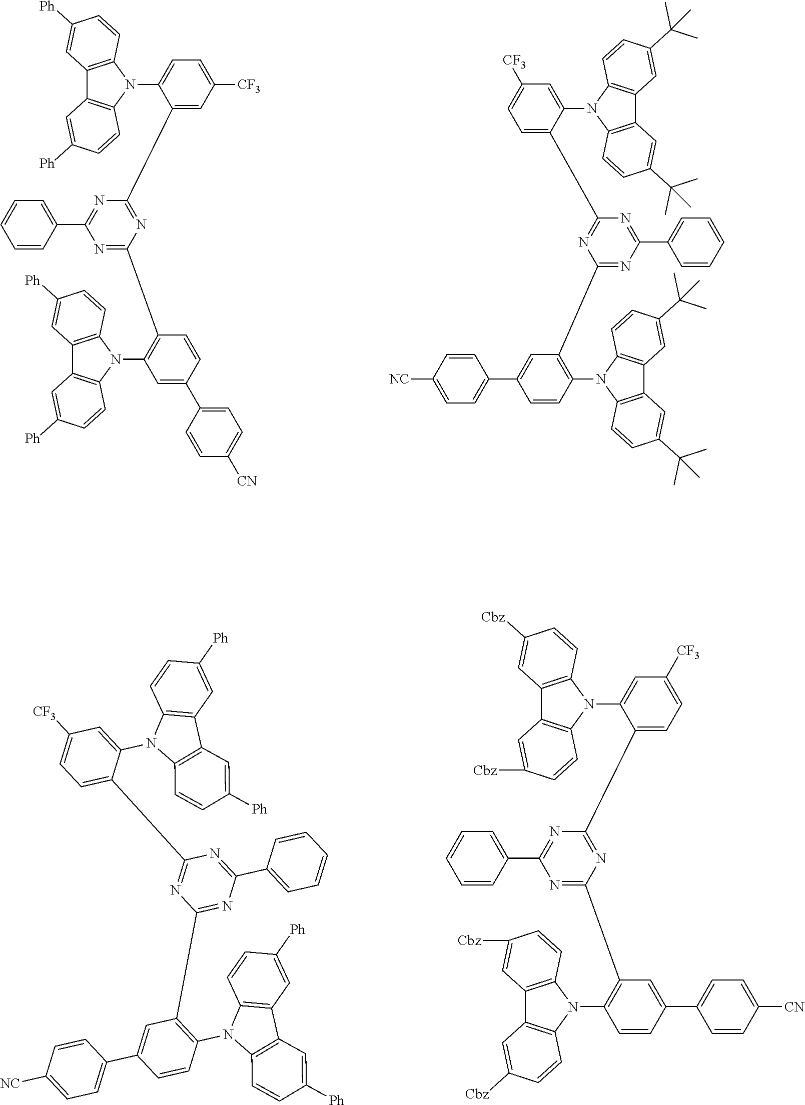

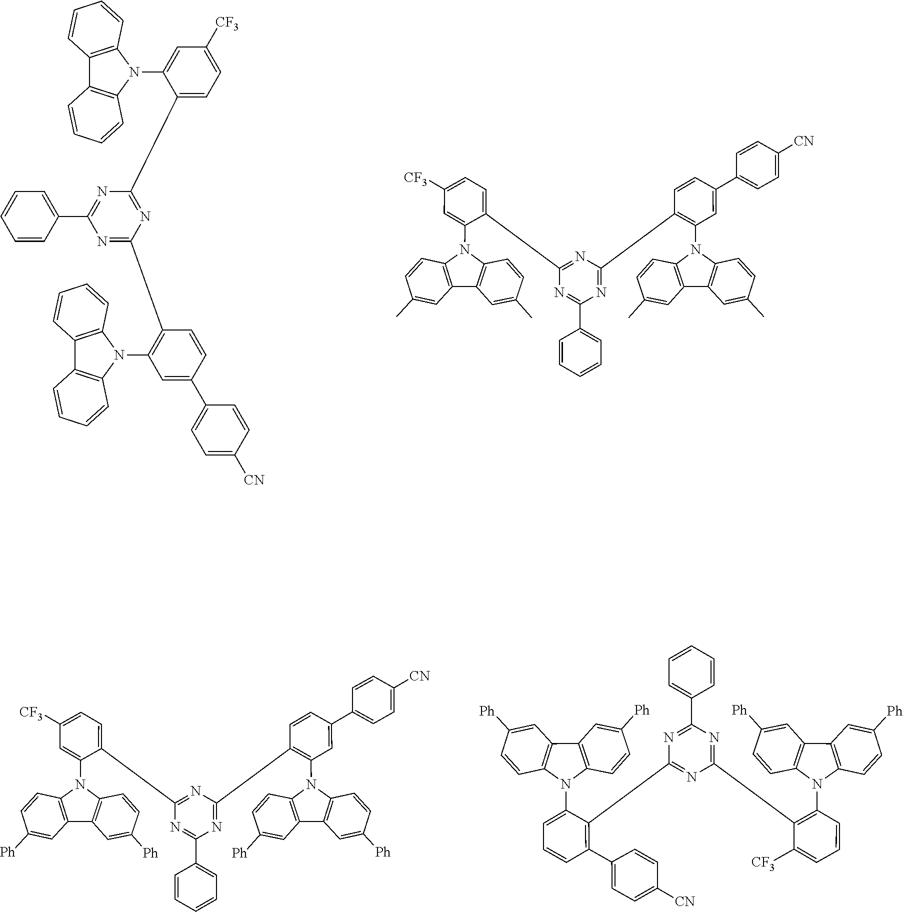

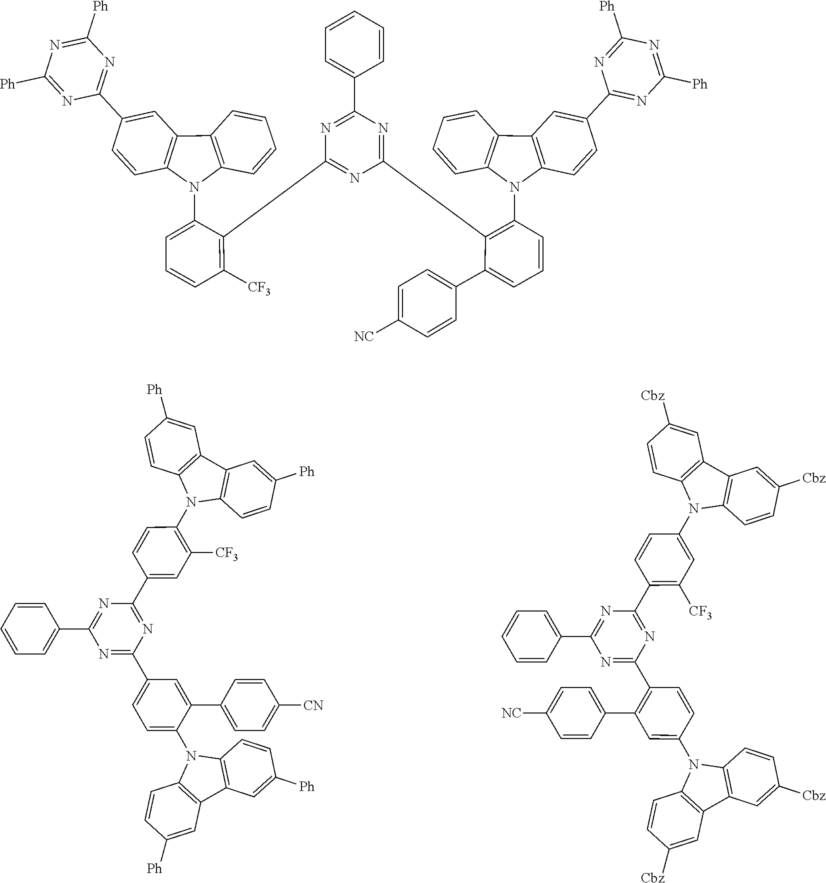

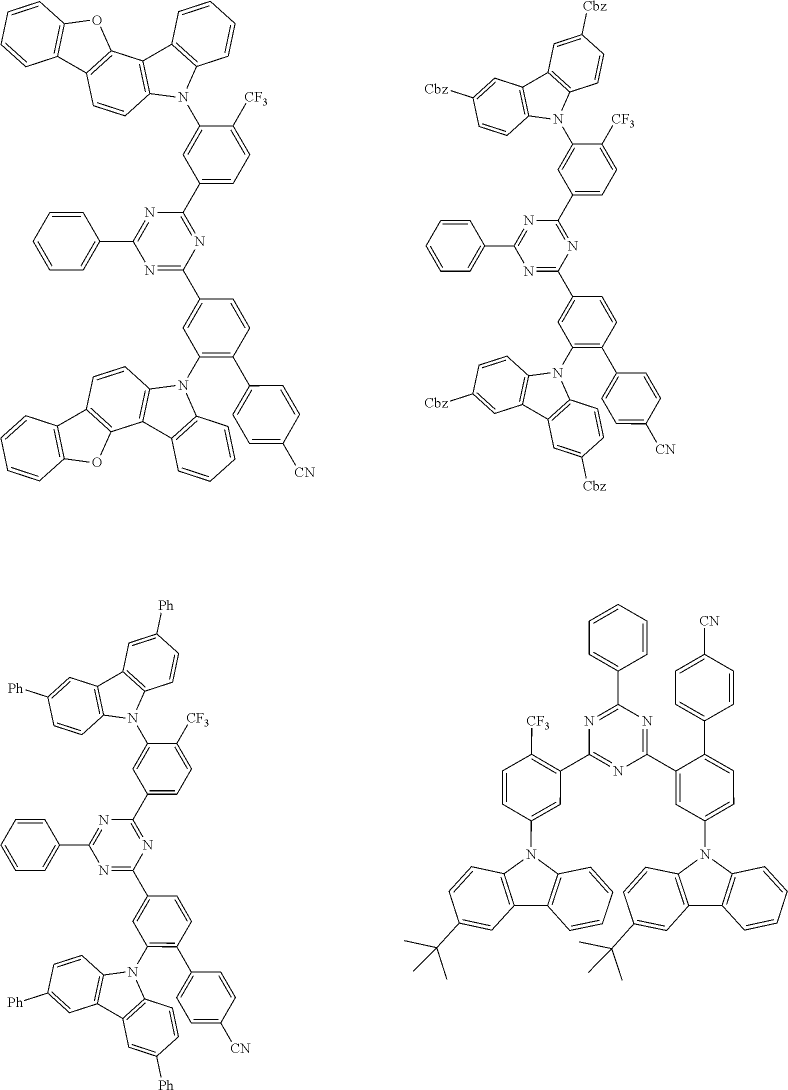

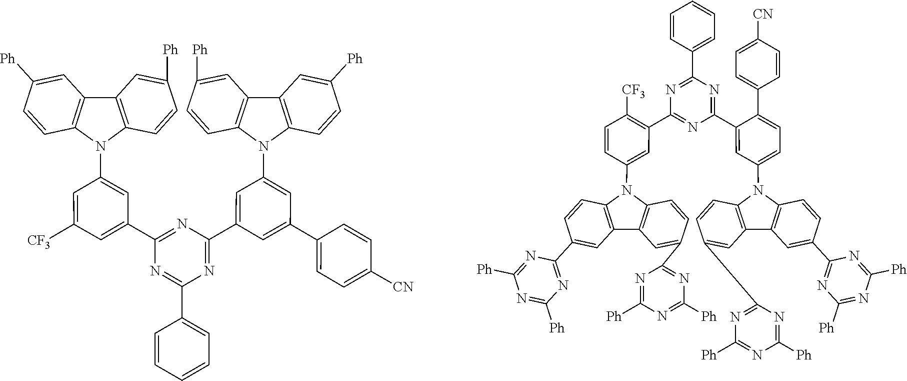 [SMILES: N#Cc1ccc(-c2cc(-c3nc(-c4ccccc4)nc(-c4cc(-n5c6ccc(-c7ccccc7)cc6c6cc(-c7ccccc7)ccc65)cc(C(F)(F)F)c4)n3)cc(-n3c4ccc(-c5ccccc5)cc4c4cc(-c5ccccc5)ccc43)c2)cc1 ; N#Cc1ccc(-c2ccc(-n3c4ccc(-c5nc(-c6ccccc6)nc(-c6ccccc6)n5)cc4c4cc(-c5nc(-c6ccccc6)nc(-c6ccccc6)n5)ccc43)cc2-c2nc(-c3ccccc3)nc(-c3cc(-n4c5ccc(-c6nc(-c7ccccc7)nc(-c7ccccc7)n6)cc5c5cc(-c6nc(-c7ccccc7)nc(-c7ccccc7)n6)ccc54)ccc3C(F)(F)F)n2)cc1]

### FIGURES

FIG. 1 Emission spectrum of example 1 (10% by weight) in PMMA.

FIG. 2 Emission spectrum of example 2 (10% by weight) in PMMA.

FIG. 3 Emission spectrum of example 3 (10% by weight) in PMMA.

FIG. 4 Emission spectrum of example 4 (10% by weight) in PMMA.

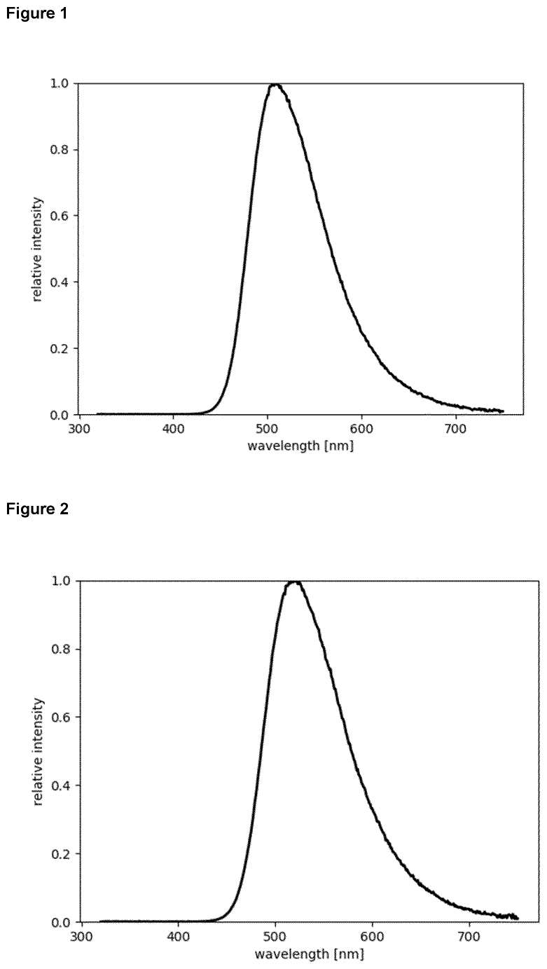

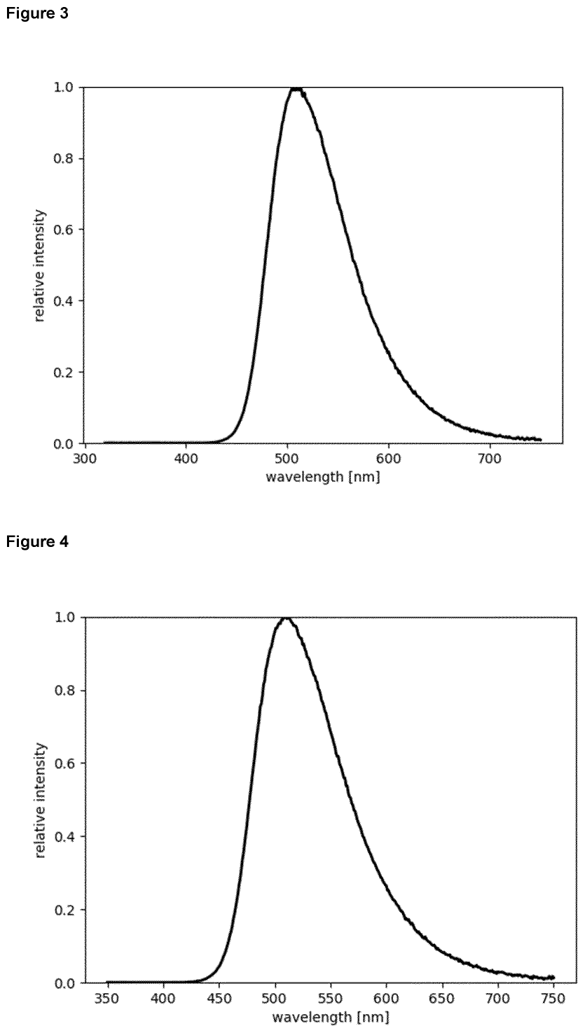

1.-15. (canceled)

16. An organic molecule, comprising
a first chemical moiety comprising or consisting of a structure of Formula I, 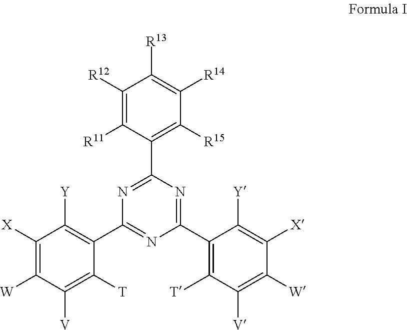
and
two second chemical moieties, each independently comprising or consisting of a structure of Formula II, 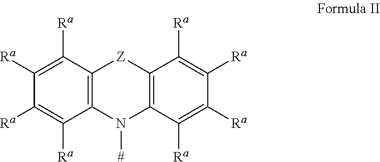
wherein each of the second chemical moieties is linked to the first chemical moiety via a single bond;
wherein
T is a binding site of the single bond linking the first chemical moiety to one of the two second chemical moieties, or is R^{I};
V is the binding site of the single bond linking the first chemical moiety to one of the two second chemical moieties, or is R^{I};
W is the binding site of the single bond linking the first chemical moiety to one of the two second chemical moieties, or is selected from the group consisting of R^{I} and R^{A};
X is selected from the group consisting of R^{I} and R^{A};
Y is selected from the group consisting of R^{I} and R^{A};
T′ is the binding site of the single bond linking the first chemical moiety to one of the two second chemical moieties, or is R^{II};
V′ is the binding site of the single bond linking the first chemical moiety to one of the two second chemical moieties, or is R^{II};
W′ is the binding site of the single bond linking the first chemical moiety to one of the two second chemical moieties, or is selected from the group consisting of R^{II}, CN, and CF₃;
X′ is selected from the group consisting of R^{II}, CN, and CF₃;
Y′ is selected from the group consisting of R^{II}, CN, and CF₃;
Z is at each occurrence independently from one another selected from the group consisting of a direct bond, CR³R⁴, C═CR³R⁴, C═O, C═NR³, NR³, O, SiR³R⁴, S, S(O) and S(O)₂;
#represents the binding site of the first chemical moiety to the second chemical moiety;
R^{A} comprises or consists of a structure of Formula BN-I, 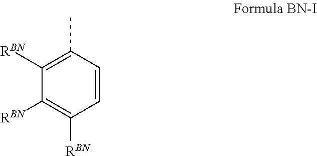
which is bonded to the structure of Formula I via a position marked by the dashed line and wherein exactly one R^{BN} group is CN while the other two R^{BN} groups are each hydrogen;
R^{I} is at each occurrence independently from one another selected from the group consisting of:
hydrogen; deuterium;
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₆-C₁₈-aryl;
R^{II} is at each occurrence independently from one another selected from the group consisting of:
hydrogen; deuterium;
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₆-C₁₈-aryl;
R¹¹, R¹², R¹³, R¹⁴ and R¹⁵ are at each occurrence independently selected from the group consisting of:
hydrogen; deuterium; CN; CF₃; phenyl;
C₁-C₅-alkyl,
wherein one or more hydrogen atoms are optionally substituted by deuterium;
C₂-C₈-alkenyl,
wherein one or more hydrogen atoms are optionally substituted by deuterium;
C₂-C₈-alkynyl,
wherein one or more hydrogen atoms are optionally substituted by deuterium; and
C₆-C₁₈-aryl;
R^{a}, R³, and R⁴ are at each occurrence independently selected from the group consisting of:
hydrogen; deuterium; N(R⁵)₂; OR⁵; Si(R⁵)₃; B(OR⁵)₂; OSO₂R⁵; CF₃; CN; F; Br; I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵;
R⁵ is at each occurrence independently from one another selected from the group consisting of:
hydrogen; deuterium; N(R⁶)₂; OR⁶; Si(R⁶)₃; B(OR⁶)₂; OSO₂R⁶; CF₃; CN; F; Br; I;
C₁-C₄₀-alkyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-alkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₁-C₄₀-thioalkoxy,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkenyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₂-C₄₀-alkynyl,
which is optionally substituted with one or more substituents R⁶ and
wherein one or more non-adjacent CH₂-groups are optionally substituted by R⁶C═CR⁶, C≡C, Si(R⁶)₂, Ge(R⁶)₂, Sn(R⁶)₂, C═O, C═S, C═Se, C═NR⁶, P(═O)(R⁶), SO, SO₂, NR⁶, O, S or CONR⁶;
C₆-C₆₀-aryl,
which is optionally substituted with one or more substituents R⁶; and
C₃-C₅₇-heteroaryl,
which is optionally substituted with one or more substituents R⁶;
R⁶ is at each occurrence independently from one another selected from the group consisting of:
hydrogen; deuterium; OPh; CF₃; CN; F;
C₁-C₅-alkyl,
wherein one or more hydrogen atoms are optionally, independently from each other substituted by deuterium, CN, CF₃, or F;
C₁-C₅-alkoxy,
wherein one or more hydrogen atoms are optionally, independently from each other substituted by deuterium, CN, CF₃, or F;
C₁-C₅-thioalkoxy,
wherein one or more hydrogen atoms are optionally, independently from each other substituted by deuterium, CN, CF₃, or F;
C₂-C₅-alkenyl,
wherein one or more hydrogen atoms are optionally, independently from each other substituted by deuterium, CN, CF₃, or F;
C₂-C₅-alkynyl,
wherein one or more hydrogen atoms are optionally, independently from each other substituted by deuterium, CN, CF₃, or F;
C₆-C₁₈-aryl,
which is optionally substituted with one or more C₁-C₅-alkyl substituents;
C₃-C₁₇-heteroaryl,
which is optionally substituted with one or more C₁-C₅-alkyl substituents;
N(C₆-C₁₈-aryl)₂;
N(C₃-C₁₇-heteroaryl)₂; and
N(C₃-C₁₇-heteroaryl)(C₆-C₁₈-aryl);
wherein, optionally, R^{a}, R³, R⁴ and/or R⁵ independently form a mono- or polycyclic, aliphatic, aromatic and/or benzo-fused ring system with one or more other substituents R^{a}, R³, R⁴ and/or R⁵; and
wherein
exactly one substituent selected from the group consisting of W, X, and Y is R^{A}, and
exactly one substituent selected from the group consisting of T, V, and W represents the binding site of the single bond linking the first chemical moiety and one of the two second chemical moieties;
exactly one substituent selected from the group consisting of W′, X′, and Y′ is CN or CF₃, and
exactly one substituent selected from the group consisting of T′, V′, and W′ represents the binding site of the single bond linking the first chemical moiety and one of the two second chemical moieties.

17. The organic molecule according to claim 16, wherein
T and T′ are each a binding site of the single bond linking the first chemical moiety to one of the two second chemical moieties, and
either X is R^{A} and X′ is CN or CF₃;
or W is R^{A} and X′ is CN or CF₃.

18. The organic molecule according to claim 16, wherein
R^{I}, R^{II}, R¹¹, R¹², R¹³, R¹⁴, and R¹⁵ are at each occurrence independently selected from the group consisting of hydrogen, methyl, iso-propyl, tert-butyl, mesityl, xylyl, tolyl, and phenyl.

19. The organic molecule according to claim 16, wherein the second chemical moiety comprises or consists of a structure of Formula IIa:

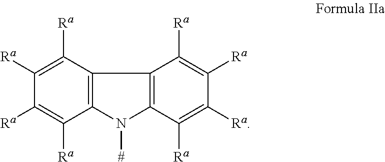

20. The organic molecule according to claim 16, wherein the second chemical moiety comprises or consists of a structure of Formula IIb: 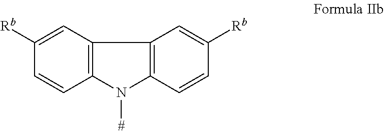
wherein
R^{b} is at each occurrence independently from one another selected from the group consisting of:
hydrogen; deuterium; N(R⁵)₂; OR⁵; Si(R⁵)₃; B(OR⁵)₂; OSO₂R⁵; CF₃; CN; F; Br; I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵.

21. The organic molecule according to claim 16, wherein the second chemical moiety comprises or consists of a structure of Formula IIc: 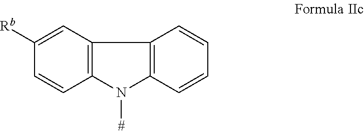
wherein
R^{b} is at each occurrence independently selected from the group consisting of:
hydrogen; deuterium; N(R⁵)₂; OR⁵; Si(R⁵)₃; B(OR⁵)₂; OSO₂R⁵; CF₃; CN; F; Br; I;
C₁-C₄₀-alkyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-alkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₁-C₄₀-thioalkoxy,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkenyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₂-C₄₀-alkynyl,
which is optionally substituted with one or more substituents R⁵ and
wherein one or more non-adjacent CH₂-groups are optionally substituted by R⁵C═CR⁵, C≡C, Si(R⁵)₂, Ge(R⁵)₂, Sn(R⁵)₂, C═O, C═S, C═Se, C═NR⁵, P(═O)(R⁵), SO, SO₂, NR⁵, O, S or CONR⁵;
C₆-C₆₀-aryl,
which is optionally substituted with one or more substituents R⁵; and
C₃-C₅₇-heteroaryl,
which is optionally substituted with one or more substituents R⁵.

22. The organic molecule according to claim 20, wherein R^{b} is at each occurrence independently from one another selected from the group consisting of:
Me;
ⁱPr;
^{t}Bu;
CN;
CF₃;
Ph, which is optionally substituted with one or more substituents independently from each other selected from the group consisting of Me, ⁱPr, ^{t}Bu, CN, CF₃ and Ph;
pyridinyl, which is optionally substituted with one or more substituents independently from each other selected from the group consisting of Me, ⁱPr, ^{t}Bu, CN, CF₃ and Ph;
pyrimidinyl, which is optionally substituted with one or more substituents independently from each other selected from the group consisting of Me, ⁱPr, ^{t}Bu, CN, CF₃ and Ph;
carbazolyl, which is optionally substituted with one or more substituents independently from each other selected from the group consisting of Me, ⁱPr, ^{t}Bu, CN, CF₃ and Ph;
triazinyl, which is optionally substituted with one or more substituents independently from each other selected from the group consisting of Me, ⁱPr, ^{t}Bu, CN, CF₃, and Ph; and
N(Ph)₂.

23. A method for preparing the organic molecule according to claim 16, the method comprising providing a substituted 2,4-dichloro-6-phenyltriazine as a reactant.

24. An optoelectronic device comprising the organic molecule according to claim 16, as a luminescent emitter and/or a host material and/or an electron transport material and/or a hole transport material and/or a hole injection material and/or a hole blocking material.

25. The optoelectronic device according to claim 24, wherein the optoelectronic device is at least one selected from the group consisting of:
organic light-emitting diodes (OLEDS),
light-emitting electrochemical cells,
OLED-sensors,
organic diodes,
organic solar cells,
organic transistors,
organic field-effect transistors,
organic lasers, and
down-conversion elements.

26. A composition, comprising:
(a) the organic molecule according to claim 16, as an emitter and/or a host, and
(b) an emitter and/or a host material, which differs from the organic molecule, and
(c) optionally, one or more dyes and/or one or more solvents.

27. The composition according to claim 26, comprising:
(i) 1-50% by weight of the organic molecule;
(ii) 5-98% by weight of one host compound H;
(iii) 1-30% by weight of at least one further emitter molecule with a structure differing from the structure of the organic molecule; and
(iv) optionally, 0-94% by weight of at least one further host compound D with a structure differing from the structure of the organic molecule; and
(v) optionally, 0-94% by weight of a solvent.

28. An optoelectronic device comprising a layer formed from the composition according to claim 26,
wherein the device is at least one selected from the group consisting of organic light-emitting diodes (OLEDs), light-emitting electrochemical cells, OLED-sensors, organic diodes, organic solar cells, organic transistors, organic field-effect transistors, organic lasers, and down-conversion elements.

29. The optoelectronic device according to claim 24, comprising
a substrate,
an anode, and
a cathode, wherein the anode or the cathode is on the substrate, and
a light-emitting layer between the anode and the cathode and comprising the organic molecule.

30. A method for producing an optoelectronic device, the method comprising depositing the organic molecule according to claim 16 by a vacuum evaporation method or from a solution.

31. The optoelectronic device according to claim 28, comprising:
a substrate,
an anode, and
a cathode, wherein the anode or the cathode is on the substrate, and
a light-emitting layer between the anode and the cathode and comprising the composition.

32. A method for producing an optoelectronic device, the method comprising depositing the composition according to claim 26 by a vacuum evaporation method or from a solution.
